SEQUENCE LISTING (1 sequence)

SEQ ID NO: 1 - protein, 566 aa, Influenza A virus (A/Netherlands/602/2009(H1N1))
  Hemagglutinin
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

(19) United States
(12) Patent Application Publication
(10) Pub. No.: US 2023/0293447 A1
(43) Pub. Date: Sep. 21, 2023

(54) CLEAVABLE LIPIDIC COMPOUNDS, COMPOSITIONS CONTAINING THEREOF, AND USES THEREOF

(71) Applicant: SANOFI PASTEUR, Lyon (FR)

(72) Inventors: Jean HAENSLER, Paris (FR); Luc EVEN, Paris (FR); Manon RIPOLL, Paris (FR)

(21) Appl. No.: 18/016,616

(22) PCT Filed: Jul. 16, 2021
(86) PCT No.: PCT/EP21/70020

§ 371 Date: Jan. 17, 2023

(30) Foreign Application Priority Data
Jul. 17, 2020 (EP) 20305824.3

(51) Int. Cl. A61K 9/51; C07C 229/12; C07C 271/16; C07C 233/18; C07C 229/16; A61K 39/145

(52) U.S. Cl. CPC A61K 9/5123; C07C 229/12; C07C 271/16; C07C 233/18; C07C 229/16; A61K 39/145; A61K 2039/6018

(57) ABSTRACT

The disclosure relates to novel lipidic compounds, method of manufacturing lipid nanoparticles (LNPs) containing thereof, lipid nanoparticles (LNPs) containing thereof, and the use of the LNPs for the delivery of nucleic acid. The lipidic compounds as disclosed herein is a cleavable lipidic compound comprising at least one terminal radical of formula (I): Y—(CHR)n-Z—(CHR′)p-Q*(I) wherein: —* is the end linked, directly or not, to one C₁₀ to C₅₅ lipophilic or hydrophobic tail-group; —Y is a radical selected in the group consisting of methyl, methoxy, trifluoromethyl, imidazolyl, or is one hydrogen; —Z is a radical —NH-CH2-CO—O—**or a radical —CR″(NH2)-CO—O—** with ** that is the end closest to Q and R″ that is selected in the group consisting of hydrogen, methyl radical and trifluoromethyl radical; —Q is a radical —NH-CH2-CO—O—*** or a radical —CR″(NH2)-CO—O—*** with R″ selected in the group consisting of hydrogen, methyl radical and trifluoromethyl radical and *** that is the end linked, directly or not, to said lipophilic or hydrophobic tail-group; —R et R′ are, independently one from the other, one hydrogen, one methyl radical or one trifluoromethyl CA radical; —n et p are independently one from the other 0, 1 or 2; or one of its pharmaceutically acceptable salts and with said compound being in all the possible racemic, enantiomeric and diastereoisomeric isomer forms.

### TECHNICAL FIELD

The present disclosure relates to novel lipidic compounds which can be used to form lipid nanoparticles for delivery of therapeutic agents, such as nucleic acid, for instance in combination with other lipids, such as neutral lipids, steroids and polymer conjugated lipids. The formulations prepared with the lipidic compounds as described herein enable to induce an immune response after administration of antigen-coding nucleic acid.

### TECHNICAL BACKGROUND

The polynucleotide therapeutics field has seen remarkable progress over the recent years. Polynucleotides include various nucleic acids-based compounds such as messenger RNA (mRNA), antisense oligonucleotides, ribozymes, DNAzymes, plasmids, or immune stimulating nucleic acids. Some nucleic acids, such as mRNA, plasmids and ssDNA, can be used to induce the expression of specific cellular products useful in the treatment of, for example, diseases related to a deficiency of a protein or enzyme, or for the expression of a vaccine antigen to induce specific immune responses. The therapeutic applications of translatable nucleotide delivery are extremely broad as constructs can be synthesized to produce any chosen protein sequence, whether or not indigenous to the system. The expression products of the nucleic acid can augment existing levels of protein, replace missing or non-functional versions of a protein, introduce new protein and associate functionality in a cell or organism or expose to a foreign protein in order to induce a specific immune response.

However, there are many challenges associated with the delivery of polynucleotides to affect a desired response in a biological system and the effective delivery of polynucleotides to their intracellular sites of action remains a major issue. To be efficiently delivered to their site of action, the polynucleotides must be (i) protected from enzymatic and non-enzymatic degradation, (ii) appropriately distributed in the biologic compartment of interest, (iii) effectively and efficiently internalized by the targeted cells, and then (iv) delivered to the intracellular compartment where the relevant translation machinery resides.

Lipid nanoparticles formed from cationic lipids formulated with other lipid components, such as neutral lipids, cholesterol, and PEGylated lipids have been used to protect the polynucleotide from degradation and facilitate its cellular uptake.

While lipid nanoparticles-based vehicles that comprise a cationic lipid component have shown promising results with regards to encapsulation, stability and site localization, there remains a great need for improvement of lipid nanoparticles-based delivery systems. Indeed, many of the cationic lipids that are employed to construct such lipid nanoparticles may be toxic to the targeted cells, and accordingly may be of limited use, notably in quantities necessary to successfully deliver encapsulated materials to such target cells.

Therefore, there remains a need for improved lipid nanoparticles that demonstrate improved pharmacokinetic properties, and which are capable of delivering various types of nucleic acids to a wide variety cell types and tissues with enhanced efficiency.

In contrast to positively charged lipid nanoparticles, neutral or negatively lipid nanoparticles have generally relatively improved pharmacokinetic properties. However, they usually yield low encapsulation efficiency. Therefore, there remains a need for novel lipids able to combine the high efficiency of polynucleotides encapsulation rate associated with cationic lipid and the pharmacokinetic properties of neutral or lowly charged lipid nanoparticles. In prior art, this has been achieved with ionizable cationic lipids displaying a cationic charge at low pH and a neutral charge at neutral pH. However, such ionizable lipids may display some toxic effects, either locally, systemically or both, upon in vivo administration.

Hence, there remains a particular need for novel lipidic compounds having reduced toxicity and are capable of efficiently encapsulating polynucleotides and delivering encapsulated polynucleotides to targeted cells, tissues and organs. Improved lipids and lipid nanoparticles for the delivery of polynucleotides would also provide optimal polynucleotide(s)/lipid(s) ratios, protect the polynucleotides from degradation and clearance in serum, be suitable for systemic or local delivery, and provide intracellular delivery of the polynucleotide. In addition, the lipid-polynucleotide particles should be well-tolerated and provide an adequate therapeutic index, such that patient treatment at an effective dose of the polynucleotide is not associated with unacceptable toxicity and/or risk to the patient.

The present disclosure provides these and related advantages.

### SUMMARY

Accordingly, one of the objects of the present disclosure relates to new cleavable lipidic compounds comprising at least one terminal radical of formula (I):

Y—(CHR)ₙ—Z—(CHR′)ₚ-Q * (I)
wherein:
* is the end linked, directly or not, to one C₁₀ to C₆₀ and preferably to C₁₀ to C₅₅ lipophilic or hydrophobic tail-group,
Y is a radical selected in the group consisting of a C₁-C₅ alkyl, a C₁-C₅ alkoxy, a C_{I}-C₅ acyl, a C₁-C₅ hydroxyalkyl, a C₁-C₅ aminoalkyl, a C₁-C₅ alkylcarboxyl ester, an acetamido, a N,N—C₁-C₅ alkylamido, a C₁-C₅ fluoroalkyl, for example a C₁-C₅ perfluoroalkyl, for example a trifluoromethyl, an imidazolyl, a triazolyl, a squaramidyl, an ureido, a cyano or is one hydrogen;
Z is a radical —NH—CH₂—CO—O—** or a radical —CR″(NH₂)—CO—O—** with ** that is the end closest to Q and R″ that is selected in the group consisting of hydrogen, methyl radical and trifluoromethyl radical
Q is a radical —NH—CH₂—CO—O—*** or a radical —CR″(NH₂)—CO—O—*** with R″ that is selected in the group consisting of hydrogen, methyl radical and trifluoromethyl radical and *** that is the end linked, directly or not, to said lipophilic or hydrophobic tail-group;
R et R′ are, independently one from the other, one hydrogen, one methyl radical or one trifluoromethyl radical;
n et p are, independently one from the other, 0, 1 or 2;

or one of the pharmaceutically acceptable salts of said radical; and with said lipid compound that is in all the possible racemic, enantiomeric and diastereoisomeric isomer forms.

According to one embodiment, the compound as disclosed herein is a compound of formula (II)

Y—(CHR)ₙ—Z—(CHR′)ₚ-Q-A-R₁ (II)
wherein:
   Y, R, n, Z, R′, p and Q are as defined herein;
   R₁ is a C₁₀ to C₆₀ and preferably to C₁₀ to C₅₅ lipophilic or hydrophobic tail-group; and
   A is a spacer arm having from 2 to 24, for example from 2 to 18, for example from 4 to 12 carbon atoms, or for example from 2 to 12 carbon atoms, in a branched or unbranched linear, saturated or unsaturated hydrocarbon chain, said chain being interrupted by one or several atoms of oxygen and/or moieties selected among —S—S—; (C═O)—O—; —O—(O═C)—; —(C═O)—NH—; —NH—(C═O)—O—;—S—;and —O—(O═C)—NH—, and/or having a terminal atom of oxygen or one moiety like —(C═O)—O—; —NH—(C═O)—O— or —NH— to its end to be linked to the lipophilic or hydrophobic tail-group,
or one of the pharmaceutically acceptable salts of said compound of formula (II); and any of its racemic, enantiomeric and diastereoisomeric isomer forms.

According to another embodiment, the compound according to the disclosure is a compound of formula (IIa)

Y—(CHR)ₙ—NH—CH₂—CO—O—(CHR′)ₚ—NH—CH₂—CO—O-A-R₁ (IIa)
wherein:
   Y, R, n, R′ and p are as previously defined;
   R₁ is a C₁₀ to C₆₀ and preferably to C₁₀ to C₅₅ lipophilic or hydrophobic tail-group;
   and
   A is a spacer arm as previously defined;
or one of the pharmaceutically acceptable salts of said compound of formula (II); and any of its racemic, enantiomeric and diastereoisomeric isomer forms.

According to another object, the disclosure relates to a method for manufacturing lipid nanoparticles containing a nucleic acid, wherein the method comprises at least the steps of:
a) solubilizing, in a water miscible organic solvent, at least one lipidic compound as described herein,
b) mixing the organic solvent obtained at step a) with at an aqueous solvent buffered at a pH ranging from about 3.0 to about 4.5 and comprising at least one nucleic acid, and
c) obtaining said nucleic acid containing lipid nanoparticles in the aqueous solvent.

In one embodiment, the lipid nanoparticles manufacturing method as described herein further comprises a step d) of increasing the pH of the aqueous solvent containing the lipid nanoparticles obtained at step c) at a pH ranging about 5.0 to about 8.5, for example from about 5.5 to about 8.0, for example from about 6.0 to about 7.5, and for example from about 6.5 to about 7.0.

According to another embodiment, the disclosure relates to lipid nanoparticles obtainable according the manufacturing method as disclosed herein.

According to another embodiment, the disclosure relates to a method for manufacturing a pharmaceutical composition comprising at least the steps of:
i) manufacturing at least one lipid nanoparticle according to the method as described herein, and
ii) combining the lipid nanoparticles obtained at step i) with at least one pharmaceutically acceptable excipient or carrier.

According to another embodiment, the disclosure relates to a method for manufacturing an immunogenic composition comprising at least the steps of:
i) manufacturing at least one lipid nanoparticle according to the method as described herein, said lipid nanoparticle containing at least one nucleic acid encoding for at least one antigen, and
ii) combining the lipid nanoparticles obtained at step i) with at least one pharmaceutically acceptable excipient or carrier.

According to another embodiment, the disclosure relates to lipid nanoparticles obtainable according to a method as disclosed herein.

According to another embodiment, the disclosure relates to a lipid nanoparticle comprising at least one lipidic compound of formula (IV):

HO-L (IV)
wherein L is a lipophilic or hydrophobic tail-group as described herein, for instance a C₁₀ to C₅₅ lipophilic or hydrophobic tail-group,
and at least one nucleic acid.

According to another embodiment, the disclosure relates to a pharmaceutical composition comprising at least one lipid nanoparticle as described herein, and at least one pharmaceutically acceptable excipient or carrier.

According to another embodiment, the disclosure relates to an immunogenic composition comprising at least one lipid nanoparticle as disclosed herein wherein the least one nucleic acid encodes for at least one antigen.

According to another embodiment, the disclosure relates to a composition comprising at least one lipid nanoparticle as disclosed herein as a medicament.

According to another embodiment, the disclosure relates to a composition comprising at least one lipid nanoparticle as disclosed herein, for use in a therapeutic method for preventing and/or treating a disease selected in a group consisting of infectious diseases, allergies, autoimmune diseases, rare blood disorders, rare metabolic diseases, rare neurologic diseases, and tumour or cancer diseases.

The term “rare diseases” is used herein according to its meaning acknowledge in the art to mean diseases with an average prevalence threshold between 40 and 50 cases/100,000 people (Richter et al., Value Health. 2015 Sep. 18(6):906-14).

According to another embodiment, the disclosure relates to a composition comprising at least one lipid nanoparticle as disclosed herein for use as an immunogenic composition.

According to another embodiment, the disclosure also relates to a method of preventing and/or treating a disease in an individual in need thereof, wherein the method comprises administering an effective amount of at least one lipid nanoparticle as disclosed herein, to said individual. A method as disclosed herein may be for preventing and/or treating infectious diseases, allergies, autoimmune diseases, rare blood disorders, rare metabolic diseases, rare neurologic diseases, and tumor or cancer diseases.

In some embodiments, the disclosure also relates to a use of at least one lipid nanoparticle as disclosed herein for the manufacture of a medicament for preventing and/or treating infectious diseases, allergies, autoimmune diseases, rare blood disorders, rare metabolic diseases, rare neurologic diseases, and tumor or cancer diseases.

### DESCRIPTION OF THE FIGURES

FIG. 1: Hemagglutination inhibiting antibody mean titers (HI titers) measured in serum from mice post-2 immunization either with LNPs L319 or with LNPs Lip. (III) (manufactured with lipidic compound of formula (III)) each loaded with 5 g of mRNA encoding full-length hemagglutinin (HA) of influenza virus strain A/Netherlands/602/2009 (H1N1). 2 groups of 8 BALBc/ByJ mice (8 week-old at DO) received two intramuscular (IM) injections, given three weeks apart (DO and D21) with each the 2 LNPs formulations. As negative control group, 4 mice were immunized with PBS buffer and as positive control group, 8 mice received 10 μg of monovalent Flu vaccine A/California/07/2009 (H1N1) strain derived from Vaxigrip™.

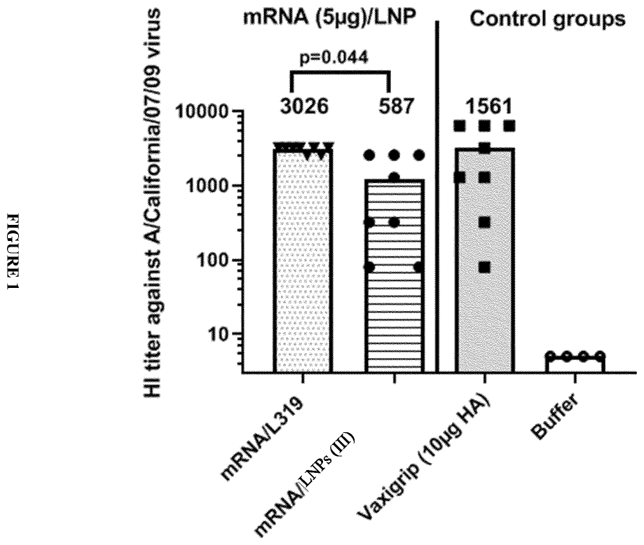

FIG. 2: Bioluminescence signal acquisition monitoring protein expression in injected site (quadriceps) following intramuscular administration of LNPs 319 or LNPs Lip. (III) loaded with 5 μg of mRNA encoding Luciferase (mRNA-Luc) in female BALB/c ByJ mice. The luminescence level was evaluated by an ROI applied to the injection site zone at 6 h, 24, 48 h and 72 h and the results are expressed as total flux (ph/s) in function of time (hours) post the injection of LNPs/mRNA-Luc. A buffer Tris/sucrose was used as control.

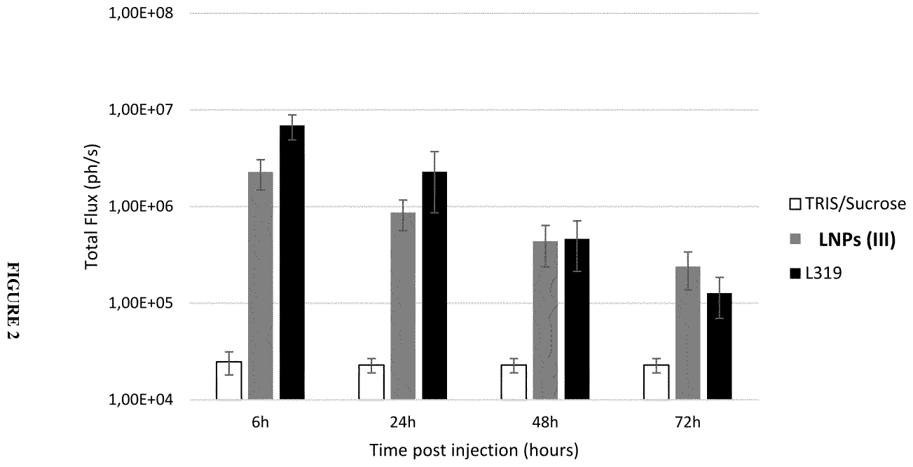

FIG. 3: shows scheme (4) of synthesis of compound (VI).

FIG. 4: shows scheme (5) of synthesis of compound (VII).

FIG. 5: shows scheme (7) of synthesis of compound (VHI).

FIG. 6: shows scheme (8) of synthesis of compound (XV).

FIG. 7: shows scheme (9) of synthesis of compound (XVII).

FIG. 8: shows scheme (10) of synthesis of compound (XIX).

FIG. 9: shows scheme (11) of synthesis of compound (XXIII).

FIG. 10: shows scheme (12) of synthesis of compound (XXIX).

FIG. 11: shows scheme (13) of synthesis of compound (XXXI).

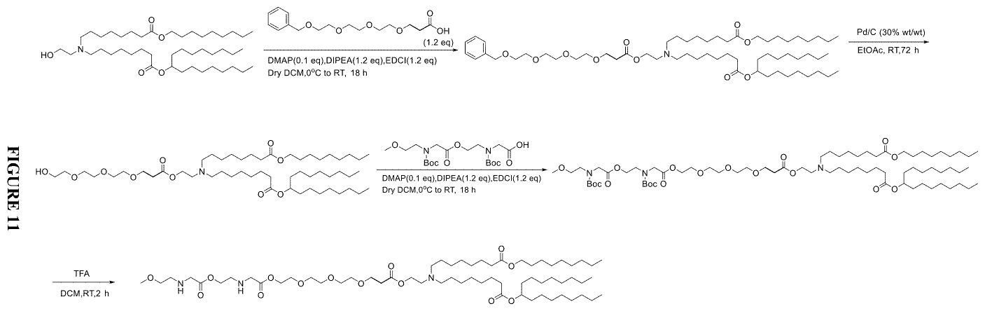

FIG. 12: shows scheme (14) illustrating the cleavage of lipidic compound of formula (III) (DOG-Cleave) through a cyclisation process generating successively “DOG-cleave transient” and the uncharged lipid “DOG-OH” in the final LNP formulation.

FIG. 13: shows a chromatogram illustrating the separation of the lipidic compound of formula (III) (DOG-Cleave), DOG-Cleave transient, DOG-OH, DSPC, Chol and DMG-PEG2000 on the C18-HPLC column.

FIG. 14: shows the HI responses induced by LNPs comprising influenza HA mRNA (1MpU-modified from Amptec) in non-human primates immunized twice four weeks apart (DO, D28) with 50 μg of mRNA in LNPs (LNPs (III)/DOG-CLEAVE or LNPs-L319)) injected IM.

FIG. 15: Hemagglutination inhibiting antibody mean titers (HI titers) measured in sera collected at D21 in mice immunized at DO and D21 with LNPs L319, LNPs (III) [DOG-Cleave], LNPs (XXI), and LNPs (XIX) containing DSPC as neutral lipid and loaded with mRNA encoding full-length hemagglutinin (HA) of influenza virus strain A/Netherlands/602/2009 (H1N1).

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them. The following definitions are provided for the present specification, including the claims.

The term “cleavable radical” means that said radical, when covalently linked to another functional group for forming a compound, for example a lipidic compound as disclosed herein, is capable of being cleaved from the rest of the molecule, upon exposure to biological conditions, and such as in the context of the instant disclosure, upon exposure to a pH greater than 5 and, for example greater than 6.

The term “terminal radical” means that said radical is a head-group or a tail-group.

The term “pharmaceutically acceptable salts” includes for example acid addition salts of compounds as disclosed herein derived from the combination of such compounds with non-toxic acid.

The term “acid addition salts” include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

The pharmaceutically acceptable salts of compounds as disclosed herein can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present disclosure.

In the context of the present disclosure the chemical terms below have the following meanings:
Ct-Cz: a carbon chain that can have from t to z carbon atoms, where t and z may have the values from 1 to 7; for example, C₁-C₄ is a carbon chain that may have from 1 to 4 carbon atoms;
a heteroatom is understood to mean nitrogen, oxygen or sulphur;
an heteroaromatic ring denotes a 5- or 6-membered aromatic ring comprising 1 or 2 heteroatoms;
an aromatic ring refers to a mono or polycyclic, such as a monocyclic, aromatic hydrocarbon radical of 6-20 atoms, for example of 6 atoms, derived by the removal of one hydrogen from a carbon atom of a parent aromatic ring system.

An aromatic ring according to the disclosure is for example a phenyl group; As used in this specification and the appended claims, the singular forms “a”, “an” and “the” include plural referents unless the content clearly dictates otherwise.

The term “about” or “approximately” as used herein refer to the usual error range for the respective value readily known to the skilled person in this technical field. Reference to “about” a value or parameter herein includes (and describes) embodiments that are directed to that value or parameter per se. In some embodiments, the term “about” refers to ±10% of a given value. However, whenever the value in question refers to an indivisible object, such as a nucleotide or other object that would lose its identity once subdivided, then “about” refers to ±1 of the indivisible object.

The term “antigen” comprises any molecule, for example a peptide or protein, which comprises at least one epitope that will elicit an immune response and/or against which an immune response is directed. For example, an antigen is a molecule which, optionally after processing, induces an immune response, which is for example specific for the antigen or cells expressing the antigen. After processing, an antigen may be presented by MHC molecules and reacts specifically with T lymphocytes (T cells). Thus, an antigen or fragments thereof should be recognizable by a T cell receptor and should be able to induce in the presence of appropriate co-stimulatory signals, clonal expansion of the T cell carrying the T cell receptor specifically recognizing the antigen or fragment, which results in an immune response against the antigen or cells expressing the antigen.

According to the present disclosure, any suitable antigen may be envisioned which is a candidate for an immune response. An antigen may correspond to or may be derived from a naturally occurring antigen. Such naturally occurring antigens may include or may be derived from allergens, viruses, bacteria, fungi, parasites and other infectious agents and pathogens or an antigen may also be a tumor antigen.

As used herein, the term “aqueous solution” or “aqueous solvent” refers to a composition comprising water.

Within the disclosure the terms “cationic” refers to an ion or group of ions having a positive charge.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. Depending on the context, the term “comprise” may also specify strictly the stated features, integers, steps or components, and therefore in such case it may be replaced with “consist”.

The term “charged lipid” refers to any of a number of lipid species that exist in either a positively charged or negatively charged form within a useful physiological range e.g. pH˜3 to pH˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. DOTAP, DOTMA), dialkyldimethylaminopropanes, ethyl phosphocholines, dimethylaminoethane carbamoyl sterols (e.g. DC-Chol).

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

The term “neutral lipid” refers to any of a number of lipid species that is either not ionizable or is a neutral zwitterionic compound at a selected pH, for example at physiological pH. Such lipids include, but are not limited to, phosphatidylcholines, phosphatidylethanolamines sphingomyelins (SM), or neutral sphingolipids such as ceramides. Neutral lipids may be synthetic or naturally derived.

As used herein, the term “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

The term “lipid” refers to a group of organic compounds that include, but are not limited to, esters of fatty acids and are generally characterized by being poorly soluble in water, but soluble in many organic solvents. Lipid is a generic term encompassing fats, fatty oils, essential oils, waxes, phospholipids, glycolipids, sulfolipids, aminolipids, chromolipids (lipochromes), and fatty acids. Within the disclosure, “lipid” encompasses neutral lipids, steroid alcohol or ester thereof, and PEGylated lipids.

The term “lipid nanoparticle” (LNP) refers to particles having at least one dimension on the order of nanometers (e.g., 1-1 000 nm) which may be formulated with at least one of the lipidic compound as disclosed herein. In some embodiments, lipid nanoparticles are included in a formulation that can be used to deliver an active agent or therapeutic agent, such as a nucleic acid to a target site of interest (e.g., cell, tissue, organ, tumor, and the like). Such lipid nanoparticles typically comprise a lipidic compound as disclosed herein, or one lipid derived from the hydrolysis of a lipidic compound as disclosed herein, and at least one ingredient selected from neutral lipids, steroid alcohols or esters thereof, and polymer conjugated lipids.

As used herein, “lipid encapsulated” refers to a lipid nanoparticle that provides an active agent or therapeutic agent, such as a nucleic acid with full encapsulation, partial encapsulation, or both. In an embodiment, the polynucleotide is fully encapsulated in the lipid nanoparticle.

It should be noted that the terms “head-group” and “tail-group” as used in the instant specification, describe parts of the compounds of the present disclosure, such as functional groups of such compounds. They are used to describe the orientation of one or more functional groups relative to other functional groups in said compounds. They are both “end group”.

As used herein, the terms “lipophilic or hydrophobic tail-group” indicate in qualitative terms that the tail the tail has an affinity for lipids (and typically is lipid-soluble) and is water-avoiding (and typically is not water soluble).

The term “PEGylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

Within the disclosure the term “nucleic acids”, “polynucleotide”, and “oligonucleotides” are used interchangeably. They refer to a polymeric form of at least two nucleotides, either deoxyribonucleotides or ribonucleotides, or analogs thereof. Nucleic acids may have any three-dimensional structure, and may perform any function, known or unknown. They may be linear or cyclic. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, closed-ended DNA (ceDNA), self-amplifying RNA (saRNA), stranded DNA (ssDNA), small interfering RNA (siRNA) and micro RNA (miRNA), recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “complement of a polynucleotide” denotes a polynucleotide molecule having a complementary base sequence and reverse orientation as compared to a reference sequence, such that it could hybridize with a reference sequence with complete fidelity. “Recombinant” as applied to a polynucleotide means that the polynucleotide is the product of various combinations of in vitro cloning, restriction and/or ligation steps, and other procedures that result in a construct that can potentially be expressed in a host cell.

The term “steroid alcohol” or “sterol” refers to a group of lipids comprised of a sterane core bearing a hydroxyl moiety. As example of steroid alcohol, one may cite cholesterol, campesterol, sitosterol, stigmasterol and ergosterol. Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ alkyl group. In other embodiments, the carboxylic acid may be a fatty acid.

As used herein, the terms “prevent”, “preventing” or “delay progression of” (and grammatical variants thereof) with respect to a disease or disorder relate to prophylactic treatment of a disease, e.g., in an individual suspected to have the disease, or at risk for developing the disease. Prevention may include, but is not limited to, preventing or delaying onset or progression of the disease and/or maintaining at least one symptom of the disease at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature.

As used herein, “target cells” or “targeted cells” refer to cells of interest. The cells may be found in vitro, in vivo, in situ or in the tissue or organ of an organism. The organism may be an animal, such as a mammal, for example a human, and for example a human patient.

The terms “treat” or “treatment” or “therapy” in the present text refers to the administration or consumption of a composition as disclosed herein with the purpose to cure, heal, alleviate, relieve, alter, remedy, ameliorate, improve, or affect a disorder, the symptoms of the condition, or to prevent or delay the onset of the symptoms, complications, or otherwise arrest or inhibit further development of the disorder in a statistically significant manner.

As used herein, the terms “therapeutically effective amount” and “prophylactically effective amount” refer to an amount that provides a therapeutic benefit in the treatment, prevention, or management of pathological processes considered. The specific amount that is therapeutically effective can be readily determined by an ordinary medical practitioner and may vary depending on factors such as the type and stage of pathological processes considered, the patient's medical history and age, and the administration of other therapeutic agents.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” . . . list of items . . . “and combinations and mixtures thereof.” Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

Detailed Definition of Radicals of Formula (I) and Lipidic Compounds of the Disclosure

The lipidic compounds of the disclosure are ionizable, and for example are cationic lipidic compounds.

The lipidic compounds of the present disclosure may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, that is included in the present disclosure. In addition, the cationic lipids disclosed herein may exist as tautomers and both tautomeric forms are intended to be encompassed by the scope of the disclosure, even though only one tautomeric structure is depicted.

The pharmaceutically acceptable salts of lipidic compounds of the present disclosure have one or several counter ions which are generally physiologically acceptable. As possible counter ions may be cited halides, phosphate, trifluoroacetate, sulfite, nitrate, gluconate, glucuronate, galacturonic acid radical, alkylsulfonate, alkylcarboxylate, propionic sulfonate and methanesulfonic acid radical.

The lipidic compounds as disclosed herein and the pharmaceutically acceptable salts thereof can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present disclosure.

For example, the lipidic compounds as disclosed herein have one hydrophilic head group formed by one radical of formula (I) also named terminal radical to illustrate the fact that it is linked, directly or not, to an end of one hydrophobic or lipophilic tail also forming said lipidic compounds.

The radical of formula (I) has the following definition:

Y—(CHR)ₙ—Z—(CHR′)ₚ-Q * (I)
wherein:
* is the end linked, directly or not, to one C₁₀ to C₆₀ and preferably to C₁₀ to C₅₅ lipophilic or hydrophobic tail-group,
Y is a radical selected in the group consisting of a C₁-C₅ alkyl, a C₁-C₅ alkoxy, a C₁-C₅ acyl, a C₁-C₅ hydroxyalkyl, a C₁-C₅ aminoalkyl, a C₁-C₅ alkylcarboxyl ester, an acetamido, an N,N—C₁-C₅ alkylamido, a C₁-C₅ fluoroalkyl, for example a C₁-C₅ perfluoroalkyl, for example a trifluoromethyl, an imidazolyl, a triazolyl, a squaramidyl, an ureido, a cyano or is one hydrogen and preferably is a C₁-C₅ alkoxy and more preferably a methoxy;
Z is a radical —NH—CH₂—CO—O—** or a radical —CR″(NH₂)—CO—O—** with ** that is the end closest to Q and R″ that is selected in the group consisting of hydrogen, methyl radical and trifluoromethyl radical,
Q is a radical —NH—CH₂—CO—O—*** or a radical —CR″(NH₂)—CO—O—*** with R″ selected in the group consisting of hydrogen, methyl radical and trifluoromethyl radical and *** that is the end linked, directly or not, to said lipophilic or hydrophobic tail-group in particular as described herein;
R et R′ are, independently one from the other, one hydrogen, one methyl radical or one trifluoromethyl radical;
n et p are independently one from the other 0, 1 or 2;

or one of its pharmaceutically acceptable salts and with said lipidic compound being in all the possible racemic, enantiomeric and diastereoisomeric isomer forms.

The radical of formula (I) exists under a protonated and stabilized form at a pH lower than 6.0 and for example lower than 5.5 and for example lower than 5. In contrast, when exposed to a pH greater than the one stabilizing the protonated form, such a radical of formula (I), present in a lipidic compound according to the disclosure, advantageously undergoes a chemical rearrangement. As shown in the following scheme 1, this chemical rearrangement leads to its cleavage from the rest of the lipidic molecule. This ability to exist in a positively charged form is for example efficient for immobilizing negatively charged nucleic acids and charging them in specific chemical vehicles dedicated to promoting the, in vitro or in vivo, targeted release of said nucleic acids. In contrast, when such vehicle is exposed to a pH greater than 6.0, the radical of formula (I) may then be cleaved. By this way, the encapsulated nucleic acids to be released are advantageously free from this radical.

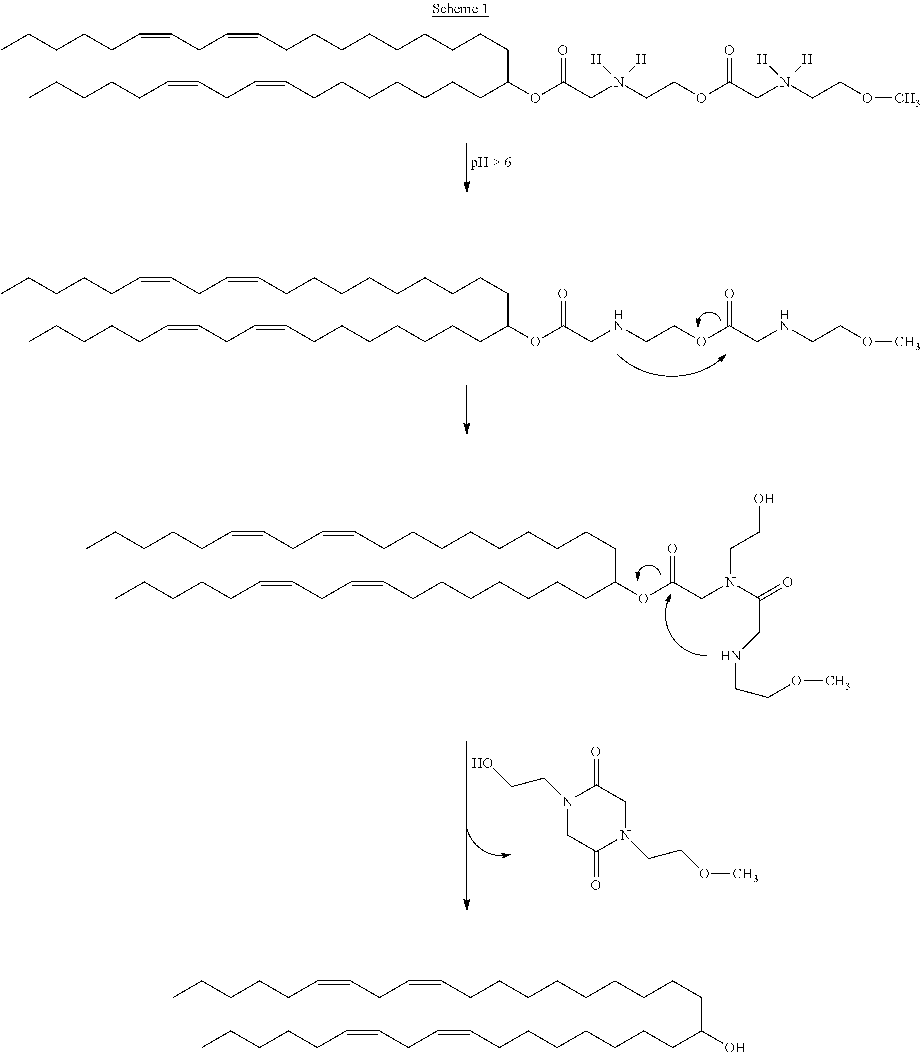

According to one embodiment, Z and Q are both one radical —NH—CH₂—CO—O—, and for example n and p are both 2.

According to another embodiment, Z and Q are different and one of them is one radical —NH—CH₂—CO—O— and the other one is one radical —CH(NH₂)—CO—O—, and for example n and p are different and equal to 1 or 2. In this specific embodiment, Z is preferably —CR″(NH₂)—CO—O— and Q is —NH—CH₂—CO—O—.

According to another embodiment, Z and Q are both one radical —CR″(NH₂)—CO—O— with preferably R″ being an hydrogen and for example n and p are both 1.

As previously stated, one radical of formula (I) is directly or not, attached to a hydrophobic (lipophilic) tail group (e.g, a covalent bond).

The hydrophobic or lipophilic tail is generally in C₁₀ to C₅₅ but may be also in C₁₀ to C₆₀.

For example, it is an optionally substituted branched or unbranched linear saturated or unsaturated C₁₀ to C₆₀ and preferably C₁₀ to C₅₅ hydrocarbon radical, and which hydrocarbon skeleton that is optionally interrupted by one or several atoms of oxygen or nitrogen and/or one or several —O—CO— or —CO—O— groups and which one nitrogen atom present on the skeleton can be, linked, directly or not, to the radical represented by formula (I).

In particular, the hydrophobic or lipophilic tail is an optionally substituted branched or unbranched linear saturated or unsaturated C₁₀ to C₆₀ and preferably C₁₀ to C₅₅ hydrocarbon radical, and which hydrocarbon skeleton that is optionally interrupted by one or several atoms of oxygen and/or one or several —O—CO— or —CO—O— groups and if one nitrogen atom is present in the skeleton it is present under a form that cannot be protonated and is linked, directly or not, and preferably directly linked to the spacer, A. In particular, it may form an amide moiety with the —C═O terminal moiety of said spacer like for example in compound (XXVII).

For example, the hydrophobic or lipophilic tail comprises at least two, three or more hydrocarbon chains each one independently being selected from optionally substituted C₈-C₂₄, for example C₁₀-C₂₀, alkyl chain, optionally substituted variably saturated or unsaturated C₈-C₂₄, for example C₁₀-C₂₀, alkenyl chain and optionally substituted, saturated, variably saturated or unsaturated C₈-C₂₄, for example C₁₀-C₂₀, acyl chain with said alkyl, alkenyl or acyl chains can be interrupted by one or several atoms of oxygen and/or one or several moieties like —O—CO— or —CO—O—.

Each hydrocarbon chain may be substituted by at least one radical selected from —OH, CO₂H and alkyl group in C₁ to C₄ and preferably being unsubstituted.

According to a one embodiment, the hydrophobic or lipophilic tail is selected in the group consisting of

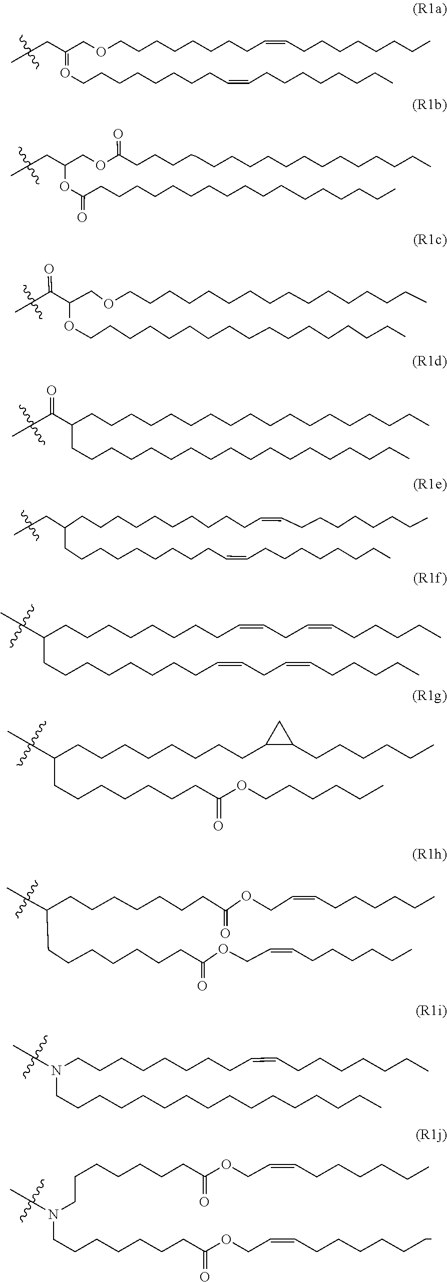

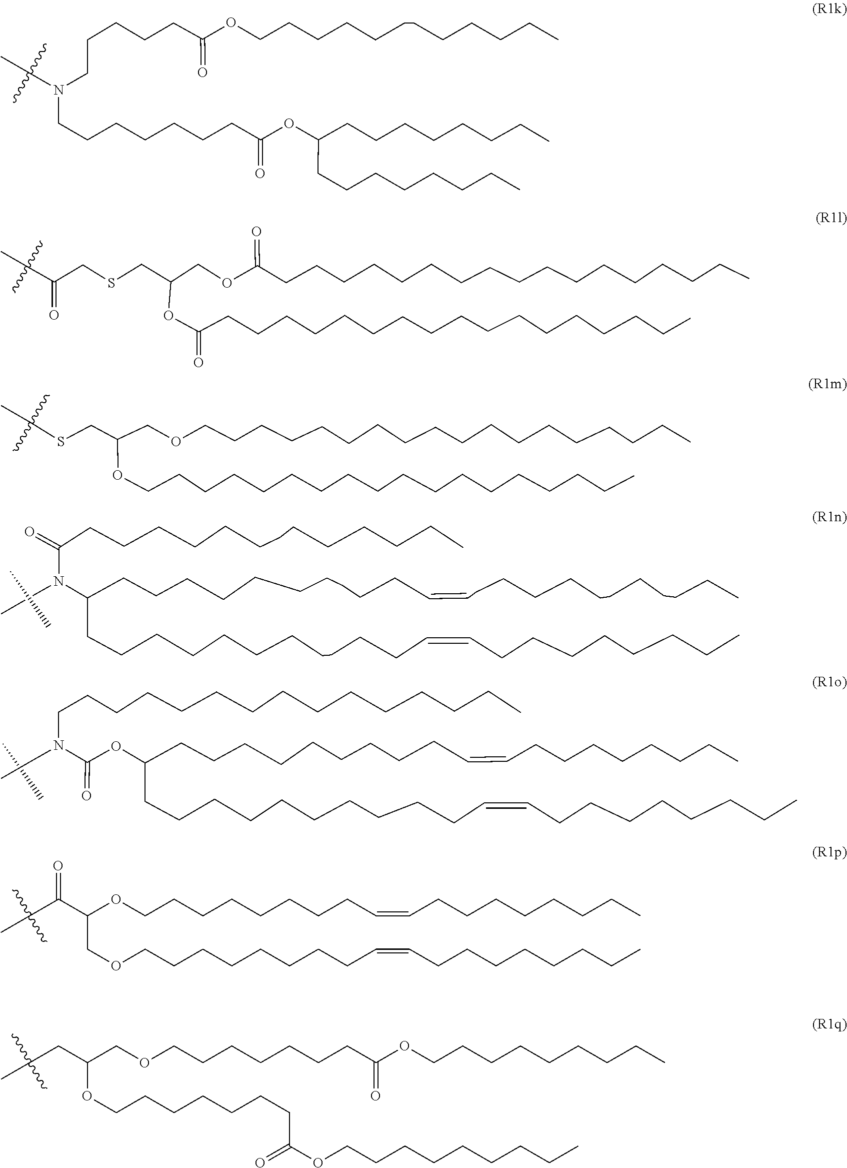

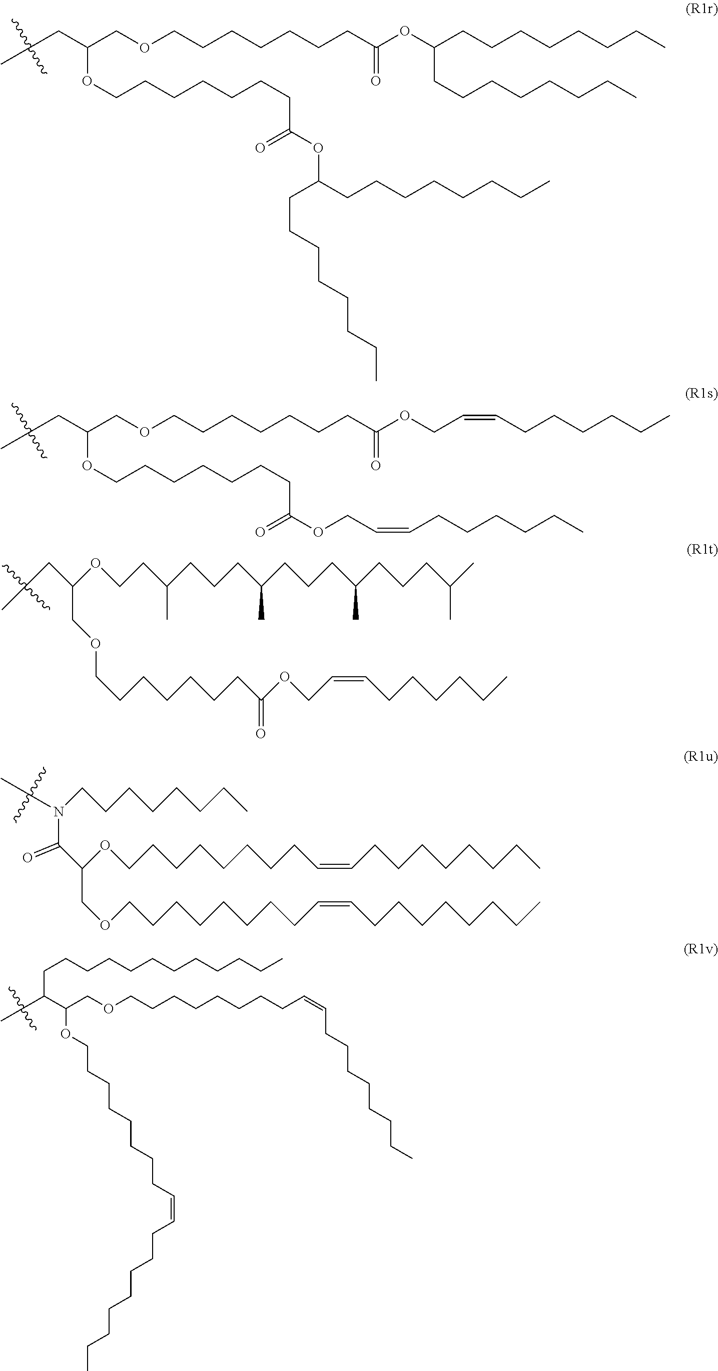

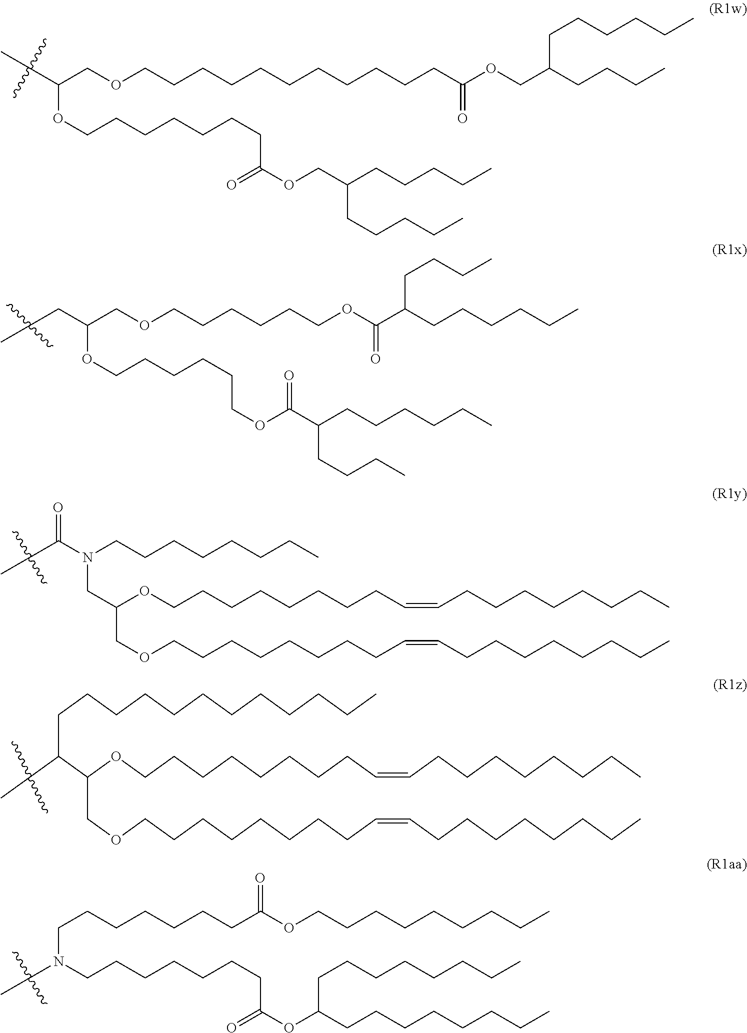

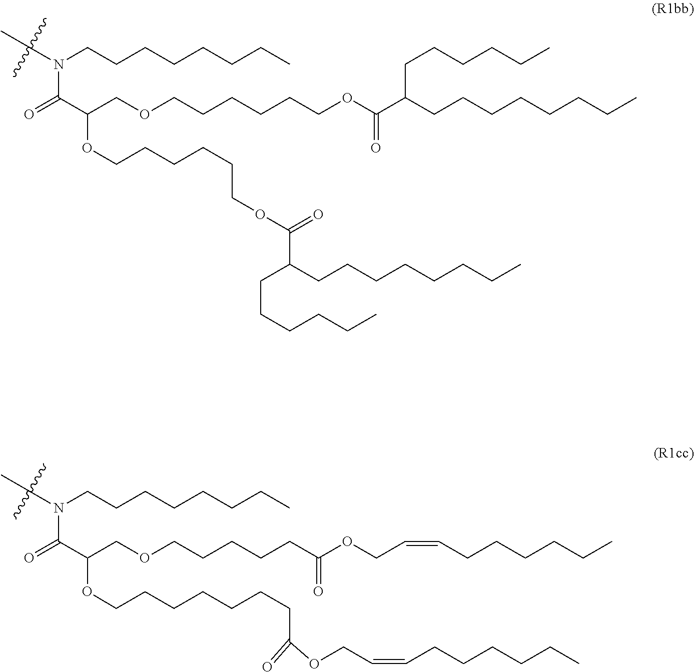 [SMILES: *N(CCCCCCCC)C(=O)C(COCCCCCCOC(=O)C(CCCCCC)CCCCCCCC)OCCCCCCOC(=O)C(CCCCCC)CCCCCCCC ; *N(CCCCCCCC)C(=O)C(COCCCCCC(=O)OC/C=C\CCCCCC)OCCCCCCCC(=O)OC/C=C\CCCCCC]

According to a particular embodiment, the hydrophobic or lipophilic tail comprises at least three, four or more hydrocarbon chains, each one independently being selected from optionally substituted C₄-C₂₄, for example C₅-C₂₀, alkyl chain and optionally substituted C₄-C₂₄, for example C₁₀-C₂₀, alkenyl chain.

According to a particular embodiment, the hydrophobic or lipophilic tail comprises at least two, three, four or more hydrocarbon C₄-C₂₄ chains, with at least one chain and preferably at least two chains being interrupted by at least one oxygen atom and/or at least one moiety selected among —O—(O═C)— and —(C═O)—O—.

According to a particular embodiment, the hydrophobic or lipophilic tail comprises at least three, four or more hydrocarbon chains, with at least two chains being optionally substituted C₄-C₂₄, for example C₅-C₂₀ alkylene chain with optionally each one being or not independently interrupted by at least one moiety selected among —O—(O═C)— and —(C═O)—O—.

According to a particular embodiment, the hydrophobic or lipophilic tail comprises at least three, four or more hydrocarbon chains, with all chains being optionally substituted C₄-C₂₄, for example C₅-C₂₀ alkyl chains with optionally each one being or not independently interrupted by at least one moiety selected among —O—(O═C)— and —(C═O)—O—.

In one embodiment, the hydrophobic or lipophilic tail is the tail (R1a) or (R1b) also respectively named DOG alkyl or DOG ether.

According to another embodiment, the cationic and/or ionizable lipidic compound of the disclosure is a compound of formula (II)

Y—(CHR)ₙ—Z—(CHR′)ₚ-Q-A-R₁ (II)
wherein:
   Y, R, n, Z, R′, p and Q are as defined in claim 1;
   R₁ is a C₁₀ to C₆₀ and preferably a C₁₀ to C₅₅ lipophilic or hydrophobic tail-group in particular as described herein; and

A is a spacer arm having from 2 to 24, for example from 2 to 18, for example from 4 to 12 carbon atoms, or for example from 2 to 12 carbon atoms, in a branched or unbranched linear saturated or unsaturated hydrocarbon chain, said chain being interrupted by one or several atoms of oxygen and/or moieties selected among —S—S—; —(C═)—O—; —O—(O═C)—; —(C═O)—NH—; —NH—(C═O)—O—; —S—; —NH—(O═C)—; and —O—(O═C)—NH—; and/or optionally having a terminal atom of oxygen or one moiety like —(C═O)—O—; —O—(O═C)—; —NH—(C═O)—; —NH—(C═O)—O— or —O—(O═C)—NH—;to its end to be linked to the lipophilic or hydrophobic tail-group, or one of its pharmaceutically acceptable salts and any of its racemic, enantiomeric and diastereoisomeric isomer forms.

According to one embodiment, Z and Q are both one radical —NH—CH₂—CO—O—.

According to another embodiment, Z and Q are different and one of them is one radical —NH—CH₂—CO—O— and the other one is one radical —CH(NH₂)—CO—O— and preferably Z is —CR″(NH₂)—CO—O— with R″ being preferably a hydrogen atom, and Q is —NH—CH₂—CO—O—.

According to another embodiment, Z and Q are both one radical —CR″(NH₂)—CO—O—.

For example, according to one embodiment, the lipidic compound as disclosed herein is a compound of formula (IIa)

Y—(CHR)ₙ—NH—CH₂—CO—O—(CHR′)ₚ—NH—CH₂—CO—O-A-R₁ (IIa)
wherein:
   Y, R, n, R′, R₁, A and p are as previously defined in formula (I) or (II);
or one of its pharmaceutically acceptable salts and any of its racemic, enantiomeric and diastereoisomeric isomer forms.

In a specific embodiment, n and p are both 2.

In another specific embodiment, Y is a radical methoxy.

Regarding the spacer arm A of formula (II) and (IIa), it is like the ones conventionally considered in the field of the lipidic compounds. Accordingly, the choice of such spacer arm does not raise any difficulty for the man skilled in the art. Naturally, it needs to be inert or not prejudicial to the efficiency of the lipidic compound.

Generally, the spacer arm A has 2 to 24 and for example from 2 to 12 carbon atoms, or for example from 4 to 10 carbon atoms, comprises at least one or several ethylene oxide units and optionally one or several moieties selected among —OCO—; COO—, —NHCOO—, —OCONH— and —S—S—.

According to a specific embodiment, the spacer arm A may be of formula (A1)

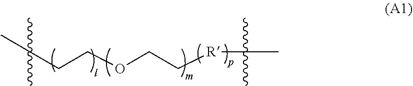

Which right end being the one linked to the hydrophobic or lipophilic tail and wherein: l is 0 or 1;
m ranges from 2 to 12, preferably from 2 to 10 or for example is 2, 3, 4, 5, 6, 7, 8, or 9
p is 0 or 1; and
R′ represents, when p is 1, a group selected from —O—(O═C)—; —(C═O)—O—;—NH—(C═O)—O— or —O—(O═C)—NH—; —NH—(C═O)—O—, -; O—CH₂ C(═O)—O—; —O—C(═O)—(CH₂)₂—C(═O)— and —S—S—.

As examples of spacer arms convenient for the disclosure, it may be cited the following ones and which right end being the one to be linked to the lipophilic or hydrophobic tail-group:

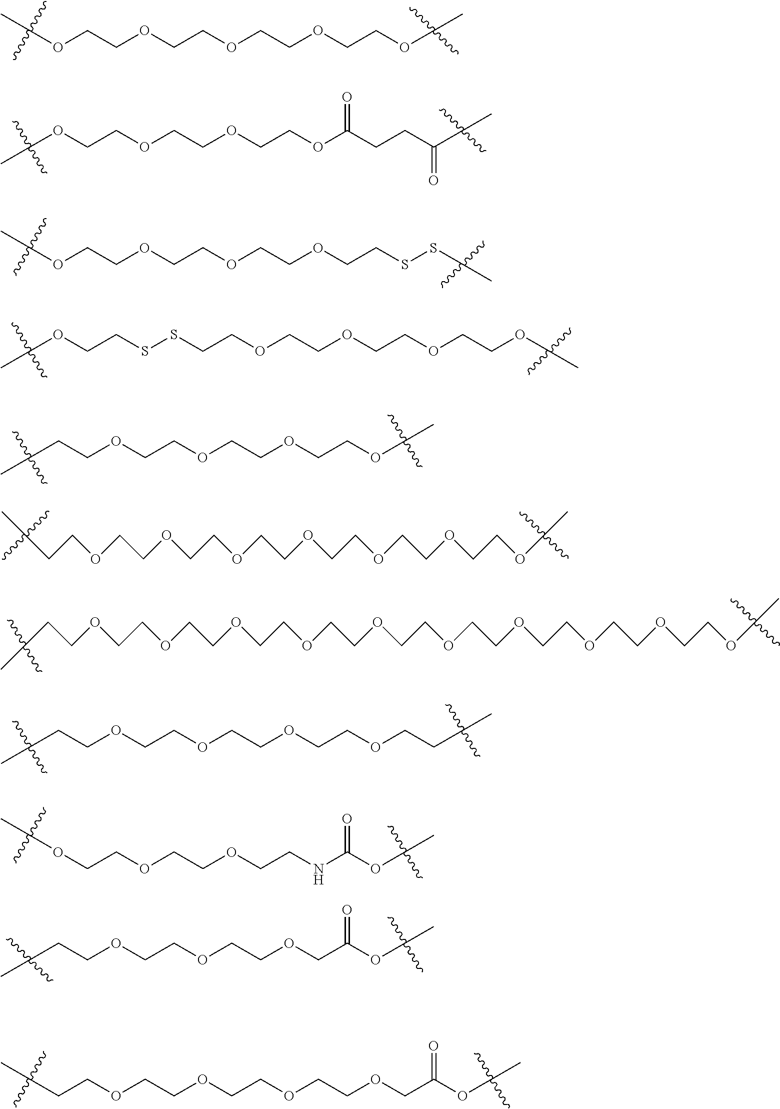

According to one embodiment, the spacer consists in ethylene oxide units. In this embodiment, the spacer is a poly(ethylene oxide) (aka polyethylene glycol—PEG).

In this embodiment, the spacer may comprise 1 to 24 ethylene oxide units and for example 2, 3, 4, 5, 6, 7, 8, 10, 12 and 24 ethylene oxide units.

According to another embodiment, the spacer comprises a poly(ethylene oxide) moiety and further includes at least one moiety selected among —COO—, —OCO—, —NHCOO—, —OCONH— and —CH₂CH₂—.

According to one embodiment, the compound is of formula (II) wherein Z is one radical —CH(NH₂)—CO—O—. and Q is one radical —NH—CH₂—CO—O— and in particular is the following compound (VI):

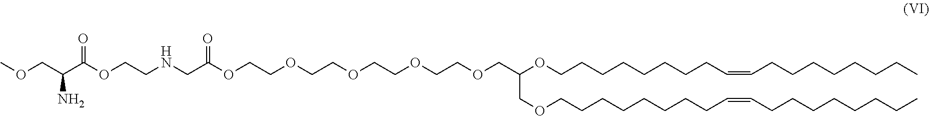 [SMILES: CCCCCCCC/C=C\CCCCCCCCOCC(COCCOCCOCCOCCOC(=O)CNCCOC(=O)[C@@H](N)COC)OCCCCCCCC/C=C\CCCCCCCC]

or one of its salts, for example its trifluoroacetate salt, and or one of its racemic, enantiomeric and diastereoisomeric isomer forms.

According to another embodiment, the compound is of formula (II) wherein Z and Q are both one radical —NH—CH₂—CO—O—.

In this specific embodiment, the compound of formula (II) may be selected among the following compounds (III) and (VI) to (XXXII), and for example among compounds (III), (VI), (XVII), (XIX), (XXI), (XXII), (XXIV), (XXVII) and (XXVIII), or for example among compounds (III), (XIX) or (XXI), and for example may be compound of formula (III) (also named DOG-CLEAVE) and their salts, for example their trifluoroacetate salt, and or their racemic, enantiomeric and diastereoisomeric isomer forms:

In remark, in the following developed formula, a secondary amino moiety may be indifferently written —NH— or —N—.

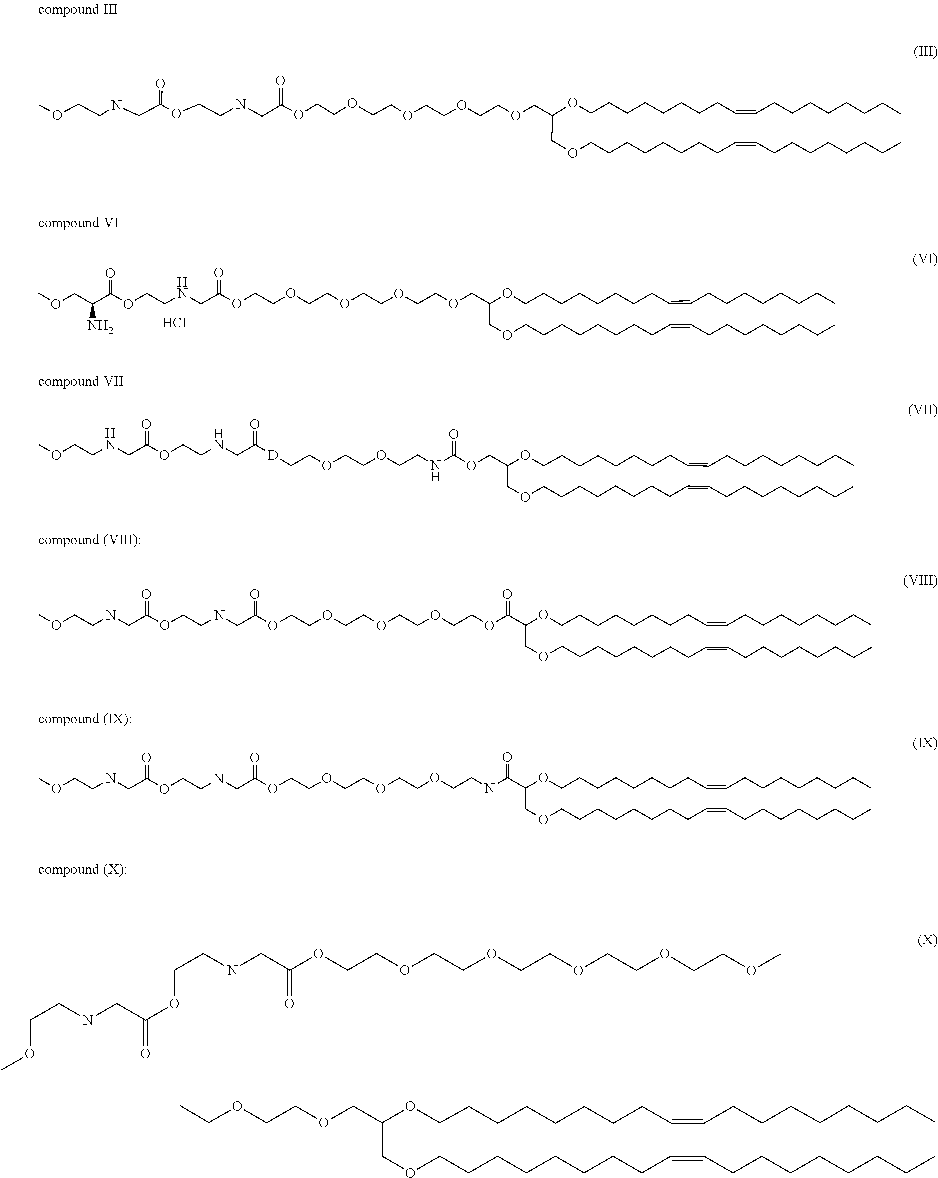

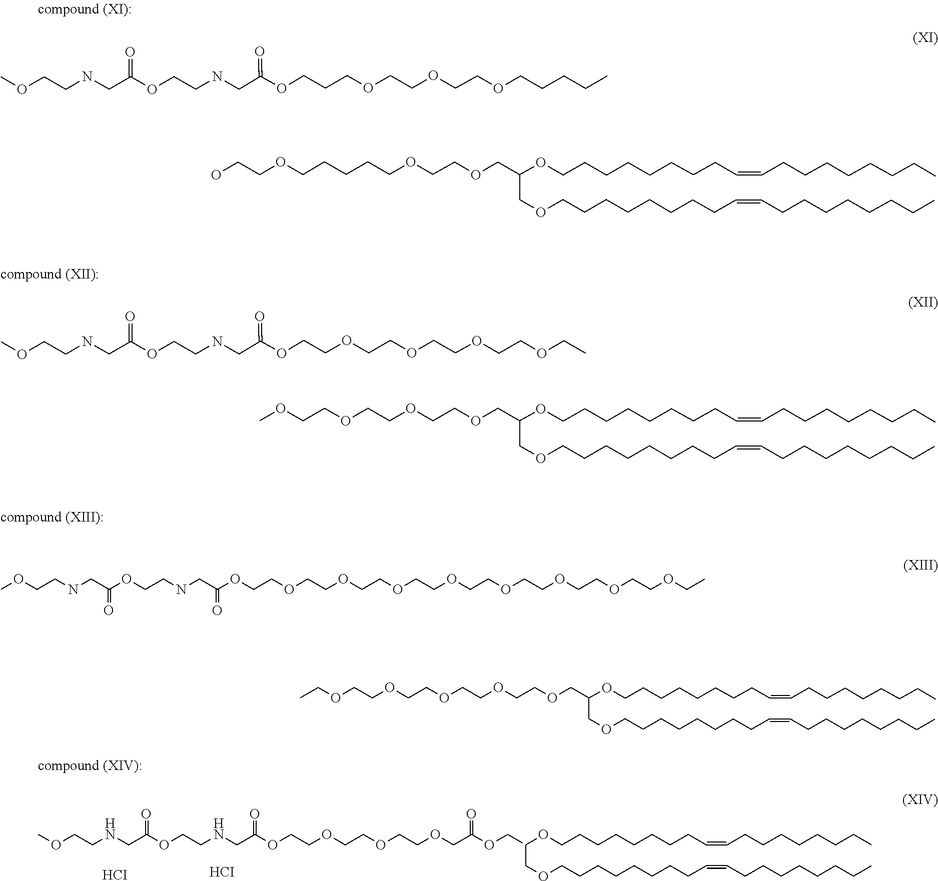

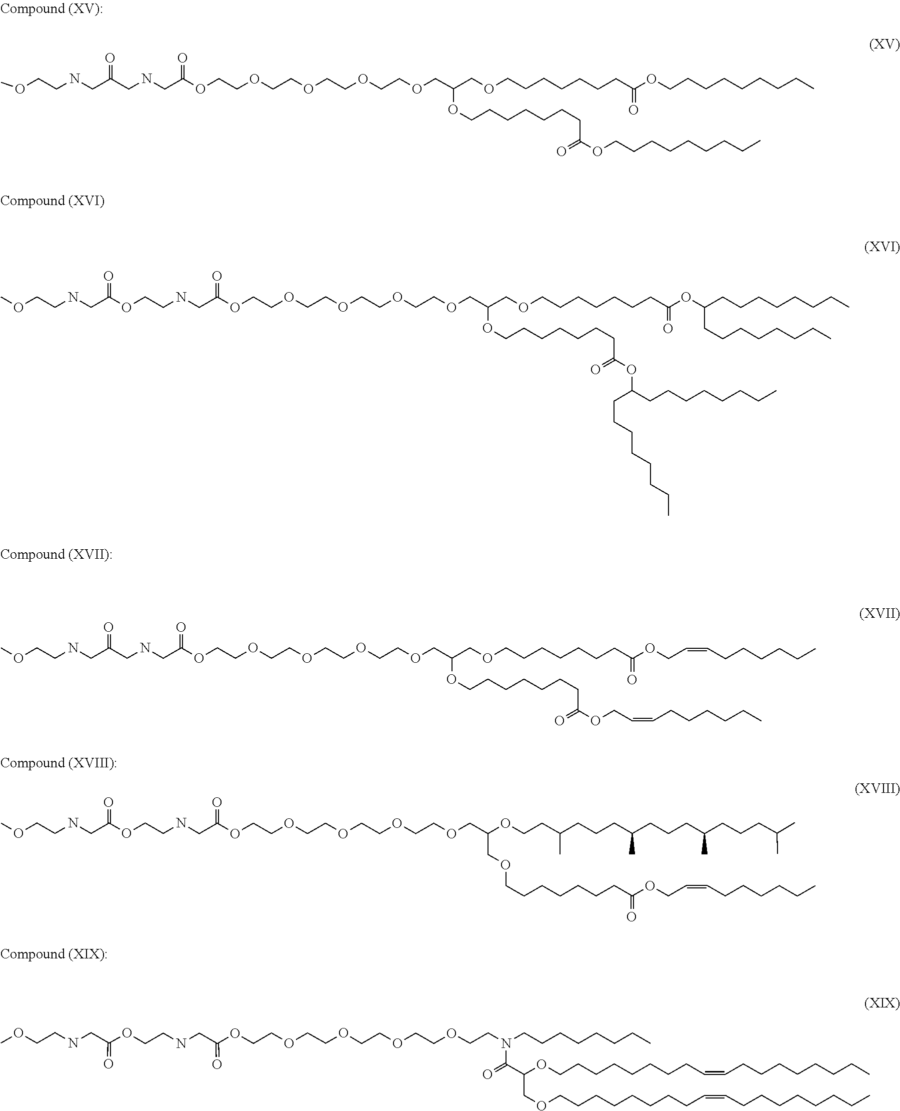

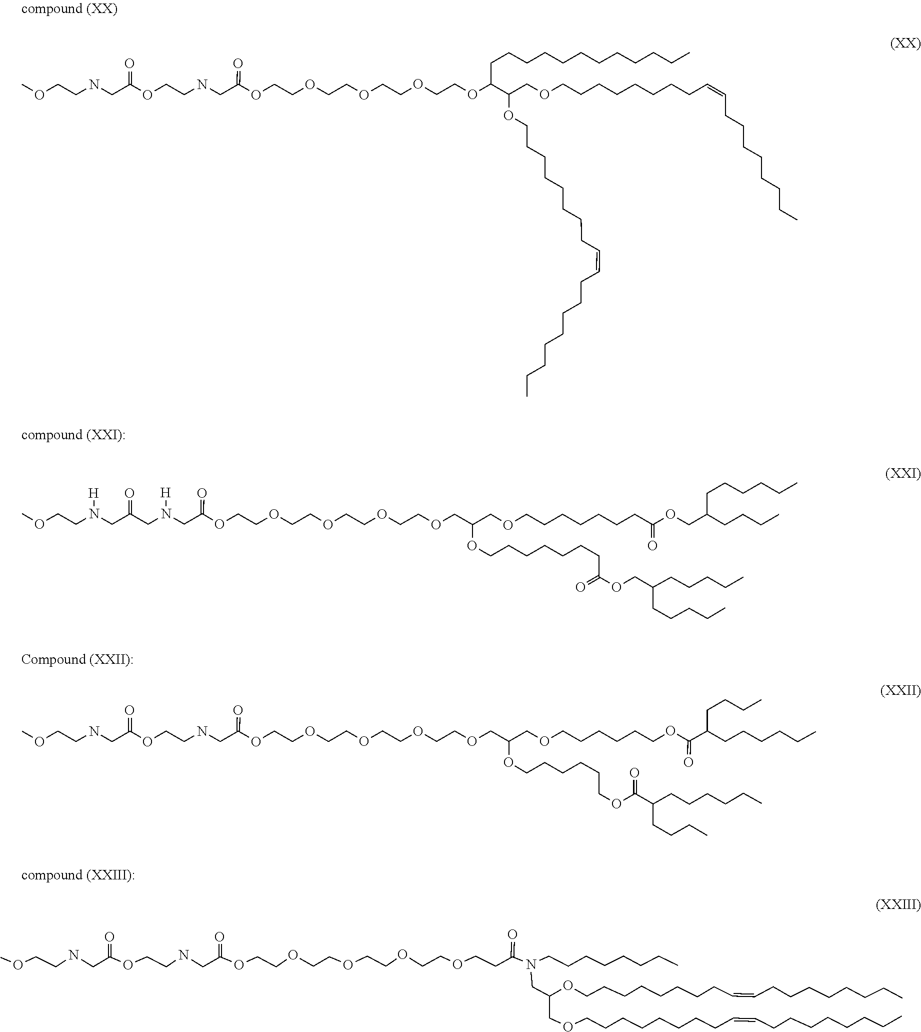

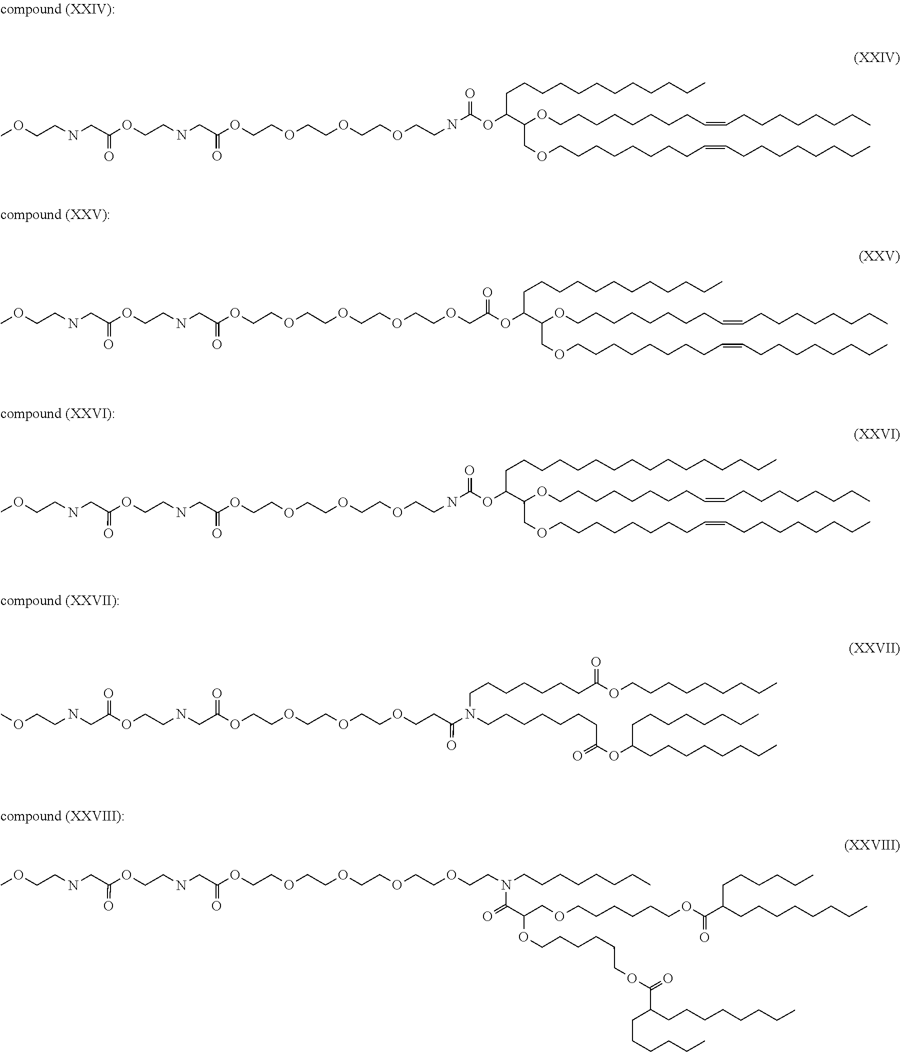

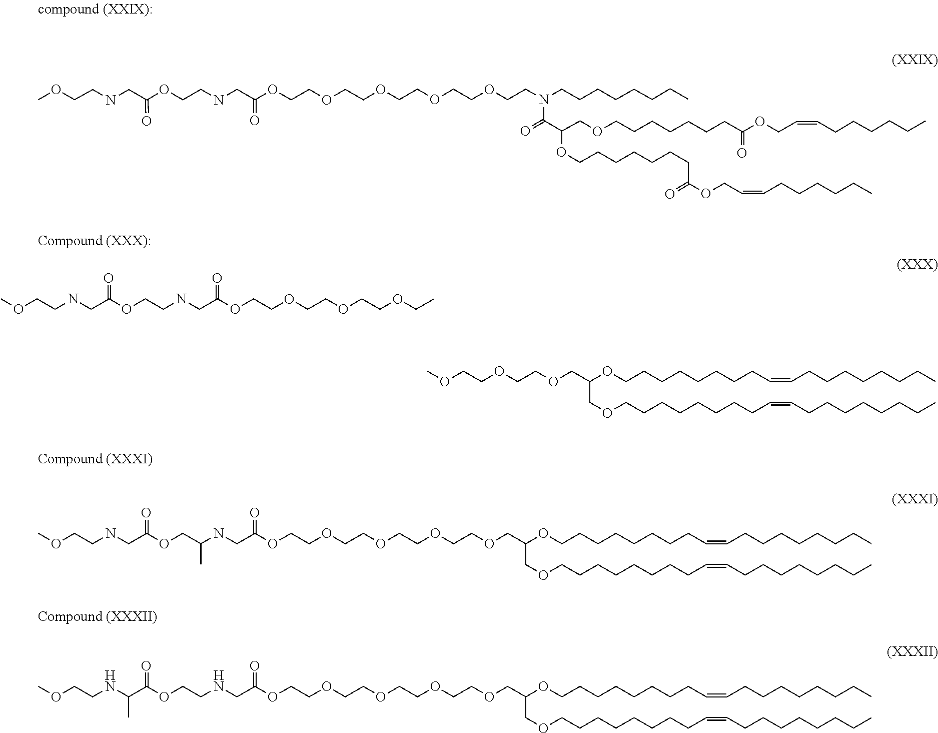

As shown in the Examples section, the compounds (II), (XVII), (XIX), (XXI), (XXII), (XXIV), (XXVII) and (XXVIII), are for example efficient for the formulation of stable LNPs capable of delivering a functional mRNA into target tissues after parenteral administrations and for the induction of the expression of a protein such as EPO or of an immune response in the case the delivered mRNA codes for an antigen.

Preparation of Lipidic Compounds

The lipidic compounds according to the disclosure can be prepared from readily commercially available or described in the literature starting materials using methods and procedures known from the skilled person.

For example, these lipidic compounds may be obtained by covalent coupling, between a precursor of the radical of formula (I) and a lipidic compound or derivative thereof having a terminal reactive group able to react with said precursor.

This terminal reactive group may be located directly on the end of the hydrophobic or lipophilic part of the lipidic compound to transform or on the end of a spacer arm already linked to the hydrophobic or lipophilic part of the lipid compound.

The choice of the convenient precursor of the radical of formula (I) intended to react with the lipidic compound for forming the expected covalent linkage is clearly within the competence of the man skilled in the art. The precursor only needs to have a group able to chemically react with the one of the lipidic compound for forming the covalent linking.

Regarding these starting compounds i.e., precursor of the radical of formula (I) and the lipidic compound or derivative thereof to transform they may be easily produced by a man skilled in the art, for example according to the methods of preparation submitted in the following examples.

As representative of a convenient precursor for a radical of formula (I) it can be cited the [2-[2(2-methoxyethyl)amino]acetyl]oxyethyl]amino]acetic acid or also named 2-[tert-butoxycarbonyl-[2-[2-[tert-butoxycarbonyl(2-methoxyethyl)amino]acetyl]oxyethyl]amino]acetic acid, and for example its protected form of formula

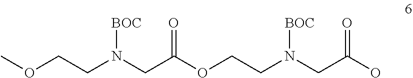

One specific approach for preparing such a precursor of the radical of formula (I) is depicted in Scheme 1 below.

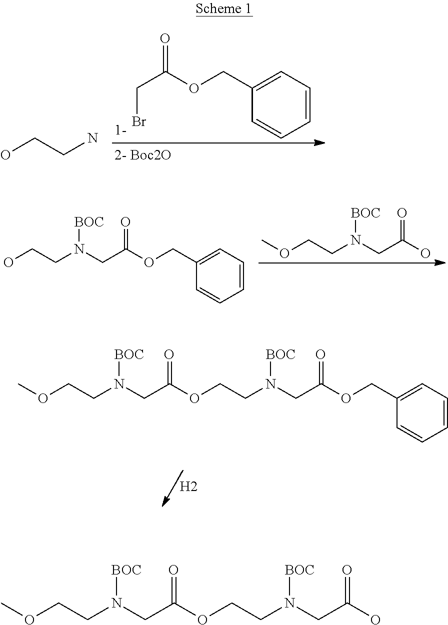

The covalent coupling between such a precursor may be further performed according to methods that are known to those skilled in the art in regard of the chemical nature of the reactive group of the precursor of the radical of formula (I) and the one on the lipidic compound or derivative thereof to be transformed.

Generally, the covalent linking may be formed for example by esterification, amidation, or carbamation.

One specific approach for this covalent coupling is depicted in Scheme 2 below for the synthesis of the trifluorocetate salt of the lipidic compound (III) wherein the coupling is obtained with an esterification reaction.

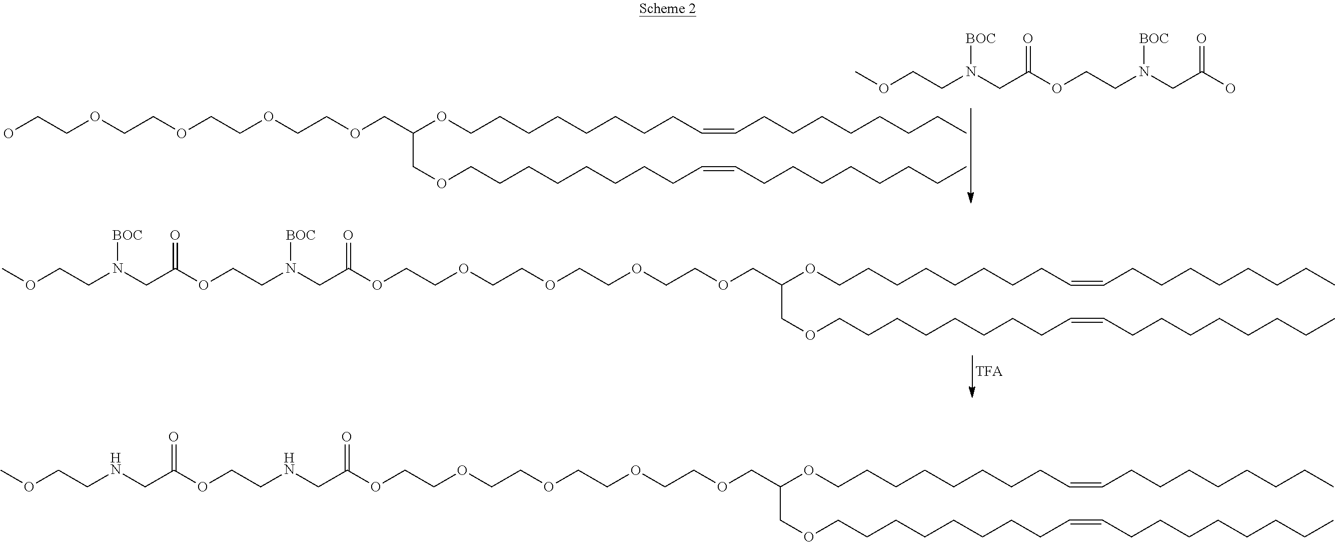

It will be appreciated that where typical or specific experimental conditions (i.e. reaction temperatures, time, moles of reagents, solvents etc.) are given, other experimental conditions can also be used unless otherwise stated. Optimum reaction conditions may vary with the specific reactants or solvents used, but such conditions can be determined by the person skilled in the art, using routine optimisation procedures.

The optional salification may be performed by conventional way like for example the one disclosed in the following examples.

The coupling reaction may advantageously be followed by subsequent steps of purifying and/or isolating of the final products obtained. Convenient methods of purification are detailed in the following examples. For example, the purification of compounds may be performed by preparative high-performance liquid chromatography (HPLC).

The instant disclosure will be better understood from the examples that follow, all of which are intended for illustrative purposes only and are not meant to limit the scope of the instant disclosure in any way.

Lipid Nanoparticles (LNPs) Manufacturing Methods

The present disclosure relates to methods for manufacturing lipid nanoparticles using the lipidic compounds as disclosed herein.

In a one embodiment, the disclosure relates to a method for manufacturing lipid nanoparticles containing a nucleic acid, wherein the method comprises at least the steps of:
a) solubilizing, in a water miscible organic solvent, at least one lipidic compound as disclosed herein,
b) mixing the organic solvent obtained at step a) with an aqueous solvent buffered at a pH ranging from about 3.0 to about 4.5 and comprising at least one nucleic acid, and
c) obtaining said lipid nanoparticles containing the nucleic acid in the aqueous solvent.

In one embodiment, a method for manufacturing lipid nanoparticles as disclosed herein may comprise at least steps of:
a) solubilizing, in a water miscible organic solvent, at least one lipidic compound as disclosed herein and at least one lipid selected from the group consisting of neutral lipids, steroid alcohols or esters thereof, and PEGylated lipids,
b) mixing the organic solvent obtained at step a) with an aqueous solvent comprising at least one nucleic acid, and
c) obtaining lipid nanoparticles containing the nucleic acid in the aqueous solvent.

The lipidic compound of the disclosure may be present in an amount sufficient to structure the lipid nanoparticles and to encapsulate any loads to be encapsulated. The amount of ionizable lipidic compound to be used in the lipid nanoparticles may be determined by the skilled person according to any known techniques and is adapted according to the nature and amount of the load, and nature and amount of other lipids susceptible to be present.

In one embodiment, the step a) further comprises solubilizing in the organic solvent at least one lipid selected from the group consisting of neutral lipids, steroid alcohols or esters thereof, and PEGylated lipids.

Neutral lipids, steroid alcohols or esters thereof, and PEGylated lipids suitable for the disclosure may be as described below.

In one embodiment, the step a) may further comprise solubilizing in the organic solvent at least one neutral lipid, at least one steroid alcohol or an ester thereof, and at least one PEGylated lipid, and wherein said lipidic compound, said neutral lipid, said steroid alcohol or an ester thereof, and said PEGylated lipid are present in the organic solvent at a molar amount of about 30% to about 70% of lipidic compound, of about 0% to about 50% of neutral lipid, of 20% to about 50% of steroid alcohol or an ester thereof, and of about 1% to about 15% of PEGylated, relative to the total amount of lipid and lipidic compound.

Useful water-miscible organic solvents may be any water-miscible organic solvent capable to solubilize the lipidic compound as disclosed herein and any other added lipids. As example of suitable organic solvents, one may cite ethanol or methanol, 1-propanol, isopropanol, t-butanol, THF, DMSO, acetone, acetonitrile, diglyme, DMF, 1-4 dioxane, ethylene glycol, glycerine, hexamethylphosphoramide, hexamethylphosphorous triamide. In one embodiment, the organic solvent may be ethanol and isopropanol.

Aqueous solvents usable at step b) include aqueous buffered solutions.

As examples of suitable aqueous buffered solution, one may mention acidic buffer, such as include citrate buffer, sodium acetate buffer, succinate buffer, borate buffer or a phosphate buffer. For example, an aqueous buffered solvent may be a citrate buffered solution or an acetate buffered solution.

The pH of the aqueous solvent may range from about 3.0 to about 4.5, for example from about 3.5 to about 4.5, and for example at about 4.0.

At step b), the organic and aqueous solvents may be mixed at a ratio organic solvent:aqueous solvent ranging from about 1:1 to about 1:6. In one embodiment, the ratio may range from about 1:2 to about 1:4, and for example may be a ratio of about 1:3.

According to one embodiment, the water miscible organic solvent and the aqueous solvent may be mixed at step b) at a flow rate ranging from about 0.01 ml/min to about 12 ml/min. In some embodiments, the flow rate may range from about 0.02 ml/min to about 10 ml/min, from about 0.5 ml/min to about 8 ml/min, from about 1 ml/min to about 6 ml/min, or at about 4 ml/min.

The step of mixing may be carried by any known method in the art. For instance, both solvents may be mixed with a T-tube or a Y-connector. Alternatively, the mixing may be carried out by laminar flow mixing with a microfluidic micromixer as described by Belliveau et al. (2012).

As indicated, the aqueous solvent at step b) comprises a nucleic acid. In one embodiment, a nucleic acid may encode at least one antigen. A suitable nucleic acid may be for example as detailed herein.

The method may further comprise a step of increasing the pH from acidic to neutral or slightly above neutral.

In a further embodiment, the method may comprise a step d) of increasing the pH of the aqueous solvent containing the lipid nanoparticles obtained at step c) at a pH ranging from about 5.0 to about 8.5, for example from about 5.5 to about 8.0, for example from about 6.0 to about 7.5, and for example from about 6.5 to about 7.0.

The step of increasing the pH may be carried by any known method in the art.

For example, the change in pH may carried by a dialyzing or diafiltration step.

According to one embodiment, step d) of the method of the disclosure may further comprise at least one step of dialyzing or diafiltrating the lipid nanoparticles. The dialysis or diafiltration step may be made against an aqueous solvent with a pH ranging from about 5.0 to about 8.5, for example from about 5.5 to about 8.0, for example from about 6.0 to about 7.5, and for example from about 6.5 to about 7.0.

The increase of the pH from acidic (i.e., from about 3.0 to about 4.5) to more neutral, or slightly above neutral, pH (i.e., from about 5.0 to about 8.5) advantageously results into the cleavage and self-rearrangement of the ionizable lipidic compounds as disclosed herein into lipidic compounds of formula (IV), (Va) or (Vb) as detailed below. That allows the lipid nanoparticles as disclosed herein to display a more neutral surface charge, which favors their distribution in the organism of the individuals into which the lipid nanoparticles are administered, to reach the targeted cells.

An aqueous solvent usable at step d) may further contain a carbohydrate to improve stabilization of the lipid nanoparticles and osmolarity of the solution. Suitable carbohydrate may be sucrose, mannitol, glucose, dextrose or trehalose. The carbohydrate may be present in an amount, relative to the total amount of the aqueous solvent, of about 5% to about 10%, and for example at about 8%.

According to another embodiment, step d) of the method as disclosed herein may comprise at least two steps of dialyzing the lipid nanoparticles. A first dialyzing step may be made against a similar aqueous solvent (similar in terms of pH and content) and may remove the organic solvent. A second dialysis step may be made against a different aqueous solvent (different in term of pH and possibly in term of content). In such case a pH of the dialyzing solution may range from about 5.5 to about 7.5, for example from about 6.0 to about 7.0, for example from about 6.5 to about 7.0, and for example at about 6.5. The dialyzing solution of the second dialysis may be a buffer solution, for example a phosphate buffer, a TRIS buffer, a Hepes buffer, a histidine buffer, or a glycine buffer. Osmolarity of the buffer may be adjusted with a salt, such as NaCl, or with a carbohydrate, such as glycerol, sucrose, mannitol, glucose, dextrose or trehalose.

In one embodiment, osmolarity is adjusted to reach a final osmolality close to 290 mOsmol/kg as to inject isotonic solution into the body.

Further to step c) and/or d), a method may comprise any further step suitable to harvest, purify, concentrate and/or sterilize the lipid nanoparticles to further formulate them as a pharmaceutical composition, for example as an immunogenic composition.

According to one embodiment, the disclosure relates to lipid nanoparticles obtainable according to a manufacturing method as disclosed herein.

According to another embodiment, the disclosure relates to a method for manufacturing a pharmaceutical composition comprising at least the steps of:
i) manufacturing at least one lipid nanoparticle according to the method as disclosed herein, and
ii) combining the lipid nanoparticles obtained at step i) with at least one pharmaceutically acceptable excipient or carrier.

According to another embodiment, the disclosure relates to a method for manufacturing an immunogenic composition comprising at least the steps of:
i) manufacturing at least one lipid nanoparticle according to the method as disclosed herein, said lipid nanoparticle containing at least one nucleic acid encoding for at least one antigen, and
ii) combining the lipid nanoparticles obtained at step i) with at least one pharmaceutically acceptable excipient or carrier.

The pharmaceutical and immunogenic compositions suitable for the disclosure are more detailed thereafter.

In one embodiment, the lipid nanoparticles of the disclosure may be manufactured with a lipidic compound of formula (I), (II) or (IIa), and for example of formula (III) to (XXXII), for example of formula (III), (VI), (XVII), (XIX), (XXI), (XXII), (XXIV), (XXVII) and (XXVIII), and for example of formula (III), (XIX) or (XXI), and for example with a lipidic compound of formula (III).

In another embodiment, the lipid nanoparticles as disclosed herein may be manufactured with a neutral lipid that is DSPC or DOPE, a steroid alcohol that is cholesterol, and a PEGylated lipid that is PEG-PE (PEG2000-PE) or DMG-PEG (DMG- PEG2000).

In another embodiment, the lipid nanoparticles as disclosed herein may be manufactured with a lipidic compound of formula (III) to (XXXII), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (III), (VI), (XVII), (XIX), (XXI), (XXII), (XXIV), (XXVII) and (XXVIII), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (III), (XIX) or (XXI), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (III), a neutral lipid that is DSPC, a steroid alcohol that is cholesterol, and a PEGylated lipid that is PEG-PE (PEG2000-PE).

Lipid Nanoparticles

Lipid Nanoparticles (LNPs)

The disclosure relates to lipid nanoparticles comprising at least lipidic compound of formula (IV):

HO-L (IV)
wherein L is a lipophilic or hydrophobic tail-group as described herein, for example a C₁₀ to C₅₅ lipophilic or hydrophobic tail-group, and at least one nucleic acid.

In some embodiments, the lipid nanoparticles as disclosed herein may comprise at least one lipidic compound of formula (Va) or (Vb):

HO—R₁ (Va) or

OH-A-R₁ (Vb)
wherein R₁ and A are as defined previously.

Further, the lipid nanoparticles as disclosed herein may comprise at least one lipid selected from the group consisting of neutral phospholipids or sphingolipids, steroid alcohols or esters thereof, and PEGylated lipids.

The lipidic compounds formula (IV), (Va) or (Vb) result from the cleavage and self-rearrangement of the ionizable lipidic compound as disclosed herein when the lipid nanoparticles are brought from acidic pH (i.e. from about 3.0 to about 4.5) to more neutral or slightly above neutral pH (i.e. from about 5.0 to about 8.5). Those lipidic compounds are neutral, non-ionizable compounds.

The lipid nanoparticles may have a diameter making them suitable for systemic, for example parenteral, or for intramuscular, intradermic, or subcutaneous administration. Typically, the lipid nanoparticles have a Z-average size of less than 600 nanometers (nm), for example of less than 400 nm.

In one embodiment, the LNPs have a Z-average size of less than 200 nm. Such size is advantageously compatible with sterile filtration and most appropriate for migration through the lymphatic vessels after intramuscular or subcutaneous administration. This size is also appropriate for intravenous administration, since larger particle injection could induce capillary thrombosis.

In some embodiments, the lipid nanoparticles may have a Z-average diameter size in the range of from about 20 nm to about 300 nm, for example from about 20 nm to about 250 nm, for example about 30 nm to about 200 nm, about 40 nm to about 180 nm, from about 60 nm to about 170 nm, from about 80 to about 160 nm, and from about 90 to about 150 nm. In one embodiment, the nanoparticles may have a diameter in the range of about 90 to about 150 nm.

The “Z-average size” of the lipid nanoparticles may be determined by dynamic light scattering (DLS). The Z-Average size or Z-Average mean used in dynamic light scattering is a parameter also known as the cumulants mean. It is the primary and most stable parameter produced by the technique. The Z-Average mean is defined as the ‘harmonic intensity averaged particle diameter’. A Z-average size may be measured with a zeta sizer Nano ZS light scattering instrument (Malvern Instruments). For accurate particle sizing with the Nano ZS, the viscosity of the buffer and the refractive index of the material had to be provided to the equipment software (PBS: v=1.02 cP, RI=1.45).

As minor variations in size may arise during the manufacturing process, a variation up to 20-30% of the stated measurement is acceptable and considered to be within the stated size. Alternatively, size may be determined by filtration screening assays. For example, a particle preparation is less than a stated size, if at least 90%, for example at least 95%, and for example at least 97% of the particles pass through a “screen-type” filter of the stated size.

The “polydispersity index” (PI) is a measurement of the homogeneous or heterogeneous size distribution of the individual lipid nanoparticles in a lipid nanoparticles mixture and indicates the breadth of the particle distribution in a mixture. The PI can be determined, for example, as described herein.

In one embodiment, the polydispersity index of the nanoparticles described herein as measured by dynamic light scattering is 0.5 or less, for example 0.4 or less, for example 0.3 or less, or even for example 0.2 or less.

In one embodiment, the lipid nanoparticles are colloidally stable in the sense that no, or substantially no, aggregation, precipitation or increase of size and polydispersity index as measured by dynamic light scattering may be observed over a given period of time, e.g. over at least two hours to over several months, for example at least 1, 2, 3, 4, 5, 6 or 12 months.

The lipid nanoparticles may comprise or encapsulate at least one nucleic acid.

The nucleic acid may be encapsulated in and/or adsorbed on an exterior surface of the lipid nanoparticles. The lipidic compound of formula (IV), (Va) or (Vb) may form a complex with and/or encapsulates the nucleic acid. Alternatively, the lipidic compound may be comprised in a vesicle encapsulating the nucleic acid.

The lipid nanoparticles have a global surface charge which is the sum of the negative and positive electric charges at the surface of the particles, and which is represented by the zeta potential. The zeta potential is the potential difference between the dispersion medium and the stationary layer of fluid attached to the dispersed particle. Zeta potential is widely used for quantification of the magnitude of the electrical charge at the double layer.

Zeta potential can be calculated using theoretical models and experimentally determined using electrophoretic mobility or dynamic electrophoretic mobility measurements. Electrophoresis may be used for estimating zeta potential of particulates. In practice, the zeta potential of a dispersion can be measured by applying an electric field across the dispersion. Particles within the dispersion with a zeta potential will migrate toward the electrode of opposite charge with a velocity proportional to the magnitude of the zeta potential. This velocity may be measured using the technique of the Laser Doppler Anemometer. The frequency shift or phase shift of an incident laser beam caused by these moving particles may be measured as the particle mobility, and this mobility may be converted to the zeta potential by inputting the dispersant viscosity and dielectric permittivity, and the application of the Smoluchowski theories. Electrophoretic velocity is proportional to electrophoretic mobility, which is the measurable parameter. There are several theories that link electrophoretic mobility with zeta potential.

Suitable systems such as the Nicomp 380 ZLS system or the Malvern nanoZS can be used for determining the zeta potential. Such systems usually measure the electrophoretic mobility and stability of charged particles in liquid suspension. These values are a predictor of the repulsive forces being exerted by the particles in suspension and are directly related to the stability of the colloidal system.

At pH neutral, the zeta potential of the lipid nanoparticles as disclosed herein is close to neutral. Indeed, at pH neutral, or slightly above neutral, (from 5.0/5.5 to 8.5), the ionizable, cleavable lipidic as disclosed herein has undergone a self-rearrangement resulting into the leave of the radical of formula (I) and to the remaining of the neutral, non-charged, hydrophobic or lipophilic tail group.

In one advantage, to have a zeta potential close to zero facilitates particle mobility in the body, reduces opsonization and augment access to target tissues.

In one embodiment, at pH from 6.0 to 7.5, the zeta potential of the lipid nanoparticles may be from about −3 mV to about +3 mV, for example from about −1 mV to about +1 mV, and for example from about −0.5 mV to about +0.5 mV.

The lipid nanoparticles described herein can be formed by adjusting, at the time of the preparation, a positive to negative charge, depending on the charge ratio of the ionizable lipidic compound as disclosed herein (cationic charges from the quaternary ammonium: N of the terminal radical of formula (I)) to the nucleic acid (anionic charges from the phosphate: P) and mixing the nucleic acid and the lipidic compound. The charges of the ionizable lipidic compound and of the nucleic acid are charges at a selected pH, such as a pH of the formulating process, which is from about 3.0 to about 4.5.

The +/−(N/P) charge ratio of the lipidic compound as disclosed herein to the nucleic acids can be calculated by the following equation. (+/−charge ratio)=[(cationic lipid amount (mol))*(the total number of positive charges in the cationic lipid)]: [(nucleic acid amount (mol))*(the total number of negative charges in nucleic acid)].

The nucleic acid amount and the lipidic compound amount can be easily determined by one skilled in the art in view of a loading amount upon preparation of the nanoparticles.

In one embodiment, the calculated charge ratio of positive charges to negative charges may range from about 1:1 to about 14:1, for example from about 2:1 to about 12:1, for example from about 4:1 to about 10:1, and for example from about 6:1 to about 8:1, and for example is about 6:1.

In one embodiment lipid nanoparticles as disclosed herein encapsulating a nucleic acid may have a Z-average size of about 80-180 nm and a calculated charge ratio N/P of about 6-12:1, for example of about 3-9:1.

In one embodiment, the lipid nanoparticles as disclosed herein may comprise at least one cleavable lipidic compound as disclosed herein.

Indeed, even when subjecting the lipid nanoparticles obtained according to the method as disclosed herein to a neutral pH, or slightly above neutral pH, (5.0/6.0 to 8.5) to cleave the ionizable cleavable cationic lipidic compound as disclosed herein, not all those compounds may be cleaved. For example, the lipidic compound located within the lipid nanoparticles, that in the core of the LNPs, may be protected from the change of pH and may not undergo to the cleaving process. The presence of the remaining cleavable cationic and/or ionizable lipidic compound as disclosed herein may be observed by known method in the art, such as TLC or HPLC analysis.

The lipid nanoparticles as disclosed herein may further comprise at least one lipid selected from the group consisting of neutral lipids, steroid alcohols or ester thereof, and PEGylated lipids.

Neutral Lipids

A composition or lipid nanoparticles as disclosed herein may include a neutral lipid. The presence of neutral lipids may improve structural stability of the lipid nanoparticles. The neutral lipid can be appropriately selected in view of the delivery efficiency of nucleic acid.

The neutral lipids are distinct from the lipidic compound of formula (IV), (Va) or (Vb). Neutral lipids are either not ionizable or are neutral zwitterionic compounds at a selected pH.

Neutral lipids useful for the disclosure may be selected from the group consisting of phosphatidylcholines, phosphatidylethanolamines, sphingomyelins, and ceramides.

Phosphatidylcholines and phosphatidylethanolamines are zwitterionic lipids. Sphingomyelins and ceramides are not ionizable lipids.

As examples of phosphatidylcholines useful for the disclosure, one may mention DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine).

As examples of phosphatidylethanolamines useful for the disclosure one may mention DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DSPE (1,2-distearoyl-s/i-glycero-3-phosphoethanolamine), DLPE (1,2-dilauroyl-SM-glycero-3-phosphoethanolamine), 16-O-monomethyl PE, 16-O-dimethyl PE, 18-1-trans PE, or 1-stearoyl-2-oleoyl-phosphatidyethanolamine (SOPE).

A neutral lipid may be selected from the group consisting of phosphatidylcholines, such as DSPC, DPPC, DMPC, POPC, DOPC; phosphatidylethanolamines, such as DOPE, DPPE, DMPE, DSPE, DLPE; sphingomyelins; and ceramides.

In one embodiment, a neutral lipid suitable for the disclosure may be DSPC, DOPC, and DOPE, and for example may be DSPC or DOPE.

Neutral lipids may be present at step a) of the method for formulating the lipid nanoparticles as disclosed herein in a molar amount ranging from about 0% to about 50%, for example from about 5% to about 45%, for example from about 8% to about 40%, and for example from about 10% to about 30% relative to the total molar amount of the lipid and lipidic compound as disclosed herein.

Neutral lipids may be present in the lipid nanoparticles as disclosed herein in a molar amount ranging from about 0% to about 50%, for example from about 5% to about 45%, for example from about 8% to about 40%, and for example from about 10% to about 30% relative to the total molar amount of the lipid and lipidic compound of formula (IV), (Va) or (Vb) which may be present in the lipid nanoparticles.

Neutral lipids may be present in lipid nanoparticles as disclosed herein in a molar ratio lipidic compound of formula (IV), (Va) or (Vb):neutral lipid which may range from about 70:1 to about 1:2, for example from about 30:1 to about 1:1, for example from about 15:1 to about 2:1, for example from about 10:1 to about 4:1, and for example is about 5:1.

Steroid Alcohols or Esters Thereof

A composition or lipid nanoparticles as disclosed herein may include a steroid alcohol (or sterol) or an ester thereof. The presence of sterol or an ester of sterol may improve structural stability of the lipid nanoparticles.

Sterols or steroid alcohols useful for the disclosure may be selected from the group consisting of cholesterol or its derivatives, ergosterol, desmosterol (36-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3B-ol).

Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ saturated or unsaturated, linear or branched, alkyl group, for example a C₂-C₁₈, for example a C₄-C₁₆, for example C₈-C₁₂ saturated or unsaturated, linear or branched, alkyl group, In other embodiments, the carboxylic acid may be a fatty acid. For example, a fatty acid may be caprylic acid, capric acid, lauric acid, stearic acid, margaric acid, oleic acid, linoleic acid, or arachidic acid.

In one embodiment, an ester of sterol suitable for the disclosure may be a cholesteryl ester.

Esters of sterol or of steroid alcohol useful for the disclosure may be selected from the group consisting of cholesteryl margarate (cholest-5-en-3β-yl heptadecanoate), cholesteryl oleate, and cholesteryl stearate.

Sterols or steroid alcohols or esters thereof useful for the disclosure may be selected from the group consisting of cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol, dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-36-ol), campestanol (5a-campestan-3b-ol), 24-methylene cholesterol (5,24(28)-cholestadien-24-methylen-3B-ol), cholesteryl margarate (cholest-5-en-3β-yl heptadecanoate), cholesteryl oleate, and cholesteryl stearate.

Alternatively, a sterol useful for the disclosure may be a cholesterol derivative such as an oxidized cholesterol.

Oxidized cholesterols suitable for the disclosure may be 25-hydroxycholesterol, 27-hydroxycholesterol, 20-hydroxycholesterol, 6-keto-5α-hydroxycholesterol, 7-keto-cholesterol, 7β,25-hydroxycholesterol and 7β-hydroxycholesterol. For example, oxidized cholesterols may be 25-hydroxycholesterol and 20α-hydroxycholesterol, and for example it may be 20α-hydroxycholesterol.

In one embodiment, a sterol or steroid alcohol, or ester thereof, suitable for the disclosure may be cholesterol, a cholesteryl ester, or a cholesterol derivative, for example an oxidized cholesterol. In one embodiment, a sterol or steroid alcohol, or ester thereof, suitable for the disclosure may be cholesterol or a cholesteryl ester, and for example may be cholesterol.

Sterols or steroid alcohols, or esters thereof, may be present at step a) of the method for formulating the lipid nanoparticles as disclosed herein in molar amount ranging from about 0 to about 60%, for example from about 10% to about 50%, and for example from about 20% to about 50% relative to the total molar amount of the lipid and ionizable lipidic compound as disclosed herein.

Sterols or steroid alcohols, or esters thereof, may be present in the lipid nanoparticles as disclosed herein in molar amount ranging from about 0 to about 60%, for example from about 10% to about 50%, and for example from about 20% to about 50% relative to the total molar amount of the lipid and lipidic compound of formula (IV), (Va) or (Vb) which may be present in the lipid nanoparticles.

Sterols or steroid alcohols, or esters thereof, may be present in lipid nanoparticles as disclosed herein in a molar ratio lipidic compound of formula (IV), (Va) or (Vb):steroid alcohol, or ester thereof, which may range from about 4:1 to about 1:2, for example from about 3.5:1 to about 1:1.8, for example from about 2:1 to about 1:1.5, for example from about 1.5:1 to about 1:1.2, and for example is about 1.3:1 to about 1:1.3.

PEGylated Lipids

A composition or lipid nanoparticles as disclosed herein may include a PEGylated (or PEG-) lipid.

Contemplated PEG-modified lipids include, but are not limited to, a polyethylene glycol chain of up to 5 kDa in length covalently attached to a lipid with alkyl chain(s) of C₆-C₂₀ length. The addition of PEG-modified lipids to a composition of lipid nanoparticles as disclosed herein may prevent complex aggregation and may also provide a means for increasing circulation lifetime and increasing the delivery of the composition or lipid nanoparticles to the target cells.

A suitable PEGylated lipid may be, for example, a pegylated diacylglycerol (PEG-DAG), such as 1-(monomethoxy-polyethyleneglycol)-2,3-dimyristoylglycerol (DMG-PEG), a pegylated phosphatidylethanoloamine (PEG-PE), a PEG succinate diacylglycerol (PEG-S-DAG) such as 4-0-(2′,3′-di(tetradecanoyloxy)propyl-l-0-(co-methoxy(polyethoxy) ethyl)butanedioate (PEG-S-DMG), a pegylated ceramide (PEG- cer), or a PEG dialkoxypropylcarbamate, such as w-methoxy(polyethoxy)ethyl-N-(2,3-di(tetradecanoxy) propyl)carbamate, 2,3-di(tetradecanoxy)propyl-N-(co-methoxy(polyethoxy)ethyl) carbamate, or mPEG-N,N-ditetradecylacetamide (also known as 2-[(polyethylene glycol)-2000]-N,N-ditetradecylacetamide or ALC-0159).

In one embodiment, a PEGylated lipid suitable for the disclosure may be selected from the group consisting of PEG-DAG, DMG-PEG, PEG-PE, PEG-S-DAG, PEG-S-DMG, PEG-cer, or mPEG-N,N-ditetradecylacetamide, or a PEG-dialkyoxypropylcarbamate.

For example, a PEGylated lipid suitable for the disclosure may be DMG-PEG, PEG-PE, or mPEG-N,N-ditetradecylacetamide.

In some embodiment, a PEGylated lipid suitable for the disclosure may be DMG-PEG or PEG-PE.

In some embodiment, a PEGylated lipid suitable for the disclosure may be mPEG-N,N-ditetradecylacetamide.

PEGylated lipid may be present at step a) of the method for formulating the lipid nanoparticles as disclosed herein in molar amount ranging from about 1% to about 10%, for example from about 1% to about 5%, and for example from about 1% to about 3.5% relative to the total molar amount of the lipid and ionizable lipidic compound.

PEGylated lipid may be present in the lipid nanoparticles as disclosed herein in molar amount ranging from about 1% to about 10%, for example from about 1% to about 5%, and for example from about 1% to about 3.5% relative to the total molar amount of the lipid and lipidic compound of formula (IV), (Va) or (Vb) which may be present in the lipid nanoparticles.

PEGylated lipid and lipidic compound of formula (IV), (Va) or (Vb) may be present in the lipid nanoparticles in a molar ratio ionizable lipidic compound to PEGylated lipid from about 70:1 to about 4:1, for example from about 40:1 to about 10:1, for example from about 35:1 to about 15:1, and for example is about 33:1 or about 14:1.

In one embodiment, lipid nanoparticles may comprise, further to the lipidic compound of formula (IV), (Va) or (Vb), at least one neutral lipid, at least one steroid alcohol, or an ester thereof, and at least one PEGylated lipid.

The neutral lipids, the steroid alcohol, or ester thereof, and the PEGylated lipids may be as described herein.

In one embodiment, the lipid nanoparticles described herein may comprise a lipidic compound of formula (IV), (Va) or (Vb), a neutral lipid, a steroid alcohol, or an ester thereof, and a PEGylated lipid in a molar amount of about 30% to about 70% of lipidic compound, of about 0% to about 50% of neutral lipid, of 20% to about 50% of steroid alcohol or an ester thereof, and of about 1% to about 15% of PEGylated, relative to the total amount of lipid and lipidic compound.

In one embodiment, the lipid nanoparticles described herein may comprise a lipidic compound of formula (IV), (Va) or (Vb), a neutral lipid, a steroid alcohol or an ester thereof, and a PEGylated lipid in a molar amount of about 30% to about 60% of lipidic compound, of about 5% to about 30% of neutral lipid, of about 30% to about 48% of steroid alcohol or an ester thereof, and of about 1.5% to about 5% of PEGylated, relative to the total amount of lipid and lipidic compound.

In one embodiment, the lipid nanoparticles described herein may comprise a lipidic compound of formula (IV), (Va) or (Vb), a neutral lipid, a steroid alcohol or an ester thereof, and a PEGylated lipid in a molar amount of about 35% to about 50% of lipidic compound, of about 10% to about 16% of neutral lipid, of about 38.5% to about 46.5% of steroid alcohol or an ester thereof, and of about 1.5% of PEGylated, relative to the total amount of lipid and lipidic compound.

As one embodiment, the lipid nanoparticles as disclosed herein may comprise about 35% of lipidic compound of formula (IV), (Va) or (Vb), about 16% of neutral lipid, about 46.5% of steroid alcohol, or an ester thereof, and of about 1.5% of PEGylated, relative to the total amount of lipid and lipidic compound.

As another embodiment, the lipid nanoparticles as disclosed herein may comprise about 50% of lipidic compound of formula (IV), (Va) or (Vb), about 10% of neutral lipid, about 38.5% of steroid alcohol or an ester thereof, and of about 1.5% of PEGylated, relative to the total amount of lipid and lipidic compound.

In one embodiment, the molar ratio of the lipidic compound of formula (IV), (Va) or (Vb) and of the neutral lipid, the steroid alcohol or an ester thereof, and the PEGylated lipid may be of about 35/16/46.5/1.5, of about 50/10/38.5/1.5, of about 57.2/7.1/34.3/1.4, of about 40/15/40/5, of about 50/10/35/4.5/0.5, of about 50/10/35/5, of about 40/10/40/10; of about 35/15/40/10, of about 52/13/30/5.

In one embodiment, the molar ratio of the lipidic compound of formula (IV), (Va) or (Vb) and of the neutral lipid, the steroid alcohol, or an ester thereof, and the PEGylated lipid may be of about 35/16/46.5/1.5 or about 50/10/38.5/1.5.

Nucleic Acids

The lipid nanoparticles as disclosed herein may comprise at least one, anionic or polyanionic, therapeutic agent. A therapeutic agent suitable for the disclosure may be a nucleic acid.

A nucleic acid according to the disclosure may be a deoxyribonucleic acid (DNA) or a ribonucleic acid (RNA), for example RNA, for example an in vitro transcribed RNA (IVT RNA) or synthetic RNA.

Nucleic acids according to the disclosure include genomic DNA, cDNA, mRNA, recombinantly produced and chemically synthesized molecules. A nucleic acid may be in the form of a molecule which is single stranded or double stranded and linear or closed covalently to form a circle. A nucleic can be employed for introduction into, i.e. transfection of, cells, for example, in the form of RNA which can be prepared by in vitro transcription from a DNA template. The RNA can moreover be modified before application by stabilizing sequences, capping, and polyadenylation.

A nucleic acid may be of eukaryotic or prokaryotic origin, and for example of human, animal, plant, bacterial, yeast or viral origin and the like. It may be obtained by any technique known to persons skilled in the art and for example by screening libraries, by chemical synthesis or alternatively by mixed methods including chemical or enzymatic modification of sequences obtained by screening libraries. They may be chemically modified.

Nucleic acids may be comprised in a vector. Vectors are known to the skilled person and may include plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or PI artificial chromosomes (PAC). The vectors include expression as well as cloning vectors. Expression vectors comprise plasmids as well as viral vectors and generally contain a desired coding sequence and appropriate DNA sequences necessary for the expression of the operably linked coding sequence in a specific host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment, the nucleic acid may be selected from a group consisting of a double stranded RNA (dsRNA); a single stranded RNA (ssRNA); a double stranded DNA (dsDNA); a single stranded DNA (ssDNA); and combinations thereof.

In one embodiment, the nucleic acid may be selected from a group consisting of messenger RNA (mRNA); an antisense oligonucleotide (ASO); a short interference RNA (siRNA): a self-amplifying RNA (saRNA); a micro RNA (miRNA); a small nuclear RNA (snRNA); a small nucleolar RNA (snoRNA); self-amplifying RNA (saRNA); a plasmid DNA (pDNA); closed-ended DNA (ceDNA), and combinations thereof.

In another embodiment, the nucleic acid may be selected from a group consisting of messenger RNA (mRNA); an antisense oligonucleotide (ASO); a short interference RNA (siRNA): a self-amplifying RNA (saRNA); a micro RNA (miRNA); a plasmid DNA (pDNA); and combinations thereof.

In another embodiment, the nucleic acid may be selected from a group consisting of messenger RNA (mRNA); a short interference RNA (siRNA): a self-amplifying RNA (saRNA); a micro RNA (miRNA); and combinations thereof.

In another embodiment, the nucleic acid may be a messenger RNA (mRNA).

In one embodiment, the nucleic acid is an mRNA. In certain embodiments, the nucleic acid may be a RNA encoding a protein or an enzyme. Such polynucleotides may be used as a therapeutic that is capable of being expressed by target cells to facilitate the production of a functional enzyme or protein. For example, in certain embodiments, upon the expression of at least one polynucleotide by target cells the production of a functional enzyme or protein in which a cell or an individual is deficient.

The target cells are cells to which a composition or lipid nanoparticles as disclosed herein are to be directed or targeted. The target cells may comprise a specific tissue or organ. In some embodiments, the target cells may be hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, antigen presenting cells such as dendritic cells, reticulocytes, leukocytes, granulocytes and tumor cells.

mRNA

The term “RNA” relates to a molecule which comprises ribonucleotide residues and for example being entirely or substantially composed of ribonucleotide residues. “Ribonucleotide” relates to a nucleotide with a hydroxyl group at the 2′-position of a j-D-ribofuranosyl group.

The term includes double stranded RNA, single stranded RNA, isolated RNA such as partially purified RNA, essentially pure RNA, synthetic RNA, or recombinantly produced RNA.

These may be sequences of natural or artificial origin, and for example mRNA (messenger RNA), tRNA (transfer RNA), rRNA (ribosomal RNA), siRNA (silencing RNA), miRNA (micro RNA), mtRNA (mitochondrial RNA), shRNA (short hairpin RNA), tmRNA (transfer-messenger RNA), vRNA (viral RNA), single-stranded, double-stranded and/or base-paired RNA (ssRNA; dsRNA and bpRNA respectively), blunt-ended RNA or not, mature and immature mRNAs, coding and non-coding RNAs, hybrid sequences or synthetic or semisynthetic sequences of oligonucleotides, modified or otherwise, and mixtures thereof.

Accordingly, these may be messenger RNAs (mRNA), which includes mature and immature mRNAs, such as precursor mRNAs (pre-mRNA) or heterogeneous nuclear mRNAs (hnRNA) and mature mRNAs. Thus, RNA molecules as disclosed herein also encompass monocistronic and polycistronic messenger RNAs.

For the sake of clarity, a mRNA encompasses any coding RNA molecule, which may be translated by a eukaryotic host into a protein. A coding RNA molecule generally refers to a RNA molecule comprising a sequence coding for a protein of interest and which may be translated by the eukaryotic host, said sequence starting with a start codon (ATG) and for example terminated by a stop codon (i.e. TAA, TAG. TGA).

A RNA may be a naturally occurring RNA or a modified RNA that differs from naturally occurring RNA by the addition, deletion, substitution and/or alteration of at least one nucleotide. Such alterations can include addition of non-nucleotide material, such as to the end(s) of a RNA or internally, for example at least one nucleotide of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally occurring RNA.

In one embodiment, the RNA is a mRNA (messenger RNA). A mRNA may be a transcript which may be produced using DNA as template and encodes a peptide or a protein. mRNA typically comprises 5′Cap, a 5′ non translated region (5-UTR), a protein or a peptide coding region and a 3′ non translated region (3′-UTR), and a 3′ polyA tail. mRNA has a limited halftime in cells and in vitro. For example, a mRNA is produced by in vitro transcription using a DNA template. Alternatively, the RNA may be obtained by chemical synthesis. The in vitro transcription methodology is known to the skilled person. For example, there is a variety of in vitro transcription kits commercially available.

The RNA may be in vitro synthesized in a cell-free system, using appropriate cell extracts and an appropriate DNA template. For example, cloning vectors are applied for the generation of transcripts. The promoter for controlling transcription can be any promoter for any RNA polymerase. Some examples of RNA polymerases are the T7, T3, and SP6 RNA polymerases. A DNA template for in vitro transcription may be obtained by cloning of a nucleic acid, for example a cDNA, and introducing it into an appropriate vector for in vitro transcription. The cDNA may be obtained by reverse transcription of RNA. For example, cloning vectors are used for producing transcripts which generally are designated transcription vectors.

In one embodiment, the RNA may encode for a protein or a peptide. That is, if present in the appropriate environment, for example within a cell, such as an antigen-presenting cell, for example a dendritic cell, the RNA can be expressed to produce a protein or peptide it encodes.

The stability and translation efficiency of a RNA may be modified as required. A modification of a RNA within as disclosed herein refers to any modification of RNA which is not naturally present in said RNA.

According to a general embodiment, a mRNA as disclosed herein may comprise or consist of the following general formula:

[5′Cap]w-[5′UTR]x-[Gene of Interest]-[3′UTR]y-[PolyA]z
wherein [5′UTR] and [3′UTR] are untranslated regions (UTR),
wherein [5′UTR] contains a Kozak sequence,
wherein [Gene of Interest] is any gene coding for a protein of interest,
wherein [5′Cap] contains a methyl guanine nucleotide linked to mRNA via a 5′ to 5′ linkage,
wherein [PolyA] is a poly(A) tail, and
wherein w, x, y, and z, are identical or different, and equal to 0 or 1.

According to one embodiment, a mRNA as disclosed herein may consist of the following general formula:

[5′Cap]-[5′UTR]-[Gene of Interest]-[3′UTR]-[PolyA]
wherein [5′UTR] and [3′UTR] are untranslated regions,
wherein [5′UTR] contains a Kozak sequence,
wherein [Gene of Interest] is any nucleic acid coding for a protein of interest,
wherein [5′Cap] contains a methyl guanine nucleotide linked to mRNA via a 5′ to 5′ linkage, and
wherein [PolyA] is a poly(A) tail.

It is reminded that a Kozak sequence refers to a sequence, which is generally a consensus sequence, occurring on eukaryotic mRNAs and which plays a major role in the initiation of the translation process. Kozak sequences and Kozak consensus sequences are well known in the art.

It is also reminded that a poly(A) tail consists of multiple adenosine monophosphates that is well known in the art. A poly(A) tail is generally produced during a step called polyadenylation that is one of the post-translation modifications which generally occur during the production of mature messenger RNAs; such poly(A) tail contribute to the stability and the half-life of said mRNAs, and can be of variable length. For example, a poly(A) tail may be equal or longer than 10 A nucleotides, which includes equal or longer than 20 A nucleotides, which includes equal or longer than 100 A nucleotides, and for example about 120 A nucleotides.

The [3′UTR] does not express any proteins. The purpose of the [3′UTR] is to increase the stability of the mRNA. According to a one embodiment, the a-globin UTR is chosen because it is known to be devoid of instability.

Advantageously, the sequence corresponding to the gene of interest may be codon-optimized in order to obtain a satisfactory protein production within the host which is considered.

RNA molecules as disclosed herein may be of variable length. Thus, they may be short RNA molecules, for instance RNA molecules shorter than about 100 nucleotides, or long RNA molecules, for instance longer than about 100 nucleotides, or even longer than about 300 nucleotides.

RNA, such as mRNAs, may encompass synthetic or artificial RNA molecules, but also naturally occurring RNA molecules.

According to the disclosure, a RNA molecule, such as a mRNA, may encompasse the following species:
(i) capped unmodified RNA molecule;
(ii) capped modified RNA molecule;
(iii) uncapped unmodified RNA molecule;
(iv) uncapped modified RNA molecule.

Capped and Uncapped RNA Molecules

According to a most general embodiment, a “capped RNA molecule” refers to a RNA molecule of which the 5′end is linked to a guanosine or a modified guanosine, for example a 7-methylguanosine (m⁷G), connected to a 5′ to 5′ triphosphate linkage or analog. This definition is commensurate with the most widely-accepted definition of a 5′cap, for example of a naturally-occurring and/or physiological cap.

In the sense of the disclosure, “cap analogs” include caps which are biologically equivalent to a 7-methylguanosine (m⁷G), connected to a 5′ to 5′ triphosphate linkage, and which can thus be also substituted without impairing the protein expression of the corresponding messenger RNA in the eukaryotic host.

As example of caps, one may mention m⁷GpppN, m⁷GpppG, m⁷GppₛpG, m⁷GppₛpₛpG, m⁷GppₛpₛpG, m⁷Gppppm⁷G, m₂^{7′,3′-O}GpppG, m₂^{7′,2′-O}GpppG, m₂^{7′,2′-O}GppₛpₛG, or m₂^{7′,2′-O}GpppₛpₛG.

Examples of synthetic caps and/or cap analogs can be selected in a list consisting of: glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranos 1 nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Other examples of synthetic caps or cap analogs include ARCA cap analogs, N1-methyl-guanosine, 2′-fluoro-guanosine, 7-deaza-guanosine, 8-oxo-guanosine, 2-amino-guanosine, LNA-guanosine, and 2-azido-guanosine.

Of note, among synthetic caps, some of the above-mentioned caps are suitable as analogs, but not others which may on the contrary hinder protein expression. Such distinction is understood by the man skilled in the art.

For reference, and in a non-limitative manner, the structure of an Anti Reverse Cap Analog (ARCA) 3′-O-Me-m7G(5′)ppp(5′)G Cap analog is presented herebelow:

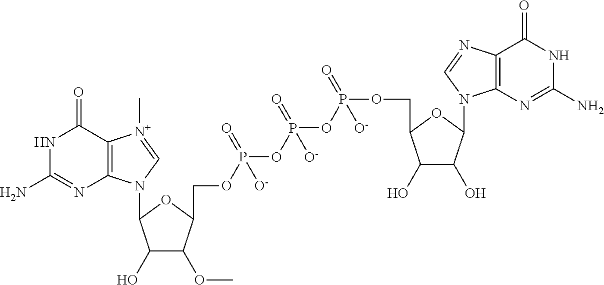 [SMILES: COC1C(COP(=O)([O-])OP(=O)([O-])OP(=O)([O-])OCC2OC(n3cnc4c(=O)[nH]c(N)nc43)C(O)C2O)OC(n2c[n+](C)c3c(=O)[nH]c(N)nc32)C1O]

The ARCA cap analog is, for instance, an example of cap analog used during in vitro transcription: it is a modified cap in which the 3′ OH group (closer to m⁷ G) is replaced with —OCH₃. However, 100% of the transcripts synthesized with ARCA at the 5′ end are translatable leading to a strong stimulatory effect on translation.

Providing a RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

An “uncapped RNA molecule” refers to any RNA molecule that does not belong to the definition of a “capped RNA molecule”.

Thus, according to a general embodiment, an “uncapped mRNA” may refer to a mRNA of which the 5′end is not linked to a 7-methylguanosine, through a 5′ to 5′ triphosphate linkage, or an analog as previously defined.

An uncapped RNA molecule, such as a messenger RNA, may be an uncapped RNA molecule having a (5′)ρρρ(5′), a (5′)ρρ(5′), a (5′)ρ(5′) or even a (5′)OH extremity. Such RNA molecules may be respectively abbreviated as 5′ρρρRNA; 5′ρρRNA; 5′ρRNA; 5′_{OH}RNA. For example, an uncapped RNA molecule as disclosed herein is a messenger 5′ρρρRNA.

Thus, when the RNA molecule is a single-stranded RNA molecule, it may be respectively abbreviated as _{5′ppp}ssRNA; _{5′pp}ssRNA; _{5′p}ssRNA; _{5′OH}ssRNA.

Thus, when the RNA molecule is a double-stranded RNA molecule, it may be respectively abbreviated as _{5′ppp}dsRNA; _{5′pp}dsRNA; _{5′p}dsRNA; _{5′OH}dsRNA.

In one embodiment, an uncapped mRNA as disclosed herein is an uncapped single-stranded mRNA.

According to one embodiment, an uncapped single-stranded mRNA may be an uncapped messenger _{5′ppp}ssRNA.

In a non-limitative manner, the first base of said uncapped RNA molecule may be either an adenosine, a guanosine, a cytosine, or an uridine.

Thus, an uncapped RNA molecule may be an uncapped RNA molecule having a (5′)ppp(5′), a (5′)pp(5′), a (5′)p(5′) or even a blunt-ended 5′ guanosine extremity.

In one embodiment of the disclosure, the RNA may not have uncapped 5′-triphosphates. Removal of such uncapped 5′-triphosphates can be achieved by treating RNA with a phosphatase.

Modified and Unmodified RNA Molecules

The RNA may comprise further modifications. For example, a further modification of the RNA used in the present disclosure may be an extension or truncation of the naturally occurring poly(A) tail or an alteration of the 5′- or 3′-untranslated regions (UTR) such as introduction of an UTR which is not related to the coding region of said RNA, for example, the exchange of the existing 3-UTR with or the insertion of at least one, for example two copies of a 3-UTR derived from a globin gene, such as alpha 2-globin, alpha 1-globin, beta-globin, for example beta-globin, and for example human beta-globin.

Within the disclosure, a “modified RNA molecule” refers to a RNA molecule which contains at least one modified nucleotide, nucleoside or base, such as a modified purine or a modified pyrimidine. A modified nucleoside or base can be any nucleoside or base that is not A, U, C or G (respectively Adenosine, Uridine, Cytidine or Guanosine for nucleosides; and Adenine, Uracil, Cytosine or Guanine when referring solely to the sugar moiety).

Accordingly, an “unmodified RNA molecule” refers to any RNA molecule that is not commensurate with the definition of a modified RNA molecule.

In the sense of the disclosure, the terms “modified and unmodified” are considered distinctly from the terms “capped and uncapped”, as the latter specifically relates to the base at the 5′-end of a RNA molecule in the sense of the disclosure.

In one embodiment, a nucleic acid, for example a RNA, may comprise at least one modified nucleotide, for example a modified ribonucleotide. The presence of modified nucleotide may increase the stability and/or decrease cytotoxicity of the nucleic acid.

The term stability of RNA relates to the half-life of RNA, that is the period of time which is needed to eliminate half of the activity, amount, or number of molecules. In the context of the present disclosure, the half-life of a RNA is indicative for the stability of said RNA. The half-life of RNA may influence the duration of expression of the RNA. It can be expected that RNA having a long half-life will be expressed for an extended time period.

According to one embodiment, a “modified RNA molecule” refers to a RNA molecule, such as a mRNA, which contains at least one base or sugar modification as described above, and for example at least one base modification as described herein.

For example, in one embodiment, in a RNA suitable for the disclosure 5-methylcytidine may be substituted partially or completely, for example completely, for cytidine. Alternatively, or additionally, in one embodiment, it may be substituted partially or completely, for example completely, for uridine.

In a non-limitative manner, examples of modified nucleotides, nucleosides and bases are disclosed in WO 2015/024667A1.

Thus, a modified RNA molecule may contain modified nucleotides, nucleosides or bases, including backbone modifications, sugar modifications or base modifications.

A backbone modification in connection with the present disclosure includes modifications, in which phosphates of the backbone of the nucleotides contained in a RNA molecule as defined herein are chemically modified

A sugar modification in connection with the present disclosure includes chemical modifications of the sugar of the nucleotides of the RNA molecule as defined herein.

A base modification in connection with the present disclosure includes chemical modifications of the base moiety of the nucleotides of the RNA. In this context nucleotide analogues or modifications are for example selected from nucleotide analogues which are suitable for transcription and/or translation of the RNA molecule in an eukaryotic cell.

Sugar modifications may consist in replacement or modification of the 2′ hydroxy (OH) group, which can be modified or replaced with a number of different “oxy” or “deoxy” substituents.

Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises at least one of the atoms C, N, and O

The sugar group can also contain at least one carbon that possess the opposite stereochemical configuration than that of the corresponding carbon in ribose. Thus, a modified RNA can include nucleotides containing, for instance, arabinose as the sugar.

The phosphate backbone may further be modified and incorporated into the modified RNA molecule, as described herein. The phosphate groups of the backbone can be modified by replacing at least one of the oxygen atoms with a different substituent. Further, the modified nucleosides and nucleotides can include the full replacement of an unmodified phosphate moiety with a modified phosphate as described herein.

Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

The modified nucleosides and nucleotides, which may be incorporated into the modified RNA molecule, as described herein, can further be modified in the nucleobase moiety. For example, the nucleosides and nucleotides described herein can be chemically modified on the major groove face. In some embodiments, the major groove chemical modifications can include an amino group, a thiol group, an alkyl group, or a halo group.

For examples, the nucleotide analogues/modifications are selected from base modifications selected in a list consisting of: 2-amino-6-chloropurineriboside-5′-triphosphate, 2-aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, Ni-methyladenosine-5′-triphosphate, Ni-methylguanosine-5′-triphosphate, N6-methyladenosine-5-triphosphate, 06-methylguanosine-5′-triphosphate, pseudouridine-5′-triphosphate, or puromycin-5′-triphosphate, and xanthosine-5′-triphosphate.

In some embodiments, modified nucleosides may be selected from a list consisting of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridinei 4-methoxy-pseudouridine, and 4-methoxy-2-thio-pseudouridine.

In some embodiments, modified nucleosides and nucleotides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

Modified bases and/or modified RNA molecules are known in the art and are, for instance, further taught in Warren et al. (“Highly Efficient Reprogramming to Pluripotency and Directed Differentiation of Human Cells with Synthetic Modified mRNA”; Cell Stem Cell; 2010).

In view of the above, a modified base may be a modified purine base or a modified pyrimidine base.

In a non-limitative manner, examples of modified purine bases include modified adenosine and/or modified guanosine, such as hypoxanthine; xanthine; 7-methylguanine; inosine; xanthosine and 7-methylguanosine.

According to some embodiments, a modified RNA or mRNA molecule corresponds to a RNA for which each nucleoside corresponding to either Uridine, Cytidine, Adenosine and/or Ribothymidine is modified.

In a non-limitative manner, examples of modified pyrimidine bases include modified cytidine and/or modified uridine, such as 5,6-dihydrouracil; pseudouridine; 5-methylcytidine; 5-hydroxymethylcytidine; dihydrouridine and 5-methylcytidine.

In a non-limitative manner, a modified base as disclosed herein may be a modified uridine or cytidine, such a pseudouridine and 5-methylcytidine.

According to some embodiments, a modified RNA corresponds to a RNA for which at least one base corresponding to either U (for Uracile), C (for Cytosine), A (for Adenine) and/or T (for Thymine) is modified.

As example of modified bases, one may mention methyl-5 uridine (m5U), 2-thio-uridine (s2U), 2′-O-methyl-5 uridine (Ome5U), pseudouridine (Ψ), methyl-1 pseudouridine (m1Ψ), methyl-5 cytosine (m5C), 2′O-methyl-5 cytosine (Om5C), N6-methyl-adenosine (m6A), and Ni-methyl-adenosine (m6A).

According to some embodiments, a modified mRNA may comprise as modified bases 2′-O-methyl-5 uridine (Ome5U) or methyl-1 pseudouridine (mIT).

Capped and uncapped mRNAs, whether modified or unmodified, may also be obtained commercially.

RNA having an unmasked poly-A sequence is translated more efficiently than RNA having a masked poly-A sequence.

The term “poly(A) tail” or “poly-A sequence” relates to a sequence of adenyl (A) residues which typically is located on the 3′-end of a RNA molecule and “unmasked poly-A sequence” means that the poly-A sequence at the 3′ end of a RNA molecule ends with an A of the poly- A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA used according to the present disclosure, it may be modified so as to be present in conjunction with a poly-A sequence, for example having a length of 10 to 500, for example 30 to 300, for example 65 to 200 and for example 100 to 150 adenosine residues. In one embodiment the poly-A sequence has a length of approximately 120 adenosine residues. To further increase stability and/or expression of the RNA used according to the disclosure, the poly-A sequence can be unmasked.

In addition, incorporation of a 3′-non translated region (UTR) into the 3′-non translated region of a RNA molecule can result in an enhancement in translation efficiency. A synergistic effect may be achieved by incorporating two or more of such 3′-non translated regions. The 3′-non translated regions may be autologous or heterologous to the RNA into which they are introduced. In one embodiment the 3′-non translated region is derived from the human j-globin gene.

A combination of the above-described modifications, i.e. incorporation of a poly-A sequence, unmasking of a poly-A sequence and incorporation of at least one 3′-non translated region, has a synergistic influence on the stability of RNA and increase in translation efficiency.

In order to increase expression of the RNA used according to the present disclosure, it may be modified within the coding region, i.e. the sequence encoding the expressed peptide or protein, for example without altering the sequence of the expressed peptide or protein, so as to increase the GC-content to increase mRNA stability and to perform a codon optimization and, thus, enhance translation in cells.

It is understood that an uncapped RNA molecule may be either a modified RNA molecule or an unmodified RNA molecule.

Accordingly, a capped RNA molecule may be either a modified RNA molecule or an unmodified RNA molecule.

In one embodiment, a RNA molecule as disclosed herein is a messenger RNA (mRNA).

A RNA molecule as disclosed herein is for example an uncapped messenger RNA, either in a modified or in an unmodified form.

A RNA molecule as disclosed herein is for example a capped messenger RNA, either in a modified or in an unmodified form.

In a non-limitative manner, an uncapped RNA molecule, such as a messenger RNA may also be an uncapped RNA molecule having only naturally occurring bases.

According to the disclosure, a “naturally occurring base” relates to a base that can be naturally incorporated in vivo into a RNA molecule, such as a messenger RNA, by the host. Thus, a “naturally occurring base” is distinct from a synthetic base for which there would be not natural equivalent within said host. However, a “naturally-occurring base” may or may not be a modified base, as both terms shall not be confused in the sense of the disclosure.

An uncapped messenger RNA may also be an uncapped and modified messenger RNA, and thus contain at least one modified base.

Thus, an uncapped messenger RNA may also be an uncapped and modified messenger RNA having a (5′)ppp(5′) guanosine extremity and containing at least one modified base.

An uncapped messenger RNA may also be an uncapped and modified messenger RNA having a (5′)ppp(5′) guanosine extremity and containing at least one pseudo-uridine and at least one 5-methylcytosine.

A capped messenger RNA may be a messenger RNA of which the 5′end is linked to a 7-methylguanosine, or analogue, connected to a 5′ to 5′ triphosphate linkage and containing naturally occurring bases or modified bases such as pseudo-urine or 5-methyl cytosine.

It is also understood that, when both modified and unmodified RNA molecules are used within one embodiment of the disclosure, they may be used either as mixtures and/or in purified forms.

Antigens

A nucleic acid contained in a lipid nanoparticle as disclosed herein may be an antigen.

According to one embodiment, compositions as disclosed herein, such as lipid nanoparticles, may be nucleic acid immunogenic composition or nucleic acid vaccines comprising at least one polynucleotide, e.g. polynucleotide constructs, which encode at least one wild type or engineered antigen.

Antigen-containing compositions as disclosed herein may vary in their valency. Valency refers to the number of antigenic components in the composition or in the polynucleotide (e.g., RNA polynucleotide) or polypeptide. In some embodiments, the immunogenic compositions are monovalent. They may also be compositions comprising more than one valence such as divalent, trivalent or multivalent compositions. Multivalent immunogenic compositions or vaccines may comprise 2, 3, 4, 5, 6, 7, 8, 9, 10, 11, 12, 13, 14, 15, 16, 17, 18, 19, 20, or more antigens or antigenic moieties (e.g., antigenic peptides, etc.). The antigenic components may be on a single polynucleotide or on separate polynucleotides.

Compositions as disclosed herein may be used to protect, treat or cure infection arising from contact with an infectious agent, such as bacteria, viruses, fungi, protozoa and parasites.

Compositions as disclosed herein may be used to protect, treat or cure cancer diseases.

According to one embodiment, a nucleic acid may encode for at least one antigen selected in the group consisting of bacterial antigens, protozoan antigens, viral antigens, fungal antigens, parasite antigens or tumour antigens.

Bacterial Antigens

The bacterium described herein can be a Gram-positive bacterium or a Gram- negative bacterium. Bacterial antigens may be obtained from Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Proteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

Viral Antigens

Viral antigens may be obtained from adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; SARS-Cov-2 virus; Chikungunya virus; Zika virus; parainfluenza virus; Human Enterovirus; Hanta virus; Japanese encephalitis virus; Vesicular exanthernavirus; Eastern equine encephalitisor; or Banna virus.

In one embodiment, the antigen is from a strain of Influenza A or Influenza B virus or combinations thereof. The strain of Influenza A or Influenza B may be associated with birds, pigs, horses, dogs, humans or non-human primates.

The nucleic acid may encode a hemagglutinin protein or a fragment thereof. The hemagglutinin protein may be H1, H2, H3, H4, H5, H6, H7, H8, H9, H10, H11, H12, H13, H14, H15, H16, H17, H18, or a fragment thereof. The hemagglutinin protein may or may not comprise a head domain (HA1). Alternatively, the hemagglutinin protein may or may not comprise a cytoplasmic domain.

In embodiments, the hemagglutinin protein is a truncated hemagglutinin protein. The truncated hemagglutinin protein may comprise a portion of the transmembrane domain.

In some embodiments, the virus may be selected from the group consisting of HIN1, H3N2, H7N9, H5N1 and H10N8 virus or a B strain virus.

In another embodiment, the antigen is from a coronavirus such as SARS-Cov-1 virus, SARS-Cov-2 virus, or MERS-Cov virus.

Fungal Antigens

Fungal antigens may be obtained from Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

Protozoan Antigens

Protozoan antigens may be obtained from Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

Parasitic Antigens

Parasitic antigens may be obtained from Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, Wuchereria bancrofti.

Tumour Antigens

In one embodiment, an antigen may be a tumor antigen, i.e., a constituent of cancer cells such as a protein or a peptide expressed in a cancer cell. The term “tumor antigen” relates to proteins that are under normal conditions specifically expressed in a limited number of tissues and/or organs or in specific developmental stages and are expressed or aberrantly expressed in at least one tumor or cancer tissue. Tumor antigens include, for example, differentiation antigens, for example cell type specific differentiation antigens, i.e., proteins that are under normal conditions specifically expressed in a certain cell type at a certain differentiation stage and germ line specific antigens. For example, a tumor antigen is presented by a cancer cell in which it is expressed.

For example, tumor antigens include the carcinoembryonal antigen, a 1-fetoprotein, isoferritin, and fetal sulphoglycoprotein, cc2-H-ferroprotein and γ-fetoprotein.

Other examples for tumor antigens that may be useful in the present disclosure are p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl 00, HAGE, HER-2/neu, HPV-E7, HPV-E6, HAST-2, hTERT (or hTRT), LAGE, LDLR/FUT, MAGE- A, for example MAGE-A1, MAGE-A2, MAGE- A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART- 1/Melan-A, MCi R, Myosin/m, MUC1, MUM-1, -2, -3, NA88-A, NF1, NY-ESO-1, NY-BR-1, pl90 minor BCR-abL, Pm 1/RARa, PRAME, proteinase 3, PSA, PSM, RAGE, RUl or RU2, SAGE, SART-1 or SART-3, SCGB3A2, SCP 1, SCP2, SCP3, SSX, SURVrVIN, TEL/AMLl, TPI/m, TRP-1, TRP-2, TRP-2/1NT2, TPTE and WT, for example WT-1.

Adjuvants

Nucleic acid containing compositions or lipid nanoparticles as disclosed herein may further comprise, or may be co-administered with, an adjuvant or an immune potentiator.

Adjuvants useful in the present disclosure may include, but are not limited to, natural or synthetic adjuvants. They may be organic or inorganic.

Adjuvants may be selected from any of the classes (1) mineral salts, e.g., aluminium hydroxide and aluminium or calcium phosphate gels; (2) emulsions including: oil emulsions and surfactant based formulations, e.g., microfluidized detergent stabilized oil-in-water emulsion, purified saponin, oil-in- water emulsion, stabilized water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensioactive compounds; (9) carbohydrates; or combinations thereof.

Selection of appropriate adjuvants and appropriate amount of adjuvant will be evident to one of ordinary skill in the art.

Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derb/ed P40 Vaccine Adjuvant, MPL™ Adjuvant, AS04, AS02, ASO1, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™ Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hlFN-gamma/Interferon-g, Interleukin-4p, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, PAA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(LC), ISCOMATRIX®, Abisco-100 vaccine adjuvant, Albumin- heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant El 12K of Cholera Toxin mCT-El 12K, and/or Matrix-S.

Protein Expression

The compositions as disclosed herein or the lipid nanoparticles as disclosed herein encapsulating at least one nucleic acid may also be used for treating individuals deficient in a protein. Therefore, the lipid nanoparticles may be used in a method for treating individuals deficient in a protein comprising administering lipid nanoparticles comprising at least one nucleic acid, for example a mRNA, wherein the nucleic acid encodes a functional protein corresponding to the protein which is deficient in the individual. In embodiments, following expression of the nucleic acid by a target cell a functional protein is produced.

The disclosure also relates to methods of intracellular delivery of nucleic acids that are capable of correcting existing genetic defects and/or providing beneficial functions to at least one target cell. Following successful delivery to target tissues and cells, the compositions and nucleic acids of the present disclosure transfect that target cell and the nucleic acids (e.g., mRNA) can be translated into the gene product of interest (e.g., a functional protein or enzyme) or can otherwise modulate or regulate the presence or expression of the gene product of interest.

The compositions and methods provided herein are useful in the management and treatment of a large number of diseases, for example diseases which result from protein and/or enzyme deficiencies. Individuals suffering from such diseases may have underlying genetic defects that lead to the compromised expression of a protein or an enzyme, including, for example, the non-synthesis of the protein, the reduced synthesis of the protein, or synthesis of a protein lacking or having diminished biological activity.

Alternatively, the nucleic acids may encode full length antibodies or smaller antibodies (e.g., both heavy and light chains) to confer immunity to a subject. In an alternative embodiment the compositions as described herein encode antibodies that may be used to transiently or chronically effect a functional response in subjects. For example, the mRNA nucleic acids as described herein may encode a functional monoclonal or polyclonal antibody, which upon translation (and as applicable, systemic excretion from the target cells) may be useful for targeting and/or inactivating a biological target (e.g., a stimulatory cytokine such as tumor necrosis factor). Similarly, the mRNA nucleic acids as described herein may encode, for example, functional anti-nephritic factor antibodies useful for the treatment of membranoproliferative glomerulonephritis type II or acute hemolytic uremic syndrome, or alternatively may encode anti-vascular endothelial growth factor (VEGF) antibodies useful for the treatment of VEGF-mediated diseases, such as cancer.

Pharmaceutical Compositions

According to some embodiments, the disclosure relates to pharmaceutical compositions, such as immunogenic compositions.

For the purposes of administration, the lipid nanoparticles as disclosed herein comprising a therapeutic agent, such as a nucleic acid, may be administered as pharmaceutical compositions. Pharmaceutical compositions of the present disclosure comprise lipid nanoparticles as disclosed herein and at least one pharmaceutically acceptable carrier, diluent or excipient.

According to some embodiments, pharmaceutical compositions suitable for the disclosure may comprise (i) at least one nucleic acid and at least one lipidic compound as disclosed herein, or (ii) at least one composition as described herein, or (iii) at least one lipid nanoparticle as described herein, and at least one pharmaceutically acceptable excipient.

In some embodiments, a pharmaceutical composition may be an immunogenic composition. An immunogenic composition as disclosed herein may comprise at least one lipid nanoparticle as described herein, wherein the nucleic acid contained thereof encodes for at least one antigen. Further an immunogenic composition may comprise at least one adjuvant as described herein.

According to some embodiments, the disclosure relates to a composition comprising at least one lipid nanoparticle as described herein for use as a medicament. Such a medicament may be used for the prevention and/or treatment of a disease as indicated herein.

According to some embodiments, the disclosure relates to a composition comprising at least one lipid nanoparticle as described herein, for use in a therapeutic method for preventing and/or treating a disease selected in a group consisting of infectious diseases, allergies, autoimmune diseases, rare blood disorders, rare metabolic diseases, rare neurologic diseases, and tumour or cancer diseases, and for example as herein described.

According to some embodiments, a composition comprising at least one lipid nanoparticle as herein described, may be for use as an immunogenic composition.

Immunogenic compositions as disclosed herein may be used in the prevention and/or treatment of an infectious diseases as indicated herein. They may contain at least one nucleic acid encoding for at least one antigen as herein described.

In some embodiments, the lipidic compound of formula (IV), (Va) or (Vb) may be present in a pharmaceutical or immunogenic composition in an amount which is effective to form lipid nanoparticles and deliver the therapeutic agent, for example a nucleic acid, for treating a specific disease or condition of interest.

Appropriate concentrations and dosages can be readily determined by one skilled in the art.

Administration of pharmaceutical and immunogenic compositions as disclosed herein may be carried out via any of the accepted modes of administration of compositions for serving similar utilities.

The compositions as disclosed herein may be formulated into preparations in solid, semi-solid, liquid forms, such as powders, solutions, suspensions or injections. Typical routes of administering such pharmaceutical compositions include, without limitation, oral, topical, transdermal, inhalation, parenteral, sublingual, buccal, intranasal. The term parenteral as used herein includes subcutaneous injections, intravenous, intramuscular, intradermal, intrasternal injection or infusion techniques.

In some embodiment, a composition as disclosed herein may be administered by transdermal, subcutaneous, intradermal or intramuscular route.

Compositions as disclosed herein are formulated so as to allow the active ingredients contained therein to be bioavailable upon administration of the composition to a patient.

Actual methods of preparing such dosage forms are known, or will be apparent, to those skilled in this art; for example, see Remington: The Science and Practice of Pharmacy, 20th Edition (Philadelphia College of Pharmacy and Science, 2000).

The compositions may contain at least one inert diluent or carrier.

In one embodiment, the composition may be in the form of a liquid, for example, a solution, an emulsion or a suspension. The liquid may be for delivery by injection. Compositions intended to be administered by injection may contain at least one of: a surfactant, preservative, wetting agent, dispersing agent, suspending agent, buffer, stabilizer and isotonic agent may be included. The liquid compositions as disclosed herein may include at least one of: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose.

The parenteral preparation can be enclosed in ampoules, disposable syringes or multiple dose vials made of glass or plastic. An injectable pharmaceutical composition is for example sterile.

The pharmaceutical and immunogenic compositions as disclosed herein may be prepared by methodology well known in the pharmaceutical art. For example, a pharmaceutical composition intended to be administered by injection can be prepared by combining the lipid nanoparticles as disclosed herein with sterile, distilled water or other carrier so as to form a solution. A surfactant may be added to facilitate the formation of a homogeneous solution or suspension.

The compositions as disclosed herein are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the specific disorder or condition; and the subject undergoing therapy.

Compositions as disclosed herein may also be administered simultaneously with, prior to, or after administration of at least one other therapeutic agent. Such combination therapy includes administration of a single pharmaceutical dosage formulation of a composition as disclosed herein and at least one additional active agent, as well as administration of the composition as disclosed herein and each active agent in its own separate pharmaceutical dosage formulation. Where separate dosage formulations are used, the compositions as disclosed herein and at least one additional active agent can be administered at essentially the same time, i.e., concurrently, or at separately staggered times, i.e., sequentially; combination therapy is understood to include all these regimens.

Methods of Treatment

In some embodiments, the disclosure also relates to a method of preventing and/or treating a disease in an individual in need thereof, wherein the method comprises administering an effective amount of at least one lipid nanoparticle as disclosed herein, to said individual. For example, a composition containing the LNPs as disclosed herein may be for use in a therapeutic method for preventing and/or treating infectious diseases, allergies, autoimmune diseases, rare blood disorders, rare metabolic diseases, rare neurologic diseases, and tumour or cancer diseases.

In some embodiments, the disclosure also relates to a use of at least one lipid nanoparticle as disclosed herein for the manufacture of a medicament for preventing and/or treating infectious diseases, allergies, autoimmune diseases, rare blood disorders, rare metabolic diseases, rare neurologic diseases, and tumour or cancer diseases. For example, diseases which may be concerned by the disclosure may be infectious diseases such as viral infectious diseases, bacterial infectious diseases, fungal or parasitic infectious diseases. Diseases also concerned by the disclosure may be cancer or tumour diseases.

Viral infectious diseases may be acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, Covid-19, Respiratory Syncytial Virus (RSV) infection, and herpes zoster.

In one embodiment, the disease is influenza, a Respiratory Syncytial Virus (RSV) infection, or Covid-19, and for example is influenza.

Bacterial infectious diseases may be such as abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related cutaneous conditions, bartonellosis, BCG-oma, botryomycosis, botulism, Brazilian purpuric fever, Brodie abscess, brucellosis, Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fitz-Hugh-Curtis syndrome, flea-borne spotted fever, foot rot (infectious pododermatitis), Garre's sclerosing osteomyelitis, Gonorrhea, Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRS A) infection, Mycobacterium avium-intracellulare (MAI), mycoplasma pneumonia, necrotizing fasciitis, nocardiosis, noma (cancrum oris or gangrenous stomatitis), omphalitis, orbital cellulitis, osteomyelitis, overwhelming post- splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, salmonellosis, scarlet fever, sepsis, serratia infection, shigellosis, southern tick- associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse- Friderichsen syndrome, pseudotuberculosis (Yersinia) disease, and yersiniosis.

Parasitic infectious diseases may be amoebiasis, giardiasis, trichomoniasis, African Sleeping Sickness, American Sleeping Sickness, leishmaniasis (Kala-Azar), balantidiasis, toxoplasmosis, malaria, acanthamoeba keratitis, and babesiosis.

Fungal infectious diseases may be aspergilloses, blastomycosis, candidasis, coccidioidomycosis, cryptococcosis, histoplasmosis, mycetomas, paracoccidioidomycosis, and tinea pedis. Furthermore, persons with immuno-deficiencies are susceptible to disease by fungal genera such as Aspergillus, Candida, Cryptoccocus, Histoplasma, and Pneumocystis. Other fungi can attack eyes, nails, hair, and especially skin, the so-called dermatophytic fungi and keratinophilic fungi, and cause a variety of conditions, of which ringworms such as athlete's foot are common. Fungal spores are also a major cause of allergies, and a wide range of fungi from different taxonomic groups can evoke allergic reactions in some people.

Cancer or tumour diseases may be cancer or tumor diseases are for example selected from melanomas, malignant melanomas, colon carcinomas, lymphomas, sarcomas, blastomas, renal carcinomas, gastrointestinal tumors, gliomas, prostate tumors, bladder cancer, rectal tumors, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, various virus-induced tumors such as, for example, papilloma virus-induced carcinomas (e.g. cervical carcinoma=cervical cancer), adenocarcinomas, herpes virus-induced tumors (e.g. Burkitt's lymphoma, EBV-induced B-cell lymphoma), heptatitis B-induced tumors (hepatocell carcinomas), HTLV-1- and HTLV-2-induced lymphomas, acoustic neuroma, lung carcinomas (=lung cancer=bronchial carcinoma), small-cell lung carcinomas, pharyngeal cancer, anal carcinoma, glioblastoma, rectal carcinoma, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal carcinoma (=oesophageal cancer), wart involvement, tumors of the small intestine, craniopharyngeomas, ovarian carcinoma, genital tumors, ovarian cancer (=ovarian carcinoma), pancreatic carcinoma (=pancreatic cancer), endometrial carcinoma, liver metastases, penile cancer, tongue cancer, gall bladder cancer, leukaemia, plasmocytoma, lid tumor, prostate cancer (=prostate tumors).

Diseases for which the present disclosure can be useful as a therapeutic intervention include diseases such as SMN1-related spinal muscular atrophy (SMA); amyotrophic lateral sclerosis (ALS); GALT-related galactosemia; Cystic Fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR1-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type C1; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1, EIF2B2, EIF2B3, EIF2B4 and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy's disease (SBMA); Notch-3 related cerebral autosomal dominant arteriopathy with subcortical infarcts and leukoencephalopathy (CADASIL); SCN1A and SCN1B-related seizure disorders; the Polymerase G-related disorders which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; Fabry disease; and Wilson's disease.

In one embodiment, the nucleic acids, and for example mRNA, of the present disclosure may encode functional proteins or enzymes. For example, the compositions of the present disclosure may include mRNA encoding erythropoietin (EPO), al-antitrypsin, carboxypeptidase N, alpha galactosidase (GLA), ornithine carbamoyltransferase (OTC), or human growth hormone (hGH).

In other embodiments, the disclosure relates to methods of transfecting at least one isolated target cell with a nucleic acid, wherein said method comprises contacting the at least one target cell with an effective amount of at least one nucleic acid and at least one lipid nanoparticle as above described, such that the at least one target cell are transfected with said nucleic acid.

Target cells include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, antigen presenting cells such as dendritic cells, reticulocytes, leukocytes, granulocytes and tumor cells.

In one embodiment, the cells targeted may be spleen, liver, lung, heart and kidney cells. In another embodiment, the cells targeted may be spleen and kidney cells, and for example may be spleen cells.

In some embodiments, lipid nanoparticles or compositions as disclosed herein which allow avoiding hepatic clearance may be of particular interest.

Following transfection of at least one target cell by, for example, the nucleic acid encapsulated in the lipid nanoparticles, the production of a polypeptide or a protein encoded by such nucleic acid may be for example stimulated and the capability of such target cells to express the nucleic acid and produce, for example, a polypeptide or protein of interest is enhanced. For example, transfection of a target cell by a composition encapsulating mRNA will enhance (i.e., increase) the production of the protein or enzyme encoded by such mRNA.

In other embodiments, the disclosure relates to methods of producing a polypeptide in at least one target cell, wherein said method comprises contacting the at least one target cell with an effective amount of at least one nucleic acid encoding said polypeptide and at least one lipid nanoparticle as herein described, such that the at least one target cell are transfected with the nucleic acid operably encoding said polypeptide.

It is to be understood that the disclosure encompasses all variations, combinations, and permutations in which at least one limitation, element, clause, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

Materials and Methods

Nuclear Magnetic Resonance Spectroscopy (H, C NMR)
H and C NMR spectra were recorded at room temperature on the following spectrometer: Brucker Advance 400 (NMR H: 400 MHz and NMR C: 75 MHz).

Recorded shifts were reported in parts per million (6) and calibrated using residual undeuterated 3: H 7.26 ppm; C 77.16 ppm, MeOH H 3.31 ppm; C: 49.0 ppm). Data were represented as follows, chemical shift, multiplicity (s=singlet, d=doublet, t=triplet, q=quartet and m=multiplet), coupling constant (J in Hz), integration and attribution.

NMR spectra were obtained using the commercial software NMRnotebook.
High-resolution mass spectra (HRMS) were obtained using an Agilent Q-TOF (time of flight) 6520 and Low-resolution mass spectra (LCMS) using an Agilent MSD 1200 SL (ESI/APCI) with an Agilent HPLC 1200 SL.

### Example 1: Synthesis of 2-[2-[2-[2-[2-[2,3-bis[(Z)-octadec-9-enoxy] propoxy]ethoxy] ethoxy] ethoxy] ethoxy]-2-oxo-ethyl]-[2-[2-(2-methoxyethylammonio) acetyl]oxyethyl] ammonium;2,2,2-trifluoroacetate (Compound of Formula (III) in the Disclosure as Trifluoroacetate Salt—DOG-CLEAVE) and its Conversion as a Dihydrochloride Salt

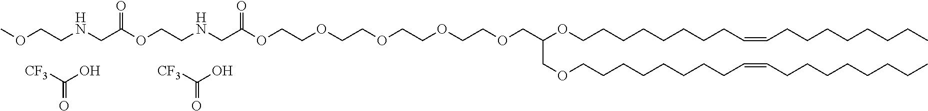

The compound 14 is prepared according to the following schema of synthesis:

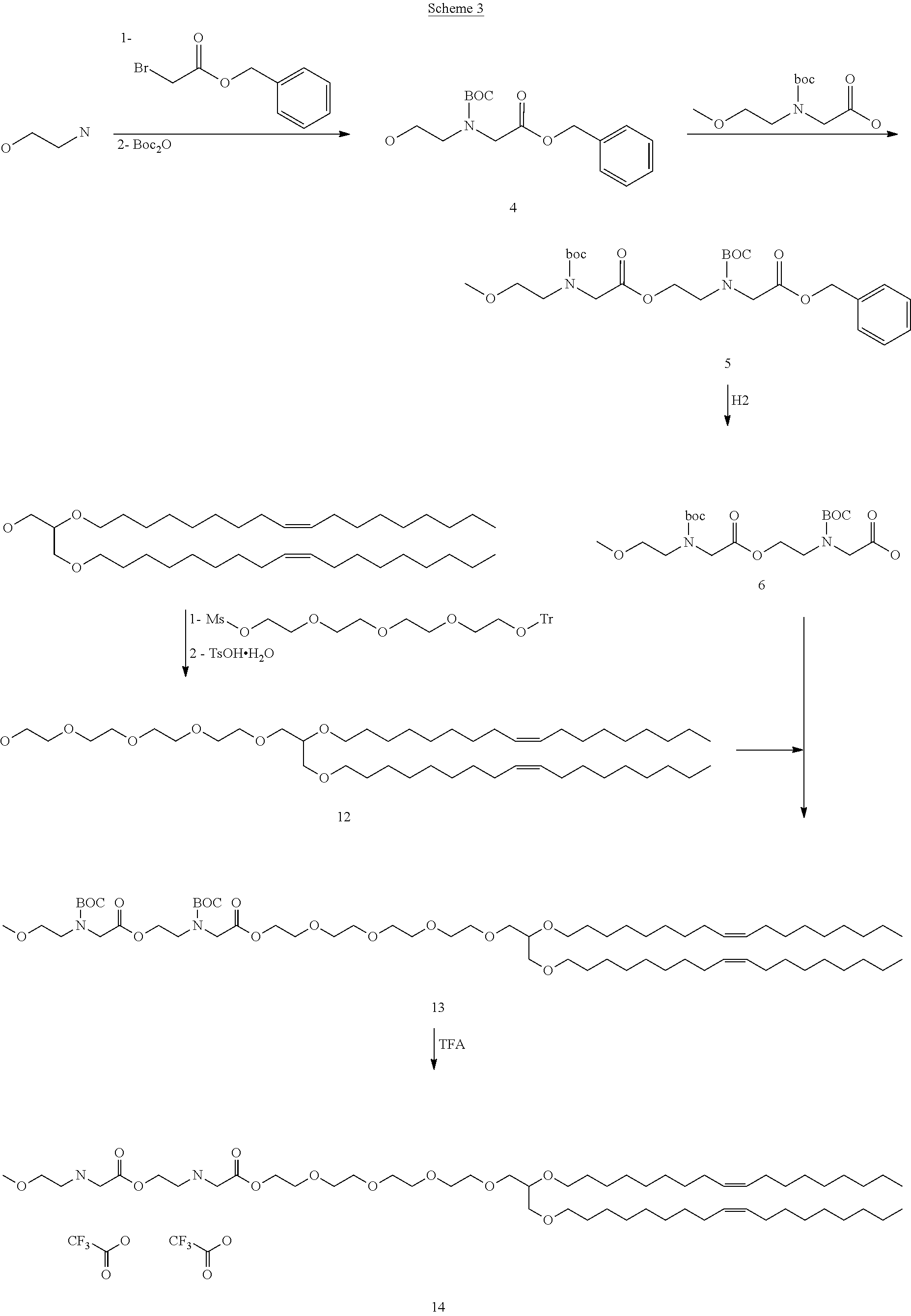

### 1.1 Synthesis of the 2-[tert-butoxycarbonyl-[2-[2-[tert-butoxycarbonyl(2-methoxyethyl)amino]acetyl]oxyethyl]amino]acetic Acid (Compound 6 of Scheme 3)

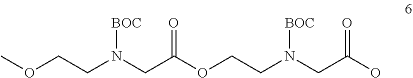 [SMILES: COCCN(CC(=O)OCCN(CC(=O)O)C(=O)OC(C)(C)C)C(=O)OC(C)(C)C]

### 1.1.1 Synthesis of the Benzyl 2-[tert-butoxycarbonyl(2-hydroxyethyl)amino]acetate (Compound 4 of Scheme 3)

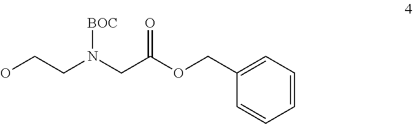 [SMILES: CC(C)(C)OC(=O)N(CCO)CC(=O)OCc1ccccc1]

To a 0° C. solution of 2-aminoethanol (4.00 g, 65.5 mmol) and K2CO3 (45.3 g, 327 mmol) in 160 mL acetonitrile was added a solution of ethyl bromoacetate (15 g, 65.5 mmol) in 6 mL of acetonitrile over a 1.5 h period. The solution was stirred for 2 h at 0° C. and filtered. To the filtrate was added Di-tert-butyl dicarbonate (14.3 g, 65.5 mmol) and the mixture was distilled under reduced pressure at 50° C. to complete the reaction (1 to 2 hours). Toluene was added (100 ml), the organic phase was washed with 1N aqueous hydrochloric acid solution, saturated sodium hydrogen carbonate solution and brine. Organic layer was dried over Na2SO4, filtered and concentrated. The residue was purified by column chromatography (0% to 100% ethyl acetate in petroleum ether) to give benzyl 2-[tert-butoxycarbonyl(2-hydroxyethyl)amino]acetate (13 g, 40 mmol, 61% yield) as a colorless oil.

1H NMR (500 MHz, CDCl3) δ 7.40-7.31 (m, 6H), 5.20 (d, J=2.7 Hz, 2H), 4.00 (d, J=30.1 Hz, 2H), 3.72 (dd, J=25.2, 4.5 Hz, 2H), 3.48-3.39 (m, 2H), 3.33-3.25 (m, 1H), 1.51-1.31 (m, 9H).

### 1.1.2 Synthesis of 2-[(2-benzyloxy-2-oxo-ethyl)-tert-butoxycarbonyl-amino]ethyl 2-[tert-butoxycarbonyl(2-methoxyethyl)amino]acetate (Compound 5 of Scheme 3)

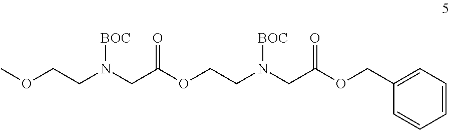 [SMILES: COCCN(CC(=O)OCCN(CC(=O)OCc1ccccc1)C(=O)OC(C)(C)C)C(=O)OC(C)(C)C]

In a 250 ml round bottom flask were dissolved benzyl 2-[tert-butoxycarbonyl(2-hydroxyethyl)amino]acetate (6.68 g, 21.6 mmol), 2-[tert-butoxycarbonyl(2-methoxyethyl)amino]acetic acid (10.1 g, 43.2 mmol), 4-methylmorpholine (4.36 g, 43.2 mmol) and N,N-dimethylpyridin-4-amine (5.27 g, 43.2 mmol) in dry dichloromethane (100 ml). The mixture was cooled to 0° C. and 3-(ethyliminomethyleneamino)-N,N-dimethyl-propan-1-amine;hydrochloride (8.27 g, 43.2 mmol) was added portion wise over 45 min. The mixture was stirred at room temperature overnight. Water was added, organic layer was separated and washed again with water (2*200 ml) then brine. The organic phase was dried over sodium sulfate, filtrated and solvent was removed under reduced. The crude product was purified by column chromatography on 120 g SI60 (n-Hexane/ethyl acetate 0%→40% on 10 VC then 40% on 11 VC) to give the coupling 2-[(2-benzyloxy-2-oxo-ethyl)-tert-butoxycarbonyl-amino]ethyl 2-[tert-butoxycarbonyl(2-methoxyethyl)amino]acetate as a colorless oil (7.92 g, 70% Yield).

LCMS:425(M-Boc+1), 100% UV214

1H NMR (400 MHz, CDCl3) δ 7.35 (d, J=3.7 Hz, 5H), 5.16 (d, J=3.6 Hz, 2H), 4.24 (d, J=5.5 Hz, 2H), 4.08-3.93 (m, 4H), 3.58-3.39 (m, 6H), 3.27 (dd, J=6.9, 2.0 Hz, 3H), 1.49-1.42 (m, 9H), 1.37 (d, J=23.8 Hz, 9H).

### 1.1.3 Synthesis of the 2-[tert-butoxycarbonyl-[2-[2-[tert-butoxycarbonyl(2-methoxyethyl)amino]acetyl]oxyethyl]amino]acetic Acid (Compound 6 of Scheme 3)

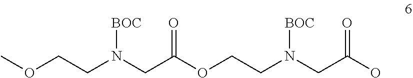 [SMILES: COCCN(CC(=O)OCCN(CC(=O)O)C(=O)OC(C)(C)C)C(=O)OC(C)(C)C]

In a 17 ml H₂ reactor was dissolved 2-[(2-benzyloxy-2-oxo-ethyl)-tert-butoxycarbonyl-amino]ethyl 2-[tert-butoxycarbonyl(2-methoxyethyl)amino]acetate (8.07 g, 15.4 mmol) in ethyl acetate (100 ml) and palladium (10%, 1.64 g, 1.54 mmol) was added. The mixture was stirred 4 h at room temperature under a 5 bar H₂ atmosphere, then filtrated on Talc, washed with ethyl acetate. Solvent was removed under reduced pressure to give 2-[tert-butoxycarbonyl-[2-[2-[tert-butoxycarbonyl(2-methoxyethyl)amino]acetyl]oxyethyl]aamino]acetic acid (5.39 g, 76.6% Yield) as colourless oil.

LCMS:335(M-Boc+1), 82% UV214

1H NMR (400 MHz, CDCl3) δ 5.70 (s, 1H), 4.33-4.20 (m, 2H), 4.10-3.94 (m, 4H), 3.61-3.43 (m, 6H), 3.32 (s, 3H), 1.45 (dd, J=17.1, 5.3 Hz, 18H).

### 1.2 Synthesis of the 2-[2-[2-[2-[2,3-bis[(Z)-octadec-9-enoxy]propoxy]ethoxy]ethoxy]ethoxy]ethanol (Compound 12 of Scheme 3)

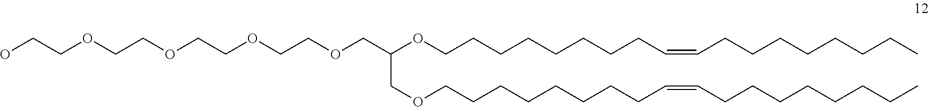

To a suspension of NaH (60% mineral oil dispersion, 355 mg, 9.27 mmol) in 15 mL of THF was added 2,3-bis[(Z)-octadec-9-enoxy]propan-1-ol (1.1 g, 1.85 mol) dissolved in 15 mL of THF. The resultant suspension was stirred for 2 h at room temperature. 2-[2-[2-(2-trityloxyethoxy)ethoxy]ethoxy]ethyl methanesulfonate (1.43 g, 2.78 mmol) was added to the suspension, and the reaction mixture was brought to reflux overnight. The reaction mixture was cooled to room temperature, and water was added. EtOAc (100 mL) was added, the mixture was shaken, the layers were separated, and the organic layer was collected. The aqueous layer was extracted with EtOAc (50 mL *2). The combined organic layers were washed with brine and dried over Na2SO4. The residue was purified by flash column chromatography on silica gel eluting with 1:8 ethyl acetate/petroleum ether to give [2-[2-[2-[2-[2,3-bis[(Z)-octadec-9-enoxy]propoxy]ethoxy]ethoxy]ethoxy]ethoxy-diphenyl-methyl]benzene (1.8 g, 95.9%) as colourless oil.

1H NMR (500 MHz, CDCl3) δ 7.46 (d, J=7.6 Hz, 7H), 7.28 (t, J=7.5 Hz, 7H), 7.21 (dd, J=15.2, 7.5 Hz, 4H), 5.39-5.30 (m, 3H), 3.74-3.58 (m, 15H), 3.59-3.37 (m, 9H), 3.23 (dd, J=9.8, 4.7 Hz, 2H), 2.07-1.92 (m, 6H), 1.54 (dd, J=13.6, 6.8 Hz, 4H), 1.40-1.19 (m, 42H), 0.88 (t, J=6.9 Hz, 6H).

To a mixture of [2-[2-[2-[2-[2,3-bis[(Z)-octadec-9-enoxy]propoxy]ethoxy]ethoxy]ethoxy]ethoxy-diphenyl-methyl]benzene (8.4 g, 8.3 mmol) in THF (50 mL)/MeOH (50 mL) was added Toluene-4-sulfonic acid (1.58 g, 8.3 mmol) and stirred overnight at room temperature. Et3N (5 mL) was added to the reaction mixture and concentrated. The residue was purified by flash column chromatography on silica gel eluting with 2:1 ethyl acetate/petroleum ether to give 2-[2-[2-[2-[2,3-bis[(Z)-octadec-9-enoxy]propoxy]ethoxy]ethoxy]ethoxy]ethanol (3.1 g, 48.5%) as colourless oil.

1H NMR (500 MHz, CDCl3) δ 5.39-5.30 (m, 3H), 3.74-3.40 (m, 25H), 2.07-1.93 (m, 7H), 1.59-1.50 (m, 4H), 1.37-1.22 (m, 44H), 0.88 (t, J=6.9 Hz, 6H).

### 1.3 Synthesis of the [2-[2-[2-[2-[2-[2,3-bis[(Z)-octadec-9-enoxy]propoxy]ethoxy]ethoxy]ethoxy]ethoxy]-2-oxo-ethyl]-[2-[2-(2-methoxyethylammonio)acetyl]oxyethyl]ammonium; Dihydrochloride i.e the Compound (III) Under the Form of its Hydrochloride Salt

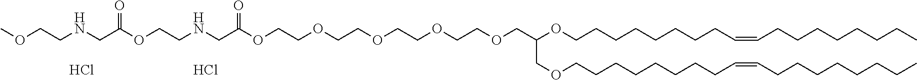 [SMILES: CCCCCCCC/C=C\CCCCCCCCOCC(COCCOCCOCCOCCOC(=O)CNCCOC(=O)CNCCOC)OCCCCCCCC/C=C\CCCCCCCC ; Cl ; Cl]

### 1.3.1 Synthesis of the 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (Compound 13 of Scheme 3)

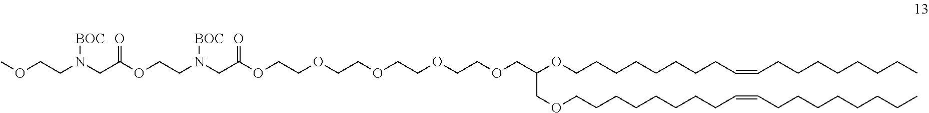 [SMILES: CCCCCCCC/C=C\CCCCCCCCOCC(COCCOCCOCCOCCOC(=O)CN(CCOC(=O)CN(CCOC)C(=O)OC(C)(C)C)C(=O)OC(C)(C)C)OCCCCCCCC/C=C\CCCCCCCC]

In a 50 mL round bottom flask, 2-[tert-butoxycarbonyl-[2-[2-[tert-butoxycarbonyl(2-methoxyethyl)amino]acetyl]oxyethyl]amino]acetic acid (200 mg, 0.46 mmol), 22-[2-[2-[2-(2,3-dihexadecoxypropoxy)ethoxy]ethoxy]ethoxy]ethanol (330 mg, 0.46 mmol) and N,N-dimethylpyridin-4-amine (84.4 mg, 0.69 mmol) were dissolved in DCM-Anhydrous (10 mL) in presence of 4-methylmorpholine (69.8 mg, 0.69 mmol). The reaction was cooled to 0° C. and 3-(ethyliminomethyleneamino)-N,N-dimethyl-propan-1-amine;hydrochloride (132 mg, 0.69 mmol) was added to the mixture. The reaction was warmed to room temperature and stirred overnight. The reaction was monitored by TLC (Hept/EtOAc 5:5, expected product rf: 0.5). Water was added and the organic layers were extracted with DCM (3×15 mL), washed with brine, dried over Na2SO4, filtered and concentrated. The residue was purified by flash column chromatography on silica gel eluting with 1:4 ethyl acetate/petroleum ether to give 22-[tert-butoxycarbonyl-[2-[2-[2-[2-[2-(2,3-dihexadecoxypropoxy)ethoxy]ethoxy]ethoxy]ethoxy]-2-oxo-ethyl]amino]ethyl 2-[tert-butoxycarbonyl(2-methoxyethyl)amino]acetate (0.38 g, 72.8%) as colourless oil.

1H NMR (500 MHz, CDCl3) δ 4.31-4.20 (m, 4H), 4.04 (d, J=7.9 Hz, 2H), 4.00-3.96 (m, 2H), 3.70 (s, 2H), 3.67-3.61 (m, 12H), 3.58-3.39 (m, 15H), 3.30 (d, J=7.0 Hz, 3H), 1.59-1.51 (m, 4H), 1.47 (s, 9H), 1.42 (s, 9H), 1.34-1.21 (m, 52H), 0.88 (t, J=6.9 Hz, 6H)

### 1.3.2 Synthesis of the [2-[2-[2-[2-[2-[2,3-bis[(Z)-octadec-9-enoxy]propoxy]ethoxy]ethoxy]ethoxy]ethoxy]-2-oxo-ethyl]-[2-[2-(2-methoxyethylammonio)acetyl]oxyethyl]ammonium:2,2,2-trifluoroacetate (Compound 14 of Scheme 3)

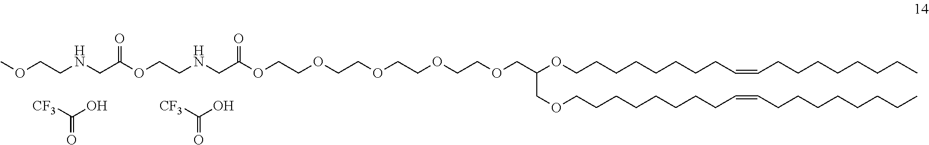 [SMILES: CCCCCCCC/C=C\CCCCCCCCOCC(COCCOCCOCCOCCOC(=O)CNCCOC(=O)CNCCOC)OCCCCCCCC/C=C\CCCCCCCC ; O=C(O)C(F)(F)F ; O=C(O)C(F)(F)F]

To a mixture of 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.32 g, 0.27 mmol) in DCM (5 mL) was added TFA (0.308 g, 2.7 mmol) at room temperature. The mixture was stirred at ambient temperature for 16 h. The mixture was concentrated to 2-[[2-[2-[2-[2-[2-[2,3-bis[(Z)-octadec-9-enoxy]propoxy]ethoxy]ethoxy]ethoxy]ethoxy]-2-oxo-ethyl]amino]ethyl 2-(2-methoxyethylamino)acetate;2,2,2-trifluoroacetic acid (0.23 g, 66.7%) as yellow oil.

1H NMR (500 MHz, CDCl3) δ 5.40-5.30 (m, 3H), 4.60 (s, 2H), 4.39-4.31 (m, 2H), 4.03 (s, 2H), 3.96 (s, 2H), 3.73-3.68 (m, 4H), 3.67-3.61 (m, 14H), 3.60-3.40 (m, 13H), 3.36 (s, 3H), 3.33-3.29 (m, 2H), 2.06-1.92 (m, 8H), 1.57-1.53 (m, 4H), 1.31-1.23 (m, 45H), 0.88 (t, J=6.9 Hz, 7H).

### 1.3.3 Synthesis of the 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]amino]ethyl 2-(2-methoxyethylamino) Acetate: Dihydrochloride

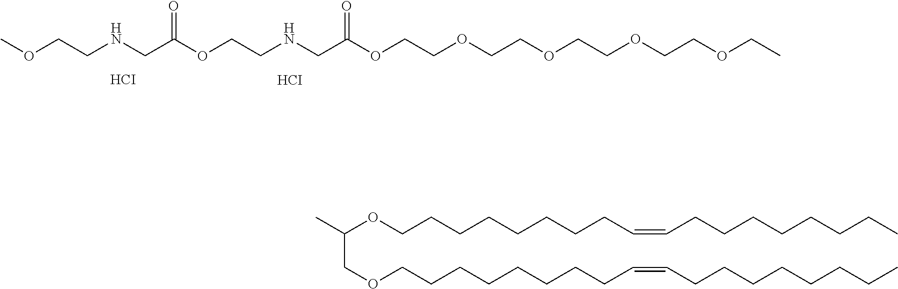

To a mixture of 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.45 g, 0.375 mmol) in DCM (5 mL) was added HCl in EtOAc (5 mL, 3 mol/L). The mixture was stirred 3 h at room temperature. The reaction mixture was concentrated to give 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]amino]ethyl 2-(2-methoxyethylamino)acetate;dihydrochloride (0.143 g, 35.6%) as a white oil.

1H NMR (500 MHz, CDCl3) δ 9.83 (s, 3H), 5.42-5.27 (m, 3H), 4.80 (s, 2H), 4.39 (s, 2H), 4.34-4.05 (m, 7H), 3.98 (s, 2H), 3.86 (s, 2H), 3.78-3.30 (m, 25H), 2.10-1.92 (m, 8H), 1.60-1.48 (m, 4H), 1.35-1.23 (m, 44H), 0.88 (t, J=6.9 Hz, 6H).

### Example 2: Synthesis of 2-[[2-[2-[2-[2-[2-[2,3-bis[(Z)-octadec-9-enoxy]propoxy]ethoxy]ethoxy]ethoxy]ethoxy]-2-oxo-ethyl]amino]ethyl 2-(2-methoxyethylamino)acetate;2,2,2-trifluoroacetic Acid (Compound (III))

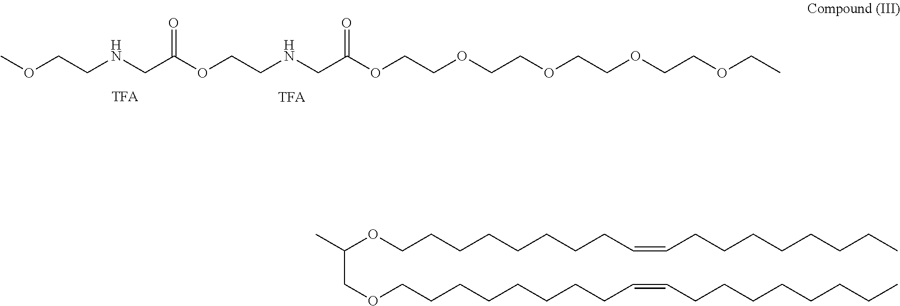

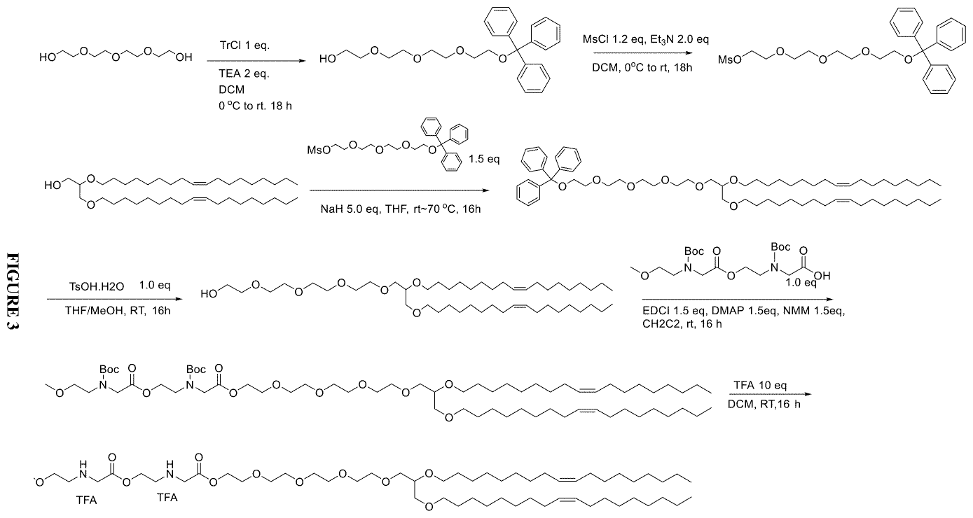

Compound (III) was synthesized based on the chemistry shown in Scheme (4) represented on FIG. 3.

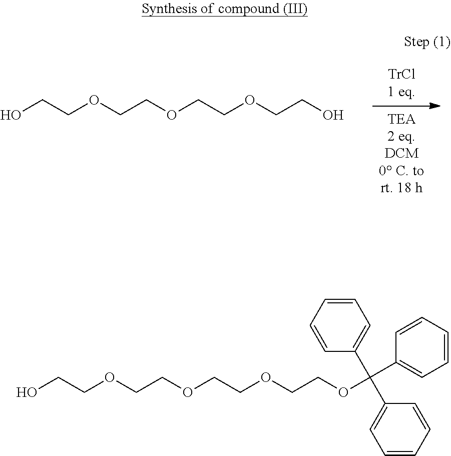 [SMILES: OCCOCCOCCOCCO ; OCCOCCOCCOCCOC(c1ccccc1)(c1ccccc1)c1ccccc1]

To a mixture of 2-[2-[2-(2-hydroxyethoxy)ethoxy]ethoxy]ethanol (20 g, 0.103 mol), N,N-dimethylpyridin-4-amine (0.629 g, 0.00515 mol) and [chloro(diphenyl)methyl]benzene (23 g, 0.0824 mol) in DCM (300 mL) cooled to 0° C. was added triethylamine (20.8 g, 0.206 mol). The reaction mixture was stirred for 18 h at ambient temperature. LCMS showed a good reaction. The mixture was poured into water (600 mL) and extracted with DCM (2*400 mL). The organic layers were washed with brine, dried over Na₂SO₄, filtered and concentrated. The residue was purified by flash column chromatography on silica gel eluted with 2:1 ethyl acetate/petroleum ether to give 2-[2-[2-(2-trityloxyethoxy)ethoxy]ethoxy]ethanol (15.6 g, 34.7%) as colorless oil.

LCMS 454 (M+18), 98% UV 214 nm.

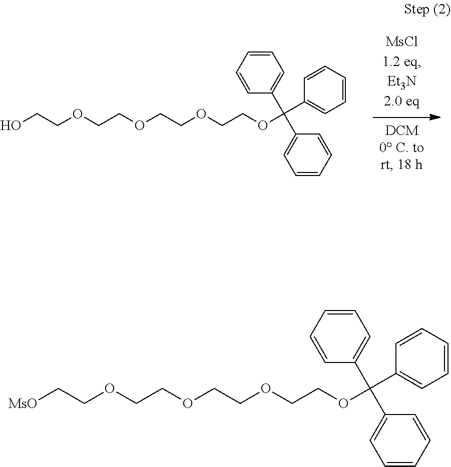 [SMILES: OCCOCCOCCOCCOC(c1ccccc1)(c1ccccc1)c1ccccc1 ; CS(=O)(=O)OCCOCCOCCOCCOC(c1ccccc1)(c1ccccc1)c1ccccc1]

To a mixture of 2-[2-[2-(2-trityloxyethoxy)ethoxy]ethoxy]ethanol (15.6 g, 0.0357 mol) and N,N-diethylethanamine (7.23 g, 0.0715 mol) in DCM (300 mL) was added methanesulfonyl chloride (4.91 g, 0.0429 mol) slowly at 0° C. The mixture was stirred overnight at room temperature. Water (150 mL) and CH2Cl2 (300 mL) were added to the solution, and the mixture was transferred to a separatory funnel. The mixture was shaken, the layers were separated, and the organic layer was collected. The aqueous layer was further extracted with CH2Cl2 (150 mL*2). The combined organic layers were then washed with 10% NaHCO₃ (150 mL) and brine (150 mL), and dried over MgSO4. Solvent was then removed to give 2-[2-[2-(2-trityloxyethoxy)ethoxy]ethoxy]ethyl methanesulfonate (18.4 g, 100%) as a yellow oil.

HNMR (EXP-20-IJ3414)

¹H NMR (400 MHz, CDCl₃) δ 7.46 (d, J=7.5 Hz, 6H), 7.29 (t, J=7.4 Hz, 7H), 7.23 (t, J=7.2 Hz, 3H), 4.36-4.28 (m, 2H), 3.76-3.72 (m, 2H), 3.67 (s, 10H), 3.23 (t, J=5.1 Hz, 2H), 2.99 (s, 3H).

LCMS 532 (M+18) 96% UV (214 nm)

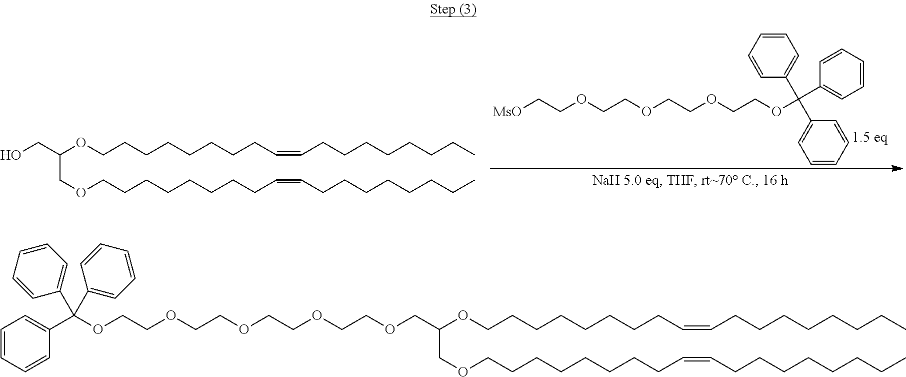 [SMILES: CCCCCCCC/C=C\CCCCCCCCOCC(CO)OCCCCCCCC/C=C\CCCCCCCC ; CS(=O)(=O)OCCOCCOCCOCCOC(c1ccccc1)(c1ccccc1)c1ccccc1 ; CCCCCCCC/C=C\CCCCCCCCOCC(COCCOCCOCCOCCOC(c1ccccc1)(c1ccccc1)c1ccccc1)OCCCCCCCC/C=C\CCCCCCCC]

To a suspension of NaH (60% mineral oil dispersion, 355 mg, 9.27 mmol) in 15 mL of THF was added 2,3-bis[(Z)-octadec-9-enoxy]propan-1-ol (1.1 g, 1.85 mol) dissolved in 15 mL of THF. The resultant suspension was stirred for 2 h at room temperature. 2-[2-[2-(2-trityloxyethoxy)ethoxy]ethoxy]ethyl methanesulfonate (1.43 g, 2.78 mmol) was added to the suspension, and the reaction mixture was brought to reflux overnight. The reaction mixture was cooled to room temperature, and water was added. EtOAc (100 mL) was added, the mixture was shaken, the layers were separated, and the organic layer was collected. The aqueous layer was extracted with EtOAc (50 mL*2). The combined organic layers were washed with brine and dried over Na2SO4. The residue was purified by flash column chromatography on silica gel eluting with 1:8 ethyl acetate/petroleum ether to give [2-[2-[2-[2-[2,3-bis[(Z)-octadec-9-enoxy]propoxy]ethoxy]ethoxy]ethoxy]ethoxy-diphenyl-methyl]benzene (1.8 g, 95.9%) as colorless oil.

¹H NMR (500 MHz, CDCl₃) δ 7.46 (d, J=7.6 Hz, 7H), 7.28 (t, J=7.5 Hz, 7H), 7.21 (dd, J=15.2, 7.5 Hz, 4H), 5.39-5.30 (m, 3H), 3.74-3.58 (m, 15H), 3.59-3.37 (m, 9H), 3.23 (dd, J=9.8, 4.7 Hz, 2H), 2.07-1.92 (m, 6H), 1.54 (dd, J=13.6, 6.8 Hz, 4H), 1.40-1.19 (m, 42H), 0.88 (t, J=6.9 Hz, 6H).

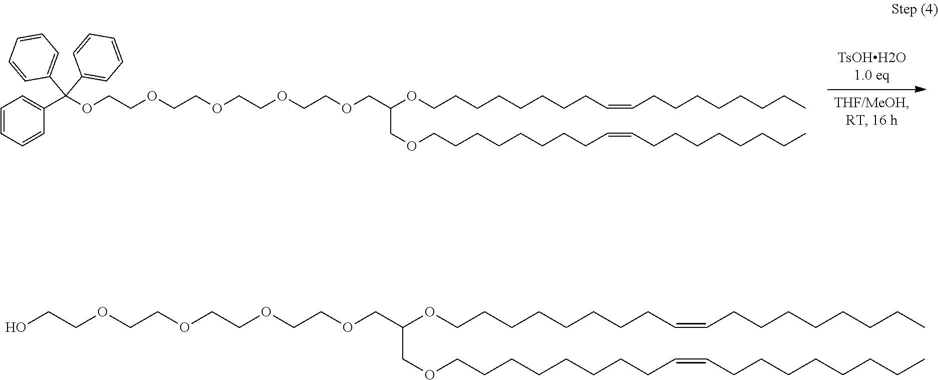 [SMILES: CCCCCCCC/C=C\CCCCCCCCOCC(COCCOCCOCCOCCOC(c1ccccc1)(c1ccccc1)c1ccccc1)OCCCCCCCC/C=C\CCCCCCCC ; CCCCCCCC/C=C\CCCCCCCCOCC(COCCOCCOCCOCCO)OCCCCCCCC/C=C\CCCCCCCC]

To a mixture of [2-[2-[2-[2-[2,3-bis[(Z)-octadec-9-enoxy]propoxy]ethoxy]ethoxy]ethoxy]ethoxy- diphenyl-methyl]benzene (1.0 g, 0.99 mmol) in THF (10 mL)/MeOH (10 mL) was added Toluene-4-sulfonic acid (0.188 g, 0.99 mmol) and stirred overnight at room temperature. Et₃N (0.3 mL) was added to the reaction mixture and concentrated. The residue was purified by flash column chromatography on silica gel eluting with 2:1 ethyl acetate/petroleum ether to give 2-[2-[2-[2-[2,3-bis[(Z)-octadec-9-enoxy]propoxy]ethoxy]ethoxy]ethoxy]ethanol (0.534 g, 70.2%) as colorless oil.

¹H NMR (400 MHz, CDCl₃) δ 5.36 (dt, J=10.2, 5.2 Hz, 3H), 3.76-3.37 (m, 25H), 2.08-1.87 (m, 12H), 1.64-1.48 (m, 4H), 1.41-1.19 (m, 44H), 0.88 (t, J=6.8 Hz, 6H).

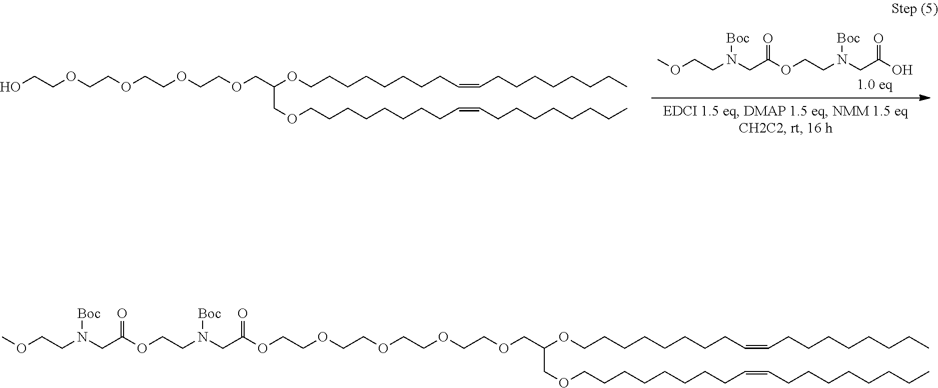

In a 50 mL round bottom flask, 2-[tert-butoxycarbonyl-[2-[2-[tert-butoxycarbonyl(2-methoxyethyl)amino]acetyl]oxyethyl]amino]acetic acid (200 mg, 0.46 mmol), 2-[2-[2-[2-[2,3-bis[(Z)-octadec-9-enoxy]propoxy]ethoxy]ethoxy]ethoxy]ethanol (354 mg, 0.46 mmol) and N,N-dimethylpyridin-4-amine (84.4 mg, 0.69 mmol) were dissolved in DCM-Anhydrous (10 mL) in presence of 4-methylmorpholine (69.8 mg, 0.69 mmol). The reaction was cooled to 0° C. and 3-(ethyliminomethyleneamino)-N,N-dimethyl-propan-1-amine;hydrochloride (132 mg, 0.69 mmol) was added to the mixture. The reaction was warmed to room temperature and stirred overnight. The reaction was monitored by TLC (petroleum ether/EtOAc 5:5, expected product rf: 0.5). Water was added and the organic layers were extracted with DCM (3×15 mL), washed with brine, dried over Na2SO4, filtered and concentrated. The residue was purified by flash column chromatography on silica gel eluting with 1:8 ethyl acetate/petroleum ether to give 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.32 g, 58.6%) as colorless oil.

¹H NMR (500 MHz, CDCl₃) δ 5.39-5.30 (m, 4H), 4.31-4.20 (m, 3H), 4.07-4.00 (m, 2H), 4.00-3.95 (m, 2H), 3.73-3.59 (m, 14H), 3.59-3.38 (m, 15H), 3.30 (d, J=7.0 Hz, 3H), 2.10-1.92 (m, 7H), 1.74 (s, 2H), 1.61-1.50 (m, 4H), 1.50-1.37 (m, 17H), 1.38-1.18 (m, 45H), 0.88 (t, J=6.9 Hz, 6H).

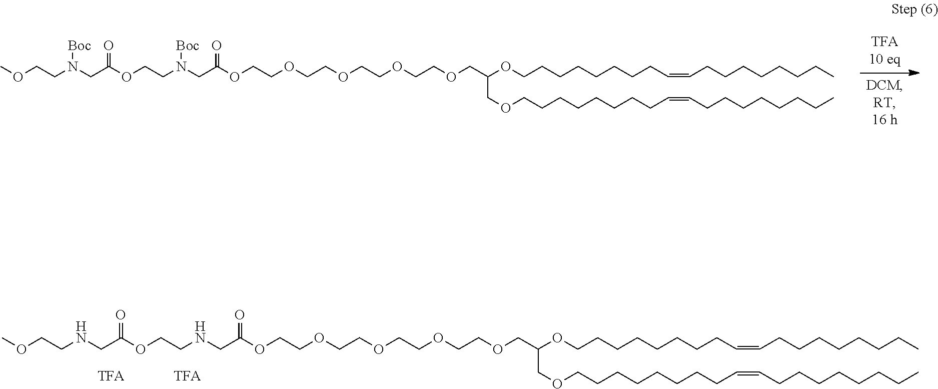

To a mixture of 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.32 g, 0.27 mmol) in DCM (5 mL) was added TFA (0.308 g, 2.7 mmol) at room temperature. The mixture was stirred at ambient temperature for 16 h. The mixture was concentrated and dried under vacuum for 2 h to give 2-[[2-[2-[2-[2-[2-[2,3-bis[(Z)-octadec-9-enoxy]propoxy]ethoxy]ethoxy]ethoxy]ethoxy]-2-oxo-ethyl]amino]ethyl 2-(2-methoxyethylamino)acetate;2,2,2-trifluoroacetic acid (0.23 g, 66.7%) as yellow oil.

¹H NMR (500 MHz, CDCl₃) δ 5.40-5.30 (m, 3H), 4.60 (s, 2H), 4.39-4.31 (m, 2H), 4.03 (s, 2H), 3.96 (s, 2H), 3.73-3.68 (m, 4H), 3.67-3.61 (m, 14H), 3.60-3.40 (m, 13H), 3.36 (s, 3H), 3.33-3.29 (m, 2H), 2.06-1.92 (m, 8H), 1.57-1.53 (m, 4H), 1.31-1.23 (m, 45H), 0.88 (t, J=6.9 Hz, 7H).

### Example 3: Synthesis of 2-[[2-[2-[2-[2-[2-[2,3-bis[(Z)-octadec-9-enoxy]propoxy]ethoxy]ethoxy]ethoxy]ethoxy]-2-oxoethyl]amino]ethyl (2S)-2-amino-3-methoxy-propanoate; Hydrochloride (Compound (VI))

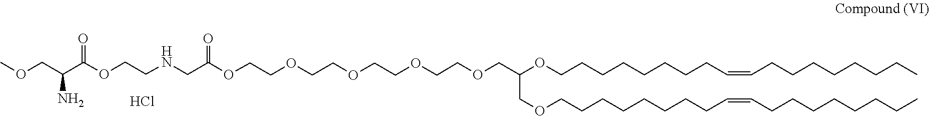 [SMILES: CCCCCCCC/C=C\CCCCCCCCOCC(COCCOCCOCCOCCOC(=O)CNCCOC(=O)[C@@H](N)COC)OCCCCCCCC/C=C\CCCCCCCC ; Cl]

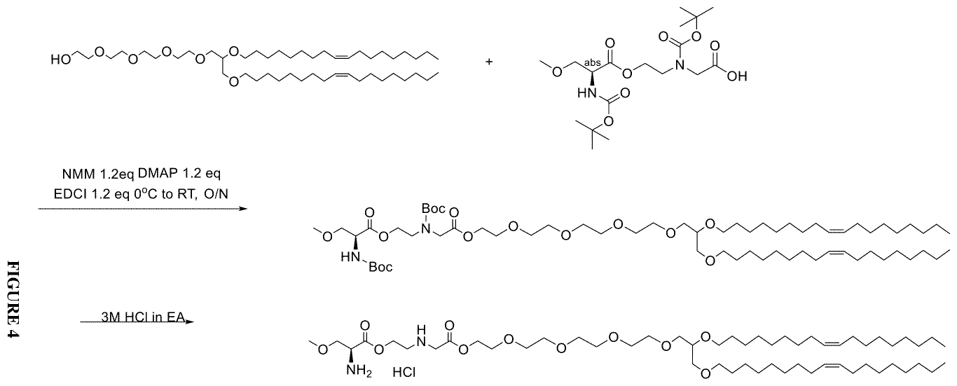

Compound (VI) was synthesized based on the chemistry shown in Scheme (5) represented on FIG. 4.

Compound (VI-A) was synthesized based on the chemistry shown in Scheme (6).

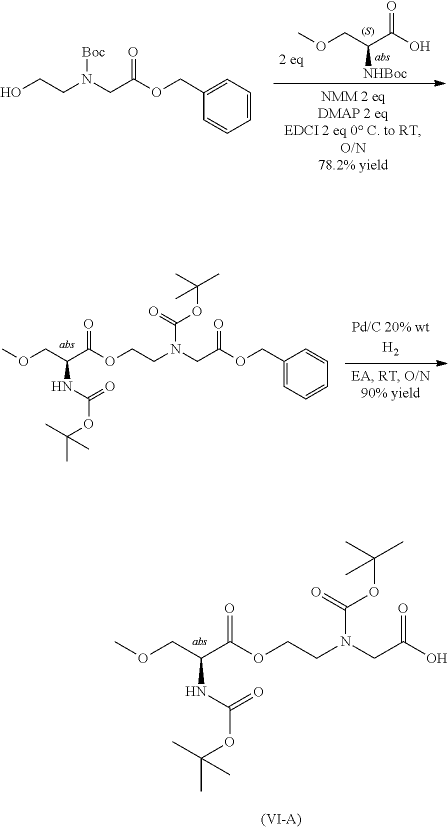

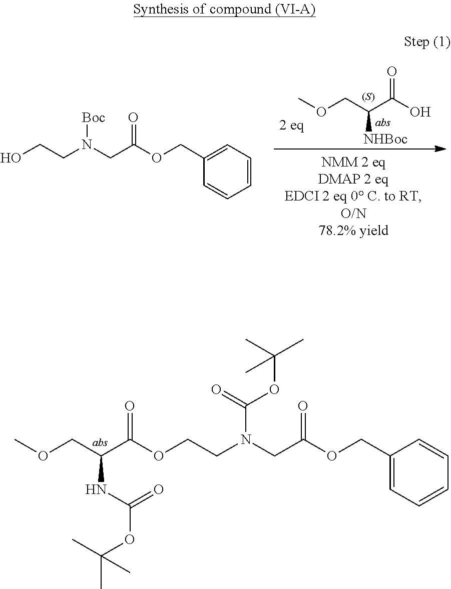

In a 250 ml round bottom flask were dissolved benzyl 2-[tert-butoxycarbon hydroxyethyl)amino]acetate (7 g, 22.6 mmol), (2S)-2-(tert-butoxycarbonylamino)-3-methoxy-propanoic acid (9.92 g, 45.3 mmol), 4-methylmorpholine (4.58 g, 45.3 mmol) and N,N-dimethylpyridin-4-amine (5.53 g, 45.3 mmol) in dry dichloromethane (100 ml). The mixture was cooled to 0° C. and 3-(ethyliminomethyleneamino)-N,N-dimethyl-propan-1-amine;hydrochloride (8.68 g, 45.3 mmol) was added portion wise over 45 min. The mixture was stirred at room temperature overnight. LCMS showed the start material was converted into the product. Water was added, organic layer was separated and washed again with water (2*100 ml) then brine. The organic phase was dried over sodium sulfate, filtrated and solvent was removed under reduced. The crude product was purified by flash chromatography on 340 g silica gel column eluted with 0% to 40% ethyl acetate in petroleum ether to give 2-[(2-benzyloxy-2-oxo-ethyl)-tert-butoxycarbonyl-amino]ethyl (2S)-2-(tert-butoxycarbonylamino)-3-methoxy-propanoate as a colorless oil (9.51 g, 78.2% Yield).

LC-Mass Method: Mobile Phase: A: water (10 mmol NH4HCO3) B: Acetonitrile Gradient from 10 to 95% of B in 1.5 min Flow Rate: 1.5 mL/min Column: XBridge C18 (4.6×50 mm, 3.5 um); LC purity: 69% (214 nm); Mass: find peak 411 (product-Boc+1)* at 2.197 min.

¹H NMR (400 MHz, CDCl₃) δ 7.35 (d, J=3.9 Hz, 5H), 5.35 (d, J=4.6 Hz, 1H), 5.30 (s, 1H), 5.17 (d, J=3.6 Hz, 2H), 4.41-4.25 (m, 3H), 4.07 (s, 1H), 3.97 (s, 1H), 3.80-3.75 (m, 1H), 3.56 (d, J=5.5 Hz, 3H), 3.31 (d, J=1.5 Hz, 3H), 1.54 (s, 1H), 1.45 (dd, J=8.9, 3.5 Hz, 13H), 1.35 (s, 5H).

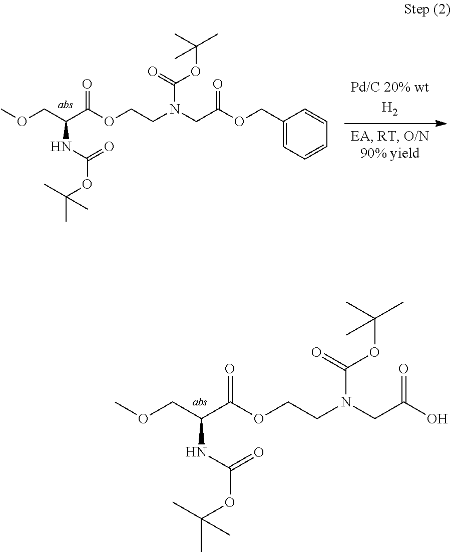 [SMILES: COC[C@H](NC(=O)OC(C)(C)C)C(=O)OCCN(CC(=O)OCc1ccccc1)C(=O)OC(C)(C)C ; COC[C@H](NC(=O)OC(C)(C)C)C(=O)OCCN(CC(=O)O)C(=O)OC(C)(C)C]

To the solution of 2-[(2-benzyloxy-2-oxo-ethyl)-tert-butoxycarbonyl-amino]ethyl (2S)-2-(tert-butoxycarbonylamino)-3-methoxy-propanoate (8.51 g, 16.7 mmol) in ethyl acetate (90 ml) was added palladium (10%, 1.77 g, 1.67 mmol). The mixture was stirred at room temperature under hydrogen atmosphere for 16 h. LCMS showed the start material was converted into the product. The mixture was filtered through celite, wash with ethyl acetate. Solvent was removed under reduced pressure to give 2-[tert-butoxycarbonyl-[2-[(2S)-2-(tert-butoxycarbonylamino)-3-methoxy-propanoyl]oxyethyl]amino]acetic acid (7.50 g, 90% Yield) as colorless oil.

LC-Mass Method: Mobile Phase: A: water (10 mmol NH4HCO3) B: Acetonitrile Gradient from 10 to 95% of B in 1.5 min Flow Rate: 1.5 mL/min Column: XBridge C18 (4.6×50 mm, 3.5 um); LC purity: 71% (214 nm); Mass: find peak 321 (product-Boc+1)* at 1.579 min.

¹H NMR (400 MHz, CDCl₃) δ 8.12 (s, 1H), 5.45 (d, J=7.3 Hz, 111), 5.30 (s, 1H), 4.50-4.31 (m, 2H), 4.27-4.06 (m, 2H), 3.99-3.74 (m, 2H), 3.72-3.54 (m, 2H), 3.45 (d, J=15.3 Hz, 1H), 3.34 (s, 3H), 1.53-1.42 (m, 18H).

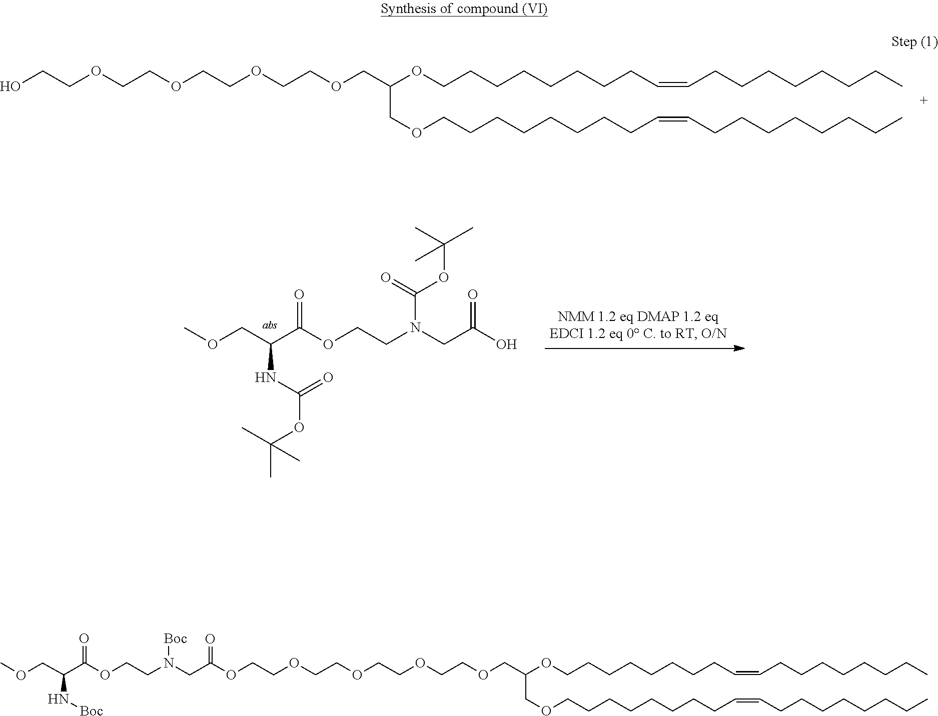

In a 50 ml round bottom flask were dissolved 2-[2-[2-[2-[2,3-bis[(Z)-octadec-9-enoxy]propoxy]ethoxy]ethoxy]ethoxy]ethanol (200 mg, 0.26 mmol), 2-[tert-butoxycarbonyl-[2-[(2S)-2-(tert-butoxycarbonylamino)-3-methoxy-propanoyl]oxyethyl]amino]acetic acid (131 mg, 0.312 mmol), 4-methylmorpholine (32 mg, 0.312 mmol) and N,N-dimethylpyridin-4-amine (38 mg, 0.312 mmol) in dry dichloromethane (5 ml). The mixture was cooled to 0° C. and 3-(ethyliminomethyleneamino)-N,N-dimethyl-propan-1-amine;hydrochloride (60 mg, 0.312 mmol) was added portion wise over 15 min. The mixture was stirred at room temperature overnight. LCMS showed the start material was converted into the product. Water was added, organic layer was separated and washed again with water (2*20 ml) then brine. The organic phase was dried over sodium sulfate, filtrated and solvent was removed under reduced. The crude product was purified by column chromatography on 40 g silica gel column (petroleum ether/ethyl acetate 0%→40% on 10 VC then 40% on 11 VC) to give 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl (2S)-2-(tert-butoxycarbonylamino)-3-methoxy-propanoate as a colorless oil (EXP-20-IJ2428, EXP-20-IJ0405-1, 200 mg, 62% Yield).

¹H NMR (400 MHz, CDCl₃) δ 5.34 (s, 4H), 4.39 (s, 1H), 4.29 (d, J=5.8 Hz, 4H), 4.05 (s, 1H), 3.97 (s, 1H), 3.79 (d, J=9.5 Hz, 2H), 3.72-3.62 (m, 13H), 3.60-3.39 (m, 13H), 3.34 (s, 2H), 2.01 (dd, J=14.9, 9.2 Hz, 8H), 1.54 (d, J=6.8 Hz, 6H), 1.45 (t, J=10.5 Hz, 18H), 1.38-1.20 (m, 43H), 0.88 (s, 6H).

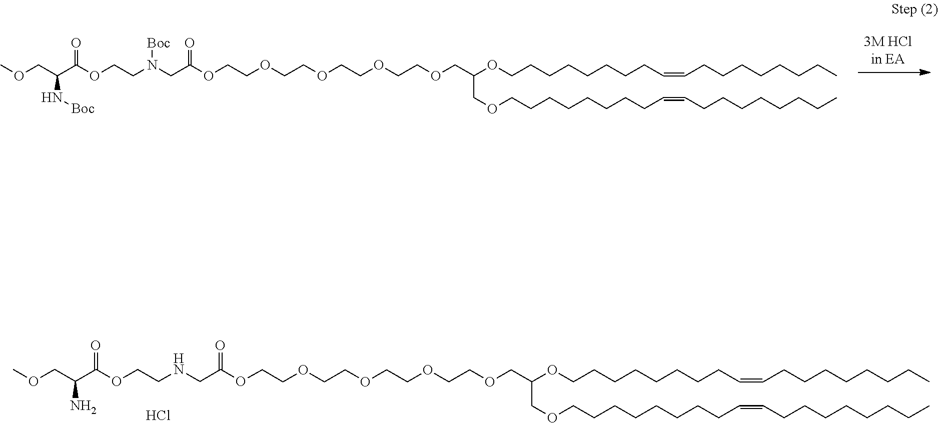

A solution of 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl (2S)-2-(tert-butoxycarbonylamino)-3-methoxy-propanoate (200 mg, 0.171 mmol) in 3 M HCl in ethyl acetate (2 ml) was stirred for 16 hrs at room temperature. The mixture was concentrated to give 2-[[2-[2-[2-[2-[2-[2,3-bis[(Z)-octadec-9-enoxy]propoxy]ethoxy]ethoxy]ethoxy]ethoxy]-2-oxo-ethyl]amino]ethyl (2S)-2-amino-3-methoxy-propanoate;hydrochloride as colorless oil (EXP-20-IJ2430, EXP-20-IJ0405, 125.9 mg, 72.1% yield).

¹H NMR (400 MHz, CDCl₃) δ 10.11 (s, 2H), 8.74 (s, 3H), 5.41-5.29 (m, 3H), 5.19-3.91 (m, 10H), 3.55 (ddd, J=36.6, 17.1, 3.8 Hz, 27H), 2.02 (dd, J=21.3, 15.7 Hz, 8H), 1.55 (s, 5H), 1.49-0.95 (m, 44H), 0.88 (t, J=6.8 Hz, 6H).

### Example 4: Synthesis of 2-[[2-[2-[2-[2-[2,3-bis[(Z)-octadec-9-enoxy]propoxycarbonylamino]ethoxy]ethoxy]ethoxy]-2-oxoethyl]amino]ethyl 2-(2-methoxyethylamino)acetate;2,2,2-trifluoroacetic Acid (Compound (VII))

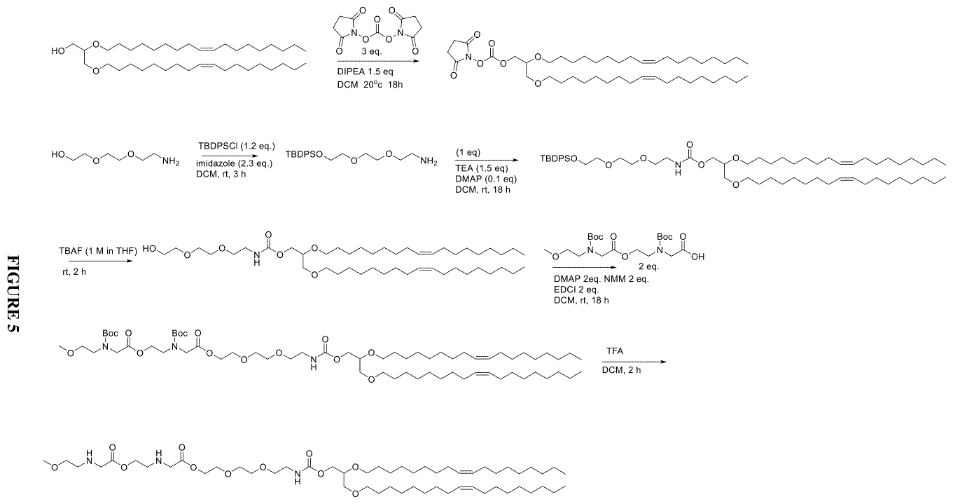

Compound (VII) was synthesized based on the chemistry shown in Scheme (7) represented on FIG. 5.

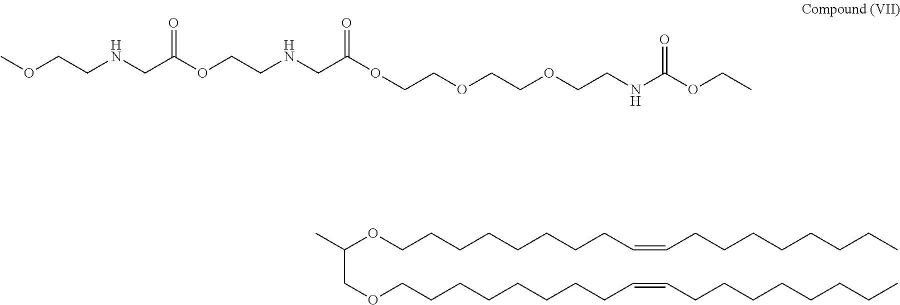

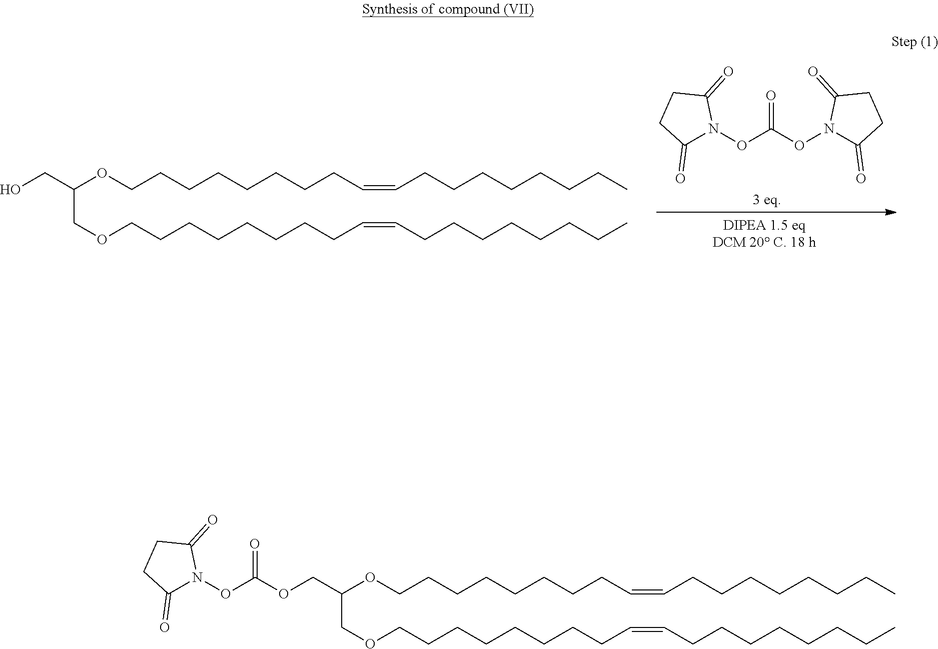 [SMILES: CCCCCCCC/C=C\CCCCCCCCOCC(CO)OCCCCCCCC/C=C\CCCCCCCC ; O=C(ON1C(=O)CCC1=O)ON1C(=O)CCC1=O ; CCCCCCCC/C=C\CCCCCCCCOCC(COC(=O)ON1C(=O)CCC1=O)OCCCCCCCC/C=C\CCCCCCCC]

To a stirred solution of 2,3-bis[(Z)-octadec-9-enoxy]propan-1-ol (500 mg, 0.83 mmol) in N,N-Dimethylformamide (5 mL) was added bis(2,5-dioxopyrrolidin-1-yl) carbonate (668 mg, 2.48 mmol) and 4-Dimethylaminopyridine (0.1 g, 0.83 mmol). The solution mixture was stirred at 20′C for 18 h. TLC indicated that the starting material was consumed up and the product was the major one. The reaction was partitioned between ethyl acetate and water. The aqueous layer was extracted with ethyl acetate again. The combined organic layers were washed with brine, dried over sodium sulfate, filtered and concentrated. The residue was purified by flash chromatography eluted with 10% to 20% ethyl acetate in petroleum ether to give 2,3-bis[(Z)-octadec-9-enoxy]propyl (2,5-dioxopyrrolidin-1-yl) carbonate (550 mg, 0.73 mmol, 88% yield) as colorless oil.

¹H NMR (500 MHz, CDCl₃) δ 5.40-5.31 (m, 4H), 4.46 (dd, J=11.1, 3.8 Hz, 1H), 4.35 (dd, J=11.1, 6.3 Hz, 1H), 3.74-3.65 (m, 1H), 3.60-3.49 (m, 3H), 3.49-3.41 (m, 3H), 2.83 (s, 4H), 2.06-1.92 (m, 8H), 1.55 (dd, J=14.4, 7.2 Hz, 4H), 1.38-1.21 (m, 44H), 0.88 (t, J=6.9 Hz, 6H).

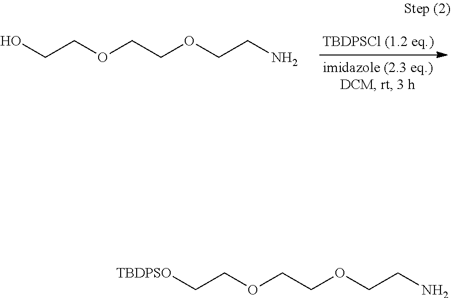

To a solution of 2-[2-(2-aminoethoxy)ethoxy]ethanol (1.0 g, 6.7 mmol) and imidazole (1.08 g, 15.4 mmol) in CH₂Cl₂ (20 mL) was added TBDPSCl (2.18 g, 7.71 mmol). The reaction was stirred for 18 hrs at room temperature. The mixture was diluted with CH₂Cl₂ (30 mL) and washed the resulting mixture with brine. The organic layer was dried over Na₂SO₄, filtered and concentrated. The residue was purified by column chromatography (SiO₂, CH₂Cl₂/MeOH/NH₄OH 80:20:0.25) to obtain 2-[2-[2-[tert-butyl(diphenyl)silyl]oxyethoxy]ethoxy]ethanamine (1.3 g, 3.35 mmol, 50% yield) as colorless oil.

MS (ESI+) m/z 388.2 (M+H)⁺

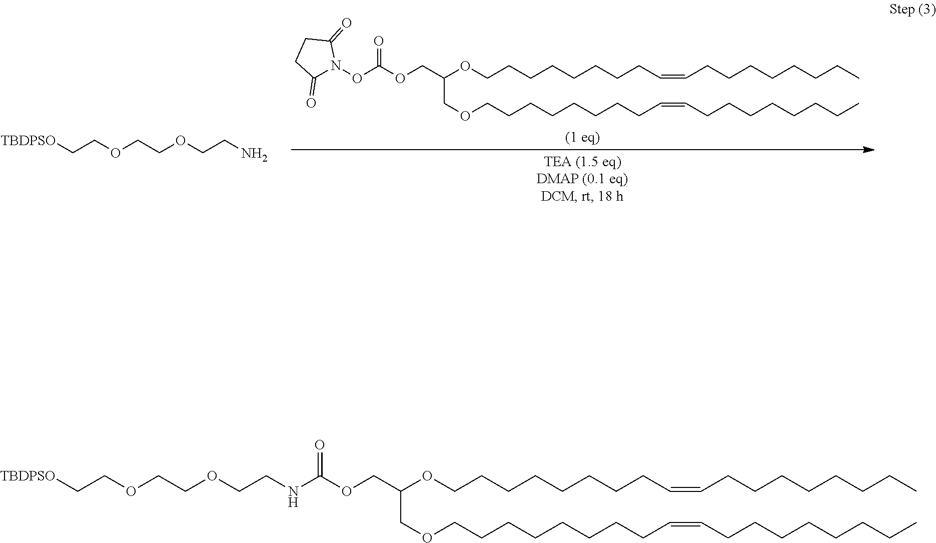

To a solution of 2,3-bis[(Z)-octadec-9-enoxy]propyl (2,5-dioxopyrrolidin-1-yl) carbonate (450 mg, 0.58 mmol) in Dichloromethane (20 ml) was added 2-[2-[2-[tert-butyl(diphenyl)silyl]oxyethoxy]ethoxy]ethanamine (226 mg. 0.58 mmol) followed by Triethylamine (88.4 mg, 0.87 mmol) and 4-Dimethylaminopyridine (7 mg, 0.06 mmol). The mixture was stirred at 25° C. for 18 hr. TLC indicated that the starting material was consumed. The mixture was concentrated and purified by flash chromatography eluted with 1% to 5% methanol in dichloromethane to give 2,3-bis[(Z)-octadec-9-enoxy]propyl N-[2-[2-[2-[tert-butyl(diphenyl)silyl]oxyethoxy]ethoxy]ethyl]carbamate (500 mg, 0.47 mmol, 81% yield) as colorless oil.

¹H NMR (500 MHz, CDCl₃) δ 7.68 (dd, J=7.9, 1.4 Hz, 4H), 7.45-7.35 (m, 6H), 5.41-5.31 (m, 3H), 5.23-5.17 (m, 1H), 4.18 (dd, J=11.4, 4.1 Hz, 1H), 4.09 (dd, J=11.4, 5.4 Hz, 1H), 3.81 (t, J=5.3 Hz, 2H), 3.65-3.50 (m, 11H), 3.46 (d, J=5.3 Hz, 2H), 3.42 (t, J=6.7 Hz, 2H), 3.38-3.31 (m, 2H), 2.06-1.92 (m, 7H), 1.59-1.49 (m, 4H), 1.36-1.20 (m, 46H), 1.05 (s, 9H), 0.88 (t, J=6.9 Hz, 6H).

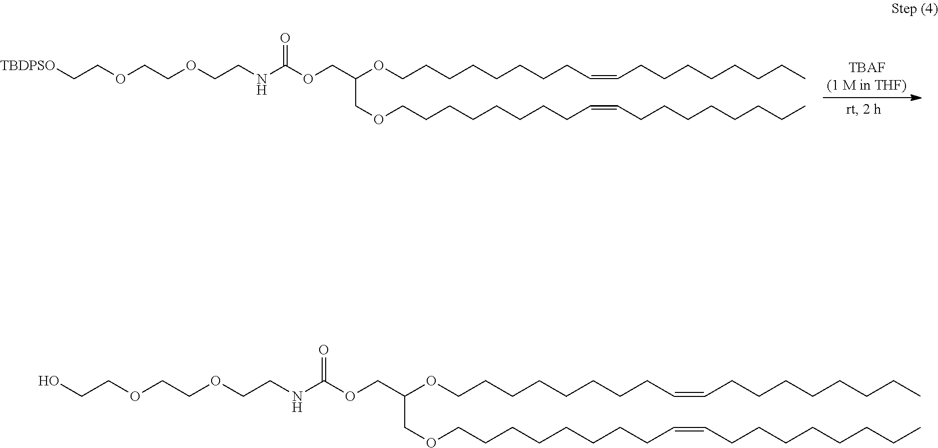

The mixture of 2,3-bis[(Z)-octadec-9-enoxy]propyl N-[2-[2-[2-[tert-butyl(diphenyl)silyl]oxyethoxy]ethoxy]ethyl]carbamate (500 mg, 0.47 mmol) and TBAF (1 M in THF, 3 mL) was stirred at 25° C. for 2 h. TLC (ethyl acetate/petroleum ether 1/1) indicated that the starting material was consumed and a new spot was observed. The mixture was concentrated and combined with above batch, and purified by flash chromatography eluted with 20% to 50% ethyl acetate in petroleum ether to give 2,3-bis[(Z)-octadec-9-enoxy]propyl N-[2-[2-(2-hydroxyethoxy)ethoxy]ethyl]carbamate (0.32 g, 0.4 mmol, 84% yield) as colorless oil.

¹H NMR (400 MHz, CDCl₃) δ 5.43 (s, 1H), 5.40-5.29 (m, 4H), 4.27-4.05 (m, 2H), 3.75 (s, 2H), 3.68-3.31 (m, 18H), 2.57 (s, 1H), 2.08-1.88 (m, 7H), 1.58-1.50 (m, 4H), 1.39-1.19 (m, 49H), 0.88 (t, J=6.8 Hz, 6H).

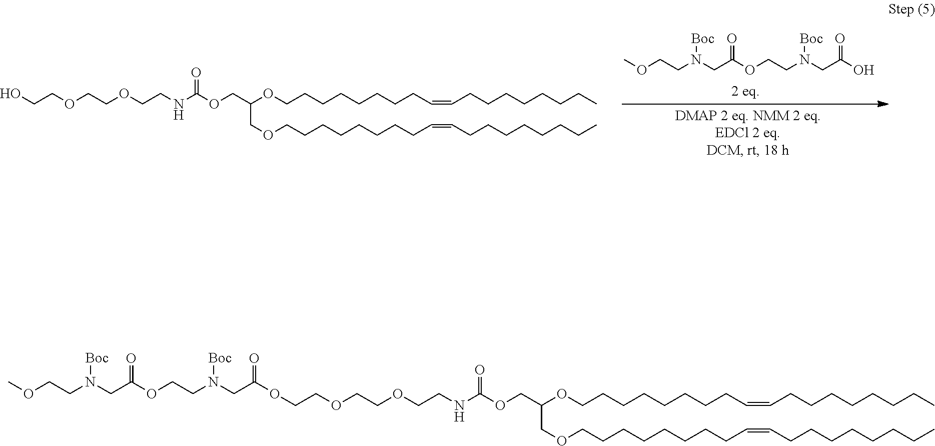

In a 50 mL round bottom flask, 2-[tert-butoxycarbonyl-[2-[2-[tert-butoxycarbonyl(2-methoxyethyl)amino]acetyl]oxyethyl]amino]acetic acid (170 mg, 0.39 mmol), 2,3-bis[(Z)-octadec-9-enoxy]propyl N-[2-[2-(2-hydroxyethoxy)ethoxy]ethyl]carbamate (316 mg, 0.39 mmol) and N,N-dimethylpyridin-4-amine (52.6 mg, 0.43 mmol) were dissolved in DCM-Anhydrous (10 mL) in presence of 4-methylmorpholine (43.5 mg, 0.43 mmol). The reaction was cooled to 0° C. and 3-(ethyliminomethyleneamino)-N,N-dimethyl-propan-1-amine hydrochloride (82.5 mg, 0.43 mmol) was added to the mixture. The reaction was warmed to room temperature and stirred overnight. The reaction was monitored by TLC (petroleum ether/EtOAc 5:5, expected product rf: 0.5). The solvent was removed and the residue was purified by flash column chromatography on silica gel eluting with 1:2 to 1:1 ethyl acetate/petroleum ether to give 2-[[2-[2-[2-[2-[2,3-bis[(Z)-octadec-9-enoxy]propoxycarbonylamino]ethoxy]ethoxy]ethoxy]-2-oxo-ethyl]-tert-butoxycarbonyl-amino]ethyl 2-[tert-butoxycarbonyl(2-methoxyethyl)amino]acetate (0.19 g, 0.156 mmol, 40%) as colorless oil.

¹H NMR (500 MHz, CDCl₃) δ 5.40-5.32 (m, 3H), 5.22 (s, 1H), 4.32-4.27 (m, 2H), 4.27-4.15 (m, 3H), 4.14-4.07 (m, 1H), 4.07-4.01 (m, 2H), 3.98 (t, J=5.5 Hz, 2H), 3.73-3.67 (m, 2H), 3.65-3.58 (m, 5H), 3.58-3.52 (m, 5H), 3.52-3.46 (m, 6H), 3.43 (t, J=6.6 Hz, 3H), 3.40-3.34 (m, 2H), 3.30 (d, J=6.9 Hz, 3H), 2.06-1.92 (m, 8H), 1.58-1.51 (m, 4H), 1.44 (d, J=22.4 Hz, 18H), 1.35-1.22 (m, 44H), 0.90-0.85 (m, 6H).

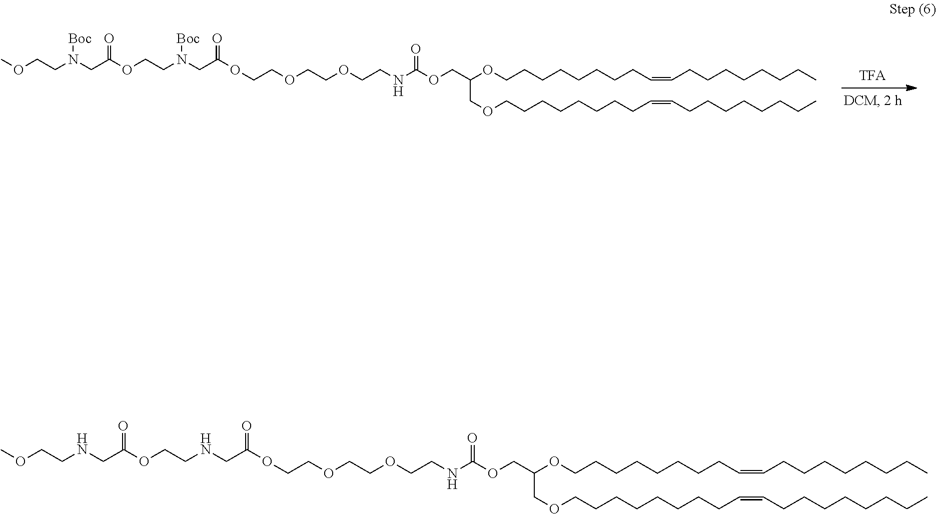

To the solution of 2-[[2-[2-[2-[2-[2,3-bis[(Z)-octadec-9-enoxy]propoxycarbonylamino]ethoxy]ethoxy]ethoxy]-2-oxo-ethyl]-tert-butoxycarbonyl-amino]ethyl 2-[tert-butoxycarbonyl(2-methoxyethyl)amino]acetate (0.19 g, 0.156 mmol) in DCM (5 mL) at room temperature was added 2,2,2-trifluoroacetic acid (1 mL, 13.5 mmol) and the reaction was stirred at room temperature for 2 hrs. The reaction was monitored by TLC (petroleum ether/EtOAc 5:5, starting material rf: 0.5) which indicated that the starting material was consumed completely. The solvent was removed and the material was azeotroped with dichloromethane several times and then dried under vacuum to give 2-[[2-[2-[2-[2-[2,3-bis[(Z)-octadec-9-enoxy]propoxycarbonylamino]ethoxy]ethoxy]ethoxy]-2-oxo-ethyl]amino]ethyl 2-(2-methoxyethylamino)acetate;2,2,2-trifluoroacetic acid (0.19 g, 0.156 mmol, quant.) as light yellow oil.

¹H NMR (500 MHz, CDCl₃) δ 5.40-5.27 (m, 4H), 4.60 (s, 2H), 4.38 (s, 2H), 4.19 (s, 2H), 4.14-4.06 (m, 2H), 4.04-3.95 (m, 5H), 3.71 (s, 4H), 3.62 (s, 5H), 3.57-3.52 (m, 4H), 3.50-3.42 (m, 6H), 3.39-3.28 (m, 7H), 2.05-1.91 (m, 7H), 1.59-1.50 (m, 4H), 1.36-1.20 (m, 47H), 0.88 (t, J=6.9 Hz, 6H).

MS (ESI+) m/z 984.8 (M+H)+

### Example 5: Synthesis of 2-[2-[2-[2-[2-[2-[2-(2-methoxyethylamino)acetyl]oxyethylamino]acetyl]oxyethoxy]ethoxy]ethoxy]ethyl 2,3-bis[(Z)-octadec-9-enoxy]propanoate;2,2,2-trifluoroacetic Acid (Compound (VIII))

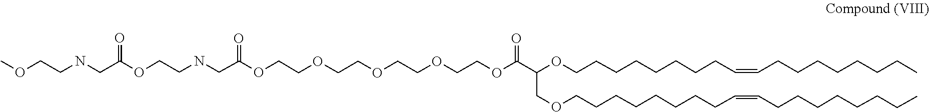

Synthesis of Compound (VIII)

To the solution of 2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethyl 2,3-bis[(Z)-octadec-9-enoxy]propanoate (0.35 g, 0.277 mmol) in DCM-Anhydrous (6 mL) at room temperature was added TFA (1 mL) and the mixture was stirred at room temperature for 2 h. The reaction was done monitored by TLC (petroleum ether/EtOAc 5:5, SM rf: 0.5). The solvent was removed and the residue was azeotroped with dichloromethane several times. Then the material was dried under vacuum for 2 h to give 2-[2-[2-[2-[2-[2-[2-(2-methoxyethylamino)acetyl]oxyethylamino]acetyl]oxyethoxy]ethoxy]ethoxy]ethyl 2,3-bis[(Z)-octadec-9-enoxy]propanoate;2,2,2-trifluoroacetic acid (0.312 g, 96% yield) as a light yellow oil.

¹H NMR (500 MHz, CDCl₃) δ 5.40-5.31 (m, 4H), 4.63-4.57 (m, 2H), 4.39-4.34 (m, 2H), 4.34-4.27 (m, 2H), 4.09-4.05 (m, 1H), 4.01 (d, J=19.8 Hz, 4H), 3.75-3.59 (m, 17H), 3.52-3.39 (m, 5H), 3.37 (s, 3H), 3.35-3.30 (m, 2H), 2.06-1.90 (m, 8H), 1.63-1.50 (m, 4H), 1.36-1.22 (m, 44H), 0.88 (t, J=6.9 Hz, 6H).

MS (ESI+) m/z 999.8 (M+H)+

### Example 6: Synthesis of 2-[[2-[2-[2-[2-[2-[2,3-bis[(Z)-octadec-9-enoxy]propanoylamino]ethoxy]ethoxy]ethoxy]ethoxy]-2-oxo-ethyl]amino]ethyl 2-(2-methoxyethylamino)acetate;2,2,2-trifluoroacetic Acid (Compound (IX))

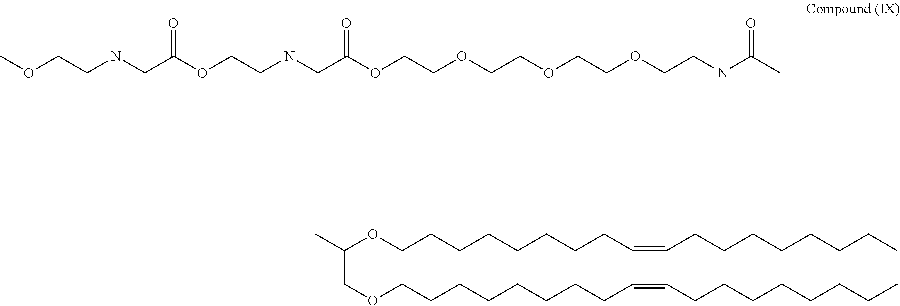

Synthesis of Compound (IX)

A solution of 2-[[2-[2-[2-[2-[2-[2,3-bis[(Z)-octadec-9-enoxy]propanoylamino]ethoxy]ethoxy]ethoxy]ethoxy]-2-oxoethyl]-tert-butoxycarbonyl-amino]ethyl 2-[tert-butoxycarbonyl(2-methoxyethyl)amino]acetate (0.25 g, 0.2 mmol) in dichloromethane (10 ml) was treated with 2,2,2-trifluoroacetic acid (1 mL) and the mixture was stirred for 2 h at RT. TLC (10% methanol in dichloromethane) indicated that the starting material was disappeared. The reaction was concentrated and the product was azeotroped with dichloromethane several times and dried under vacuum for 5 h to give 2-[[2-[2-[2-[2-[2-[2,3-bis[(Z)-octadec-9-enoxy]propanoylamino]ethoxy]ethoxy]ethoxy]ethoxy]-2-oxoethyl]amino]ethyl 2-(2-methoxyethylamino)acetate;2,2,2-trifluoroacetic acid (0.263 g, 0.2 mmol, quant.) as light yellow oil.

¹H NMR (400 MHz, CDCl₃) δ 7.22-7.17 (m, 1H), 5.45-5.27 (m, 4H), 5.11-4.57 (m, 2H), 4.39-4.25 (m, 2H), 4.06-3.88 (m, 5H), 3.78-3.69 (m, 5H), 3.65-3.28 (m, 25H), 3.20-3.14 (m, 3H), 2.11-1.87 (m, 8H), 1.65-1.50 (m, 4H), 1.34-1.22 (m, 44H), 0.88 (t, J=6.8 Hz, 6H)

### Example 7: Synthesis 2-[[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic Acid (Compound (X))

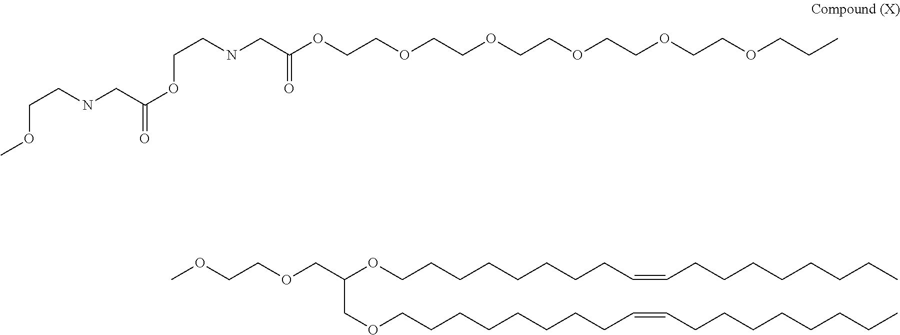

Compound (X) was synthesized according to the following reaction scheme:

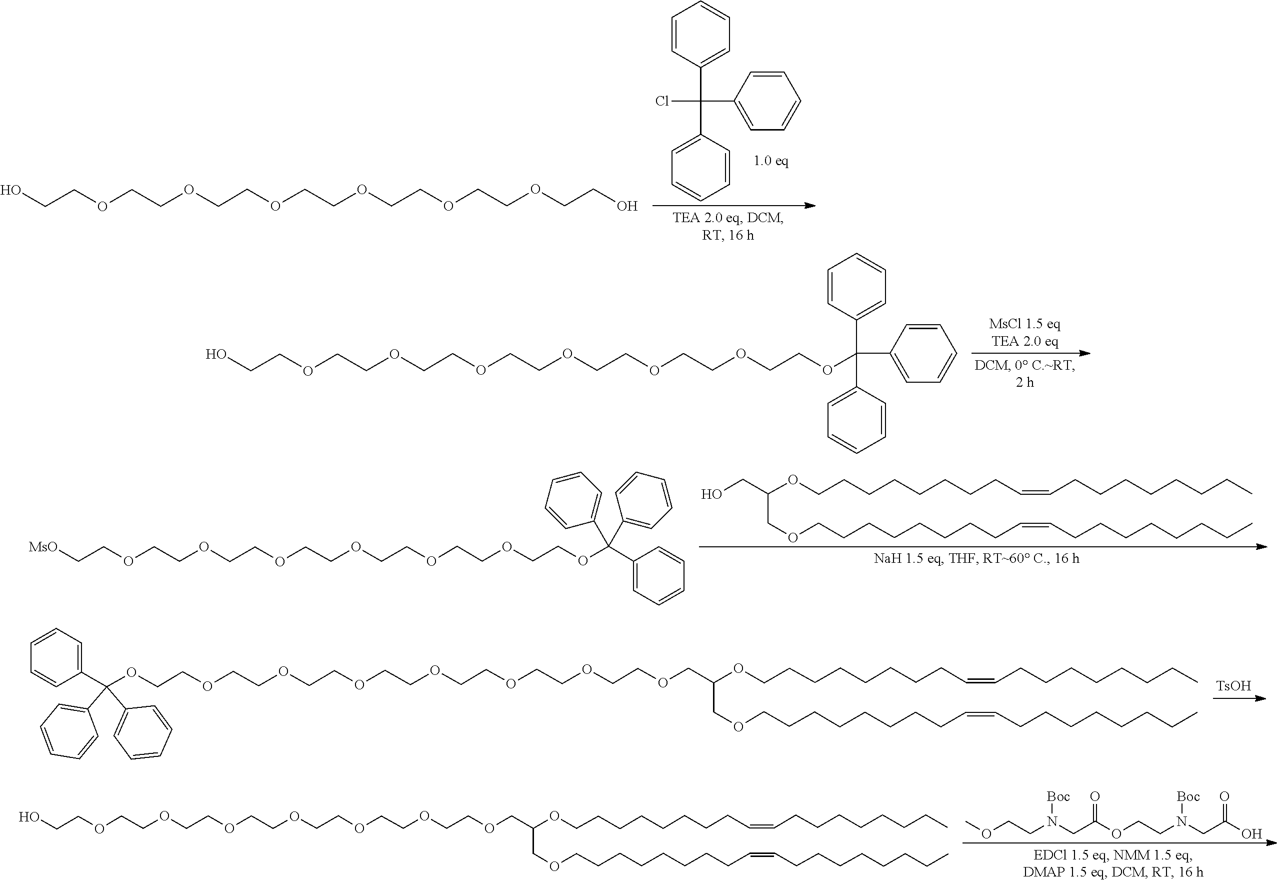

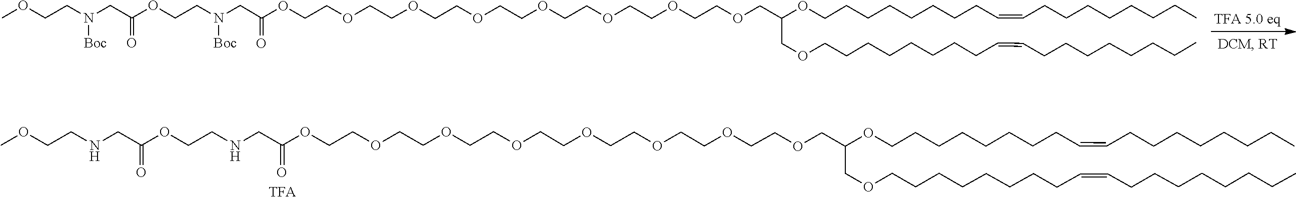

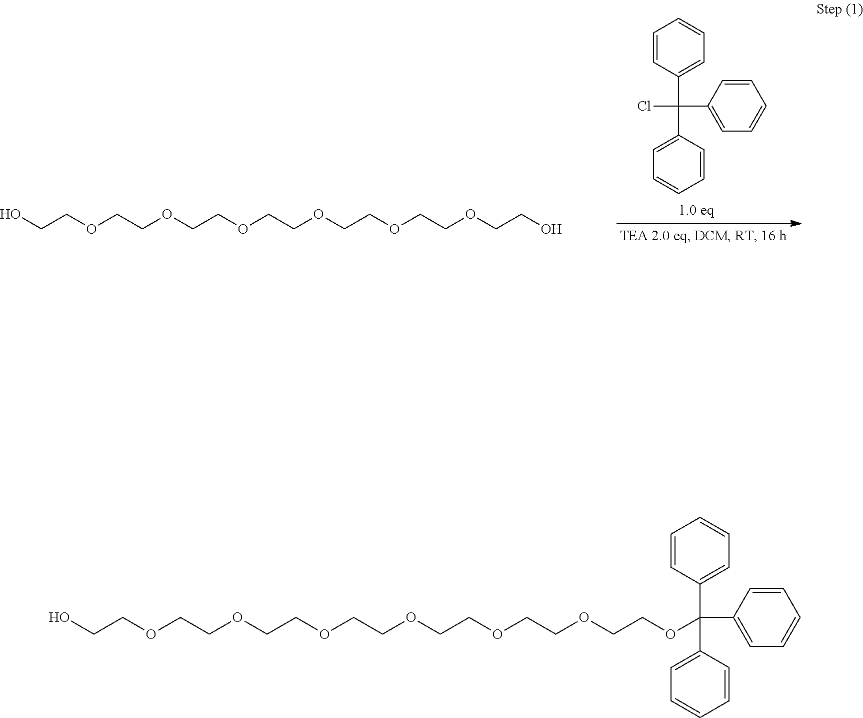 [SMILES: ClC(c1ccccc1)(c1ccccc1)c1ccccc1 ; OCCOCCOCCOCCOCCOCCOCCO ; OCCOCCOCCOCCOCCOCCOCCOC(c1ccccc1)(c1ccccc1)c1ccccc1]

A mixture of 2-[2-[2-[2-[2-[2-(2-hydroxyethoxy)ethoxy]ethoxy]ethoxy]ethoxy]ethoxy]ethanol (5.0 g, 15.3 mmol) and N,N-diethylethanamine (3.1 g, 30.6 mmol) in DCM (100 mL) was added [chloro(diphenyl)methyl]benzene (3.42 g, 12.3 mmol). The mixture was stirred for 16 h at room temperature. The mixture was added DCM (100 mL) and washed with water, brine, dried over Na₂SO₄ and concentrated. The residue was purified by column chromatography on silica gel eluting with 0%-20% MeOH in DCM to afford 2-[2-[2-[2-[2-[2-(2-trityloxyethoxy)ethoxy]ethoxy]ethoxy]ethoxy]ethoxy]ethanol (3.3 g, 41.3%) as colourless oil.

LCMS 586 (M+18), 99% UV:214 nm

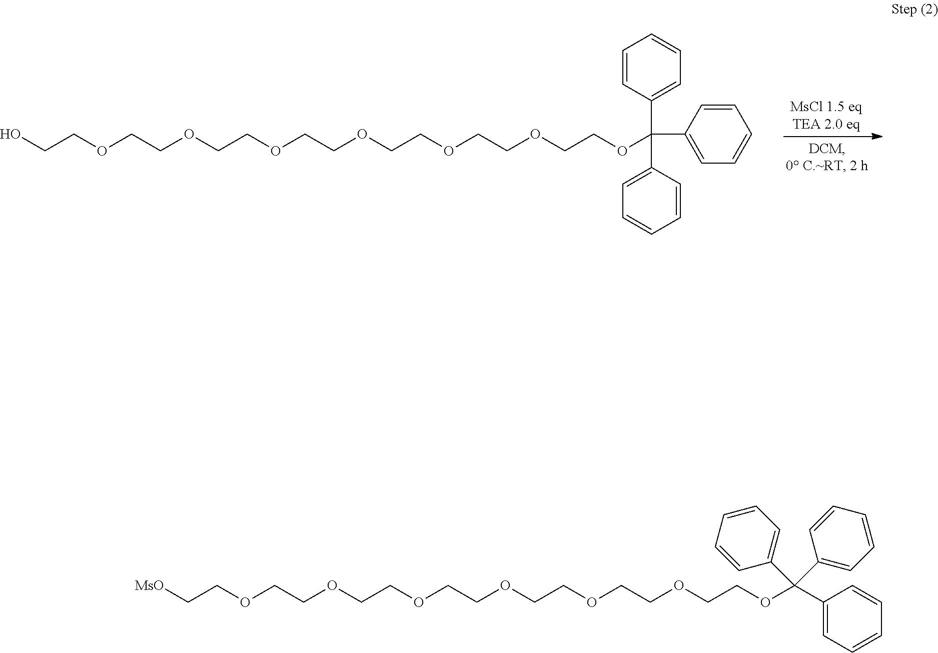 [SMILES: OCCOCCOCCOCCOCCOCCOCCOC(c1ccccc1)(c1ccccc1)c1ccccc1 ; CS(=O)(=O)OCCOCCOCCOCCOCCOCCOCCOC(c1ccccc1)(c1ccccc1)c1ccccc1]

To a mixture of 2-[2-[2-[2-[2-[2-(2-trityloxyethoxy)ethoxy]ethoxy]ethoxy]ethoxy]ethoxy]ethanol (3.6 g, 6.33 mmol) and N,N-diethylethanamine (1.28 g, 12.7 mmol) in DCM (50 mL) cooled to 0° C. was added methanesulfonyl chloride (1.09 g, 9.5 mmol). The reaction mixture was stirred for 3 h at room temperature. The mixture was added DCM (100 mL) and washed with water, brine, dried over Na₂SO₄ and concentrated to give 2-[2-[2-[2-[2-[2-(2-trityloxyethoxy)ethoxy]ethoxy]ethoxy]ethoxy]ethoxy]ethyl methanesulfonate (3.8 g, 93%) as yellow oil.

LCMS 664 (M+18), 97% UV:214 nm

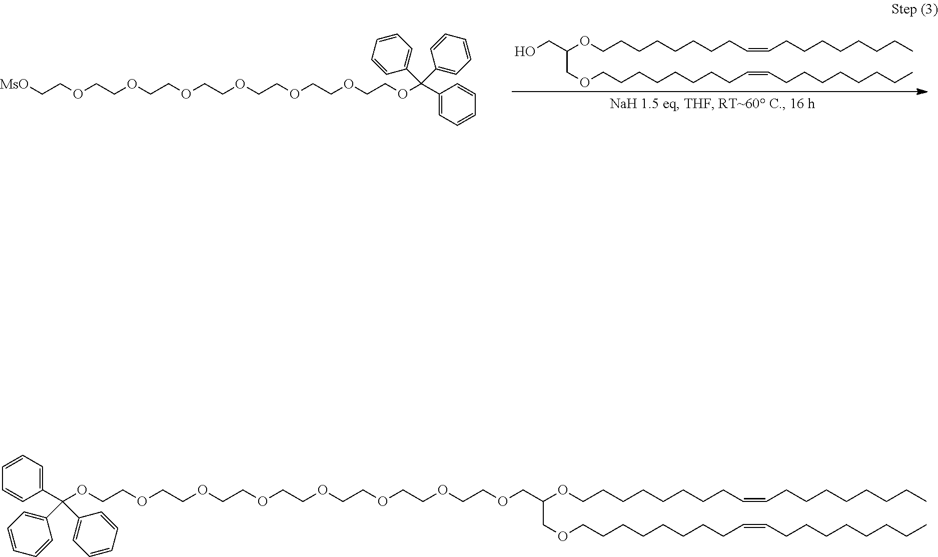 [SMILES: CS(=O)(=O)OCCOCCOCCOCCOCCOCCOCCOC(c1ccccc1)(c1ccccc1)c1ccccc1 ; CCCCCCCC/C=C\CCCCCCCCOCC(CO)OCCCCCCCC/C=C\CCCCCCCC ; CCCCCCCC/C=C\CCCCCCCCOCC(COCCOCCOCCOCCOCCOCCOCCOC(c1ccccc1)(c1ccccc1)c1ccccc1)OCCCCCCCC/C=C\CCCCCCCC]

To a suspension of NaH (60% mineral oil dispersion, 105 mg, 2.63 mmol) in 15 mL of THF was added 2,3-bis[(Z)-octadec-9-enoxy]propan-1-ol (0.78 g, 1.32 mmol) dissolved in 15 mL of THF. The resultant suspension was stirred for 2 h at room temperature. 2-[2-[2-[2-[2-[2-(2-trityloxyethoxy)ethoxy]ethoxy]ethoxy]ethoxy]ethoxy]ethyl methanesulfonate (1.28 g, 1.97 mmol) was added to the suspension, and the reaction mixture was brought to reflux overnight. The reaction mixture was then cooled to room temperature, and water (40 mL) was added. The organic phase was collected and the aqueous phase was extracted with 3×40 ml EtOAc. The organic phases were combined and washed successively with 40 ml of 1N HCl, 40 ml of 5% (w/v) NaHCO₃ and 40 ml of brine and dried on MgSO₄. The solvent was evaporated under reduced pressure and the resulting oil was purified on a silica gel column eluted with Petroleum ether/AcOEt to give [2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy-diphenyl-methyl]benzene (1.1 g, 73%) as a colorless oil.

¹H NMR (500 MHz, CDCl₃) δ 7.49-7.43 (m, 6H), 7.32-7.26 (m, 6H), 7.25-7.20 (m, 3H), 5.39-5.30 (m, 3H), 3.71-3.60 (m, 26H), 3.60-3.39 (m, 10H), 3.23 (t, J=5.2 Hz, 2H), 2.08-1.92 (m, 7H), 1.54 (dd, J=13.3, 6.6 Hz, 4H), 1.28 (t, J=14.4 Hz, 46H), 0.88 (t, J=6.9 Hz, 6H).

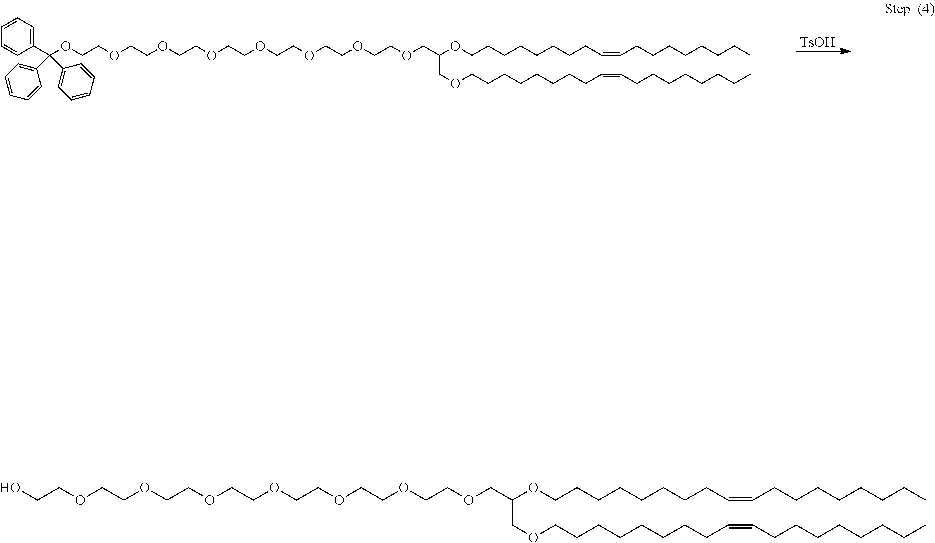 [SMILES: CCCCCCCC/C=C\CCCCCCCCOCC(COCCOCCOCCOCCOCCOCCOCCOC(c1ccccc1)(c1ccccc1)c1ccccc1)OCCCCCCCC/C=C\CCCCCCCC ; "TsOH" ; CCCCCCCC/C=C\CCCCCCCCOCC(COCCOCCOCCOCCOCCOCCOCCO)OCCCCCCCC/C=C\CCCCCCCC]

To a mixture of [2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy-diphenyl-methyl]benzene (1.1 g, 0.962 mmol) in THF (5 mL)/MeOH (5 mL) was added Toluene-4-sulfonic acid (0.915 g, 4.81 mmol) and stirred overnight at room temperature. Et₃N (1 mL) was added to the reaction mixture and concentrated. The residue was purified by flash column chromatography on silica gel eluted with 2:1 ethyl acetate/petroleum ether to give 2-[2-[2-[2-[2-[2-[2-[2,3-bis[(Z)-octadec-9-enoxy]propoxy]ethoxy]ethoxy]ethoxy]ethoxy]ethoxy]ethoxy]ethanol (0.7 g, 81%) as colorless oil.

¹H NMR (500 MHz, CDCl₃) δ 5.34 (td, J=16.2, 8.2 Hz, 3H), 3.74-3.70 (m, 2H), 3.69-3.39 (m, 37H), 3.22 (s, 1H), 2.12-1.93 (m, 12H), 1.60-1.51 (m, 4H), 1.37-1.20 (m, 46H), 0.88 (t, J=6.9 Hz, 6H).

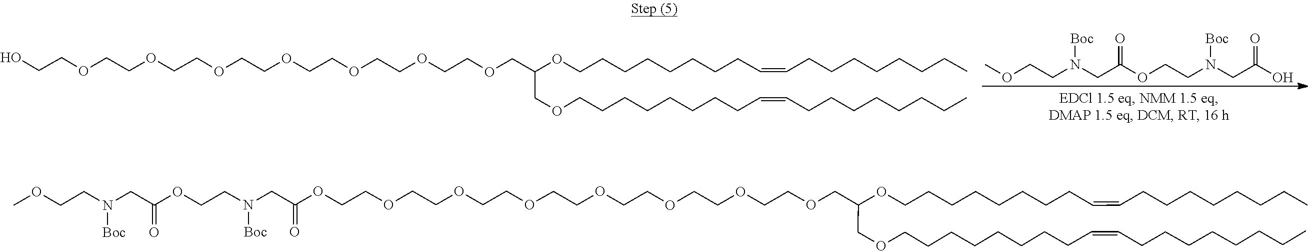

In a 100 mL round bottom flask, 2-[tert-butoxycarbonyl-[2-[2-[tert-butoxycarbonyl(2-methoxyethyl)amino]acetyl]oxyethyl]amino]acetic acid (400 mg, 0.921 mmol), 2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethanol (747 mg, 0.829 mmol), N,N-dimethylpyridin-4-amine (225 mg, 1.84 mmol) were dissolved in DCM-Anhydrous (10 mL) in presence of 4-methylmorpholine (186 mg, 1.84 mmol). The reaction was cooled to 0° C. and EDC HCl (265 mg, 1.38 mmol) was added to the mixture. The reaction was warmed to room temperature and stirred overnight. Water was added and the organic layers were extracted with DCM (3×15 mL), washed with brine, dried over Na2SO4, filtered and concentrated. The residue was purified by flash column chromatography on silica gel eluted with 4:1 ethyl acetate/petroleum ether to give 2-[[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.44 g, 36.3%) as colorless oil.

¹H NMR (400 MHz, CDCl₃) δ 5.34 (t, J=5.3 Hz, 3H), 4.31-4.19 (m, 4H), 4.07-3.94 (m, 4H), 3.73-3.60 (m, 26H), 3.59-3.39 (m, 15H), 3.30 (d, J=5.4 Hz, 3H), 2.06-1.93 (m, 7H), 1.60-1.50 (m, 4H), 1.44 (d, J=18.6 Hz, 18H), 1.38-1.21 (m, 45H), 0.88 (t, J=6.8 Hz, 6H).

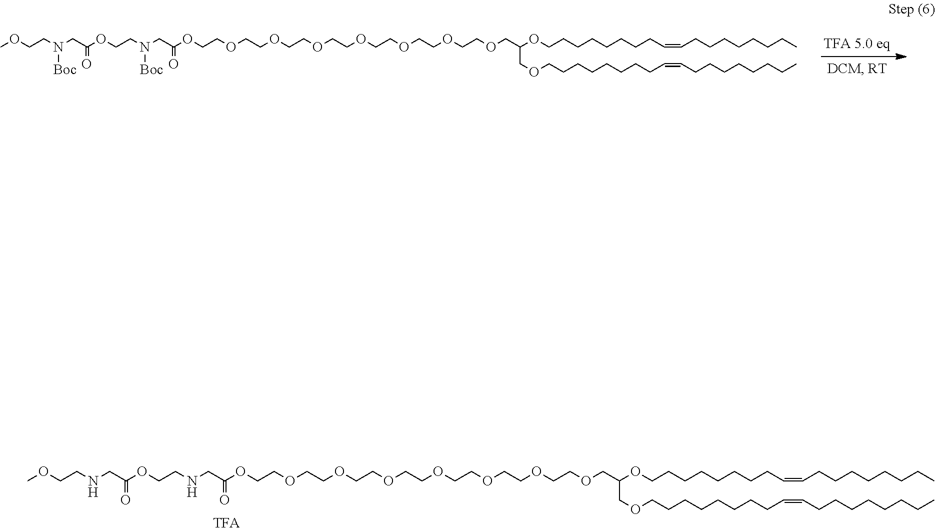

A mixture of 2-[[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.6 g, 0.334 mmol) in DCM (5 ml) was added TFA (0.381 g, 3.34 mmol). The mixture was stirred 3 h at ambient temperature. The mixture was concentrated and dried under vacuum for several hours to give 2-[[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic acid (0.302 g, 67.2%) as brown oil.

¹H NMR (500 MHz, CDCl₃) δ 5.39-5.30 (m, 3H), 4.58 (s, 2H), 4.40-4.34 (m, 2H), 4.04 (d, J=25.9 Hz, 4H), 3.74-3.40 (m, 39H), 3.39-3.30 (m, 5H), 2.06-1.91 (m, 6H), 1.59-1.50 (m, 4H), 1.37-1.18 (m, 45H), 0.88 (t, J=6.9 Hz, 6H).

### Example 8: Synthesis of 2-[[2-[2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethoxy]-2-oxoethyl]amino]ethyl 2-(2-methoxyethylamino)acetate;2,2,2-trifluoroacetic Acid (Compound (XI))

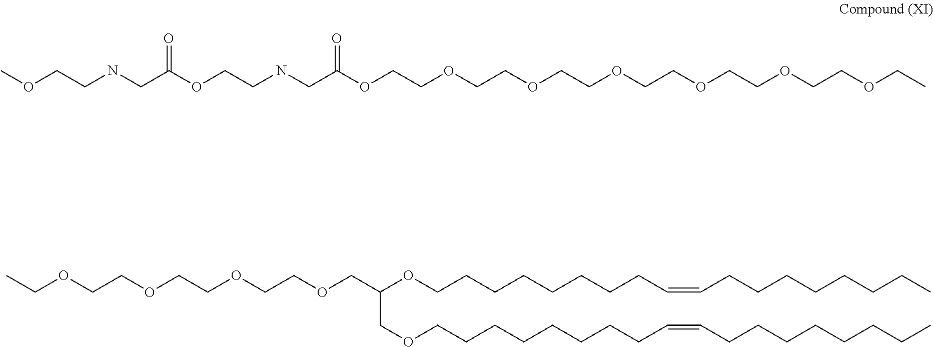

Compound (XI) was synthesized according to the following scheme reaction:

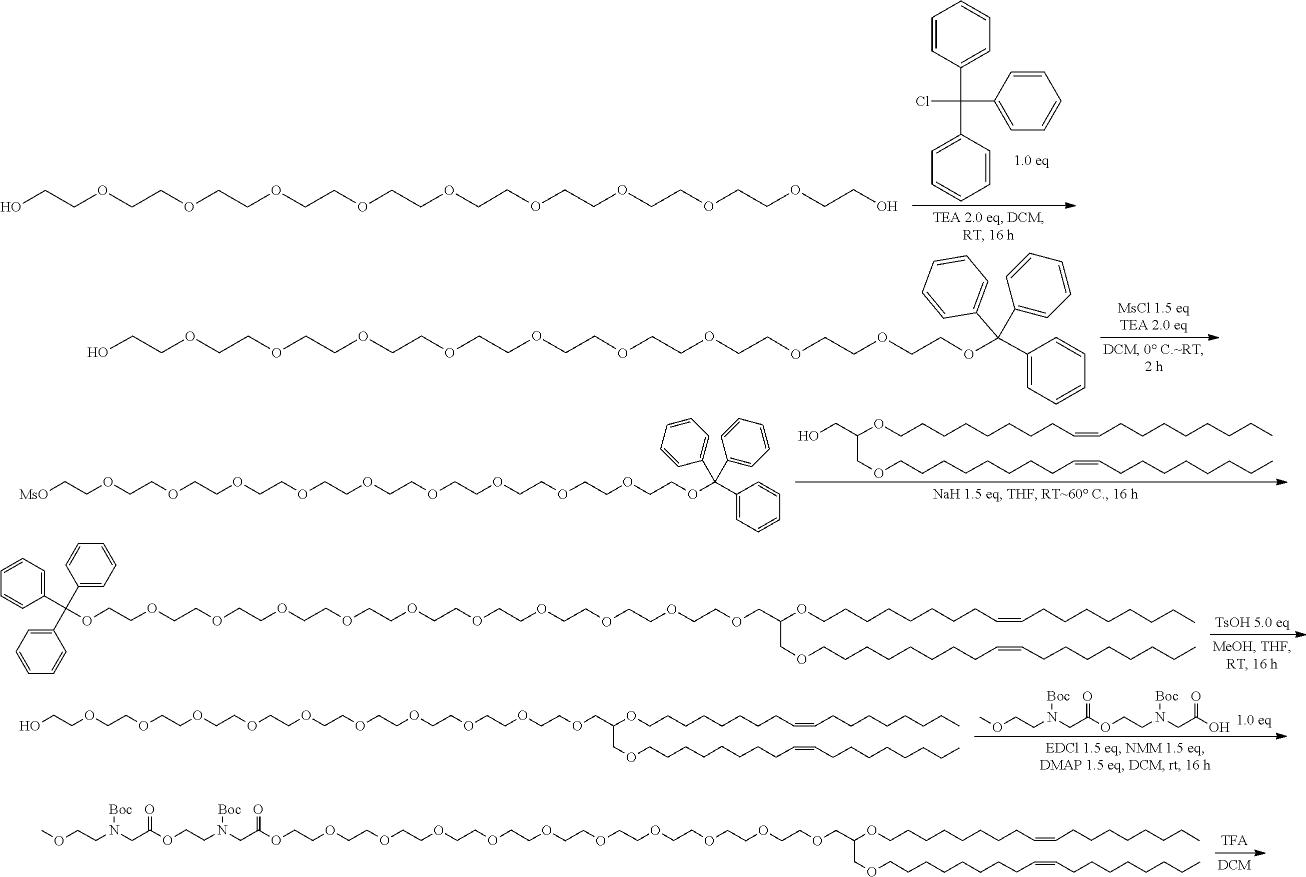

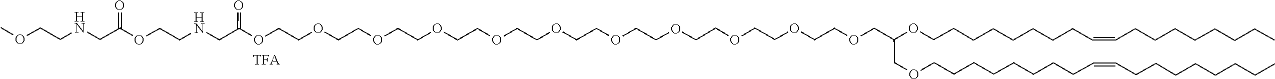 [SMILES: CCCCCCCC/C=C\CCCCCCCCOCC(COCCOCCOCCOCCOCCOCCOCCOCCOCCOCCOC(=O)CNCCOC(=O)CNCCOC)OCCCCCCCC/C=C\CCCCCCCC]

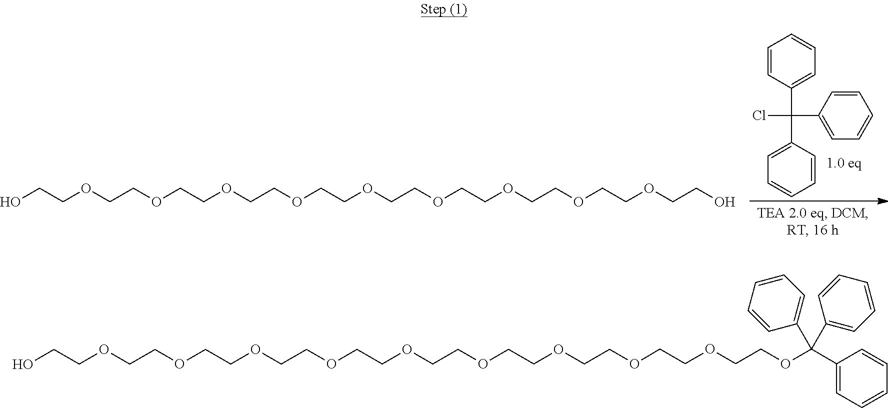 [SMILES: OCCOCCOCCOCCOCCOCCOCCOCCOCCOCCO ; ClC(c1ccccc1)(c1ccccc1)c1ccccc1 ; OCCOCCOCCOCCOCCOCCOCCOCCOCCOCCOC(c1ccccc1)(c1ccccc1)c1ccccc1]

A mixture of 2-[2-[2-[2-[2-[2-[2-[2-[2-(2-hydroxyethoxy)ethoxy]ethoxy]ethoxy]ethoxy]ethoxy]ethoxy]ethoxy]ethoxy]ethanol (4.8 g, 10.5 mmol) and N,N-diethylethanamine (2.12 g, 20.9 mmol) in DCM (100 mL) was added [chloro(diphenyl)methyl]benzene (2.33 g, 8.37 mmol). The mixture was stirred for 16 h at room temperature. The mixture was added DCM (100 mL) and washed with water, brine, dried over Na₂SO₄ and concentrated. The residue was purified by column chromatography on silica gel eluted with 0%-20% MeOH in DCM to afford 2-[2-[2-[2-[2-[2-[2-[2-[2-(2-trityloxyethoxy)ethoxy]ethoxy]ethoxy]ethoxy]ethoxy]ethoxy]ethoxy]ethoxy]ethanol (1.9 g, 26%) as colorless oil.

LCMS 718 (M+18), 97% UV:214 nm

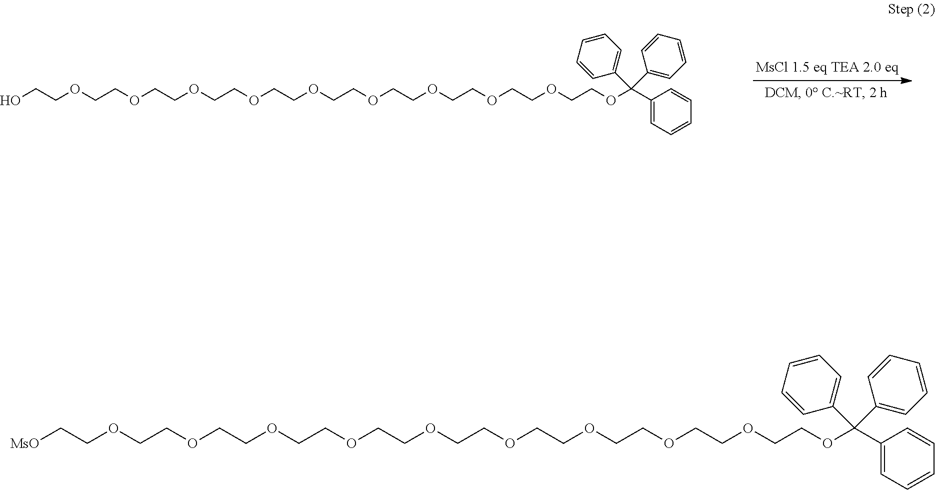 [SMILES: OCCOCCOCCOCCOCCOCCOCCOCCOCCOCCOC(c1ccccc1)(c1ccccc1)c1ccccc1 ; CS(=O)(=O)OCCOCCOCCOCCOCCOCCOCCOCCOCCOCCOC(c1ccccc1)(c1ccccc1)c1ccccc1]

To a mixture of 2-[2-[2-[2-[2-[2-[2-[2-[2-(2-trityloxyethoxy)ethoxy]ethoxy]ethoxy]ethoxy]ethoxy]ethoxy]ethoxy]ethoxy]ethanol (1.9 g, 2.71 mmol) and N,N-diethylethanamine (0.549 g, 5.42 mmol) in DCM (50 mL) cooled to 0° C. was added methanesulfonyl chloride (0.466 g, 4.07 mmol). The reaction mixture was stirred for 3 h at room temperature. TLC indicated the starting material was disappeared. The mixture was diluted with DCM (100 mL) and washed with water, brine, dried over Na2SO4 and concentrated to give 2-[2-[2-[2-[2-[2-[2-[2-[2-(2-trityloxyethoxy)ethoxy]ethoxy]ethoxy]ethoxy]ethoxy]ethoxy]ethoxy]ethoxy]ethyl methanesulfonate (2.0 g, 95%) as yellow oil.

LCMS 796 (M+18), 97% UV:214 nm

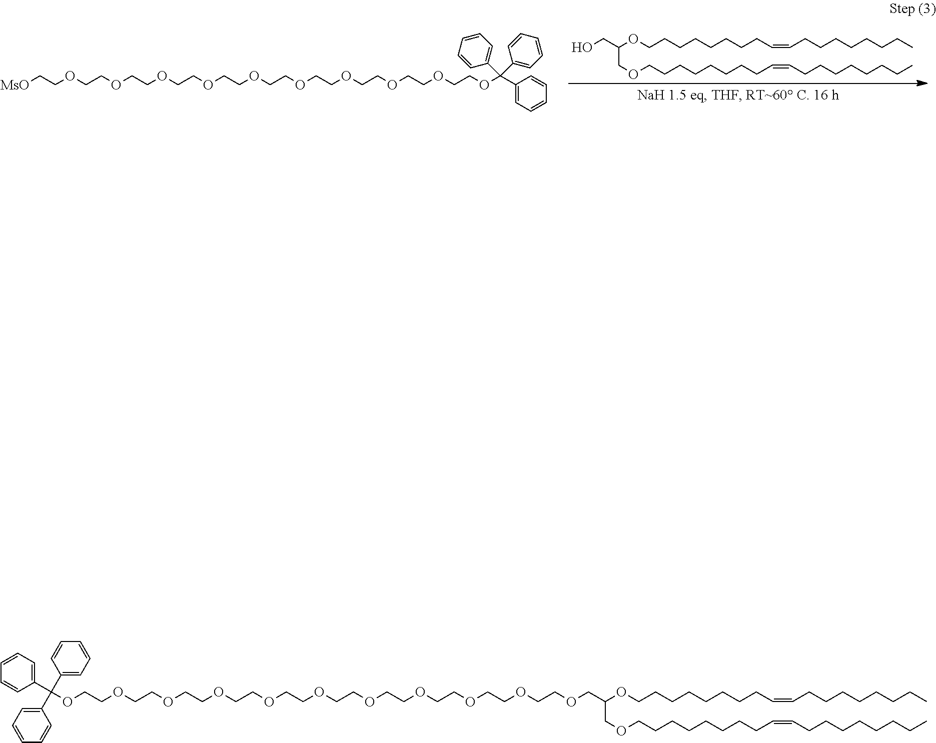 [SMILES: CS(=O)(=O)OCCOCCOCCOCCOCCOCCOCCOCCOCCOCCOC(c1ccccc1)(c1ccccc1)c1ccccc1 ; CCCCCCCC/C=C\CCCCCCCCOCC(CO)OCCCCCCCC/C=C\CCCCCCCC ; CCCCCCCC/C=C\CCCCCCCCOCC(COCCOCCOCCOCCOCCOCCOCCOCCOCCOCCOC(c1ccccc1)(c1ccccc1)c1ccccc1)OCCCCCCCC/C=C\CCCCCCCC]

To a suspension of NaH (60% mineral oil dispersion, 61 mg, 2.53 mmol) in 15 mL of THF was added 2,3-bis[(Z)-octadec-9-enoxy]propan-1-ol (0.75 g, 1.26 mol) dissolved in 15 mL of THF. The resultant suspension was stirred for 2 h at room temperature. 2-[2-[2-[2-[2-[2-[2-[2-[2-(2-trityloxyethoxy)ethoxy]ethoxy]ethoxy]ethoxy]ethoxy]ethoxy]ethoxy]ethoxy]ethyl methanesulfonate (1.18 g, 1.52 mmol) was added to the suspension, and the reaction mixture was brought to reflux overnight. The reaction mixture was then cooled to room temperature, and water (40 mL) was added. The organic phase was collected and the aqueous phase was extracted with 3×40 ml EtOAc. The organic phases were combined and washed successively with 40 ml of 1N HCl, 40 ml of 5% (w/v) NaHCO₃ and 40 ml of brine and dried on MgSO4. The solvent was evaporated under reduced pressure and the resulting oil was purified on a silica gel column eluted with Petroleum ether/AcOEt (4:1) to give [2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethoxy-diphenyl-methyl]benzene (1.1 g, 68%) as a colorless oil.

¹H NMR (400 MHz, CDCl₃) δ 7.48-7.43 (m, 6H), 7.33-7.27 (m, 6H), 7.25-7.18 (m, 3H), 5.39-5.30 (m, 3H), 3.71-3.59 (m, 39H), 3.59-3.38 (m, 9H), 3.23 (t, J=5.2 Hz, 2H), 2.07-1.92 (m, 71H), 1.80-1.69 (m, 5H), 1.63-1.48 (m, 4H), 1.40-1.18 (m, 44H), 0.88 (t, J=6.8 Hz, 6H).

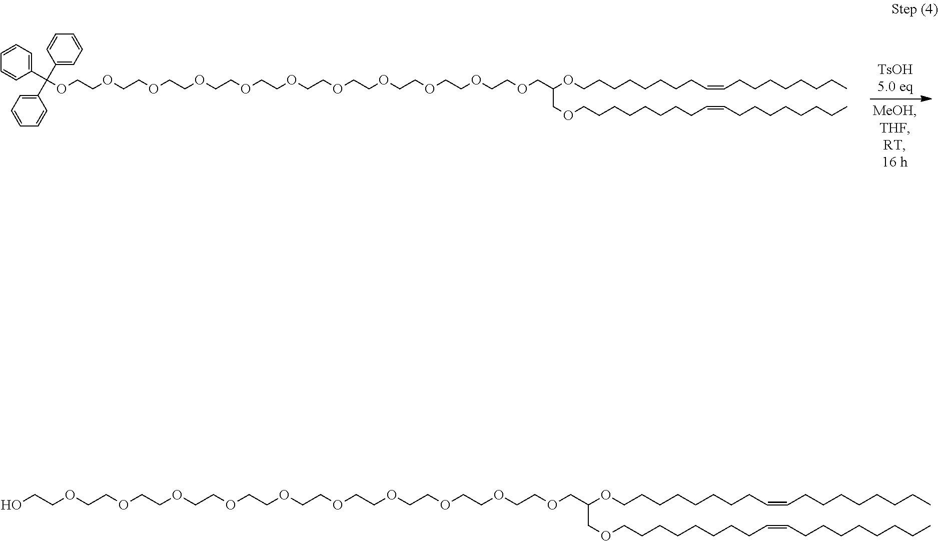 [SMILES: CCCCCCCC/C=C\CCCCCCCCOCC(COCCOCCOCCOCCOCCOCCOCCOCCOCCOCCOC(c1ccccc1)(c1ccccc1)c1ccccc1)OCCCCCCCC/C=C\CCCCCCCC ; CCCCCCCC/C=C\CCCCCCCCOCC(COCCOCCOCCOCCOCCOCCOCCOCCOCCOCCO)OCCCCCCCC/C=C\CCCCCCCC]

To a mixture of [2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethoxy-diphenyl-methyl]benzene (1.1 g, 0.862 mmol) in THF (5 mL)/MeOH (5 mL) was added Toluene-4-sulfonic acid (0.82 g, 4.31 mmol) and stirred overnight at room temperature. Et3N (1 mL) was added to the reaction mixture and concentrated. The residue was purified by flash column chromatography on silica gel eluting with 5:1 ethyl acetate/petroleum ether to give 2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethanol (1.1 g, 95%, purity:70%) as colourless oil.

¹H NMR (500 MHz, CDCl₃) δ 5.43-5.28 (m, 3H), 3.83-3.33 (m, 59H), 2.01 (d, J=5.3 Hz, 7H), 1.55 (s, 4H), 1.27 (s, 45H), 0.88 (t, J=6.4 Hz, 6H).

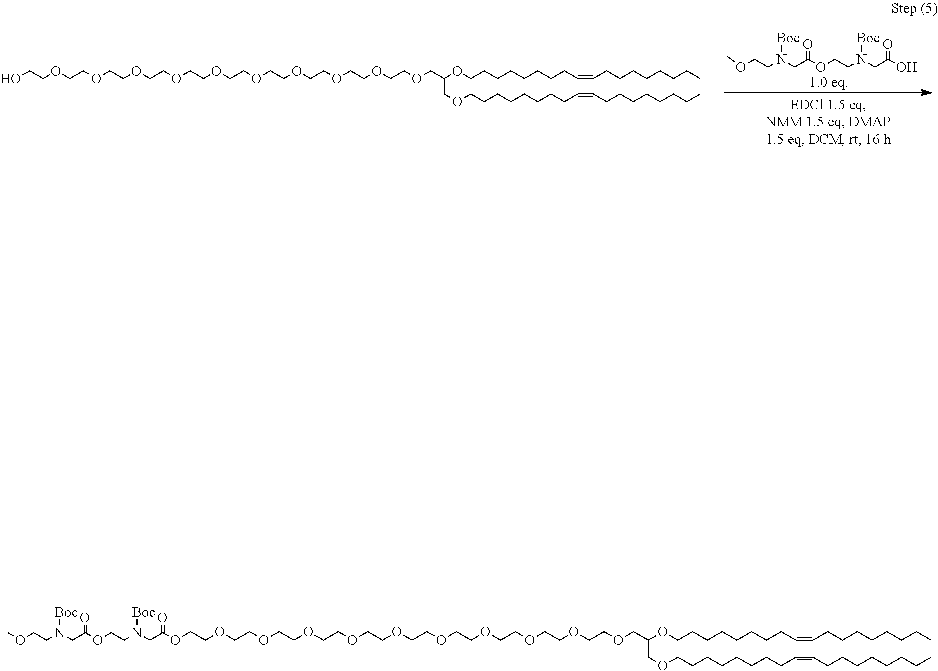

In a 100 mL round bottom flask, 2-[tert-butoxycarbonyl-[2-[2-[tert-butoxycarbonyl(2-methoxyethyl)amino]acetyl]oxyethyl]amino]acetic acid (400 mg, 0.921 mmol), 2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethanol (952 mg, 0.921 mmol), N,N-dimethylpyridin-4-amine (169 mg, 1.38 mmol) were dissolved in DCM-Anhydrous (15 mL) in presence of 4-methylmorpholine (140 mg, 1.38 mmol). The reaction was cooled to 0° C. and EDC HCl (265 mg, 1.38 mmol) was added to the mixture. The reaction was warmed to room temperature and stirred overnight. Water was added and the organic layers were extracted with DCM (3×15 mL), washed with brine, dried over Na2SO4, filtered and concentrated. The residue was purified by flash column chromatography on silica gel eluting with 4:1 ethyl acetate/petroleum ether to give 2-[[2-[2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.8 g, 59.9%) as colourless oil.

¹H NMR (400 MHz, CDCl₃) δ 5.42-5.31 (m, 3H), 4.33-4.20 (m, 4H), 4.08-3.95 (m, 4H), 3.77-3.37 (m, 57H), 3.30 (d, J=5.4 Hz, 3H), 2.08-1.92 (m, 7H), 1.59-1.51 (m, 5H), 1.51-1.39 (m, 20H), 1.37-1.21 (m, 46H), 0.88 (t, J=6.8 Hz, 6H).

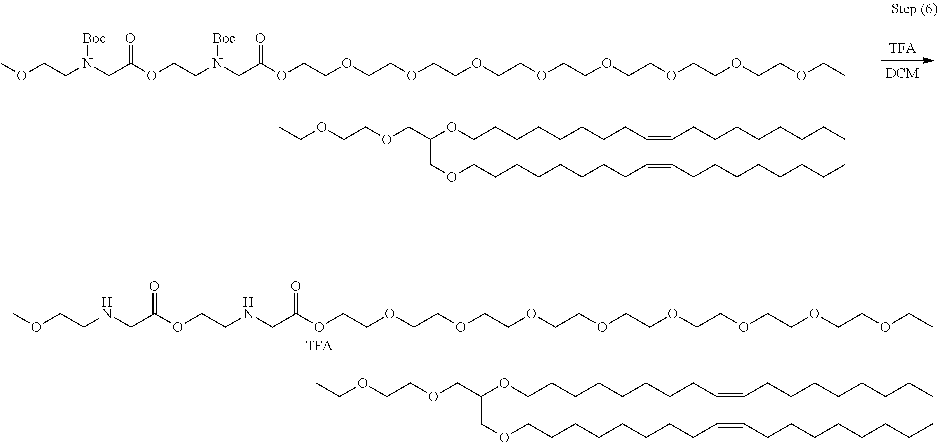

A mixture of 2-[[2-[2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.8 g, 0.552 mmol) in DCM (5 ml) was added TFA (0.629 g, 5.52 mmol). The mixture was stirred 3 h at ambient temperature. The mixture was concentrated and dried under vacuum several hours to give 2-[[2-[2-[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic acid (0.635 g, 78%) as brown oil.

¹H NMR (500 MHz, CDCl₃) δ 5.42-5.27 (m, 3H), 4.57 (s, 2H), 4.39 (s, 2H), 4.08 (d, J=24.5 Hz, 3H), 3.82-3.41 (m, 52H), 3.37 (s, 5H), 2.05-1.92 (m, 6H), 154 (d, J=6.2 Hz, 4H), 1.37-1.19 (m, 44H), 0.88 (t, J=6.9 Hz, 6H).

### Example 9: Synthesis of 2 2-[[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic Acid (Compound (XII))

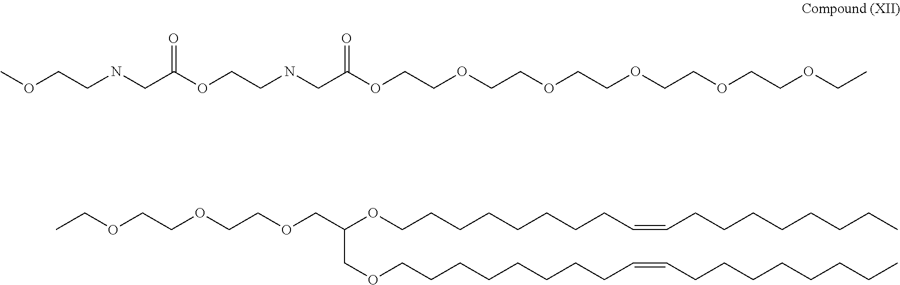

Synthesis of Compound (XII)

A mixture of 2-[[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]-2-oxo-ethyl]-tert-butoxycarbonylamino]ethyl 2-[tert-butoxycarbonyl(2-methoxyethyl)amino]acetate (0.6 g, 0.441 mmol) in DCM (5 ml) was added TFA (0.22 g, 2.51 mmol). The mixture was stirred 3 h at ambient temperature. The mixture was concentrated to give 2-[[2-[2-[2-[2-[2-[2-[2-[2-[2-[2,3-bis[(Z)-octadec-9-enoxy]propoxy]ethoxy]ethoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic acid (0.424 g, 69.3%) as brown oil.

¹H NMR (500 MHz, CDCl₃) δ 5.44-5.28 (m, 3H), 4.57 (s, 2H), 4.39 (s, 2H), 4.09 (s, 2H), 4.04 (s, 2H), 3.75-3.41 (m, 43H), 3.41-3.30 (m, 5H), 2.04-1.92 (m, 6H), 1.55 (s, 4H), 1.37-1.20 (m, 44H), 0.88 (t, J=6.9 Hz, 6H).

### Example 10: Synthesis of 2-[[2-[2-[2-[2-[2-[2-[2-[2-[2-[2-[2-[2-[2-[2-[2,3-bis[(Z)- octadec-9-enoxy]propoxy]ethoxy]ethoxy]ethoxy]ethoxy]ethoxy]ethoxy]ethoxy]ethoxy]ethoxy]ethoxy]ethoxy] ethoxy]ethoxy]-2-oxo-ethyl]amino]ethyl 2-(2-methoxyethylamino)acetate;2,2,2-trifluoroacetic Acid (Compound (XIII))

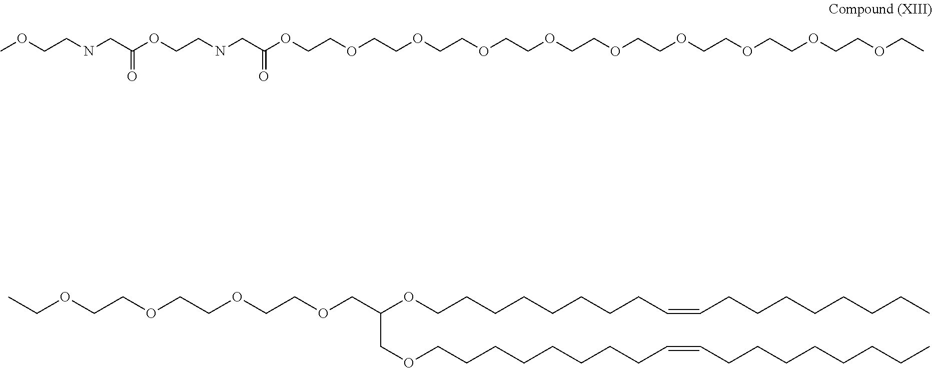

Synthesis of Compound (XIII)

A mixture of (Z)-3-(tert-butoxycarbonyl)-47-(((Z)-octadec-9-en-1-yl)oxy)-5-oxo6,9,12,15,18,21,24,27,30,33,36,39,42,45,49-pentadecaoxa-3-azaheptahexacont-58-en-1-yl N-(tertbutoxycarbonyl)-N-(2-methoxyethyl)glycinate (0.4 g, 0.253 mmol) in DCM (5 ml) was added TFA (0.288 g, 2.53 mmol). The mixture was stirred 3 hrs at ambient temperature. The mixture was concentrated to give 2-[[2-[2-[2-[2-[2-[2-[2-[2-[2-[2-[2-[2-[2-[2-[2,3-bis[(Z)-octadec- 9-enoxy]propoxy]ethoxy]ethoxy]ethoxy]ethoxy]ethoxy]ethoxy]ethoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic acid (0.134 g, 35.4%) as brown oil.

¹H NMR (500 MHz, CDCl₃) δ 5.44-5.27 (m, 4H), 4.57 (s, 2H), 4.38 (s, 2H), 4.15-3.91 (m, 4H), 3.81-3.41 (m, 68H), 3.39-3.29 (m, 6H), 2.05-1.92 (m, 7H), 1.59-1.51 (m, 4H), 1.32-1.22 (m, 44H), 0.88 (t, J=6.9 Hz, 6H)

### Example 11: Synthesis of 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]amino]ethyl 2-(2-methoxyethylamino)acetate;dihydrochloride (Compound (XIV))

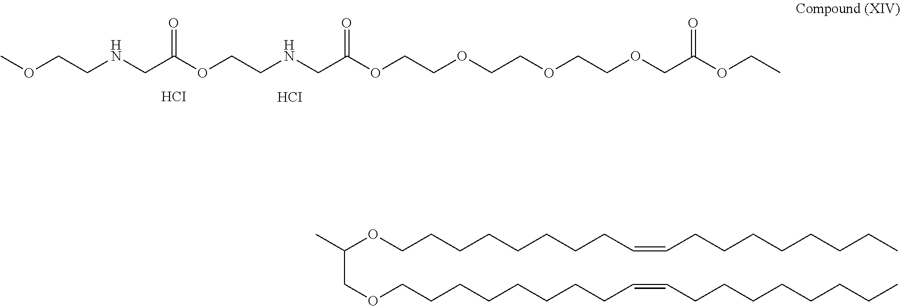

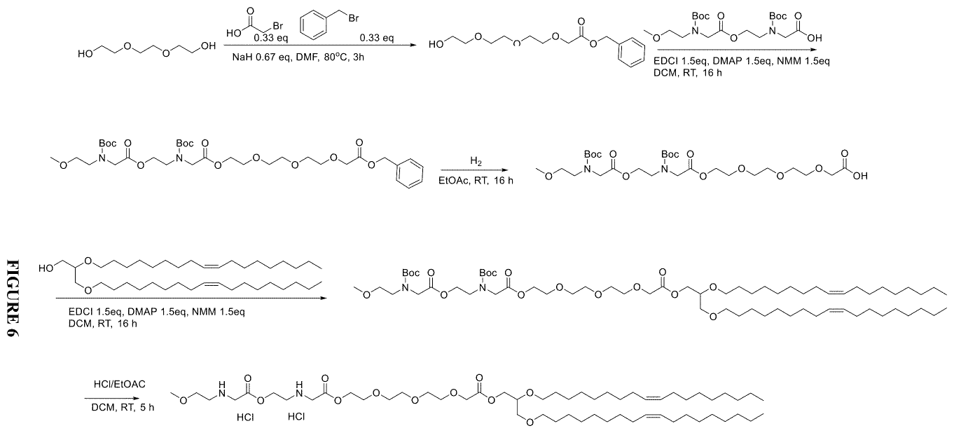

Compound (XIV) was synthesized based on the chemistry shown in Scheme (8) represented on FIG. 6.

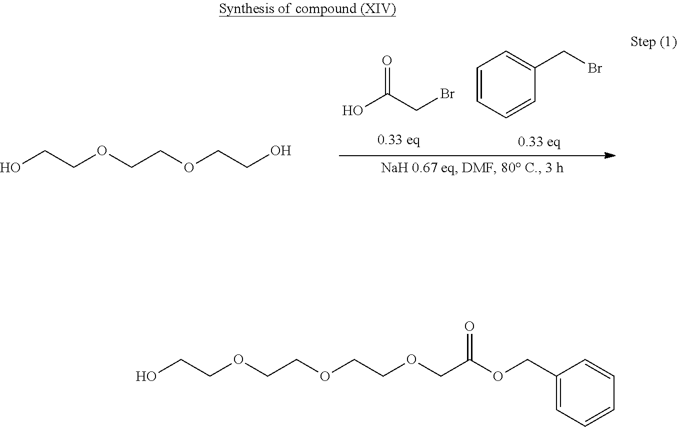

2-[2-(2-hydroxyethoxy)ethoxy]ethanol (15 g, 0.1 mol) was dissolved under argon in 200 mL of abs. DMF and cooled to 0° C. NaH (2.56 g, 0.067 mol, 60% in mineral oil) was carefully added, the ice bath removed, and stirring continued for 1 h at 80° C. The reaction mixture was cooled to ambient temperature and treated with 2-bromoacetic acid (4.58 g, 0.033 mol) which was added via dropping funnel as a DMF-solution (10 mL). After an additional 30 min. at 75° C., bromomethylbenzene (5.64 g, 0.033 mol) was added neat and esterification allowed to proceed for 30 min. Cooling, careful pouring onto crashed ice, extraction with ethyl acetate, washing with water, drying over Na₂SO₄, and evaporation of all solvents followed by flash chromatography (SiO₂, ethyl acetate/heptane=8/2) afforded benzyl 2-[2-[2-(2-hydroxyethoxy)ethoxy]ethoxy]acetate (2.2 g, 7.38%) as yellow oil.

¹H NMR (500 MHz, CDCl₃) δ 7.39-7.31 (m, 5H), 5.19 (s, 2H), 4.20 (s, 2H), 3.77-3.67 (m, 6H), 3.68-3.63 (m, 4H), 3.63-3.56 (m, 2H)

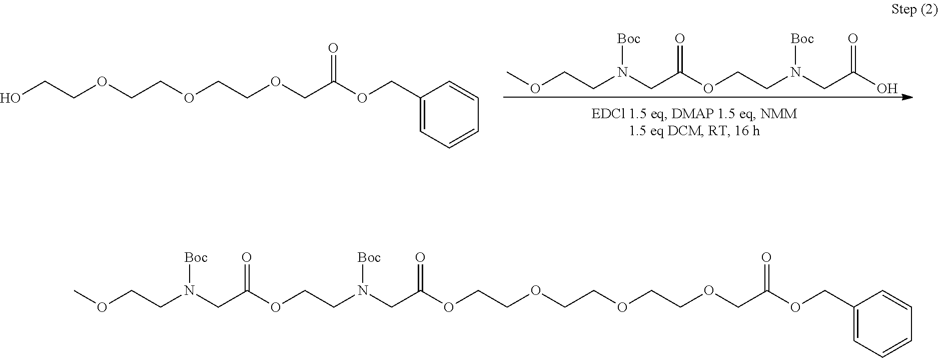

In a 50 mL round bottom flask, 2-[tert-butoxycarbonyl-[2-[2-[tert-butoxycarbonyl(2-methoxyethyl)amino]acetyl]oxyethyl]amino]acetic acid (874 mg, 2.01 mmol), benzyl 2-[2-[2-(2-hydroxyethoxy)ethoxy]ethoxy]acetate (600 mg, 2.01 mmol) and N,N-dimethylpyridin-4-amine (295 mg, 2.41 mmol) were dissolved in DCM-Anhydrous (10 mL) in presence of 4-methylmorpholine (244 mg, 2.41 mmol). The reaction was cooled to 0° C. and 3-(ethyliminomethyleneamino)-N,N-dimethyl-propan-1-amine;hydrochloride (463 mg, 2.41 mmol) was added to the mixture. The reaction was warmed to room temperature and stirred overnight. Water was added and the organic layers were extracted with DCM (3×15 mL), washed with brine, dried over Na₂SO₄, filtered and concentrated. The residue was purified by flash column chromatography on silica gel eluting with 1:1 ethyl acetate/petroleum ether to give 2-[[2-[2-[2-[2-(2-benzyloxy-2-oxo-ethoxy)ethoxy]ethoxy]ethoxy]-2-oxo-ethyl]-tert-butoxycarbonyl-amino]ethyl 2-[tert-butoxycarbonyl(2-methoxyethyl)amino]acetate (1.2 g, 83.5%) as colorless oil.

¹H NMR (500 MHz, CDCl₃) δ 7.38-7.32 (m, 5H), 5.19 (s, 2H), 4.29-4.18 (m, 6H), 4.06-4.01 (m, 2H), 4.00-3.94 (m, 2H), 3.76-3.72 (m, 2H), 3.71-3.66 (m, 4H), 3.64 (s, 4H), 3.56-3.41 (m, 6H), 3.32-3.26 (m, 3H), 1.46 (s, 9H), 1.42 (s, 9H).

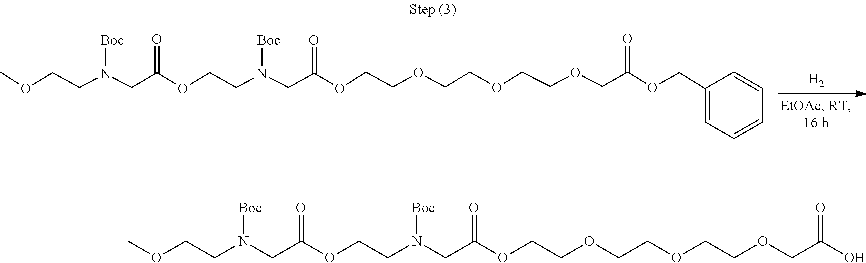 [SMILES: COCCN(CC(=O)OCCN(CC(=O)OCCOCCOCCOCC(=O)OCc1ccccc1)C(=O)OC(C)(C)C)C(=O)OC(C)(C)C ; "H2" ; COCCN(CC(=O)OCCN(CC(=O)OCCOCCOCCOCC(=O)O)C(=O)OC(C)(C)C)C(=O)OC(C)(C)C]

2-[[2-[2-[2-[2-(2-benzyloxy-2-oxo-ethoxy)ethoxy]ethoxy]ethoxy]-2-oxo-ethyl]-tert-butoxycarbonyl-amino]ethyl 2-[tert-butoxycarbonyl(2-methoxyethyl)amino]acetate (1.2 g, 1.68 mmol) and Pd/C (0.5 g) were mixed with EtOAc (30 mL) and attached to a hydrogenation apparatus. The system was evacuated and then refilled with hydrogen. The mixture was stirred at room temperature overnight. The mixture was filtered and the filtrate was concentrated to give 2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]acetic acid (0.95 g, 90.6%) as colorless oil.

¹H NMR (500 MHz, CDCl₃) δ 4.32-4.20 (m, 4H), 4.17 (d, J=2.7 Hz, 2H), 4.08-3.94 (m, 4H), 3.79-3.61 (m, 10H), 3.58-3.40 (m, 6H), 3.30 (d, J=6.1 Hz, 3H), 1.50-1.37 (m, 18H).

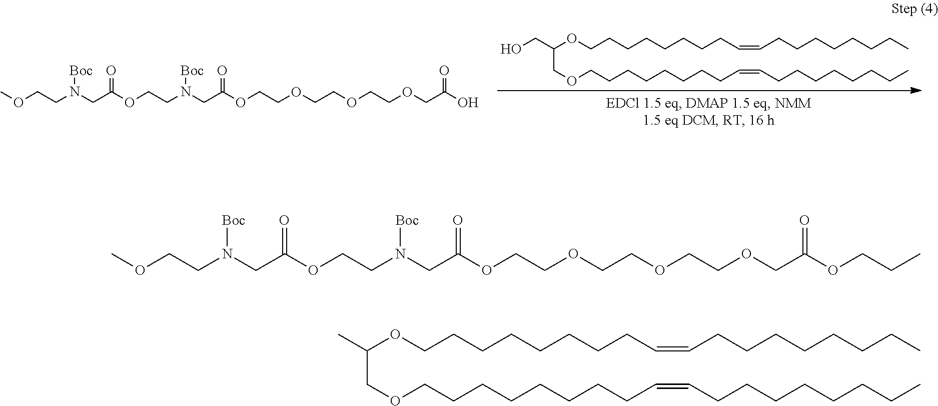

In a 50 mL round bottom flask, 2-[tert-butoxycarbonyl-[2-[2-[tert-butoxycarbonyl(2-methoxyethyl)amino]acetyl]oxyethyl]amino]acetic acid (400 mg, 0.64 mmol), 2,3-bis[(Z)-octadec-9-enoxy]propan-1-ol (380 mg, 0.64 mmol) and N,N-dimethylpyridin-4-amine (117 mg, 0.96 mmol) were dissolved in DCM-Anhydrous (15 mL) in presence of 4-methylmorpholine (97.2 mg, 0.96 mmol). The reaction was cooled to 0° C. and 3-(ethyliminomethyleneamino)-N,N-dimethyl-propan-1-amine;hydrochloride (184 mg, 0.96 mmol) was added to the mixture. The reaction was warmed to room temperature and stirred overnight. Water was added and the organic layers were extracted with DCM (3×15 mL), washed with brine, dried over Na₂SO₄, filtered and concentrated. The residue was purified by flash column chromatography on silica gel eluting with 1:3 ethyl acetate/petroleum ether to give 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.45 g, 58.6%) as colourless oil.

¹H NMR (500 MHz, CDCl₃) δ 5.43-5.24 (m, 3H), 4.33-4.07 (m, 8H), 4.07-3.93 (m, 4H), 3.76-3.37 (m, 23H), 3.30 (d, J=7.0 Hz, 3H), 2.08-1.92 (m, 7H), 1.58-1.50 (m, 4H), 1.44 (d, J=23.3 Hz, 18H), 1.36-1.20 (m, 44H), 0.88 (t, J=6.9 Hz, 6H).

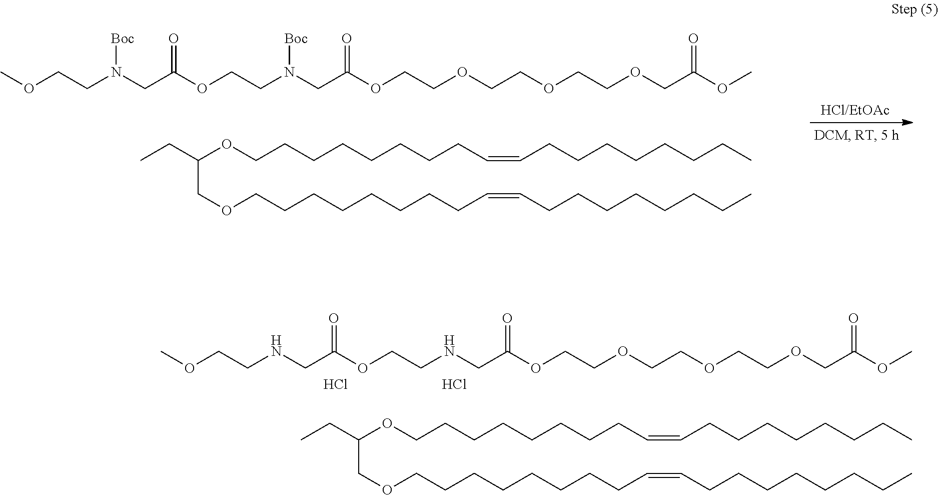

To a mixture of 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.45 g, 0.375 mmol) in DCM (5 mL) was added HCl in EtOAc (5 mL, 3 mol/L). The mixture was stirred 3 hrs at room temperature. The reaction mixture was concentrated to give 2-[[2-[2-[2-[2-[2-[2,3-bis[(Z)-octadec-9-enoxy]propoxy]-2-oxo-ethoxy]ethoxy]ethoxy]ethoxy]-2-oxo-ethyl]amino]ethyl 2-(2-methoxyethylamino)acetate;dihydrochloride (0.143 g, 35.6%) as a white oil.

¹H NMR (500 MHz, CDCl₃) δ 9.83 (s, 3H), 5.42-5.27 (m, 3H), 4.80 (s, 2H), 4.39 (s, 2H), 4.34-4.05 (m, 7H), 3.98 (s, 2H), 3.86 (s, 2H), 3.78-3.30 (m, 25H), 2.10-1.92 (m, 8H), 1.60-1.48 (m, 4H), 1.35-1.23 (m, 44H), 0.88 (t, J=6.9 Hz, 6H).

### Example 12: Synthesis of nonyl 8-[3-[2-[2-[2-[2-[2-[2-[2-(2-methoxyethylamino)acetyl]oxyethylamino]acetyl]oxyethoxy]ethoxy]ethoxy]ethoxy]-2-(8-nonoxy-8-oxo-octoxy)propoxy]octanoate;2,2,2-trifluoroacetic Acid (Compound (XV))

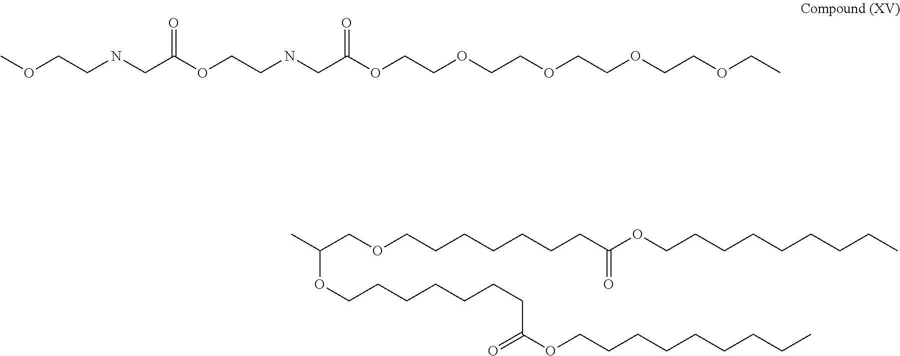

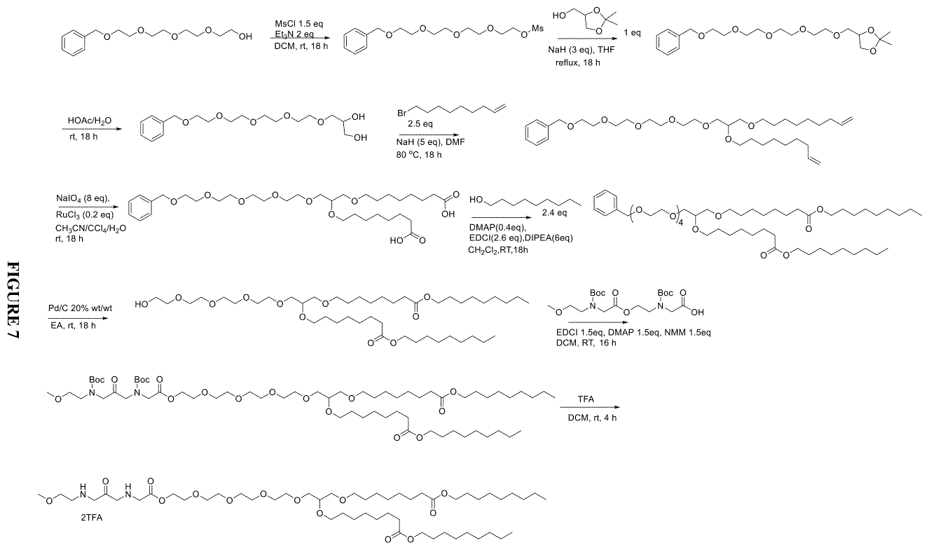

Compound (XV) was synthesized based on the chemistry shown in Scheme (9) presented on FIG. 7.

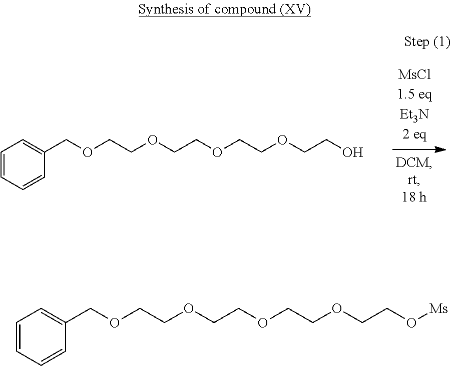 [SMILES: OCCOCCOCCOCCOCc1ccccc1 ; CS(=O)(=O)OCCOCCOCCOCCOCc1ccccc1]

2-[2-[2-(2-benzyloxyethoxy)ethoxy]ethoxy]ethanol (50 g, 176 mmol) and triethylamine (35.6 g, 352 mmol) in dry dichloromethane (500 mL) under nitrogen were cooled to −5° C. Methanesulfonyl chloride (30.2 g, 264 mmol) in dry DCM (20 mL) was added dropwise to this solution at 0° C. The mixture was allowed to warm to room temperature and stirred at room temperature for 18 hrs. Triethylamine hydrochloride was filtered off, and the DCM solution was washed with 0.1 N HCl and dried over sodium sulfate. Removing the solvent afforded 2-[2-[2-(2-benzyloxyethoxy)ethoxy]ethoxy]ethyl methanesulfonate (69.1 g, 175 mmol, quant.) as light yellow oil which was used without further purification.

¹H NMR (400 MHz, CDCl₃) δ 7.37-7.27 (m, 5H), 4.56 (s, 2H), 4.39-4.33 (m, 2H), 3.78-3.73 (m, 2H), 3.69-3.60 (m, 12H), 3.06 (s, 3H).

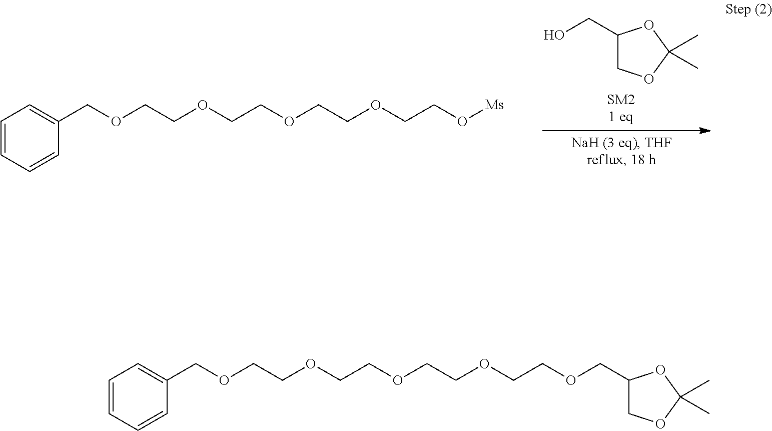

To the solution of (2,2-dimethyl-1,3-dioxolan-4-yl)methanol (24.4 g, 175 mmol) in THF (500 mL) was added NaH (14 g, 351 mmol) and the mixture was heated to reflux for 15 min. Then the reaction was cooled to room temperature and 2-[2-[2-(2-benzyloxyethoxy)ethoxy]ethoxy]ethyl methanesulfonate (69.1 g, 175 mmol) was added under nitrogen and the reaction was heated at 80° C. for 24 h. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy)ethoxy]ethoxy]ethoxymethyl]-2,2-dimethyl-1,3-dioxolane (54.4 g, 70% yield) as light yellow oil.

¹H NMR (400 MHz, CDCl₃) δ 7.38-7.27 (m, 5H), 4.57 (s, 2H), 4.28 (t, J=5.9 Hz, 1H), 4.05 (dd, J=8.3, 6.4 Hz, 1H), 3.72 (dd, J=8.3, 6.4 Hz, 1H), 3.70-3.61 (m, 16H), 3.57 (dd, J=10.0, 5.8 Hz, 1H), 3.49 (dd, J=10.0, 5.5 Hz, 1H), 1.42 (s, 3H), 1.35 (s, 3H).

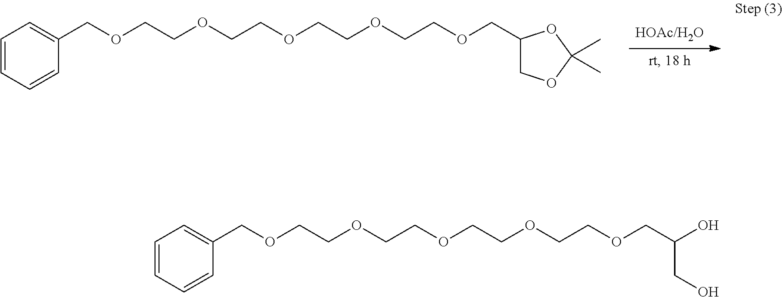 [SMILES: CC1(C)OCC(COCCOCCOCCOCCOCc2ccccc2)O1 ; OCC(O)COCCOCCOCCOCCOCc1ccccc1]

The mixture of 4-[2-[2-[2-(2-benzyloxyethoxy)ethoxy]ethoxy]ethoxymethyl]-2,2-dimethyl-1,3-dioxolane (54.4 g, 123 mmol) in AcOH (200 mL) and H2O (200 mL) was stirred at room temperature for 18 h. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy)ethoxy]ethoxy]ethyl methanesulfonate (49 g, 123 mmol, quant.) as light yellow oil was obtained which was used without further purification.

¹H NMR (400 MHz, CDCl₃) δ 7.38-7.27 (m, 5H), 4.57 (s, 2H), 3.88-3.81 (m, 1H), 3.70-3.51 (m, 21H).

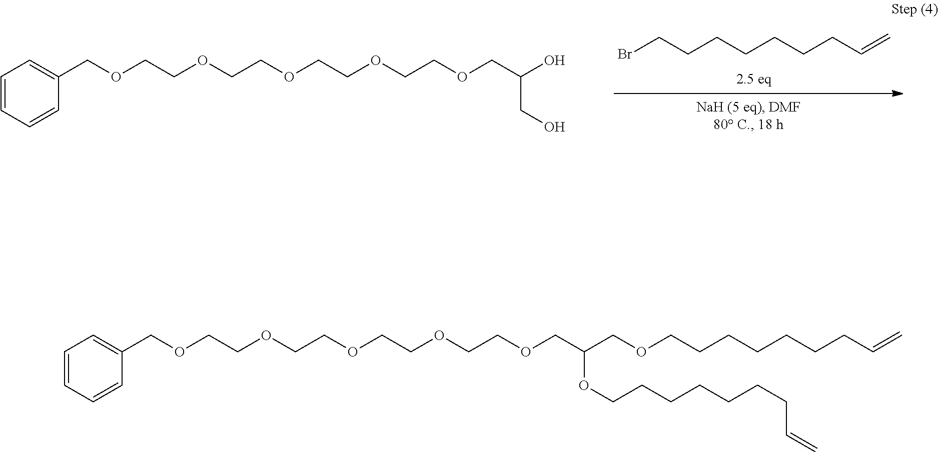

To a solution of 3-[2-[2-[2-(2-benzyloxyethoxy)ethoxy]ethoxy]ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate in petroleum ether to give 2-[2-[2-[2-[2,3-bis(non-8-enoxy)propoxy]ethoxy]ethoxy]ethoxy]ethoxymethylbenzene (9.3 g, 14.6 mmol, 24.2% yield) as light yellow oil.

¹H NMR (400 MHz, CDCl₃) δ 7.37-7.27 (m, 5H), 5.89-5.72 (m, 2H), 5.04-4.89 (m, 4H), 4.57 (s, 2H), 3.71-3.60 (m, 17H), 3.59-3.38 (m, 9H), 2.03 (q, J=6.7 Hz, 4H), 1.60-1.49 (m, 4H), 1.43-1.23 (m, 16H).

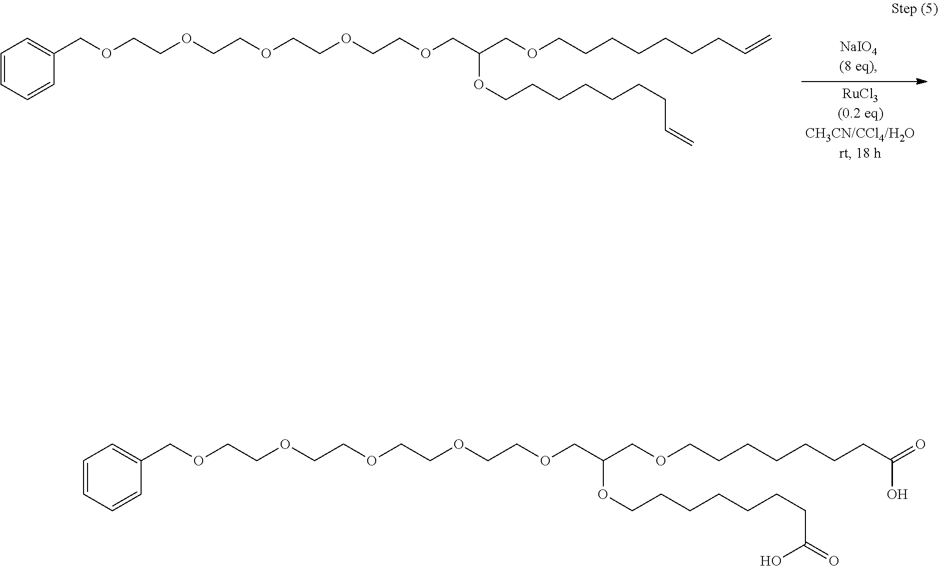 [SMILES: C=CCCCCCCCOCC(COCCOCCOCCOCCOCc1ccccc1)OCCCCCCCC=C ; O=C(O)CCCCCCCOCC(COCCOCCOCCOCCOCc1ccccc1)OCCCCCCCC(=O)O]

To a solution of 2-[2-[2-[2-[2,3-bis(non-8-enoxy)propoxy]ethoxy]ethoxy]ethoxy]ethoxymethylbenzene (9.3 g, 14.6 mmol) in MeCN (80 mL), CCl4 (80 mL) and water (80 mL) was added NaIO4 (24.9 g, 116 mmol) and RuCl3 (656 mg, 2.91 mmol). The reaction mixture was stirred at room temperature for 24 h. LCMS indicated that the title compound was the major product along with partial mono-aldehyde product. The reaction was filtered and the filtrate was diluted with ethyl acetate (800 mL) and washed with 1N aq. HCl (400 mL). The organic layer was washed with Na2S2O3 solution and then dried over sodium sulfate, filtered and concentrated to give 8-[3-[2-[2-[2-(2-benzyloxyethoxy)ethoxy]ethoxy]ethoxy]-2-(7-carboxyheptoxy)propoxy]octanoic acid (10 g, 12.4 mmol) as yellow oil which was used without further purification.

Step (6)

8-[3-[2-[2-[2-(2-benzyloxyethoxy)ethoxy]ethoxy]ethoxy]-2-(8-oxooctoxy)propoxy]octanoic acid (10 g, 8 mmol) was dissolved in t-BuOH: H2O (3:1, 160 mL), containing NaH2PO4.2H2O (3.73 g, 24 mmol), 2-methy-2-butene (40 mL) and sodium chlorite (2.71 mg, 24 mmol). The reaction was stirred for 2 h at rt and LCMS indicated that the starting material was consumed. The reaction mixture was diluted with ethyl acetate. The aqueous layer was extracted with ethyl acetate. The combined organic layers were dried over sodium sulfate and concentrated to afford 8-[3-[2-[2-[2-(2-benzyloxyethoxy)ethoxy]ethoxy]ethoxy]-2-(7-carboxyheptoxy)propoxy]octanoic acid (10 g, 3.22 mmol, quant.) as light yellow oil.

¹H NMR (400 MHz, CDCl₃) δ 7.38-7.27 (m, 5H), 4.57 (s, 2H), 3.71-3.61 (m, 17H), 3.59-3.37 (m, 9H), 2.33 (t, J=7.3 Hz, 4H), 1.69-1.51 (m, 8H), 1.39-1.28 (m, 14H).

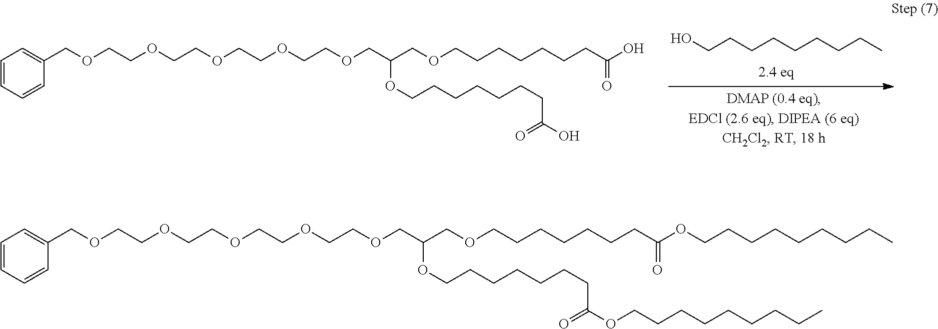

To the solution of 8-[3-[2-[2-[2-(2-benzyloxyethoxy)ethoxy]ethoxy]ethoxy]-2-(7-carboxyheptoxy)propoxy]octanoic acid (10 g, 14.8 mmol) and 1-Nonanol (5.12 g, 35.5 mmol) in dry dichloromethane (200 mL) under nitrogen were added N,N-Diisopropylethylamine (11.5 g, 88.7 mmol), DMAP (0.722 g, 5.91 mmol) and EDCI (7.37 g, 38.4 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 20% to 55% ethyl acetate in petroleum ether to give nonyl 8-[3-[2-[2-[2-(2-benzyloxyethoxy)ethoxy]ethoxy]ethoxy]-2-(8-nonoxy-8-oxo-octoxy)propoxy]octanoate (5 g, 35.9%) as colorless oil.

¹H NMR (400 MHz, CDCl₃) δ 7.38-7.27 (m, 5H), 4.57 (s, 2H), 4.05 (t, J=6.8 Hz, 4H), 3.70-3.61 (m, 16H), 3.59-3.39 (m, 9H), 2.28 (t, J=7.5 Hz, 4H), 1.67-1.50 (m, 12H), 1.37-1.21 (m, 36H), 0.88 (t, J=6.8 Hz, 6H).

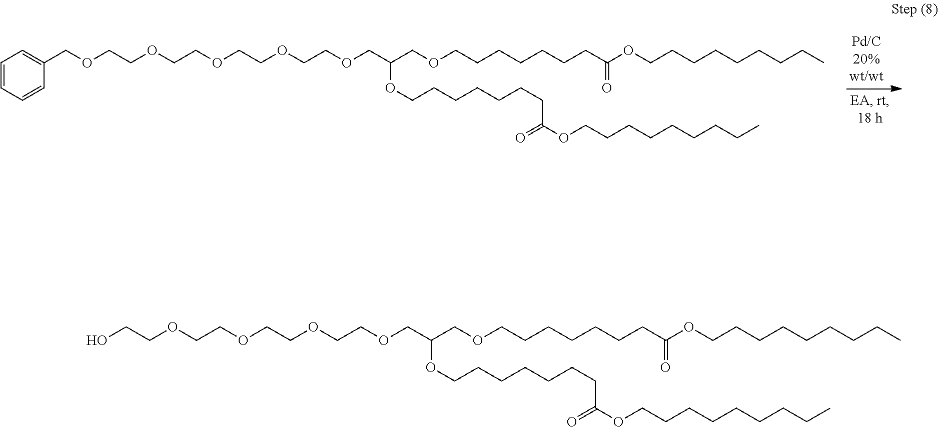 [SMILES: CCCCCCCCCOC(=O)CCCCCCCOCC(COCCOCCOCCOCCOCc1ccccc1)OCCCCCCCC(=O)OCCCCCCCCC ; CCCCCCCCCOC(=O)CCCCCCCOCC(COCCOCCOCCOCCO)OCCCCCCCC(=O)OCCCCCCCCC]

To the solution of nonyl 8-[3-[2-[2-[2-(2-benzyloxyethoxy)ethoxy]ethoxy]ethoxy]-2-(8-nonoxy-8-oxo-octoxy)propoxy]octanoate (5 g, 5.31 mmol) in ethyl acetate (100 mL) was added Pd/C (1.13 g, 20% wt/wt). The mixture was stirred at room temperature under hydrogen for 18 h. TLC (ethyl acetate/petroleum ether 1/1) indicated that the starting material was consumed. The reaction was filtered through celite and washed with ethyl acetate to give nonyl 8-[3-[2-[2-[2-(2-hydroxyethoxy)ethoxy]ethoxy]ethoxy]-2-(8-nonoxy-8-oxo-octoxy)propoxy]octanoate (4.22 g, 4.98 mmol, 93.8%) as colorless oil.

¹H NMR (400 MHz, CDCl₃) δ 4.05 (t, J=6.8 Hz, 4H), 3.74-3.38 (m, 27H), 2.28 (t, J=7.5 Hz, 4H), 1.68-1.50 (m, 12H), 1.39-1.21 (m, 37H), 0.88 (t, J=6.8 Hz, 6H).

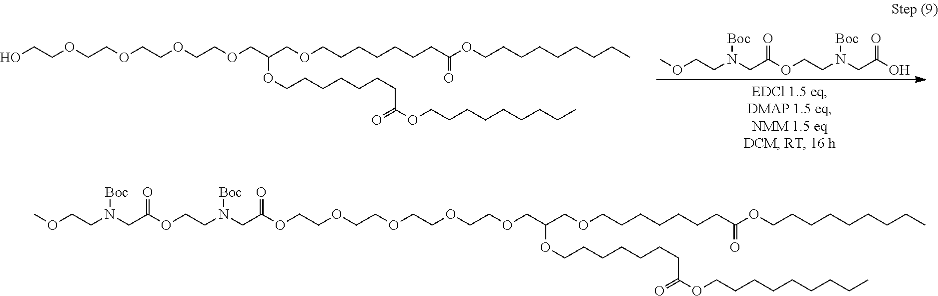

Nonyl 8-[3-[2-[2-[2-(2-hydroxyethoxy)ethoxy]ethoxy]ethoxy]-2-(8-nonoxy-8-oxo-octoxy)propoxy]octanoate (330 mg, 0.39 mmol), 2-[tert-butoxycarbonyl-[3-[tert-butoxycarbonyl(2-methoxyethyl)amino]-2-oxo-propyl]amino]acetic acid (199 mg, 0.467 mmol) and N,N-dimethylpyridin-4-amine (71.4 mg, 0.58 mmol) were dissolved in DCM-Anhydrous (15 mL) in presence of 4-methylmorpholine (59.1 mg, 0.58 mmol). The reaction was cooled to 0° C. and 3-(ethyliminomethyleneamino)-N,N-dimethyl-propan-1-amine;hydrochloride (112 mg, 0.58 mmol) was added portionwise to the mixture. The reaction was warmed to room temperature and stirred overnight. The reaction was monitored by TLC (petroleum ether/EtOAc 5:5, expected product rf: 0.5). Water was added and the organic layers were extracted with DCM (3×15 mL), washed with brine, dried over Na2SO4, filtered and concentrated. The residue was purified by flash column chromatography on silica gel eluting with 20% to 40% ethyl acetate in petroleum ether to give nonyl 8-[3-[2-[2-[2-[2-[2-[tert-butoxycarbonyl-[3-[tert-butoxycarbonyl(2-methoxyethyl)amino]-2-oxo-propyl]amino]acetyl]oxyethoxy]ethoxy]ethoxy]ethoxy]-2-(8-nonoxy-8-oxo-octoxy)propoxy]octanoate (0.23 g, 47.1%) as colorless oil.

¹H NMR (400 MHz, CDCl₃) δ 4.31-4.20 (m, 4H), 4.05 (t, J=6.8 Hz, 6H), 4.00-3.95 (m, 2H), 3.70 (s, 2H), 3.66-3.62 (m, 11H), 3.58-3.39 (m, 15H), 3.30 (d, J=5.5 Hz, 3H), 2.33-2.24 (m, 4H), 1.65-1.52 (m, 12H), 1.49-1.40 (m, 18H), 1.37-1.24 (m, 37H), 0.88 (t, J=6.9 Hz, 6H).

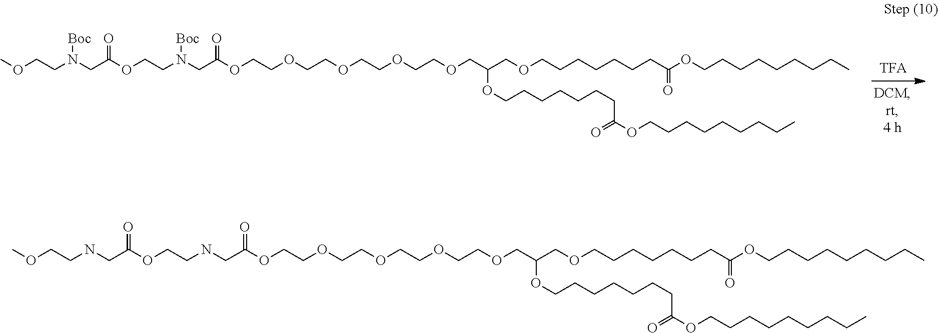

To the solution of nonyl 8-[3-[2-[2-[2-[2-[2-[tert-butoxycarbonyl-[3-[tert-butoxycarbonyl(2-methoxyethyl)amino]-2-oxo-propyl]amino]acetyl]oxyethoxy]ethoxy]ethoxy]ethoxy]-2-(8-nonoxy-8-oxo-octoxy)propoxy]octanoate (230 mg, 0.183 mmol) in DCM (5 mL) cooled to 0° C. was added TFA (1 mL) and the mixture was stirred at rt for 4 hrs. TLC (DCM/MeOH 10:1, expected product rf: 0.1) indicated that the starting material was disappeared. The solvent was removed and the product was azeotroped with dichloromethane several times and then dried under vacuum for 2 hrs to give nonyl 8-[3-[2-[2-[2-[2-[2-[[3-(2-methoxyethylamino)-2-oxo-propyl]amino]acetyl]oxyethoxy]ethoxy]ethoxy]ethoxy]-2-(8-nonoxy-8-oxo-octoxy)propoxy]octanoate;2,2,2-trifluoroacetic acid (0.226 g, quant.) as light yellow oil.

¹H NMR (400 MHz, CDCl₃) δ 4.59 (s, 2H), 4.38 (s, 2H), 4.05 (t, J=6.7 Hz, 4H), 4.01-3.90 (m, 4H), 3.75-3.69 (m, 4H), 3.64 (s, 11H), 3.59-3.49 (m, 5H), 3.49-3.40 (m, 6H), 3.37 (s, 3H), 3.32-3.28 (m, 2H), 2.29 (t, J=7.5 Hz, 4H), 1.70-1.45 (m, 11H), 1.41-0.96 (m, 37H), 0.88 (t, J=6.9 Hz, 6H).

### Example 13: Synthesis of 1-octylnonyl 8-[3-[2-[2-[2-[2-[2-[2-[2-(2-methoxyethylamino)acetyl]oxyethylamino]acetyl]oxyethoxy]ethoxy]ethoxy]ethoxy]-2-[8-(1-octylnonoxy)-8-oxo-octoxy]propoxy]octanoate;2,2,2-trifluoroacetic Acid (Compound (XVI))

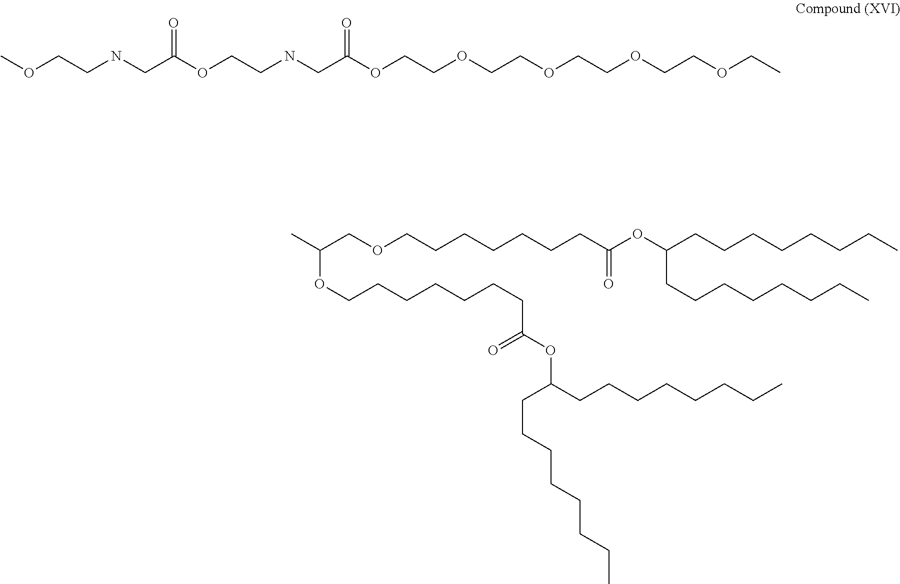

Synthesis of Compound (XVI)

A mixture of 1-octylnonyl 8-[3-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]-2-[8-(1-octylnonoxy)-8-oxooctoxy]propoxy]octanoate (0.33 g, 0.228 mmol) in DCM (5 mL) was added 2,2,2-trifluoroacetic acid (0.26 g, 0.228 mmol). The mixture was stirred for 3 h at ambient temperature. The mixture was concentrated to give 1-octylnonyl 8-[3-[2-[2-[2-[2-[2-[2-[2-(2-methoxyethylamino)acetyl]oxyethylamino]acetyl]oxyethoxy]ethoxy]ethoxy]ethoxy]-2-[8-(1-octylnonoxy)-8-oxo-octoxy]propoxy]octanoate;2,2,2-trifluoroacetic acid (0.308 g, 99%) as yellow oil.

¹H NMR (400 MHz, CDCl₃) δ 4.87 (p, J=6.0 Hz, 2H), 4.59 (s, 2H), 4.37 (s, 2H), 4.03 (d, J=19.2 Hz, 4H), 3.78-3.40 (m, 27H), 3.41-3.29 (m, 51H), 2.30 (t, J=7.5 Hz, 4H), 1.55 (dd, J=30.7, 11.0 Hz, 16H), 1.35-1.18 (m, 62H), 0.87 (t, J=6.8 Hz, 12H).

### Example 14: Synthesis of [(Z)-non-2-enyl] 8-[3-[2-[2-[2-[2-[2-[2-[2-(2-methoxyethylamino)acetyl]oxyethylamino]acetyl]oxyethoxy]ethoxy]ethoxy]ethoxy]-2-[8-[(Z)-non-2-enoxy]-8-oxo-octoxy]propoxy]octanoate;2,2,2-trifluoroacetic Acid (Compound (XVII))

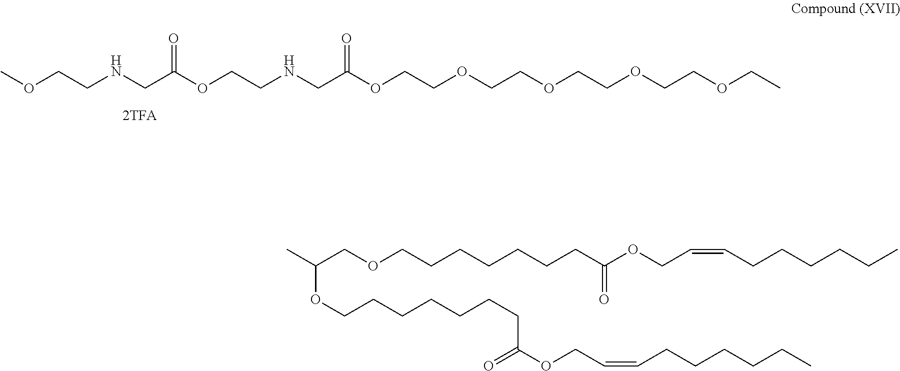

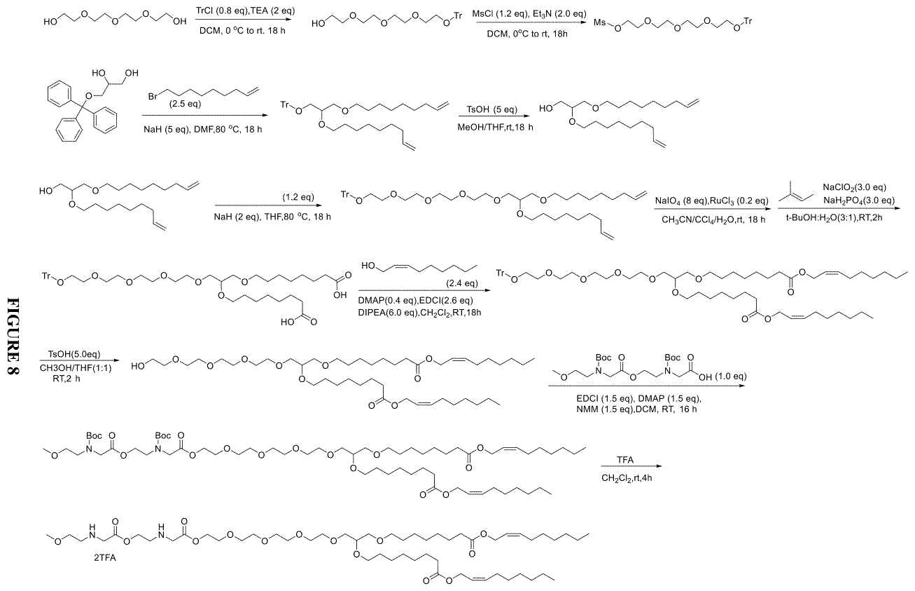

Compound (XVII) was synthesized based on the chemistry shown in Scheme (10) presented on FIG. 8.

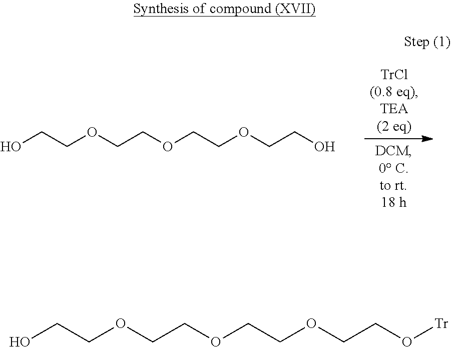

A mixture of 2-[2-[2-(2-hydroxyethoxy)ethoxy]ethoxy]ethanol (40 g, 0.206 mol), N,N-dimethyl pyridin-4-amine (1.26 g, 0.0103 mol) and [chloro(diphenyl)methyl]benzene (45.9 g, 0.165 mol) in DCM (300 mL). The mixture was cooled to 0° C., then added N,N-diethylethanamine (41.7 g, 0.412 mol). The reaction mixture was stirred for 16 hrs at ambient temperature. LCMS showed a good reaction. The mixture was poured into water (600 mL) and extracted with DCM (2*400 mL). The organic layer was washed with water, NaCl, dried over Na2SO₄ and concentrated. The residue was purified by flash column chromatography on silica gel eluting with 3:1 ethyl acetate/petroleum ether to give 2-[2-[2-(2-trityloxyethoxy)ethoxy]ethoxy]ethanol (33.0 g, 36.7%) as colorless oil.

LCMS 459 (M+23), 99% UV 214 nm

¹H NMR (400 MHz, CDCl₃) δ 7.49-7.44 (m, 6H), 7.32-7.26 (m, 6H), 7.25-7.19 (m, 3H), 3.72-3.64 (m, 12H), 3.61-3.57 (m, 2H), 3.27-3.22 (m, 2H), 2.55-2.50 (m, 1H).

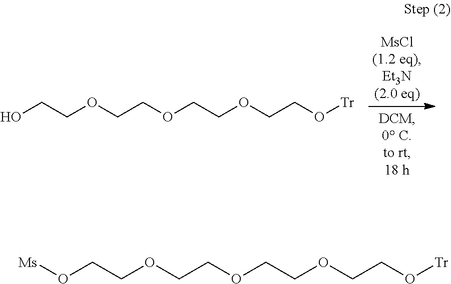

To a mixture of 2-[2-[2-(2-trityloxyethoxy)ethoxy]ethoxy]ethanol (33 g, 0.0756 mol) and N,N-diethylethanamine (15.3 g, 0.151 mol) in DCM (600 mL) was added methanesulfonyl chloride (10.4 g, 0.0907 mol) slowly at 0° C. The mixture was stirred overnight at room temperature. CH₂Cl₂ (400 mL) were added to the solution, and the mixture was washed with diluted HCl (1M, 1000 mL).The mixture was shaken, the layers were separated, and the organic layer was collected. The organic layer was further washed with Water (1000 mL) and brine (1000 mL), and dried over Na₂SO₄. Solvent was removed to give 2-[2-[2-(2-trityloxyethoxy)ethoxy]ethoxy]ethyl methanesulfonate (38.8 g, 99.8%) as a yellow oil.

LCMS 537.2 (M+23) 98% UV (214 nm)

¹H NMR (400 MHz, CDCl₃) δ 7.48-7.44 (m, 6H), 7.32-7.27 (m, 5H), 7.26-7.20 (m, 4H), 4.35-4.30 (m, 2H), 3.75-3.71 (m, 2H), 3.70-3.63 (m, 10H), 3.26-3.20 (m, 2H), 2.98 (s, 3H).

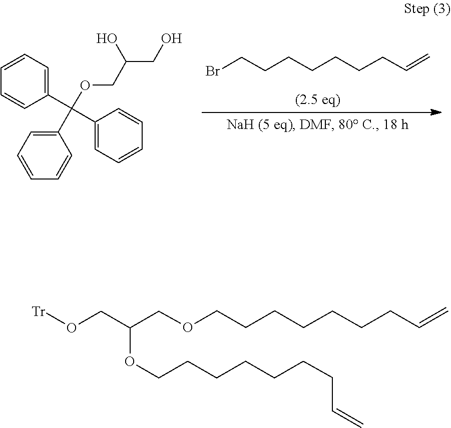

To a suspension of NaH (9.09 g, 60% in oil, 227 mmoles) in 300 ml of anhydrous DMF was added 3-trityloxypropane-1,2-diol (20 g, 56.8 mmoles). The mixture was heated at 80° C. for 15 minutes and cooled to room temperature. 9-bromonon-1-ene (30 g, 142 mmoles) in 10 ml of anhydrous DMF was added dropwise to the mixture which was then heated under 80° C. for 18 h. After cooling to RT, 500 ml of H2O were added to destroy remaining NaH. The organic phase was extracted with 3×250 ml ethyl acetate. The extract was washed successively with 2×150 ml of 1N HCl, 2×150 ml of 5% (w/v) NaHCO₃ and 150 ml of brine and dried on Na2SO4. The solvent was evaporated under reduced pressure and the resulting oil was purified on a silica gel column eluted with petroleum ether/ethyl acetate (2% to 5% ethyl acetate in petroleum ether) to yield a colorless oil (12 g; yield 34.4%).

¹H NMR (400 MHz, CDCl₃) δ 7.51-7.42 (m, 6H), 7.31-7.20 (m, 9H), 5.88-5.72 (m, 2H), 5.03-4.89 (m, 4H), 3.59-3.46 (m, 5H), 3.39 (t, J=6.7 Hz, 2H), 3.22-3.13 (m, 2H), 2.03 (td, J=8.0, 1.3 Hz, 4H), 1.57-1.45 (m, 4H), 1.44-1.20 (m, 16H).

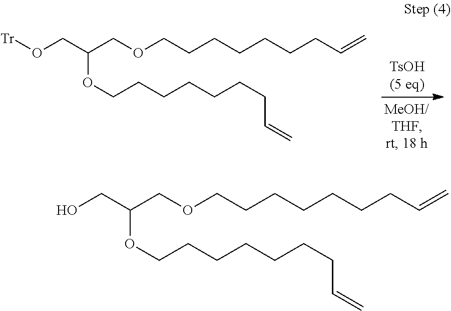

To a solution of [2,3-bis(non-8-enoxy)propoxy-diphenyl-methyl]benzene (10 g, 16.3 mmol) in methanol/THF (150 mL, 1/1 v/v) was added p-Toluenesulfonic acid (14 g, 81.5 mmol) in one portion at room temperature and the mixture was stirred at room temperature for 18 h. TLC (4% ethyl acetate in petroleum ether) indicated that the starting material was disappeared completely. 10 mL triethylamine was added to quench the reaction and the solvent was removed under vacuum. The residue was purified by flash chromatography eluted with 20% to 30% ethyl acetate in petroleum ether to give 2,3-bis(non-8-enoxy)propan-1-ol (5.1 g, 82.7%) as colorless oil.

¹H NMR (400 MHz, CDCl₃) δ 5.90-5.74 (m, 2H), 5.03-4.90 (m, 4H), 3.79-3.32 (m, 9H), 2.22 (dd, J=6.5, 5.9 Hz, 1H), 2.04 (q, J=6.9 Hz, 4H), 1.63-1.47 (m, 4H), 1.43-1.19 (m, 16H).

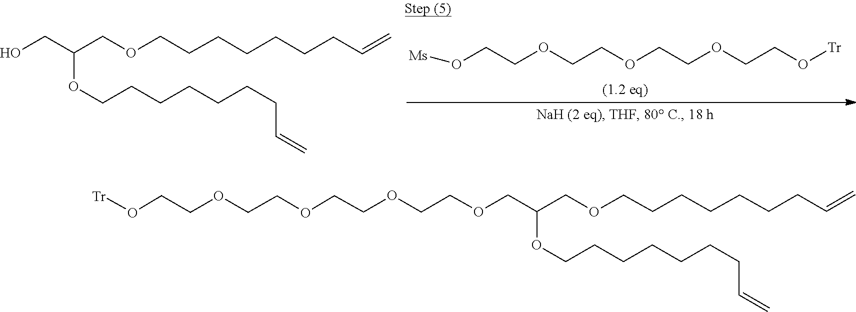

To a mixture of 2,3-bis(non-8-enoxy)propan-1-ol (5.2 g, 15.3 mmol) added NaH (60% mineral oil dispersion, 1.51 mg, 30.5 mmol) in 60 mL of dry THF then stirred 15 min at 80° C. Then after the solvent was restore room temperature added 2-[2-[2-(2-trityloxyethoxy)ethoxy]ethoxy]ethyl methanesulfonate (9.43 g, 18.3 mmol) dissolved in 20 mL of dry THF. The reaction mixture was stirred reflux (80° C.) overnight. The reaction mixture was cooled to room temperature, and water was added. EtOAc (100 mL) was added, the mixture was shaken, the layers were separated, and the organic layer was collected. The aqueous layer was extracted with EtOAc (50 mL*2). The combined organic layers were washed with brine and dried over Na₂SO₄. The residue was purified by flash column chromatography on silica gel eluting with ethyl acetate in petroleum ether (0-15%) (11%) to give target product (7.44, 64.2% yield) as pale yellow oil.

¹H NMR (400 MHz, CDCl₃) δ 7.48-7.44 (m, 6H), 7.31-7.26 (m, 6H), 7.24-7.19 (m, 3H), 5.86-5.74 (m, 2H), 5.01 (d, J=1.4 Hz, 1H), 4.96 (d, J=1.4 Hz, 1H), 4.94-4.92 (m, 1H), 4.92-4.89 (m, 1H), 3.68-3.64 (m, 9H), 3.62 (s, 4H), 3.59-3.38 (m, 10H), 3.25-3.22 (m, 2H), 2.07-2.00 (m, 4H), 1.59-1.51 (m, 4H), 1.39-1.27 (m, 16H).

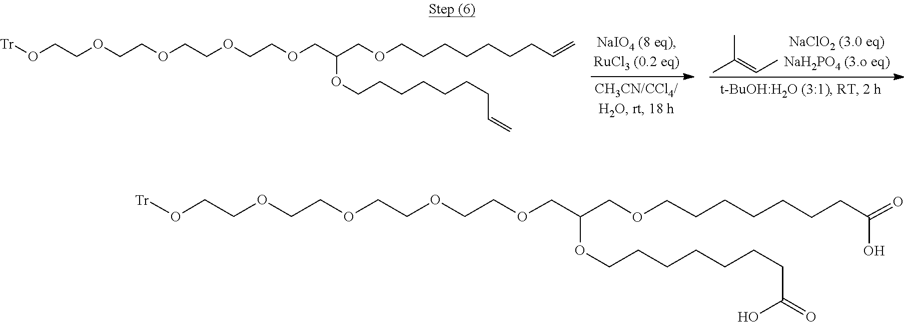

To a solution of [2-[2-[2-[2-[2,3-bis(non-8-enoxy)propoxy]ethoxy]ethoxy]ethoxy]ethoxy-diphenyl-methyl]benzene (7.44 g, 9.8 mmol) in MeCN (60 mL), CCl₄ (60 mL) and water (60 mL) was added NaIO₄ (16.8 g, 78.4 mmol) and RuCl₃ (0.407 g, 1.96 mmol). The reaction mixture was stirred at room temperature for 24 h. The reaction was filtered and the filtrate was diluted with ethyl acetate (500 mL) and washed with 1N aq. HCl (200 mL). The organic layer was washed with Na₂S₂O₃ solution and then dried over sodium sulfate, filtered and concentrated to give 8-[2-(7-carboxyheptoxy)-3-[2-[2-[2-(2-trityloxyethoxy)ethoxy]ethoxy]ethoxy]propoxy]octanoic acid (7.98 g, 6.02 mmol, 61.4% yield) as yellow oil which was used without further purification.

¹H NMR (400 MHz, CDCl₃) δ 9.75 (s, 1H), 7.47-7.44 (m, 3H), 7.35-7.26 (m, 10H), 7.23 (d, J=7.2 Hz, 2H), 3.80-3.38 (m, 24H), 3.26-3.21 (m, 1H), 2.45-2.25 (m, 4H), 1.59 (d, J=34.5 Hz, 8H), 1.33 (s, 12H).

Step (7)

8-[2-(8-oxooctoxy)-3-[2-[2-[2-(2-trityloxyethoxy)ethoxy]ethoxy]ethoxy]propoxy]octanoicacid (6.98 g, 3.58 mmol) was dissolved in t-BuOH: H₂O (3:1, 120 mL), containing NaH₂PO₄ (1.28 g, 10.8 mmol), 2-methy-2-butene (11 mL) and sodium chlorite (0.972 g, 10.8 mmol). The reaction was stirred for 2 h at rt and LCMS indicated that the starting material was consumed. The reaction mixture was diluted with H₂O. The aqueous layer was extracted with ethyl acetate. The combined extract was dried over sodium sulfate and concentrated to afford 8-[2-(7-carboxyheptoxy)-3-[2-[2-[2-(2-trityloxyethoxy)ethoxy]ethoxy]ethoxy]propoxy]octanoic acid (6.161 g, 7.75 mmol, quant.) as light yellow oil.

¹H NMR (400 MHz, CDCl₃) δ 7.47-7.44 (m, 3H), 7.34-7.26 (m, 10H), 7.25-7.19 (m, 2H), 3.76-3.39 (m, 24H), 3.25-3.21 (m, 1H), 2.43-2.27 (m, 4H), 1.68-1.49 (m, 8H), 1.32 (d, J=6.6 Hz, 12H).

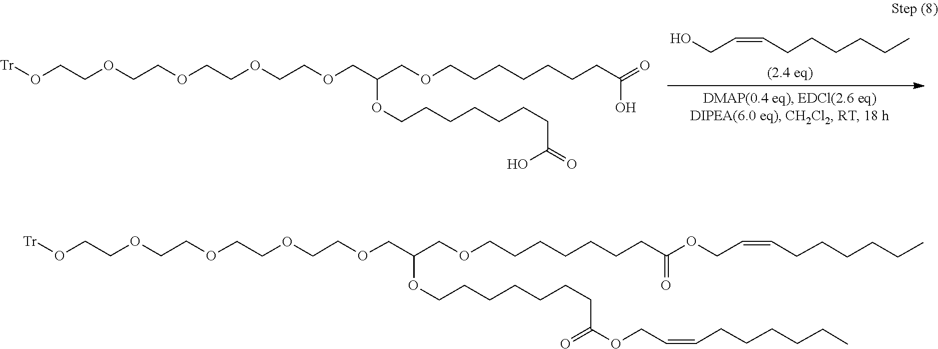

To the solution of 8-[2-(7-carboxyheptoxy)-3-[2-[2-[2-(2-trityloxyethoxy)ethoxy]ethoxy]ethoxy]propoxy]octanoic acid (6.161 g, 7.75 mmol) and (Z)-non-2-en-1-ol (2.65 g, 18.6 mmol) in dry dichloromethane (120 mL) then added DIPEA (6.01 g, 46.5 mmol), DMAP (0.379 g, 0.31 mmol) and under ice bath added EDCI (3.86 g, 20.1 mmol). The mixture was stirred at room temperature for 18 hrs. The reaction was diluted with dichloromethane and washed with brine. The organic layer was dried over sodium sulfate, filtered and concentrated. The residue was purified by flash chromatography eluted with 0% to 40% (28%) ethyl acetate in petroleum ether to give [(Z)-non-2-enyl] 8-[2-[8-[(Z)-non-2-enoxy]-8-oxo-octoxy]-3-[2-[2-[2-(2-trityloxyethoxy)ethoxy]ethoxy]ethoxy]propoxy]octanoate (1.026 g, 12.7% yield) as colorless oil.

¹H NMR (400 MHz, CDCl₃) δ 7.46 (d, J=7.4 Hz, 6H), 7.30 (d, J=7.1 Hz, 6H), 7.23 (d, J=7.2 Hz, 3H), 5.69-5.58 (m, 2H), 5.57-5.47 (m, 2H), 4.61 (d, J=6.8 Hz, 4H), 3.68-3.39 (m, 23H), 3.23 (t, J=5.2 Hz, 2H), 2.29 (t, J=7.0 Hz, 4H), 2.09 (q, J=7.1 Hz, 4H), 1.61 (s, 4H), 1.57-1.50 (m, 4H), 1.32 (td, J=13.4, 3.7 Hz, 28H), 0.88 (t, J=6.8 Hz, 6H).

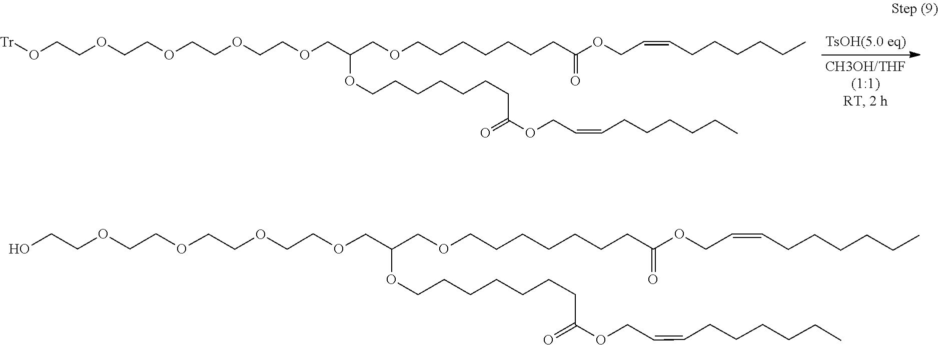

To a solution of [(Z)-non-2-enyl] 8-[2-[8-[(Z)-non-2-enoxy]-8-oxo-octoxy]-3-[2-[2-[2-(2-trityloxyethoxy)ethoxy]ethoxy]ethoxy]propoxy]octanoate (0.920 g, 0.882 mmol) in methanol/THF (60 mL, 1/1 v/v) was added p-Toluenesulfonic acid (0.839, 4.41 mmol) in one portion at room temperature and the mixture was stirred at room temperature for 2 h. TLC (30% ethyl acetate in petroleum ether) indicated that the starting material was disappeared completely. 5 mL triethylamine was added to quench the reaction and the solvent was removed under vacuum. The residue was purified by flash chromatography eluted with 0% to 90% (80%) ethyl acetate in petroleum ether to give 2,3-bis(non-8-enoxy)propan-1-ol (0.402 g,56.9%) as colorless oil.

¹H NMR (400 MHz, CDCl₃) δ 5.69-5.59 (m, 2H), 5.56-5.48 (m, 2H), 4.62 (d, J=6.8 Hz, 4H), 3.69-3.39 (m, 23H), 2.33-2.26 (m, 4H), 2.13-2.06 (m, 4H), 1.66-1.50 (m, 8H), 1.45-1.21 (m, 30H), 0.91-0.85 (m, 6H).

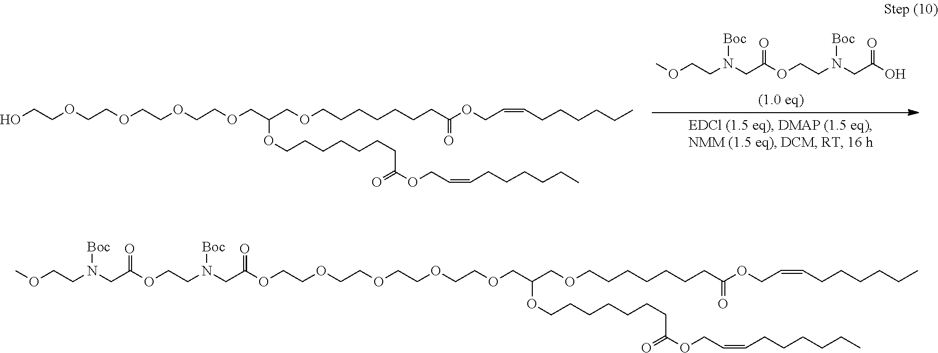

To a solution of[(Z)-non-2-enyl] 8-[3-[2-[2-[2-(2-hydroxyethoxy)ethoxy]ethoxy]ethoxy]-2-[8-[(Z)-non-2-enoxy]-8-oxo-octoxy]propoxy]octanoate (0.44 g, 0.549 mmol) in dry DCM (15 mL) was added 2-[tert-butoxycarbonyl-[2-[2-[tert-butoxycarbonyl(2-methoxyethyl)amino]acetyl]oxyethyl]amino]acetic acid (0.239 g, 0.549 mmol), 4-methylmorpholine (0.084 g, 0.824 mmol) and DMAP (0.101 g, 0.824 mmol) at room temperature. Then the mixture was cooled to 0° C. and portion wise over 15 min added EDCI (0.158 g, 0.824 mmol). The reaction was stirred for 17 hours at room temperature. TLC shows that the reaction was finished. The reaction was poured into water and extracted with DCM. The water was extracted one more time with DCM. The combined organics were washed with brine, dried over Na₂SO₄ and concentrated in vacuo. Then purified by column chromatography with acetate in petroleum (0-100%)(60%) to give target product (0.476 g,71.2% yield) as pale yellow oil.

¹H NMR (400 MHz, CDCl₃) δ 5.69-5.59 (m, 2H), 5.56-5.48 (m, 2H), 4.62 (d, J=6.8 Hz, 4H), 4.30-4.19 (m, 4H), 4.04 (d, J=6.1 Hz, 2H), 4.00-3.95 (m, 2H), 3.70 (s, 2H), 3.64 (d, J=5.4 Hz, 12H), 3.58-3.39 (m, 15H), 3.30 (d, J=5.6 Hz, 3H), 2.30 (t, J=7.4 Hz, 4H), 2.13-2.05 (m, 4H), 1.60-1.50 (m, 6H), 1.44 (d, J=18.8 Hz, 18H), 1.40-1.22 (m, 30H), 0.91-0.85 (m, 6H).

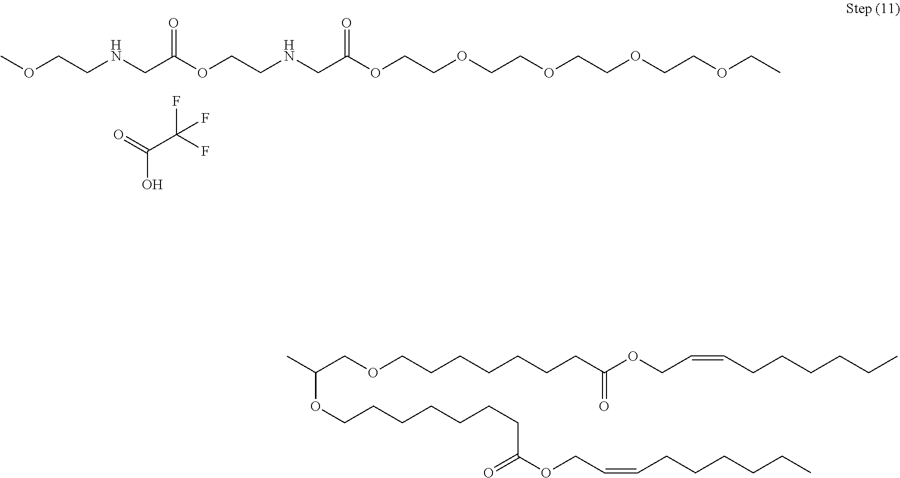

To the solution of undecyl 8-[3-(tert-butoxycarbonylamino)propyl-[8-(1-octylnonoxy)-8-oxo-octyl]amino]octanoate (0.476 g, 0.391 mmol) in CH2Cl2 (5 mL)under ice bath, was dropwise TFA (3 mL) dropwise and the mixture was stirred at room temperature for 4 h. The solvent was remove under vacuum then dissolved in CH₂Cl₂ and remove solvent several time to remove TFA and to give [(Z)-non-2-enyl] 8-[3-[2-[2-[2-[2-[2-[2-[2-(2-methoxyethylamino)acetyl]oxyethylamino]acetyl]oxyethoxy]ethoxy]ethoxy]ethoxy]-2-[8-[(Z)-non-2-enoxy]-8-oxo-octoxy]propoxy]octanoate;2,2,2-trifluoroacetaldehyde as pale yellow oil (0.530 g, yield: 112%).

¹H NMR (400 MHz, CDCl₃) δ 5.69-5.60 (m, 2H), 5.56-5.47 (m, 2H), 4.62 (d, J=6.9 Hz, 4H), 4.36 (s, 2H), 4.04 (s, 2H), 3.98 (s, 2H), 3.73-3.68 (m, 4H), 3.67-3.41 (m, 23H), 3.36 (s, 3H), 3.33 (d, J=4.5 Hz, 2H), 2.34-2.27 (m, 4H), 2.13-2.02 (m, 4H), 1.67-1.58 (m, 4H). 1.54 (s. 4H). 1.41-1.21 (m. 30H). 0.92-0.84 (m. 6H).

### Example 15: Synthesis of [(Z)-non-2-enyl] 8-[3-[2-[2-[2-[2-[2-[2-[2-(2-methoxyethylamino)acetyl]oxyethylamino]acetyl]oxyethoxy]ethoxy]ethoxy]ethoxy]-2-[(7R,11R)-3,7,11,15-tetramethylhexadecoxy]propoxy]octanoate;2,2,2-trifluoroacetic Acid (Compound (XVIII))

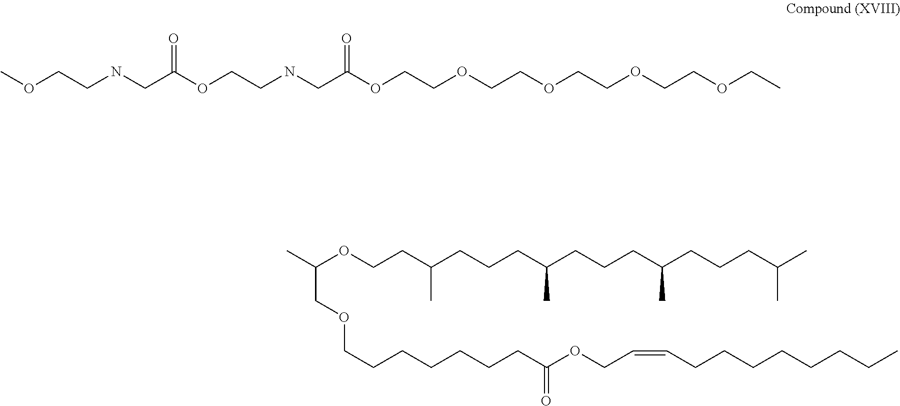

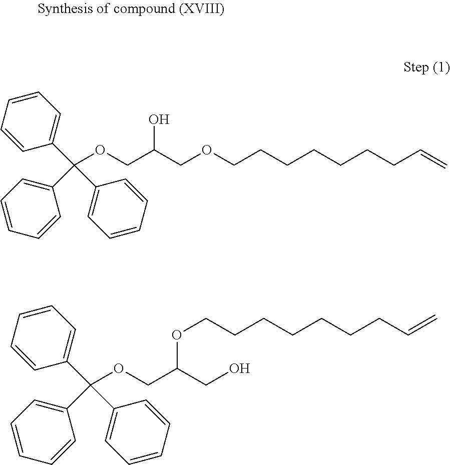 [SMILES: C=CCCCCCCCOCC(O)COC(c1ccccc1)(c1ccccc1)c1ccccc1 ; C=CCCCCCCCOC(CO)COC(c1ccccc1)(c1ccccc1)c1ccccc1]

To a suspension of NaH (15 g, 60% in oil 113.5 mmoles) in 450 ml of anhydrous DMF was added 3-trityloxypropane1,2-diol (30 g, 28.4 mmoles). The mixture was heated at 80° C. for 15 minutes and cooled to room temperature. 9-bromonon-1-ene (45 g, 142 mmoles) in 5 ml of anhydrous DMF was added dropwise to the mixture which was then heated under 80° C. for 18 h.

After cooling to RT, 1000 ml of H2O were added to destroy remaining NaH. The organic phase was extracted with 3×250 ml ethyl acetate. The extract was washed successively with 2×150 ml of 1N HCl, 2×150 ml of 5% (w/v) NaHCO₃ and 150 ml of brine and dried on Na2SO4. The solvent was evaporated under reduced pressure and the resulting oil was purified on a silica gel column eluted with petroleum ether/ethyl acetate (2% to 5% ethyl acetate in petroleum ether) to yield a colorless oil (17.5 g; yield 33.5%). There are two by-product, 1-non-8-enoxy-3-trityloxy-propan-2-ol (7.2 g; yield 18.4%) and 2-non-8-enoxy-3-trityloxy-propan-1-ol (3.77 g; yield 9.65%)

¹H NMR (400 MHz, CDCl₃) δ 7.46 (d, J=7.4 Hz, 5H), 7.28 (t, J=6.6 Hz, 5H), 7.22 (dd, J=8.2, 6.1 Hz, 3H), 5.87-5.74 (m, 2H), 4.99 (d, J=17.1 Hz, 2H), 4.93 (d, J=10.2 Hz, 2H), 3.65-3.47 (m, 5H), 3.43-3.00 (m, 4H), 2.09-2.01 (m, 4H), 1.50-1.21 (m, 17H).

¹H NMR (400 MHz, CDCl₃) δ 7.43 (d, J=7.4 Hz, 6H), 7.30 (t, J=7.5 Hz, 6H), 7.23 (t, J=7.3 Hz, 3H), 5.81 (ddt, J=16.9, 10.1, 6.6 Hz, 1H), 5.03-4.89 (m, 2H), 3.95 (dd, J=10.2, 4.7 Hz, 1H), 3.55-3.40 (m, 4H), 3.22-3.14 (m, 2H), 2.41 (d, J=4.5 Hz, 1H), 2.04 (q, J=6.9 Hz, 2H), 1.56-1.50 (m, 4H), 1.29 (s, 6H).

¹H NMR (400 MHz, CDCl₃) δ 7.44 (d, J=7.6 Hz, 6H), 7.30 (t, J=7.5 Hz, 6H), 7.26-7.20 (m, 3H), 5.81 (ddtd, J=16.9, 10.1, 6.6, 3.5 Hz, 3H), 4.99 (d, J=17.1 Hz, 3H), 4.93 (d, J=10.2 Hz, 2H), 3.80-3.10 (m, 11H), 2.13-1.97 (m, 7H), 1.59-1.51 (m, 6H), 1.45-1.18 (m, 24H).

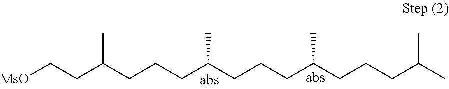 [SMILES: CC(C)CCC[C@@H](C)CCC[C@@H](C)CCCC(C)CCOS(C)(=O)=O]

To a solution of (7R,11R)-3,7,11,15-tetramethylhexadecan-1-ol (23.5 g, 78.7 mmol) in DCM (300 ml) was added Triethylamine (15.9 g, 157 mmol) and Methanesulfonyl chloride (13.5 g, 118 mmol) at 0° C. The mixture was stirred at 25° C. for 14 hr. After reaction, the mixture was concentrated and dealt with EA (250 ml), washed with 1N HCl/H₂O (250 ml×2), NaHCO₃ aq (250 ml×2), NaCl aq (250 ml) and dried over Na2SO4. The organic was concentrated to give [(7R,11R)-3,7,11,15-tetramethylhexadecyl] methanesulfonate (30.4 g, crude) as a yellow oil.

¹H NMR (400 MHz, CDCl₃) δ 4.30-4.23 (m, 2H), 3.00 (d, J=3.2 Hz, 3H), 1.79 (ddt, J=9.2, 7.2, 2.2 Hz, 1H), 1.58-1.51 (m, 2H), 1.35-1.07 (m, 21H), 0.93-0.84 (m, 15H).

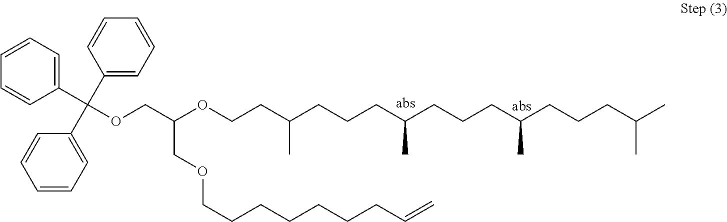 [SMILES: C=CCCCCCCCOCC(COC(c1ccccc1)(c1ccccc1)c1ccccc1)OCCC(C)CCC[C@H](C)CCC[C@H](C)CCCC(C)C]

To a solution of 1-non-8-enoxy-3-trityloxy-propan-2-ol (3.5 g,7.6 mmol) in DMF (150 ml) was added NaH (1.53 g, 38.2 mmol). The mixture was stirred at 80° C. for 1 hr. The [(7R,11R)-3,7,11,15-tetramethylhexadecyl] methanesulfonate (4.31 g, 11.4 mmol) was added to the mixture dropwise. The mixture was stirred at 80° C. for another 14 hrs. The mixture was dealt with EA (150 ml), washed with water (250 ml×2), NaCl sat.aq (150 ml) and dried over Na2SO4. The organic was concentrated and purified by flash (10% EA in PE), to give [[3-non-8-enoxy2-[(7R,11R)-3,7,11,15-tetramethylhexadecoxy]propoxy]-diphenyl-methyl]benzene (1.88 g,2.5 mmol yield 32.7%) as a colorless oil.

¹H NMR (400 MHz, CDCl₃) δ 7.46 (dd, J=10.8, 3.6 Hz, 6H), 7.34-7.26 (m, 6H), 7.23 (dd, J=9.1, 2.0 Hz, 3H), 5.81 (ddt, J=16.9, 10.2, 6.7 Hz, 1H), 5.05-4.90 (m, 2H), 3.65-3.35 (m, 8H), 3.17 (d, J=3.5 Hz, 1H), 2.03 (dd, J=14.4, 6.8 Hz, 2H), 1.51 (dd, J=13.2, 6.6 Hz, 4H), 1.34-1.13 (m, 30H), 0.87-0.83 (m, 15H).

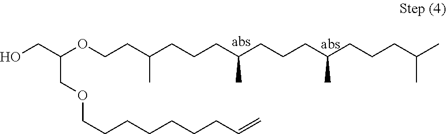 [SMILES: C=CCCCCCCCOCC(CO)OCCC(C)CCC[C@H](C)CCC[C@H](C)CCCC(C)C]

To a solution of [[3-non-8-enoxy-2-[(7R,11R)-3,7,11,15-tetramethylhexadecoxy]propoxy]-diphenylmethyl]benzene (3.5 g, 4.73 mmol) in MeOH/THF (30 ml 1:1) was added Toluene-4-sulfonic acid (4.50 g, 23.7 mmol). The mixture was stirred at 25° C. for 14 hr. The mixture was concentrated and dealt with EA (150 ml), washed with water (150 ml×2), NaCl sat.aq (150 ml) and dried over Na₂SO₄. The organic was concentrated and purified by flash (10% EA in PE), to give 3-non-8-enoxy-2-[(7R,11R)-3,7,11,15-tetramethylhexadecoxy]propan-1-ol (1.22 g, yield 50.8%) as a yellow oil.

¹H NMR (400 MHz, CDCl₃) δ 5.81 (ddt, J=16.9, 10.2, 6.7 Hz, 1H), 5.03-4.89 (m, 2H), 3.73-3.42 (m, 9H), 2.04 (dd, J=14.4, 6.8 Hz, 2H), 1.57-1.49 (m, 4H), 1.35-1.06 (m, 30H), 0.86 (dd, J=8.5, 6.3 Hz, 15H).

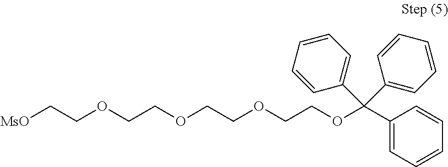 [SMILES: CS(=O)(=O)OCCOCCOCCOCCOC(c1ccccc1)(c1ccccc1)c1ccccc1]

To a mixture of 2-[2-[2-(2-trityloxyethoxy)ethoxy]ethoxy]ethanol (33 g, 0.0756 mol) and N,N-diethylethanamine (15.3 g, 0.151 mol) in DCM (600 mL) was added methanesulfonyl chloride (10.4 g, 0.0907 mol) slowly at 0° C. The mixture was stirred overnight at room temperature. CH₂Cl₂ (400 mL) were added to the solution, and the mixture was washed with diluted HCl (1M,1000 mL). The mixture was shaken, the layers were separated, and the organic layer was collected. The organic layer was further washed with Water (1000 mL) and brine (1000 mL), and dried over Na₂SO₄. Solvent was then removed to give 2-[2-[2-(2-trityloxyethoxy)ethoxy]ethoxy]ethyl methanesulfonate (38.8 g,75.4 mmol, 99.8% yield) as a yellow oil.

¹H NMR (400 MHz, CDCl₃) δ 7.48-7.44 (m, 6H), 7.32-7.27 (m, 5H), 7.26-7.20 (m, 4H), 4.35-4.30 (m, 2H), 3.75-3.71 (m, 2H), 3.70-3.63 (m, 10H), 3.26-3.20 (m, 2H), 2.98 (s, 3H). LCMS Find peak: 537.2 (M+23) at 2.198 min 98% UV (214 nm)

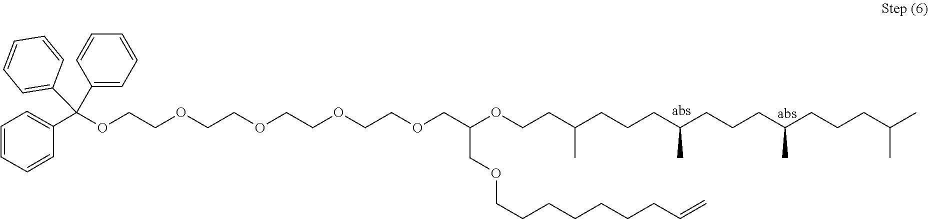 [SMILES: C=CCCCCCCCOCC(COCCOCCOCCOCCOC(c1ccccc1)(c1ccccc1)c1ccccc1)OCCC(C)CCC[C@H](C)CCC[C@H](C)CCCC(C)C]

To a solution of 3-non-8-enoxy-2-[(7R,11R)-3,7,11,15-tetramethylhexadecoxy]propan-1-ol (1.85 g, 3.72 mmol) in THF (40 ml) was added NaH (298 mg, 7.45 mmol). The mixture was stirred at 80° C. for 1 hr. Then 2-[2-[2-(2-trityloxyethoxy)ethoxy]ethoxy]ethyl methanesulfonate (1.92 g, 3.72 mmol) was added to the mixture at 25° C. The mixture was stirred at 80° C. for 18 hrs. After that, the mixture was dealt with EA (150 ml), washed with water (150 ml×2), NaCl sat.aq (150 ml) and dried over Na₂SO₄. The organic was concentrated and purified by flash (20% EA in PE), to give [2-[2-[2-[2-[3-non-8-enoxy-2-[(7R,11R)-3,7,11,15-tetramethylhexadecoxy]propoxy]ethoxy]ethoxy]ethoxy]ethoxydiphenyl-methyl]benzene (2.11 g, yield 60.7%) as a colourless oil.

¹H NMR (400 MHz, CDCl₃) δ 7.48-7.43 (m, 6H), 7.31-7.27 (m, 6H), 7.25-7.20 (m, 3H), 5.80 (ddt, J=16.9, 10.1, 6.7 Hz, 1H), 5.04-4.88 (m, 2H), 3.70-3.41 (m, 23H), 3.23 (t, J=5.2 Hz, 2H), 2.03 (dd, J=14.4, 6.8 Hz, 2H), 1.57-1.48 (m, 4H), 1.36-1.06 (m, 30H), 0.88-0.83 (m, 15H).

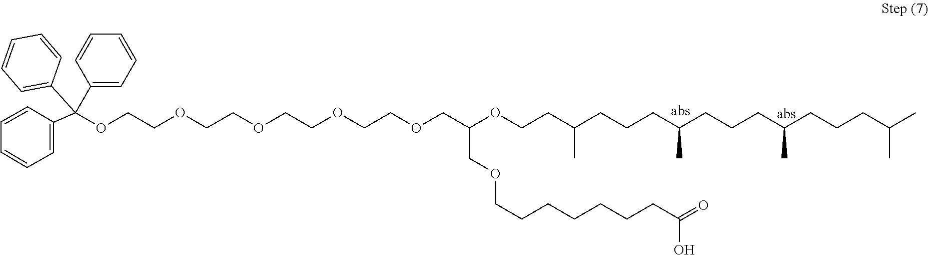 [SMILES: CC(C)CCC[C@@H](C)CCC[C@@H](C)CCCC(C)CCOC(COCCCCCCCC(=O)O)COCCOCCOCCOCCOC(c1ccccc1)(c1ccccc1)c1ccccc1]

To a solution of [2-[2-[2-[2-[3-non-8-enoxy-2-[(7R,11R)-3,7,11,15-tetramethylhexadecoxy]propoxy]ethoxy]ethoxy]ethoxy]ethoxy-diphenyl-methyl]benzene (2.11 g, 2.3 mmol) in ACN/CCl4/H2O (5 ml/5 ml/5 ml) was added NaIO4 (1.97 g, 9.2 mmol) and ruthenium(III) chloride (47.8 mg, 0.23 mmol). The mixture was stirred at 25° C. for 14 hrs. The reaction was filtered and the filtrate was diluted with ethyl acetate (250 mL) and washed with 1N aq. HCl (250 mL). The organic layer was washed with Na₂S₂O₃ solution and then dried over sodium sulfate, filtered and concentrated to give 8-[2-[(7R,11R)-3,7,11,15-tetramethylhexadecoxy]-3-[2-[2-[2-(2-trityloxyethoxy)ethoxy]ethoxy]ethoxy]propoxy]octanoic acid (1.78 g, crude) as yellow oil which was used without further purification.

¹H NMR (400 MHz, CDCl₃) δ 7.46 (d, J=8.1 Hz, 6H), 7.32-7.28 (m, 6H), 7.22 (dd, J=10.4, 4.0 Hz, 3H), 3.68-3.45 (m, 23H), 3.23 (t, J=5.1 Hz, 2H), 2.35 (dt, J=28.5, 7.4 Hz, 2H), 1.67-1.48 (m, 8H), 1.36-1.23 (m, 22H), 1.05 (d, J=7.8 Hz, 4H), 0.87-0.83 (m, 15H).

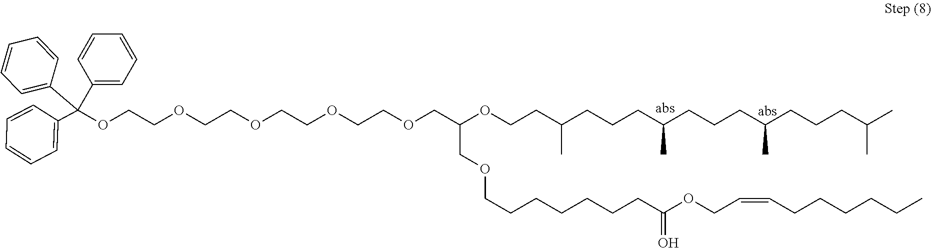 [SMILES: CCCCCC/C=C\COC(=O)CCCCCCCOCC(COCCOCCOCCOCCOC(c1ccccc1)(c1ccccc1)c1ccccc1)OCCC(C)CCC[C@H](C)CCC[C@H](C)CCCC(C)C]

To a solution of (Z)-non-2-en-1-ol (326 mg, 2.3 mmol) in DCM (20 ml) was added 8-[2-[(7R,11R)-3,7,11,15-tetramethylhexadecoxy]-3-[2-[2-[2-(2-trityloxyethoxy)ethoxy]ethoxy]ethoxy]propoxy]octanoic acid (1.78 g, 1.9 mmol), DIEA (739 mg, 5.72 mmol), 4-Dimethylaminopyridine (46.4 mg, 0.38 mmol) and EDC HCl (548 mg, 2.86 mmol). The mixture was stirred at 26° C. for 18 hrs. Then the mixture was concentrated and purified by flash (20% EA in PE), to give [(Z)-non-2-enyl] 8-[2-[(7R,11R)-3,7,11,15-tetramethylhexadecoxy]-3-[2-[2-[2-(2-trityloxyethoxy)ethoxy]ethoxy]ethoxy]propoxy]octanoate (443 mg, 0.4 mmol, yield 98%) as a colourless oil.

¹H NMR (400 MHz, CDCl₃) δ 7.50-7.42 (m, 6H), 7.32-7.27 (m, 6H), 7.25-7.20 (m, 3H), 5.63 (t, J=9.2 Hz, 1H), 5.56-5.48 (m, 1H), 4.62 (d, J=6.8 Hz, 2H), 3.71-3.39 (m, 23H), 3.23 (t, J=5.3 Hz, 2H), 2.29 (t, J=7.6 Hz, 2H), 2.09 (q, J=7.0 Hz, 2H), 1.51 (dd, J=13.3, 6.7 Hz, 4H), 1.37-1.04 (m, 38H), 0.85 (ddd, J=11.5, 6.2, 2.9 Hz, 18H)

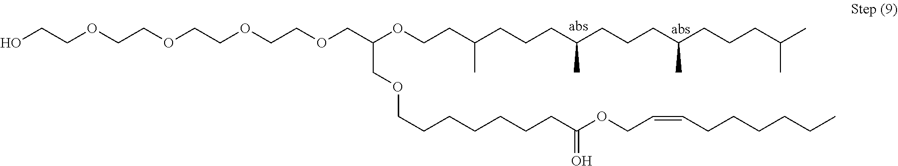 [SMILES: CCCCCC/C=C\COC(=O)CCCCCCCOCC(COCCOCCOCCOCCO)OCCC(C)CCC[C@H](C)CCC[C@H](C)CCCC(C)C]

To a solution of [(Z)-non-2-enyl] 8-[2-[(7R,11R)-3,7,11,15-tetramethylhexadecoxy]-3-[2-[2-[2-(2-trityloxyethoxy)ethoxy]ethoxy]ethoxy]propoxy]octanoate (443 mg, 0.42 mmol) in MeOH/THF (10 ml 1:1) was added Toluene-4-sulfonic acid (398 mg, 2.1 mmol).The mixture was stirred at 25° C. for 1 hr. ET3N (1 ml) was added to this mixture. The mixture was concentrated and purified by flash (5% MeOH in DCM), to give [(Z)-non-2-enyl] 8-[3-[2-[2-[2-(2-hydroxyethoxy)ethoxy]ethoxy]ethoxy]-2-[(7R,11R)-3,7,11,15-tetramethylhexadecoxy]propoxy]octanoate (240 mg, 0.29 mmol, yield 68.9%) as a colorless oil.

¹H NMR (400 MHz, CDCl₃) δ 5.64 (dt, J=10.9, 7.5 Hz, 1H), 5.56-5.47 (m, 1H), 4.62 (d, J=6.8 Hz, 2H), 3.76-3.40 (m, 25H), 2.67 (t, J=6.0 Hz, 1H), 2.30 (t, J=7.6 Hz, 2H), 2.09 (dt, J=15.6, 7.8 Hz, 2H), 1.63-1.04 (m, 42H), 0.86 (tt, J=6.6, 4.3 Hz, 18H)

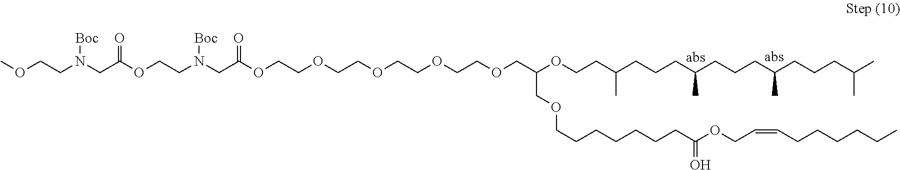 [SMILES: CCCCCC/C=C\COC(=O)CCCCCCCOCC(COCCOCCOCCOCCOC(=O)CN(CCOC(=O)CN(CCOC)C(=O)OC(C)(C)C)C(=O)OC(C)(C)C)OCCC(C)CCC[C@H](C)CCC[C@H](C)CCCC(C)C]

To a solution of [(Z)-non-2-enyl] 8-[3-[2-[2-[2-(2-hydroxyethoxy)ethoxy]ethoxy]ethoxy]-2-[(7R,11R)-3,7,11,15-tetramethylhexadecoxy]propoxy]octanoate (240 mg, 0.3 mmol) in DCM (5 ml) was added 2-[tert-butoxycarbonyl- [2-[2-[tert-butoxycarbonyl(2-methoxyethyl)amino]acetyl]oxyethyl]amino]acetic acid (192 mg, 0.44 mmol), 4-Dimethylaminopyridine (54 mg, 0.44 mmol), N-Methylmorpholine (45 mg, 0.44 mmol) and EDC HCl (84.7 mg, 0.44 mmol). The mixture was stirred at 25° C. for 18 hrs. Then the mixture was dealt with DCM (50 ml), washed with water (50 ml×2), NaCl sat.aq (50 ml) and dried over Na₂SO₄. The organic was concentrated and purified by flash (50% EA in PE), to give [(Z)-non-2-enyl] 8-[3-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]-2-[(7R,11R)-3,7,11,15-tetramethylhexadecoxy]propoxy]octanoate (211 mg, 0.17 mmol, yield 57%) as a colorless oil.

¹H NMR (400 MHz, CDCl₃) δ 5.63 (t, J=9.1 Hz, 1H), 5.51 (dd, J=10.9, 6.6 Hz, 1H), 4.62 (d, J=6.8 Hz, 2H), 4.25 (dd, J=14.0, 8.8 Hz, 4H), 4.08-3.95 (m, 4H), 3.73-3.39 (m, 28H), 3.30 (d, J=5.8 Hz, 3H), 2.30 (t, J=7.6 Hz, 2H), 2.10 (dd, J=14.1, 7.3 Hz, 2H), 1.61 (d, J=6.5 Hz, 2H), 1.57-1.01 (m, 59H), 0.86 (tt, J=6.6, 4.3 Hz, 18H).

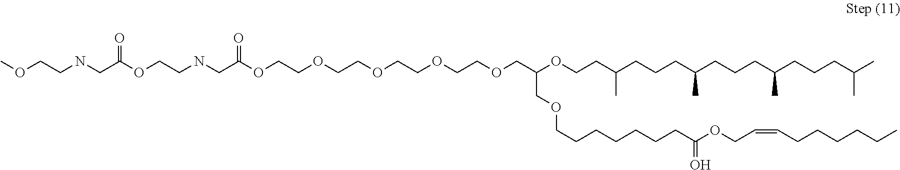 [SMILES: CCCCCC/C=C\COC(=O)CCCCCCCOCC(COCCOCCOCCOCCOC(=O)CNCCOC(=O)CNCCOC)OCCC(C)CCC[C@H](C)CCC[C@H](C)CCCC(C)C]

To a solution of [(Z)-non-2-enyl] 8-[3-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]-2-[(7R,11R)-3,7,11,15-tetramethylhexadecoxy]propoxy]octanoate (60 mg, 0.05 mmol) in DCM (1 ml) was added TFA (0.36 ml).The mixture was stirred at 25° C. for 4 hrs. Then the mixture was concentrated to give [(Z)-non-2-enyl] 8-[3-[2-[2-[2-[2-[2-[2-[2-(2-methoxyethylamino)acetyl]oxyethylamino]acetyl]oxyethoxy]ethoxy]ethoxy]ethoxy]-2-[(7R,11R)-3,7,11,15-tetramethylhexadecoxy]propoxy]octanoate;2,2,2-trifluoroacetic acid (235 mg, 0.2 mmol, 92.2% yield) as a colorless oil.

¹H NMR (400 MHz, CDCl₃) δ 5.63 (t, J=9.2 Hz, 1H), 5.51 (dd, J=10.9, 6.8 Hz, 1H), 4.62 (d, J=6.8 Hz, 2H), 4.36 (s, 2H), 3.99 (d, J=19.1 Hz, 4H), 3.74-3.29 (m, 34H), 2.30 (t, J=7.6 Hz, 2H), 2.09 (dd, J=14.5, 7.4 Hz, 2H), 1.65-1.00 (m, 44H), 0.93-0.76 (m, 18H)

### Example 16: Synthesis of 2-[[2-[2-[2-[2-[2-[2-[2,3-bis[(Z)-octadec-9-enoxy]propanoyl-octylamino]ethoxy]ethoxy]ethoxy]ethoxy]ethoxy]-2-oxo-ethyl]amino]ethyl 2-(2-methoxyethylamino)acetate;2,2,2-trifluoroacetic Acid (Compound (XIX))

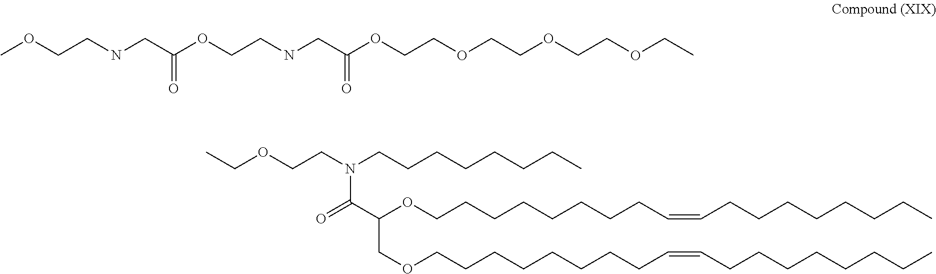

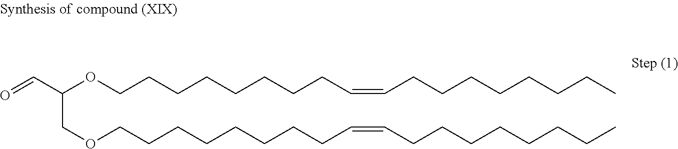 [SMILES: CCCCCCCC/C=C\CCCCCCCCOCC(C=O)OCCCCCCCC/C=C\CCCCCCCC]

To a solution of 2,3-bis[(Z)-octadec-9-enoxy]propan-1-ol (2 g, 3.37 mmol) in DCM (40 ml) was added Dess-Martin Periodinane (2.02 g, 4.05 mmol) at 0° C. for 5 min. Then the mixture was stirred at 25° C. for 2 hrs under N₂. After reaction, the mixture was dealt DCM (40 ml), washed with NaHCO₃/Na₂S₂O (1/1) (50 ml×3), NaCl sat.aq (50 ml), dried over Na₂SO₄. The organic was concentrated and purified by flash (10% EA in PE), to give 2,3-bis[(Z)-octadec-9-enoxy]propanal (1 g,1.66 mmol, yield 49.2%) as a colorless oil

¹H NMR (400 MHz, CDCl₃) δ 9.73 (d, J=1.3 Hz, 1H), 5.41-5.31 (m, 4H), 3.85-3.77 (m, 1H), 3.75-3.64 (m, 2H), 3.58 (tt, J=9.3, 4.6 Hz, 2H), 3.49-3.40 (m, 2H), 2.21-1.91 (m, 8H), 1.64 (dd, J=14.1, 7.0 Hz, 2H), 1.57-1.48 (m, 2H), 1.26 (d, J=4.4 Hz, 44H), 0.88 (dd, J=8.7, 5.0 Hz, 6H).

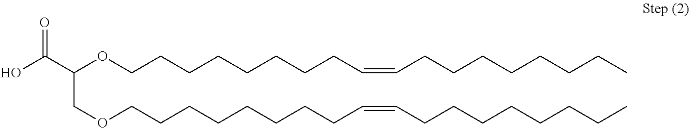 [SMILES: CCCCCCCC/C=C\CCCCCCCCOCC(OCCCCCCCC/C=C\CCCCCCCC)C(=O)O]

2,3-bis[(Z)-octadec-9-enoxy]propanal (1.48 g, 2.5 mmol) was dissolved in t-BuOH: H2O (3:1, 20 mL), containing NaH2PO4·2H2O (1.17 g, 7.51 mmol), 2-methy-2-butene (5.26 ml) and sodium chlorite (679 mg, 7.51 mmol). The reaction was stirred for 1 h at rt and was diluted with ethyl acetate (100 ml) washed with water (100 ml×2). The aqueous layer was extracted with ethyl acetate (100 ml). The combined extract was dried over sodium sulfate and concentrated to afford 2,3-bis[(Z)-octadec-9-enoxy]propanoic acid (1.35 g, 2.17 mmol, yield 86.8%) as colorless oil.

¹H NMR (400 MHz, CDCl₃) δ 5.41-5.29 (m, 4H), 4.04 (dd, J=5.0, 3.3 Hz, 1H), 3.80 (dd, J=10.5, 3.2 Hz, 1H), 3.71 (dd, J=10.5, 5.1 Hz, 1H), 3.63 (t, J=6.7 Hz, 2H), 3.52-3.45 (m, 2H), 2.08-1.91 (m, 7H), 1.66-1.55 (m, 4H), 1.30-1.22 (m, 44H), 0.87 (d, J=7.1 Hz, 6H).

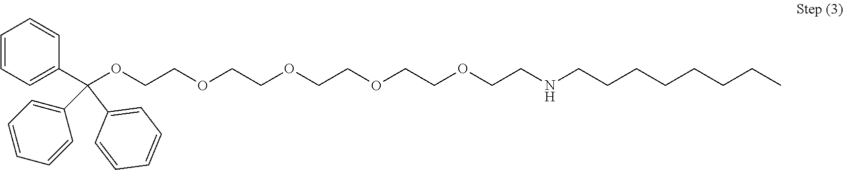 [SMILES: CCCCCCCCNCCOCCOCCOCCOCCOC(c1ccccc1)(c1ccccc1)c1ccccc1]

2-[2-[2-[2-(2-trityloxyethoxy)ethoxy]ethoxy]ethoxy]ethyl methanesulfonate (5 g, 8 mmol) was added to octan-1-amine (20 ml), and the mixture was stirred at 80° C. for 18 hrs. LCMS showed the SM was consumed and the product was formed. The mixture was dealt with EA (300 ml), washed with water (300 ml×2), NaCl sat.aq (300 ml) and dried over Na2SO4. The organic was concentrated and purified by flash (10% MeOH in DCM), to give N-[2-[2-[2-[2-(2-trityloxyethoxy)ethoxy]ethoxy]ethoxy]ethyl]octan-1-amine (2.06 g, 3.48 mmol, yield 43.2%) as a yellow oil.

¹H NMR (500 MHz, CDCl₃) δ 7.49-7.44 (m, 6H), 7.29 (dd, J=10.3, 4.8 Hz, 6H), 7.25-7.20 (m, 3H), 3.71-3.56 (m, 16H), 3.23 (t, J=5.2 Hz, 2H), 2.78 (t, J=5.2 Hz, 2H), 2.63-2.54 (m, 2H), 1.48 (dd, J=14.4, 7.3 Hz, 2H), 1.32-1.23 (m, 10H), 0.87 (t, J=6.9 Hz, 3H).

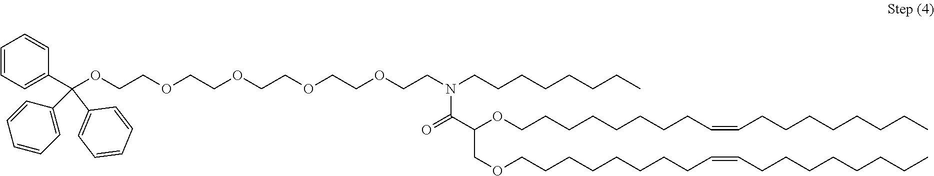 [SMILES: CCCCCCCC/C=C\CCCCCCCCOCC(OCCCCCCCC/C=C\CCCCCCCC)C(=O)N(CCCCCCCC)CCOCCOCCOCCOCCOC(c1ccccc1)(c1ccccc1)c1ccccc1]

To a solution of N-[2-[2-[2-[2-(2-trityloxyethoxy)ethoxy]ethoxy]ethoxy]ethyl]octan-1-amine (1 g, 1.52 mmol) in DCM (20 ml) was added 2,3-bis[(Z)-octadec-9-enoxy]propanoic acid (923 mg, 1.52 mmol), 4-Dimethylaminopyridine (18.6 mg, 0.15 mmol), 0-(7-Azabenzotriazol-1-yl)-N,N,N′,N′-tetramethyluronium hexafluorophosphate (867 mg, 2.28 mmol) and TEA (308 mg, 3.04 mmol). The mixture was stirred at 25° C. for 18 hrs. After reaction, the mixture was dealt with DCM (100 ml), washed with water (150 ml×2), NaCl sat.aq (150 ml) and dried over Na₂SO₄. The organic was consumed and purified by flash (10% MeOH in DCM), to give 2,3-bis[(Z)-octadec-9-enoxy]-N-octyl-N-[2-[2-[2-[2-(2-trityloxyethoxy)ethoxy]ethoxy]ethoxy]ethyl]propanamide (1079 mg, 0.9 mmol, yield 60.1%) as a yellow oil.

¹H NMR (400 MHz, CDCl₃) δ 7.49-7.43 (m, 6H), 7.29 (dd, J=10.2, 4.9 Hz, 6H), 7.22 (dd, J=8.3, 6.1 Hz, 3H), 5.40-5.31 (m, 3H), 4.42-4.30 (m, 1H), 3.74-3.34 (m, 26H), 3.23 (t, J=5.2 Hz, 2H), 2.11-1.83 (m, 7H), 1.54 (s, 4H), 1.27 (s, 56H), 0.91-0.85 (m, 9H).

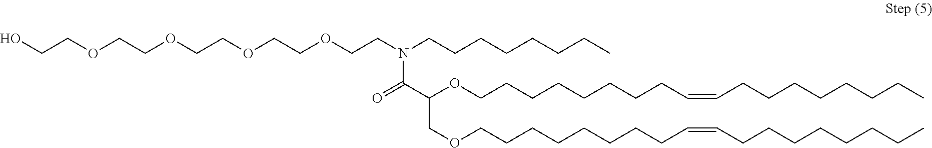 [SMILES: CCCCCCCC/C=C\CCCCCCCCOCC(OCCCCCCCC/C=C\CCCCCCCC)C(=O)N(CCCCCCCC)CCOCCOCCOCCOCCO]

To a solution of 2,3-bis[(Z)-octadec-9-enoxy]-N-octyl-N-[2-[2-[2-[2-(2-trityloxyethoxy)ethoxy]ethoxy]ethoxy]ethyl]propanamide (1079 mg, 0.9 mmol) in THF/MeOH (10 ml, 1/1) was added Toluene-4-sulfonic acid (869 mg, 4.57 mmol). The mixture was stirred at 25° C. for 2 hr. TEA (1.5 ml) was added to this mixture and the mixture was concentrated and purified by flash (10 MeOH % in DCM), to give N-[2-[2-[2-[2-(2-hydroxyethoxy)ethoxy]ethoxy]ethoxy]ethyl]-2,3-bis[(Z)-octadec-9-enoxy]-N-octyl-propanamide (750 mg, 0.8 mmol, yield 87.6%) as a colorless oil.

¹H NMR (400 MHz, CDCl₃) δ 5.40-5.31 (m, 4H), 4.44-4.31 (m, 1H), 3.75-3.37 (m, 29H), 2.11-1.90 (m, 8H), 1.56 (s, 4H), 1.27 (s, 56H), 0.91-0.86 (m, 9H).

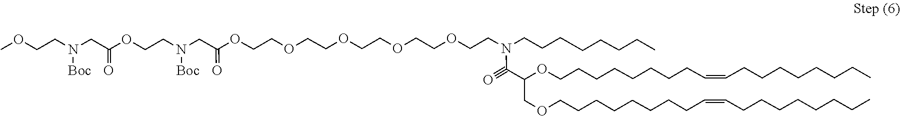

To a solution of N-[2-[2-[2-[2-(2-hydroxyethoxy)ethoxy]ethoxy]ethoxy]ethyl]-2,3-bis[(Z)-octadec-9-enoxy]-N-octylpropanamide (350 mg, 0.3 mmol) in DCM (5 ml) was added 2-[tert-butoxycarbonyl-[2-[2-[tert-butoxycarbonyl(2-methoxyethyl)amino]acetyl]oxyethyl]aamino]acetic acid (165 mg, 0.38 mmol), DMAP (58 mg, 0.48 mmol), NMM (64 mg, 0.63 mmol) and EDCI (91 mg, 0.48 mmol). The mixture was stirred at 25° C. for 18 hrs. Then the mixture was dealt with DCM (50 ml), washed with water (50 ml×2), NaCl sat.aq (50 ml). The organic was purified by flash (10% MeOH in DCM), to give 2-[[2-[2-[2-[22-[2-[2-[2,3-bis[(Z)-octadec-9-enoxy]propanoyl-octylamino]ethoxy]ethoxy]ethoxy]ethoxy]ethoxy]-2-oxo-ethyl]-tert-butoxycarbonyl-amino]ethyl 2-[tertbutoxycarbonyl(2-methoxyethyl)amino]acetate (360 mg, 0.26 mmol, yield 82.1%) as a colorless oil.

¹H NMR (400 MHz, CDCl₃) δ 5.34 (s, 4H), 4.27 (dd, J=24.6, 11.2 Hz, 5H), 4.06-3.95 (m, 4H), 3.73-3.38 (m, 32H), 3.30 (d, J=5.6 Hz, 3H), 2.07-1.93 (m, 8H), 1.58 (s, 2H), 1.54-1.50 (m, 2H), 1.44 (d, J=19.2 Hz, 18H), 1.27 (s, 56H), 0.88 (t, J=6.8 Hz, 9H).

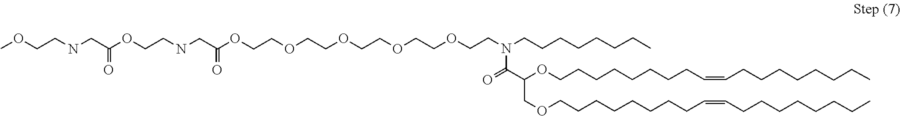

To a solution of 2-[[2-[2-[2-[2-[2-[2-[2,3-bis[(Z)-octadec-9-enoxy]propanoyl-octylamino]ethoxy]ethoxy]ethoxy]ethoxy]ethoxy]-2-oxo-ethyl]-tert-butoxycarbonyl-amino]ethyl 2-[tertbutoxycarbonyl(2-methoxyethyl)amino]acetate (360 mg, 0.27 mmol) in DCM (5 ml) was added TFA (1.97 ml). The mixture was stirred at 25° C. for 3 hrs. After reaction, the mixture was concentrated to give 2-[[2-[2-[2-[2-[2-[2-[2,3-bis[(Z)-octadec-9-enoxy]propanoyl-octyl-amino]ethoxy]ethoxy]ethoxy]ethoxy]ethoxy]-2-oxo-ethyl]amino]ethyl 2-(2-methoxyethylamino)acetate;2,2,2-trifluoroacetic acid (343 mg, 0.26 mmol, yield 96.7%) as a colorless oil.

¹H NMR (400 MHz, CDCl₃) δ 5.34 (s, 4H), 4.58 (s, 2H), 4.38 (s, 2H), 4.00 (d, J=6.0 Hz, 4H), 3.71 (s, 6H), 3.53 (d, J=59.7 Hz, 21H), 3.34 (d, J=23.0 Hz, 9H), 2.01 (d, J=6.1 Hz, 8H), 1.53 (s, 4H), 1.27 (s, 56H), 0.88 (t, J=5.1 Hz, 9H)

### Example 17: Synthesis of 2-[[2-[2-[2-[2-[2-[1-[1,2-bis[(Z)-octadec-9-enoxy]ethyl]tridecoxy]ethoxy]ethoxy]ethoxy]ethoxy]-2-oxo-ethyl]amino]ethyl 2-(2-methoxyethylamino)acetate;2,2,2-trifluoroacetic Acid (Compound (XX))

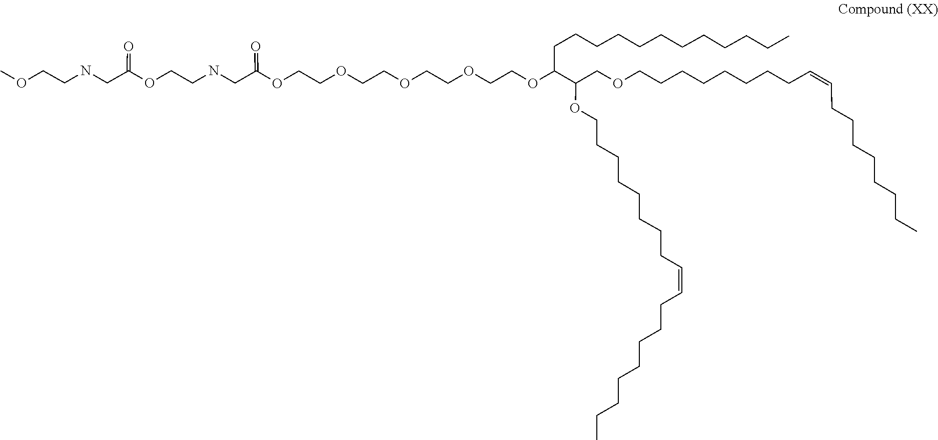

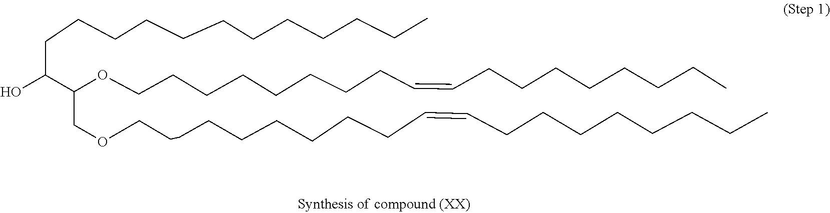 [SMILES: CCCCCCCC/C=C\CCCCCCCCCOCC(OCCCCCCCC/C=C\CCCCCCCC)C(O)CCCCCCCCCCCC]

To a solution of Mg (3.75 g) and 12 (1.31 g) in anhydrous THF (5 ml) was added 1-bromododecane (2.56 g, 10.28 mmol). The mixture was stirred at 70° C. under N₂ until the mixture as the colourless one. 1-bromododecane (10.24 g, 41.12 mmol) was added to the reaction. The mixture was stirred at 70° C. for 3 hr. Then the mixture was added to the solution of 2,3-bis[(Z)-octadec-9-enoxy]propanal (3.04 g, 5.14 mmol) in anhydrous THF (45 ml). The mixture was stirred at 7° C. for 14 hr. The mixture was dealt with EA (150 ml), washed with water (150 ml×2), NaCl sat.aq (150 ml). The organic was purified by flash (5% EA in PE), to give 1,2-bis[(Z)-octadec-9-enoxy]pentadecan-3-ol (3.97 g, 5.21 mmol, yield 100%) as a yellow oil

¹H NMR (500 MHz, CDCl₃) δ 5.40-5.31 (m, 3H), 3.76-3.36 (m, 7H), 3.31-3.23 (m, 1H), 2.03-1.94 (m, 6H), 1.64-1.57 (m, 2H), 1.54-1.50 (m, 2H), 1.27 (d, J=12.1 Hz, 66H), 0.87 (d, J=6.7 Hz, 9H).

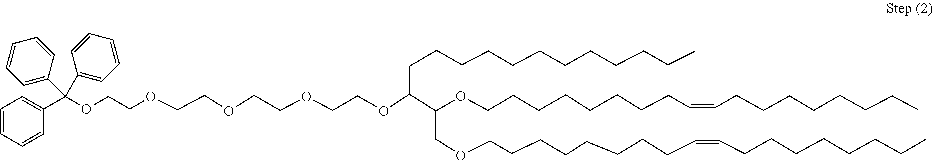

To a solution of 1,2-bis[(Z)-octadec-9-enoxy]pentadecan-3-ol (1.06 g, 1.39 mmol) in THF (20 ml) was added NaH (223 mg, 5.57 mmol). The mixture was stirred at 70° C. for 1 hr. 2-[2-[2-(2-trityloxyethoxy)ethoxy]ethoxy]ethyl methanesulfonate (1.07 g, 2.09 mmol) was added to this mixture and the mixture was stirred at 70° C. for 18 hr. The mixture was dealt with EA (150 ml), washed with water (150 ml×2), NaCl sat.aq (150 ml) and dried over Na2SO4. The organic was concentrated and purified by flash (10% EA in PE), to give [2-[2-[2-[2-[l-[1,2-bis[(Z)-octadec-9-enoxy]ethyl]tridecoxy]ethoxy]ethoxy]ethoxy]ethoxy-diphenyl-methyl]benzene (1 g, 0.8 mmol, yield 59.7%) as a colourless oil.

¹H NMR (500 MHz, CDCl₃) δ 7.46 (d, J=7.2 Hz, 6H), 7.31-7.27 (m, 6H), 7.24-7.20 (m, 3H), 5.42-5.30 (m, 3H), 3.73-3.33 (m, 22H), 3.25-3.22 (m, 2H), 2.06-1.91 (m, 6H), 1.59-1.06 (m, 70H), 0.88 (t, J=6.8 Hz, 9H)

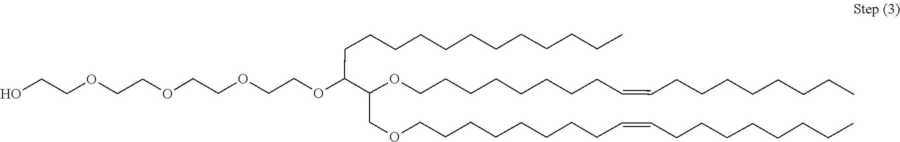 [SMILES: CCCCCCCC/C=C\CCCCCCCCOCC(OCCCCCCCC/C=C\CCCCCCCC)C(CCCCCCCCCCCC)OCCOCCOCCOCCO]

To a solution of [2-[2-[2-[2-[1-[1,2-bis[(Z)-octadec-9-enoxy]ethyl]tridecoxy]ethoxy]ethoxy]ethoxy]ethoxy-diphenylmethyl]benzene (1 g, 0.85 mmol) in THF/MeOH (20 ml 1/1) was added Toluene-4-sulfonic acid (806 mg, 4.24 mmol). The mixture was stirred at 25° C. for 18 hr. The mixture was concentrated and dealt with EA (50 ml), washed with NaHCO₃ sat.aq (50 ml×2), NaCl sat.aq (50 ml) and dried over Na2SO4. The organic was concentrated and purified by flash (50% EA in PE), to give 2-[2-[2-[2-[l-[1,2-bis[(Z)-octadec-9-enoxy]ethyl]tridecoxy]ethoxy]ethoxy]ethoxy]ethanol (550 mg, 0.58 mmol, yield 67.8%) as a colourless oil.

¹H NMR (400 MHz, CDCl₃) δ 5.51-5.31 (m, 3H), 3.76-3.33 (m, 23H), 2.59 (s, 1H), 2.02 (dt, J=12.3, 6.0 Hz, 6H), 1.69-1.02 (m, 70H), 0.88 (t, J=6.8 Hz, 9H).

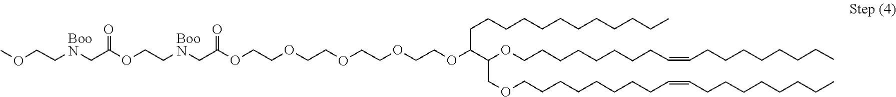

To a solution of 2-[2-[2-[2-[1-[1,2-bis[(Z)-octadec-9-enoxy]ethyl]tridecoxy]ethoxy]ethoxy]ethoxy]ethanol (100 mg, 0.1 mmol) in DCM (5 ml) was added 2-[tert-butoxycarbonyl-[2-[2-[tert-butoxycarbonyl(2-methoxyethyl)amino]acetyl]oxyethyl]amino]acetic acid (92.7 mg, 0.21 mmol), DIEA (34.5 mg, 0.27 mmol), DMAP (3 mg) and EDCI (40.9 mg, 0.21 mmol). The mixture was stirred at 25° C. for 18 hr. The mixture was dealt with DCM (50 ml), washed with water (50 ml×2), NaCl sat.aq (50 ml) and dried over Na₂SO₄. The organic was concentrated and purified by flash (35% EA in PE), to give 2-[[2-[2-[2-[2-[2-[1-[1,2-bis[(Z)-octadec-9-enoxy]ethyl]tridecoxy]ethoxy]ethoxy]ethoxy]ethoxy]-2-oxo-ethyl]-tert-butoxycarbonyl-amino]ethyl 2-[tertbutoxycarbonyl(2-methoxyethyl)amino]acetate (102 mg, 0.07 mmol, yield 68.5%) as a yellow oil.

¹H NMR (500 MHz, CDCl₃) δ 5.45-5.31 (m, 3H), 4.32-4.15 (m, 4H), 4.07-3.94 (m, 4H), 3.77-3.26 (m, 32H), 2.06-1.92 (m, 6H), 1.55-1.23 (m, 88H), 0.88 (t, J=6.9 Hz, 9H).

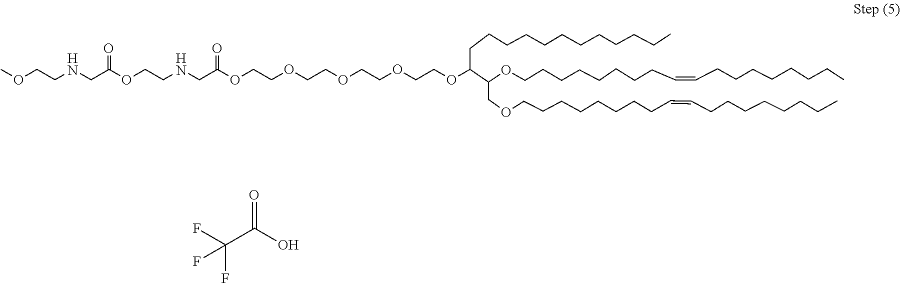 [SMILES: CCCCCCCC/C=C\CCCCCCCCOCC(OCCCCCCCC/C=C\CCCCCCCC)C(CCCCCCCCCCCC)OCCOCCOCCOCCOC(=O)CNCCOC(=O)CNCCOC ; O=C(O)C(F)(F)F]

To a solution of 2-[[2-[2-[2-[2-[2-[1-[1,2-bis[(Z)-octadec-9-enoxy]ethyl]tridecoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (332 mg, 0.24 mmol) in DCM (3 ml) was added TFA (0.5 ml). The mixture was stirred at 25° C. for 3 hr. The mixture was concentrated to give 2-[[2-[2-[2-[2-[2-[1-[1,2-bis[(Z)-octadec-9-enoxy]ethyl]tridecoxy]ethoxy]ethoxy]ethoxy]ethoxy]-2-oxo-ethyl]amino]ethyl 2-(2-methoxyethylamino)acetate;2,2,2-trifluoroacetic acid (354 mg, quantitative) as a yellow oil.

¹H NMR (400 MHz, CDCl₃) δ 5.34 (s, 4H), 4.59 (s, 2H), 4.37 (s, 2H), 4.03-3.95 (m, 4H), 3.67 (d, J=27.6 Hz, 18H), 3.40 (dt, J=36.9, 13.9 Hz, 15H), 2.01 (d, J=5.5 Hz, 8H), 1.40 (d, J=111.8 Hz, 70H), 0.88 (t, J=6.7 Hz, 9H).

### Example 18: Synthesis of 2-butyloctyl 8-[2-[8-(2-butyloctoxy)-8-oxo-octoxy]-3-[2-[2-[2-[2-[2-[2-[2-(2-methoxyethylamino)acetyl]oxyethylamino]acetyl]oxyethoxy]ethoxy]ethoxy]ethoxy]propoxy]octanoate;2,2,2-trifluoroacetic Acid (Compound (XXI))

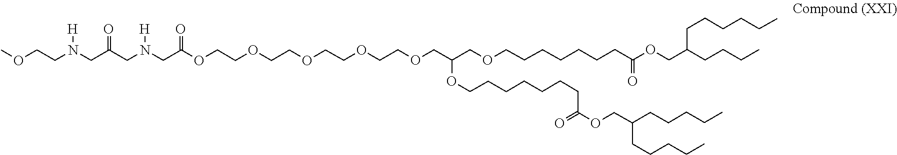 [SMILES: CCCCCCC(CCCC)COC(=O)CCCCCCCOCC(COCCOCCOCCOCCOC(=O)CNCC(=O)CNCCOC)OCCCCCCCC(=O)OCC(CCCCC)CCCCC]

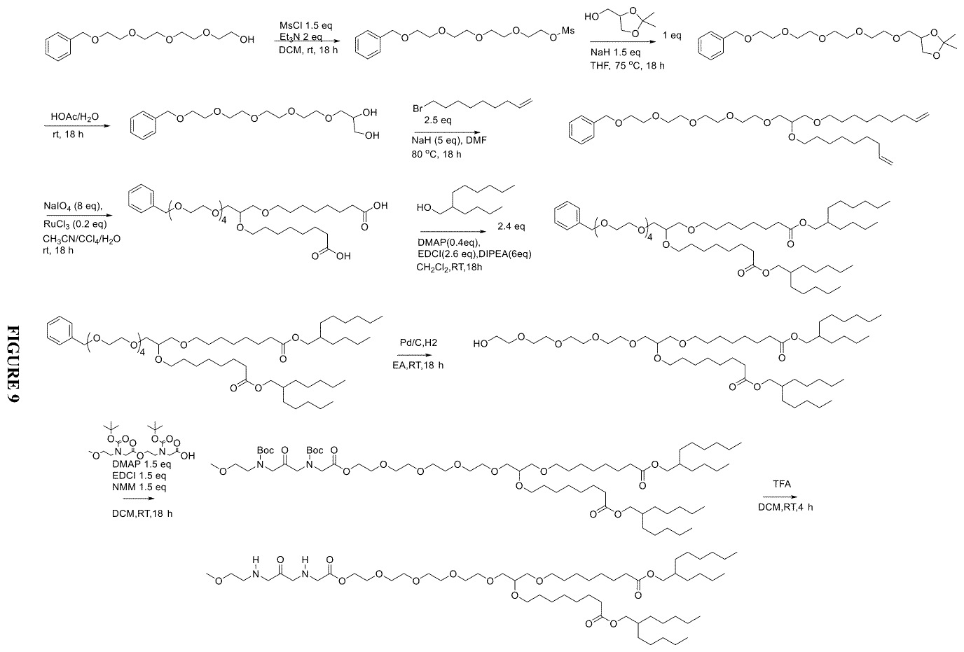

Compound (XXI) was synthesized based on the chemistry shown in Scheme (11) presented on FIG. 9.

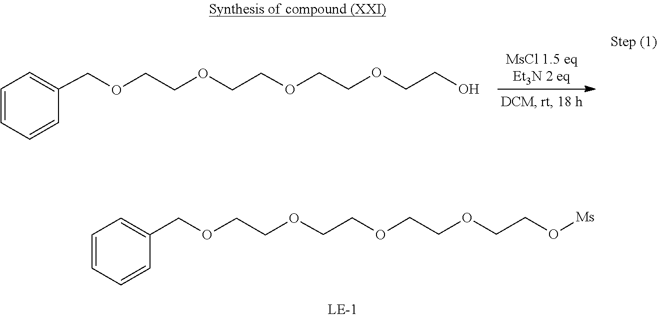 [SMILES: OCCOCCOCCOCCOCc1ccccc1 ; CS(=O)(=O)OCCOCCOCCOCCOCc1ccccc1]

2-[2-[2-(2-benzyloxyethoxy)ethoxy]ethoxy]ethanol (50 g, 0.176 mol) and triethylamine (36.2 g, 0.352 mol) in dry dichloromethane (600 mL) under nitrogen were cooled to 0° C. Methanesulfonyl chloride (30.6 g, 0.264 mol) was added dropwise to this solution at 0° C. The mixture was allowed to warm to room temperature and stirred at room temperature for 18 h. Triethylamine hydrochloride was filtered off, and the DCM solution was washed with 0.1 N HCl and dried over sodium sulfate. Removing the solvent afforded 2-[2-[2-(2-benzyloxyethoxy)ethoxy]ethoxy]ethyl methanesulfonate (62 g, 92%) as light yellow oil which was used without further purification.

LCMS MS 363 (M+1)

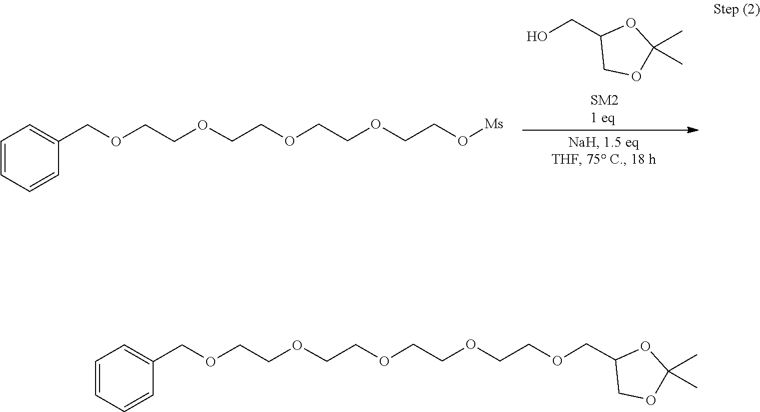

To the solution of (2,2-dimethyl-1,3-dioxolan-4-yl)methanol (62 g, 0.171 mol) in THF (600 mL) was added NaH (6.17 g, 0.257 mol) and the mixture was heated to reflux for 15 min. Then the reaction was cooled to room temperature and 2-[2-[2-(2-benzyloxyethoxy)ethoxy]ethoxy]ethyl methanesulfonate (25.0 g, 0.171 mol) was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 20 to 50% ethyl acetate in petroleum ether to give 4-[2-[2-[2-(2-benzyloxyethoxy)ethoxy]ethoxy]ethoxymethyl]-2,2-dimethyl-1,3-dioxolane (43 g, 71% yield) as light yellow oil.

LCMS MS 421(M+23)

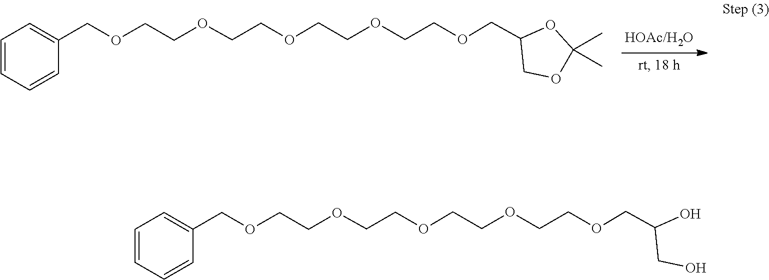 [SMILES: CC1(C)OCC(COCCOCCOCCOCCOCc2ccccc2)O1 ; OCC(O)COCCOCCOCCOCCOCc1ccccc1]

The mixture of 4-[2-[2-[2-(2-benzyloxyethoxy)ethoxy]ethoxy]ethoxymethyl]-2,2-dimethyl-1,3-dioxolane (43 g, 0.103 mol) in AcOH (200 mL) and water (200 mL). The mixture was stirred for 16 hrs at ambient temperature. Removing the solvent afforded 3-[2-[2-[2-(2-benzyloxyethoxy)ethoxy]ethoxy]ethoxy]propane-1,2-diol (36 g, 95%) as light yellow oil which was used without further purification.

LCMS MS 381(M+23)

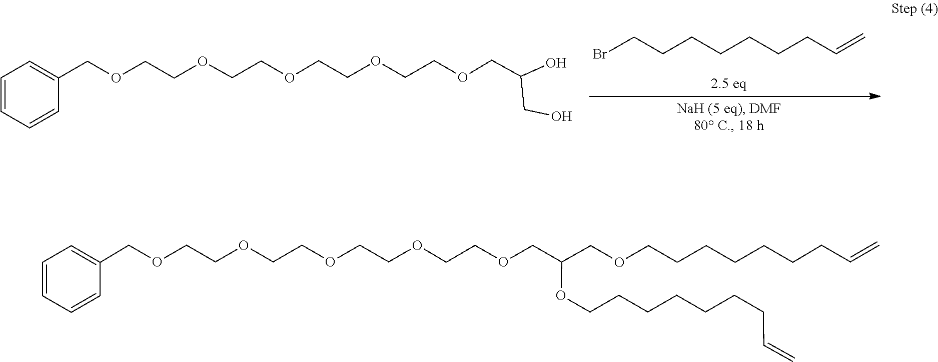

To the solution of 3-[2-[2-[2-(2-benzyloxyethoxy)ethoxy]ethoxy]ethoxy]propane-1,2-diol (20 g, 0.050 mol) in THF (200 mL) was added NaH (8.03 g, 0.201 mol) and the mixture was heated to reflux for 15 min. Then the reaction was cooled to room temperature and 9-bromonon-1-ene (26.6 g, 0.126 mol) was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 10 to 30% ethyl acetate in petroleum ether to give 2-[2-[2-[2-[2,3-bis(non-8-enoxy)propoxy]ethoxy]ethoxy]ethoxy]ethoxymethylbenzene (8.8 g, 26% yield) as light yellow oil.

¹H NMR (400 MHz, CDCl₃) δ 7.37-7.27 (m, 5H), 5.87-5.73 (m, 2H), 5.04-4.87 (m, 4H), 4.57 (s, 2H), 3.71-3.59 (m, 16H), 3.59-3.38 (m, 9H), 2.03 (q, J=6.5 Hz, 4H), 1.60-1.49 (m, 4H), 1.41-1.28 (m, 16H).

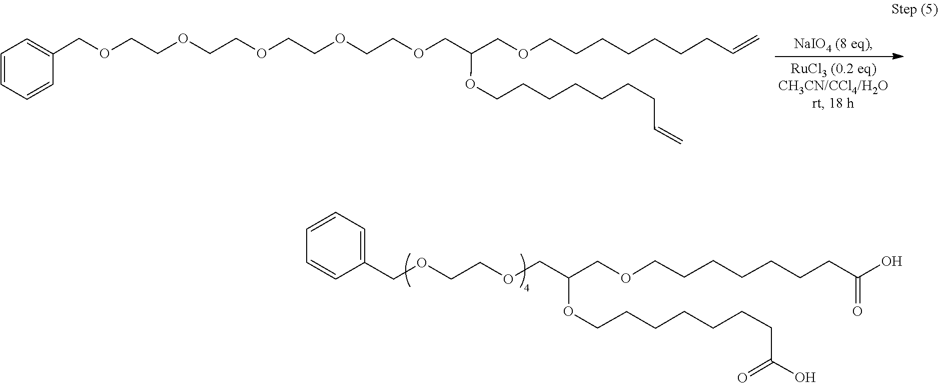

To a solution of 2-[2-[2-[2-[2,3-bis(non-8-enoxy)propoxy]ethoxy]ethoxy]ethoxy]ethoxymethylbenzene (8.5 g, 0.0140 mol) in MeCN (80 mL), CCl4 (80 mL) and water (80 mL) was added NaIO4 (24.9 g, 0.116 mol) and RuCl3 (0.66 g, 2.93 mmol). The reaction mixture was stirred at room temperature for 24 hrs. LCMS indicated that the title compound was the major product. The reaction was filtered and the filtrate was diluted with ethyl acetate (600 mL) and washed with 1N aq. HCl (200 mL). The organic layer was dried over sodium sulfate, filtered and concentrated to give 8-[3-[2-[2-[2-(2-benzyloxyethoxy)ethoxy]ethoxy]ethoxy]-2-(7-carboxyheptoxy)propoxy]octanoic acid (8.7 g, 97% yield) as yellow oil which was used without further purification.

¹H NMR (400 MHz, CDCl₃) δ 7.31 (dd, J=22.6, 3.2 Hz, 5H), 4.57 (s, 2H), 3.71-3.61 (m, 19H), 3.59-3.38 (m, 11H), 2.32 (t, J=7.4 Hz, 4H), 1.68-1.47 (m, 10H), 1.32 (s, 14H).

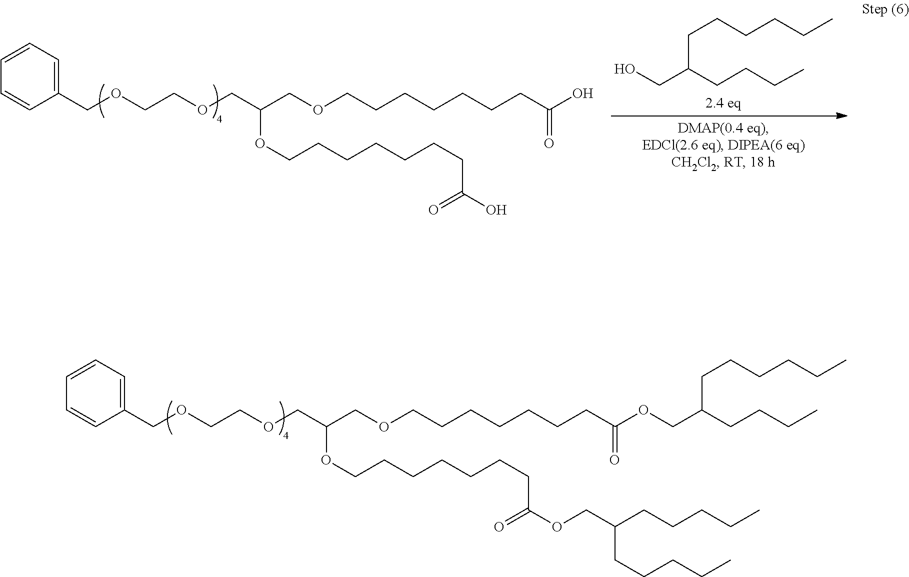

To the solution of 8-[3-[2-[2-[2-(2-benzyloxyethoxy)ethoxy]ethoxy]ethoxy]-2-(7-carboxyheptoxy)propoxy]octanoic acid (0.0135 mol, 8.7 g) and 2-butyloctan-1-ol (0.0324 mol, 6.04 g) in dichloromethane (500 mL) under nitrogen were added N,N-Diisopropylethylamine (0.081 mol, 10.47 g), 4-Dimethylaminopyridine (5.4 mmol, 0.66 g) and 1-(3-Dimethylaminopropyl)-3-ethylcarbodiimide hydrochloride (0.0351 mol, 6.73 g). The mixture was stirred at room time for 18 hrs. The reaction was diluted with dichloromethane, the organic layer was washed with 1 N HCl. Then the organic layer was washed with brine then dried over sodium sulfate, filtered and concentrated. The residue was purified by flash chromatography eluted with 0 to 60% ethyl acetate in petroleum ether to give 2-butyloctyl8-[3-[2-[2-[2-(2-benzyloxyethoxy)ethoxy]ethoxy]ethoxy]-2-[8-(2-butyloctoxy)-8-oxo-octoxy]propoxy] (2.3 g, 16.5% yield) octanoate as colorless oil.

¹H NMR (400 MHz, CDCl₃) δ 7.37-7.28 (m, 5H), 4.57 (s, 2H), 3.96 (d, J=5.8 Hz, 4H), 3.74-3.60 (m, 18H), 3.59-3.38 (m, 11H), 2.29 (t, 4H), 1.84 (s, 1H), 1.67-1.50 (m, 12H), 1.40-1.19 (m, 54H), 0.89 (t, 12H).

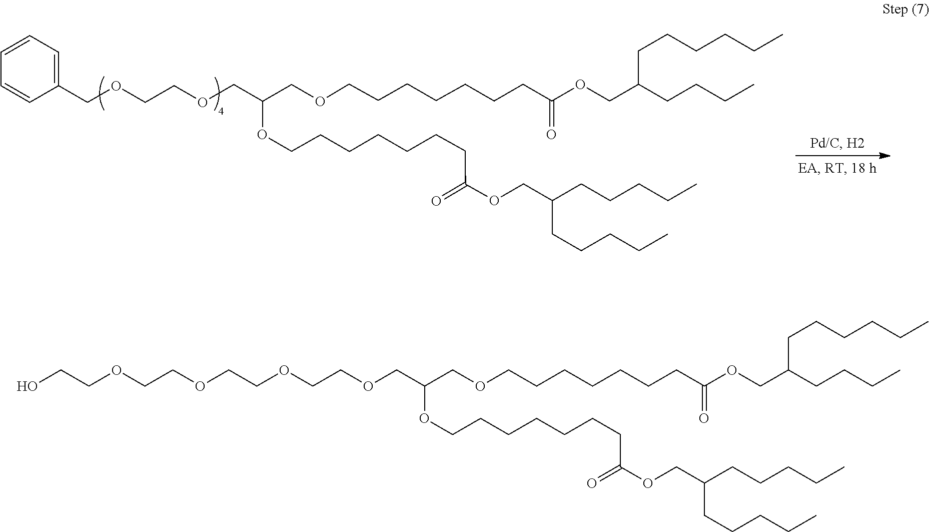

2-butyloctyl 8-[3-[2-[2-[2-(2-benzyloxyethoxy)ethoxy]ethoxy]ethoxy]-2-[8-(2-butyloctoxy)-8-oxo-octoxy]propoxy]octanoate (2.15 g, 2.2 mmol) in ethyl acetate (50 mL) was added Pd/C (500 mg, 20% wt/wt). The mixture was stirred at room temperature under hydrogen for 18 hrs. TLC (ethyl acetate/petroleum ether=1/1) indicated that the starting material was consumed. The reaction was filtered through celite and washed with ethyl acetate to give 2-butyloctyl 8-[2-[8-(2-butyloctoxy)-8-oxo-octoxy]-3-[2-[2-[2-(2-hydroxyethoxy)ethoxy]ethoxy]ethoxy]propoxy]octanoate (1.51 g, 77.1% yield) as colorless oil.

¹H NMR (400 MHz, CDCl₃) δ 3.97 (t, J=5.8 Hz, 4H), 3.74-3.40 (m, 27H), 2.29 (t, J=7.5 Hz, 4H), 1.69-1.48 (m, 11H), 1.37-1.21 (m, 48H), 0.88 (t, J=5.3 Hz, 12H).

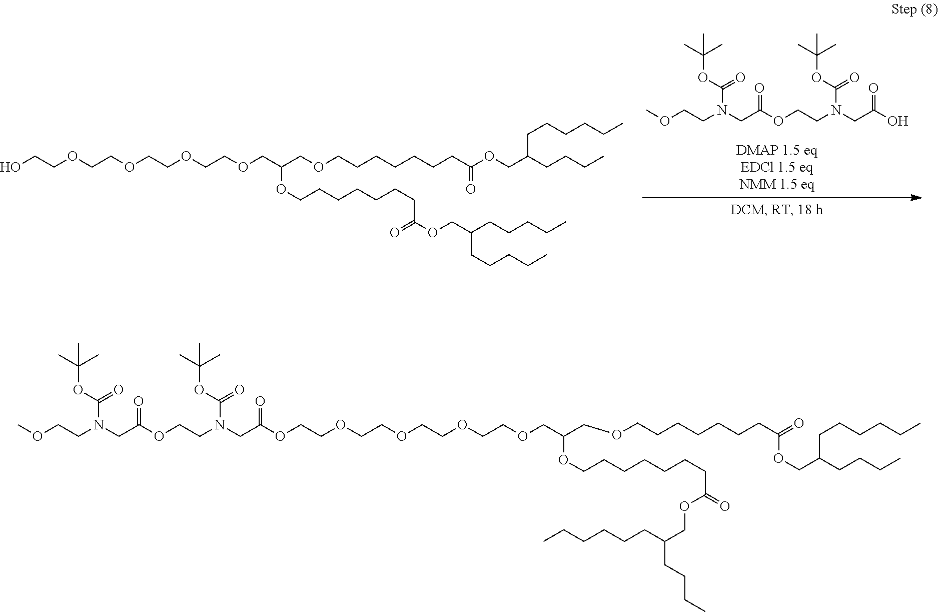

2-butyloctyl 8-[2-[8-(2-butyloctoxy)-8-oxo-octoxy]-3-[2-[2-[2-(2-hydroxyethoxy)ethoxy]ethoxy]ethoxy]propoxy]octanoate (500 mg, 0.562 mmol), 2-[tert-butoxycarbonyl-[2-[2-[tert-butoxycarbonyl(2-methoxyethyl)amino]acetyl]oxyethyl]amino]acetic acid (244 mg, 0.562 mmol) and N,N-dimethylpyridin-4-amine (77.3 mg, 0.843 mmol) were dissolved in DCM-Anhydrous (30 mL) in presence of N-Methylmorpholine (64.1 mg, 0.843 mmol). The reaction was cooled to 0° C. and 3-(ethyliminomethyleneamino)-N,N-dimethyl-propan-1-amine hydrochloride (121.2 mg, 0.843 mmol) was added portionwise to the mixture. The reaction was warmed to room temperature and stirred overnight. The reaction was monitored by TLC. Water was added and the organic layers were extracted with DCM (3*30 mL), washed with brine, dried over Na2SO4, filtered and concentrated. The residue was purified by flash column chromatography on silica gel eluting with 20% to 40% ethyl acetate in petroleum ether to give 2-butyloctyl 8-[3-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]-2-[8-(2-butyloctoxy)-8-oxo-octoxy]propoxy]octanoate (306 mg, 42% yield) as colorless oil.

¹H NMR (400 MHz, CDCl₃) δ 4.41-4.36 (m, 2H), 3.96 (t, J=5.8 Hz, 4H), 3.79-3.75 (m, 2H), 3.70-3.39 (m, 22H), 3.08 (s, 3H), 2.29 (t, J=7.5 Hz, 4H), 1.67-1.49 (m, 11H), 1.39-1.18 (m, 48H), 0.89 (t, J=6.6, 3.8 Hz, 12H).

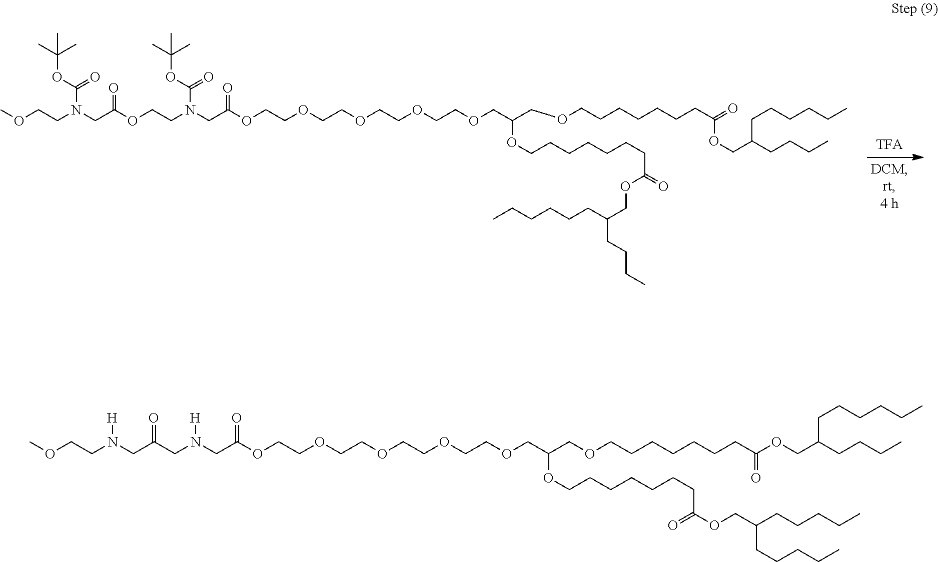

2-butyloctyl 8-[3-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]-2-[8-(2-butyloctoxy)-8-oxo-octoxy]propoxy]octanoate (306 mg, 0.234 mmol) in DCM (5 mL) cooled to 0° C. was added TFA (1 mL) and the mixture was stirred at RT for 4 h. TLC (DCM/MeOH=10:1, expected product Rf=0.1) indicated that the starting material was disappeared. The solvent was removed and the product was azeotroped with DCM several times and then dried under vacuum for 2 h to give 2-butyloctyl 8-[2-[8-(2-butyloctoxy)-8-oxo-octoxy]-3-[2-[2-[2-[2-[2-[2-[2-(2-methoxyethylamino)acetyl]oxyethylamino]acetyl]oxyethoxy]ethoxy]ethoxy]ethoxy]propoxy]octanoate (300 mg, quant.)

¹H NMR (400 MHz, CDCl₃) δ 4.60 (s, 2H), 4.36 (s, 2H), 3.97 (t, 8H), 3.74-3.68 (m, 4H), 3.64 (s, 11H), 3.60-3.51 (m, 5H), 3.51-3.40 (m, 6H), 3.35 (s, 3H), 3.35-3.28 (m, 2H), 2.30 (t, J=7.2 Hz, 4H), 1.74-1.42 (m, 11H), 1.38-1.20 (m, 46H), 0.88 (t, J=5.4 Hz, 12H).

### Example 19: Synthesis of 6-[2-[6-(2-butyloctanoyloxy)hexoxy]-3-[2-[2-[2-[2-[2-[2-[2-(2-methoxyethylamino)acetyl]oxyethylamino]acetyl]oxyethoxy]ethoxy]ethoxy]ethoxy]propoxy]hexyl 2-butyloctanoate;2,2,2-trifluoroacetic Acid (Compound (XXII))

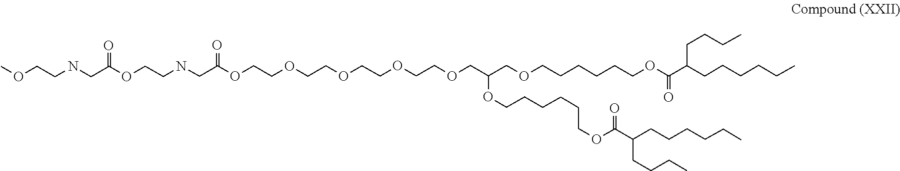

Synthesis of Compound (XXII)

6-[3-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]-2-[6-(2-butyloctanoyloxy)hexoxy]propoxy]hexyl 2-butyloctanoate (0.384 g, 0.307 mmol) was dissolved in 7 mL DCM and then added TFA (1 mL). The mixture was stirred at room temperature 2 h. The solvent was evaporated in DCM (30 mL*3) to give 6-[2-[6-(2-butyloctanoyloxy)hexoxy]-3-[2-[2-[2-[2-[2-[2-[2-(2-methoxyethylamino)acetyl]oxyethylamino]acetyl]oxyethoxy]ethoxy]ethoxy]ethoxy]propoxy]hexyl 2-butyloctanoate;2,2,2-trifluoroacetic acid (0.3641 g, 0.297 mmol, 96.8% yield) as pale yellow oil.

LCMS: Find peak at 2.36 min Mr:1050.8 (M+1),

¹H NMR (400 MHz, CDCl₃) δ 4.61 (s, 2H), 4.35 (s, 2H), 4.09-3.94 (m, 7H), 3.72-3.29 (m, 33H), 2.37-2.24 (m, 2H), 1.65-1.20 (m, 48H), 0.94-0.80 (m, 12H).

### Example 20: Synthesis of 2-[[2-[2-[2-[2-[2-[3-[2,3-bis[(Z)-octadec-9-enoxy]propyl-octyl-amino]-3-oxopropoxy]ethoxy]ethoxy]ethoxy]ethoxy]-2-oxo-ethyl]amino]ethyl 2-(2-methoxyethylamino)acetate;2,2,2-trifluoroacetic Acid (Compound (XXIII))

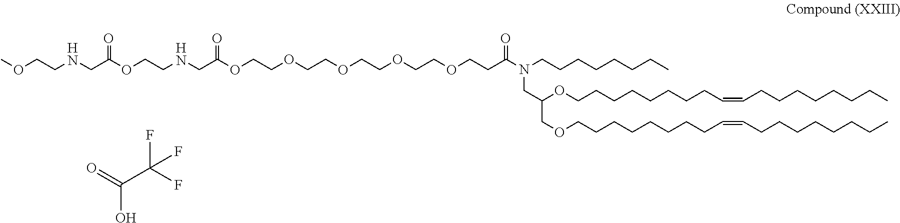 [SMILES: CCCCCCCC/C=C\CCCCCCCCOCC(CN(CCCCCCCC)C(=O)CCOCCOCCOCCOCCOC(=O)CNCCOC(=O)CNCCOC)OCCCCCCCC/C=C\CCCCCCCC ; O=C(O)C(F)(F)F]

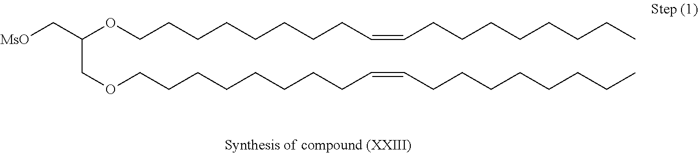 [SMILES: CCCCCCCC/C=C\CCCCCCCCOCC(COS(C)(=O)=O)OCCCCCCCC/C=C\CCCCCCCC]

To the solution of 2,3-bis[(Z)-octadec-9-enoxy]propan-1-ol (1.0 g, 1.69 mmol), Triethylamine (0.512 g, 5.06 mmol) in DCM (20 mL) was added Methanesulfonyl chloride (0.386 g, 3.37 mmol) and the mixture was stirred at rt for 2 hrs. TLC indicated that the starting material was consumed. The reaction was quenched with water and extracted with DCM. The aqueous layer was extracted with DCM again. The combined organic layers were washed with brine, dried over sodium sulfate, filtered and concentrated to give 2,3-bis[(Z)-octadec-9-enoxy]propyl methanesulfonate (1.1 g, 97%) as colorless oil.

¹H NMR (400 MHz, CDCl₃) δ 5.37-5.32 (m, 3H), 4.25 (dd, J=10.9, 5.7 Hz, 1H), 3.68 (s, 2H), 3.59-3.39 (m, 7H), 3.17-3.07 (m, 7H), 3.04 (s, 3H), 2.01 (dd, J=12.4, 6.6 Hz, 6H), 1.56 (d, J=4.5 Hz, 4H), 1.37-1.22 (m, 45H), 0.88 (t, J=6.8 Hz, 6H)

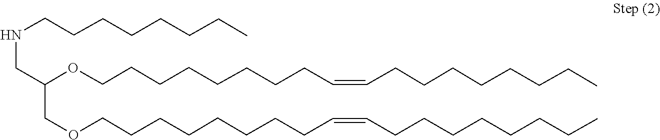 [SMILES: CCCCCCCC/C=C\CCCCCCCCOCC(CNCCCCCCCC)OCCCCCCCC/C=C\CCCCCCCC]

To the solution of 3-2,3-bis[(Z)-octadec-9-enoxy]propyl methanesulfonate (4.5 g, 6.71 mmol), octan-1-amine in (17.3 g, 134 mmol). The reaction was heated at 80° C. for 18 hrs. The reaction mixture was purified through flash chromatography eluted with 10 to 50% ethyl acetate in petroleum ether to give N-[2,3-bis[(Z)-octadec-9-enoxy]propyl]octan-1-amine (4.2 g, 89% yield) as light yellow oil.

¹H NMR (400 MHz, CDCl₃) δ 5.45-5.27 (m, 3H), 3.68-3.38 (m, 7H), 2.78-2.52 (m, 4H), 2.08-1.90 (m, 7H), 1.68-1.43 (m, 9H), 1.39-1.19 (m, 55H), 0.88 (t, J=6.6 Hz, 9H4).

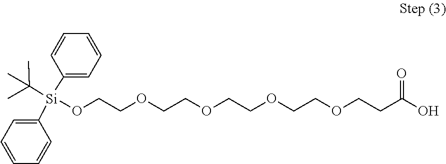 [SMILES: CC(C)(C)[Si](OCCOCCOCCOCCOCCC(=O)O)(c1ccccc1)c1ccccc1]

Tert-butyl 3-[2-[2-[2-[2-[tert-butyl(diphenyl)silyl]oxyethoxy]ethoxy]ethoxy]ethoxy]propanoate (0.6 g, 1 mmol) was dissolved in 2:1 DCM-TFA (5 mL) and the solution stirred at room temperature for 1 hour. The resulting mixture was diluted with H₂O (5 mL) and DCM (10 mL). The solution was stirred vigorously to mix the phases and the solution basified to pH 3 with 2 M NaOH. The layers were separated and the aqueous phase extracted with DCM (30 mL). The combined organics were evaporated in vacuo and the crude product purified by flash chromatography eluting with 0 to 10% MeOH in DCM to yield 3-[2-[2-[2-[2-[tertbutyl(diphenyl)silyl]oxyethoxy]ethoxy]ethoxy]ethoxy]propanoic acid (0.23 g, 0.43 mmol, 42.6%) as a colorless oil.

¹H NMR (400 MHz, CDCl₃) δ 7.72-7.63 (m, 4H), 7.45-7.33 (m, 6H), 3.84-3.73 (m, 4H), 3.71-3.55 (m, 14H), 2.60 (t, J=6.1 Hz, 2H), 1.04 (s, 9H)

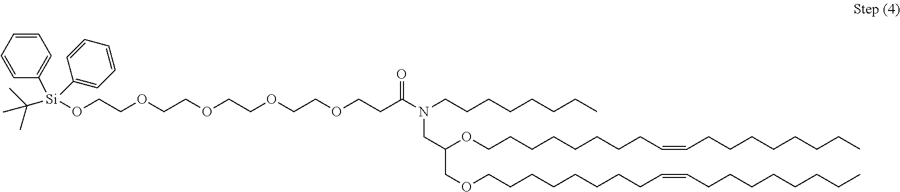 [SMILES: CCCCCCCC/C=C\CCCCCCCCOCC(CN(CCCCCCCC)C(=O)CCOCCOCCOCCOCCO[Si](c1ccccc1)(c1ccccc1)C(C)(C)C)OCCCCCCCC/C=C\CCCCCCCC]

To the mixture of 3-[2-[2-[2-[2-[tert-butyl(diphenyl)silyl]oxyethoxy]ethoxy]ethoxy]ethoxy]propanoic acid (0.23 g, 0.43 mmol) and N-[2,3-bis[(Z)-octadec-9-enoxy]propyl]octan-1-amine (0.32 g, 0.43 mmol) in DMF (2 mL) was added HATU (0.247 g, 0.65 mmol) and DIEA (0.112 g, 0.866 mmol). The reaction was stirred for 16 h at room temperature. TLC indicated that all the starting material was converted into the product. The mixture was partitioned between ethyl acetate (50 mL) and water (20 mL). The organic layers were washed brine, LiCl solution, dried over sodium sulfate, filtered and concentrated. The residue was purified by flash column chromatography on silica gel eluting with 2-5% methanol in dichloromethane to give N-[2,3-bis[(Z)-octadec-9-enoxy]propyl]-3-[2-[2-[2-[2-[tert-butyl(diphenyl)silyl]oxyethoxy]ethoxy]ethoxy]ethoxy]-N-octyl-propanamide (0.42 g, 0.335 mmol, 77%) as light yellow oil.

¹H NMR (400 MHz, CDCl₃) δ 7.71-7.63 (m, 4H), 7.45-7.32 (m, 6H), 5.40-5.31 (m, 3H), 3.83-3.27 (m, 29H), 2.72-2.61 (m, 2H), 2.05-1.95 (m, 6H), 1.60-1.46 (m, 6H), 1.37-1.19 (m, 56H), 1.04 (s, 9H), 0.91-0.83 (m, 9H).

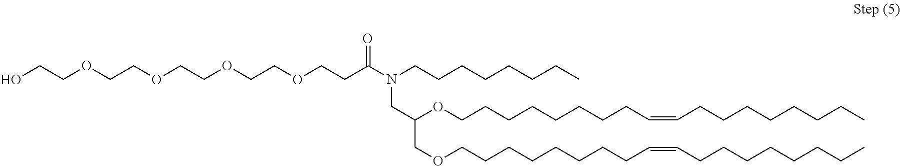 [SMILES: CCCCCCCC/C=C\CCCCCCCCOCC(CN(CCCCCCCC)C(=O)CCOCCOCCOCCOCCO)OCCCCCCCC/C=C\CCCCCCCC]

To a solution of N-[2,3-bis[(Z)-octadec-9-enoxy]propyl]-3-[2-[2-[2-[2-[tertbutyl(diphenyl)silyl]oxyethoxy]ethoxy]ethoxy]ethoxy]-N-octyl-propanamide (0.42 g, 0.317 mmol) in THF (5 mL) was added Tetra-n-butyl ammonium fluoride (1 M in THF, 0.4 mL) and the mixture was stirred for 1 hr at room temperature. TLC indicated that all the starting material was converted into the product. The solvent was removed and the sample was loaded onto silica gel and purified by flash column chromatography on silica gel eluting with 2-5% methanol in dichloromethane to give N-[2,3-bis[(Z)-octadec-9-enoxy]propyl]-3-[2-[2-[2-(2-hydroxyethoxy)ethoxy]ethoxy]ethoxy]-N-octyl-propanamide (0.31 g, 0.312 mmol, 98%) as light yellow oil.

¹H NMR (400 MHz, CDCl₃) δ 5.40-5.28 (m, 3H), 3.85-3.16 (m, 29H), 2.86-2.60 (m, 3H), 2.07-1.91 (m, 6H), 1.59-1.44 (m, 6H), 1.28 (t, J=14.6 Hz, 55H), 0.92-0.84 (m, 9H).

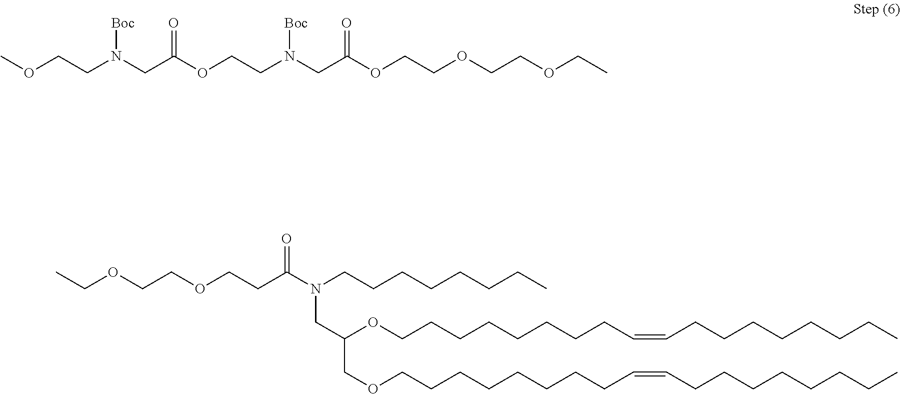

To a solution of N-[2,3-bis[(Z)-octadec-9-enoxy]propyl]-3-[2-[2-[2-(2-hydroxyethoxy)ethoxy]ethoxy]ethoxy]-N-octylpropanamide (0.31 g, 0.31 mmol) and 2-[tert-butoxycarbonyl-[2-[2-[tert-butoxycarbonyl(2-methoxyethyl)amino]acetyl]oxyethyl]amino]acetic acid (0.201 g, 0.464 mol) in dichloromethane (15 ml) was added N,N-diisopropylethylamine (0.048 g, 0.37 mmol) and DMAP (4 mg, 0.03 mmol) followed by N-(3-dimethylaminopropyl)-N′-ethylcarbodiimide hydrochloride (0.071 g, 0.037 mmol) portionwise. The reaction was allowed to stir at room temperature for 48 h. The reaction was diluted with dichloromethane and washed with saturated sodium bicarbonate. The organic layer was separated and washed with brine, and dried over Na₂SO₄. The organic layer was filtered and evaporated in vacuo. The residue was purified by silica gel chromatography (2-5% methanol in dichloromethane) to give 2-[[2-[2-[2-[2-[2-[3-[2,3-bis[(Z)-octadec-9-enoxy]propyl-octyl-amino]-3-oxo-propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.41 g, 0.288 mmol, 93% yield) as light yellow oil.

¹H NMR (400 MHz, CDCl₃) δ 5.41-5.31 (m, 3H), 4.27-3.91 (m, 7H), 3.83-3.70 (m, 4H), 3.69-3.55 (m, 16H), 3.53-3.17 (m, 17H), 2.78-2.61 (m, 2H), 2.06-1.90 (m, 7H), 1.60-1.49 (m, 5H), 1.49-1.39 (m, 18H), 1.26 (d, J=4.9 Hz, 57H), 0.93-0.83 (m, 9H).

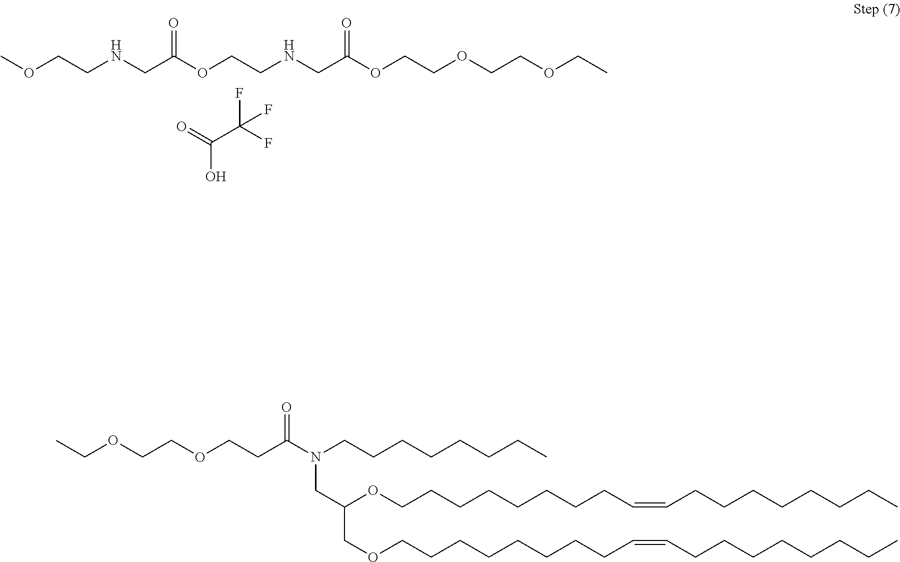

To a solution of 2-[[2-[2-[2-[2-[2-[3-[2,3-bis[(Z)-octadec-9-enoxy]propyl-octyl-amino]-3-oxopropoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.41 g, 0.29 mmol) in anhydrous dichloromethane (5 ml) was added 2,2,2-trifluoroacetic acid (0.85 mL) dropwise and the reaction was stirred at room temperature for 3 hrs. TLC indicated that all the starting material was consumed. The solvent was removed and the residue was azeotroped with DCM several times and then dried under vacuum for 2 h to give 2-[[2-[2-[2-[2-[2-[3-[2,3-bis[(Z)-octadec-9-enoxy]propyloctyl-amino]-3-oxo-propoxy]ethoxy]ethoxy]ethoxy]ethoxy]-2-oxo-ethyl]amino]ethyl 2-(2-methoxyethylamino)acetate;2,2,2-trifluoroacetic acid (0.2645 g, 0.196 mmol, 68% yield) as light yellow oil.

¹H NMR (400 MHz, CDCl₃) δ 5.43-5.31 (m, 4H), 4.62 (s, 1H), 4.54-4.46 (m, 2H), 4.31-4.21 (m, 1H), 4.02 (s, 1H), 3.91 (s, 1H), 3.85-3.13 (m, 36H), 2.82-2.65 (m, 2H), 2.10-1.86 (m, 8H), 1.60-1.48 (m, 6H), 1.28 (dd, J=14.7, 5.5 Hz, 54H), 0.90-0.85 (m, 9H).

### Example 21: Synthesis of 2-[[2-[2-[2-[2-[2-[1-[1,2-bis[(Z)-octadec-9-enoxy]ethyl]tridecoxycarbonylamino]ethoxy]ethoxy]ethoxy]ethoxy]-2-oxo-ethyl]amino]ethyl 2-(2-methoxyethylamino)acetate;2,2,2-trifluoroacetic Acid (Compound (XXIV))

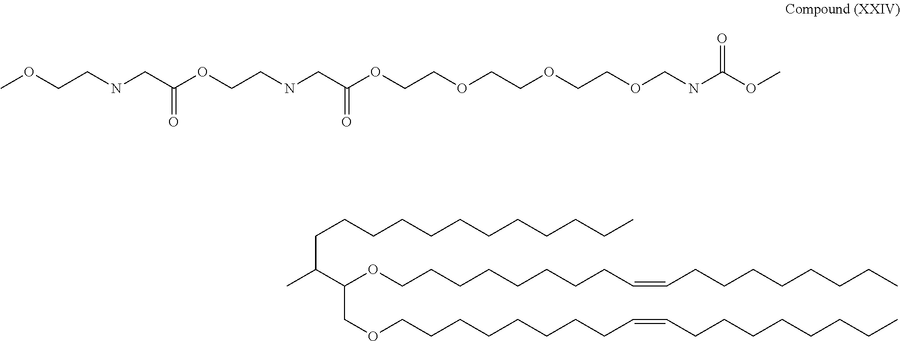

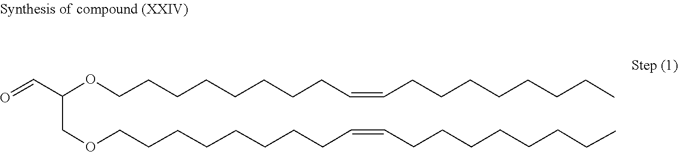 [SMILES: CCCCCCCC/C=C\CCCCCCCCOCC(C=O)OCCCCCCCC/C=C\CCCCCCCC]

To a solution of 2,3-bis[(Z)-octadec-9-enoxy]propan-1-ol (7 g, 11.8 mmol) in DCM (120 ml) was added Dess-Martin Periodinane (7.5 g, 17.7 mmol) at 0° C. in 5 min. The mixture was stirred at 25° C. for 2 hr. Then the mixture was filtered and concentrated. The crude was purified by flash (5% EA in PE), to give 2,3-bis[(Z)-octadec-9-enoxy]propanal (4.3 g, 7.13 mmol) as a colourless oil.

1H NMR (500 MHz, CDCl3) δ 9.72 (d, J=1.3 Hz, 1H), 5.40-5.30 (m, 3H), 3.87-3.76 (m, 1H), 3.75-3.54 (m, 4H), 3.50-3.35 (m, 2H), 1.63 (dt, J=13.6, 6.6 Hz, 2H), 1.55 (s, 1H), 1.52 (d, J=6.7 Hz, 1H), 1.43-1.19 (m, 44H), 0.88 (t, J=6.9 Hz, 6H)

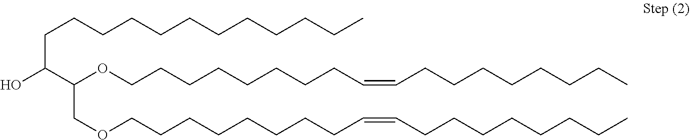 [SMILES: CCCCCCCC/C=C\CCCCCCCCOCC(OCCCCCCCC/C=C\CCCCCCCC)C(O)CCCCCCCCCCCC]

To a solution of Mg (3.78 g) and 12 (2.19 g) in anhydrous THF (50 ml) was added 1-bromododecane (2.58 g, 10.36 mmol). The mixture was stirred at 70° C. under N₂ until the mixture as the colourless one. 1-bromododecane (10.32 g, 41.44 mmol) was added to the reaction. The mixture was stirred at 70° C. for 3 hr. Then the mixture was added to the solution of 2,3-bis[(Z)-octadec-9-enoxy]propanal (5.1 g, 8.63 mmol) in anhydrous THF (50 ml). The mixture was stirred at 7° C. for 14 hr. The mixture was dealt with EA (250 ml), washed with water (250 ml×2), NaCl sat.aq (250 ml). The organic was purified by flash (5% EA in PE), to give 1,2-bis[(Z)-octadec-9-enoxy]pentadecan-3-ol (5.64 g, 7.41 mmol, yield 85.8% %) as a yellow oil.

¹H NMR (500 MHz, CDCl₃) δ 5.41-5.31 (m, 3H), 3.75-3.66 (m, 1H), 3.58 (ddt, J=15.0, 9.7, 4.8 Hz, 2H), 3.51-3.40 (m, 3H), 3.27 (dq, J=17.4, 5.0 Hz, 1H), 2.56 (dd, J=57.4, 5.0 Hz, 1H), 2.04-1.93 (m, 6H), 1.56-1.47 (m, 4H), 1.40-1.17 (m, 66H), 0.88 (t, J=6.9 Hz, 9H).

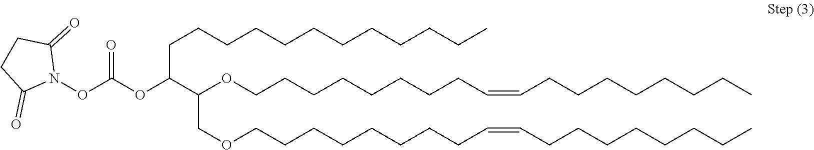 [SMILES: CCCCCCCC/C=C\CCCCCCCCOCC(OCCCCCCCC/C=C\CCCCCCCC)C(CCCCCCCCCCCC)OC(=O)ON1C(=O)CCC1=O]

To a solution of bis(2,5-dioxopyrrolidin-1-yl) carbonate (2.02 g, 7.88 mmol) in DMF (15 ml) was added 1,2-bis[(Z)-octadec-9-enoxy]pentadecan-3-ol (1.5 g, 1.97 mmol) and 4-Dimethylaminopyridine (963 mg, 7.88 mmol). The mixture was stirred at 25° C. for 48 hr. The mixture was dealt with EA (50 ml) washed with water (50×1), LiCl aq (50 ml×2) and dried over Na₂SO₄. The organic was concentrated and purified by flash (10% EA in PE), to give 1-[1,2-bis[(Z)-octadec-9-enoxy]ethyl]tridecyl (2,5-dioxopyrrolidin-1-yl) carbonate (409 mg, 0.44 mmol, yield 22.5%) as a colourless oil. 1H NMR (500 MHz, CDCl3) δ 5.41-5.31 (m, 4H), 4.95 (s, 1H), 3.66-3.59 (m, 1H), 3.56-3.38 (m, 6H), 2.82 (d, J=4.3 Hz, 4H), 2.06-1.93 (m, 7H), 1.79-1.68 (m, 2H), 1.61-1.58 (m, 1H), 1.53 (s, 1H), 1.28 (dd, J=18.3, 10.5 Hz, 66H), 0.88 (t, J=6.9 Hz, 9H).

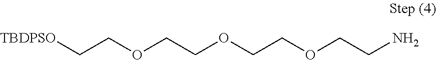

To a solution of 2-[2-(2-aminoethoxy)ethoxy]ethanol (2 g, 10.3 mmol) and imidazole (1.62 g, 23.8 mmol) in CH2Cl2 (40 mL) was added TBDPSCl (3.41 g, 12.4 mmol). The reaction was stirred for 18 h at room temperature. The mixture was diluted with CH₂Cl₂ (30 mL) and washed the resulting mixture with brine. The organic layer was dried over Na₂SO₄, filtered and concentrated. The residue was purified by column chromatography (SiO₂, CH2Cl2/MeOH/NH4OH 80:20:0.25) to obtain 2-[2-[2-[tert-butyl(diphenyl)silyl]oxyethoxy]ethoxy]ethanamine (3.1 g, 7.04 mmol, 68% yield) as colorless oil.

¹H NMR (400 MHz, CDCl₃) δ 7.75-7.61 (m, 4H), 7.49-7.32 (m, 6H), 3.87-3.79 (m, 2H), 3.73-3.51 (m, 12H), 3.15 (s, 4H), 2.92-2.82 (m, 2H), 1.05 (s, 9H).

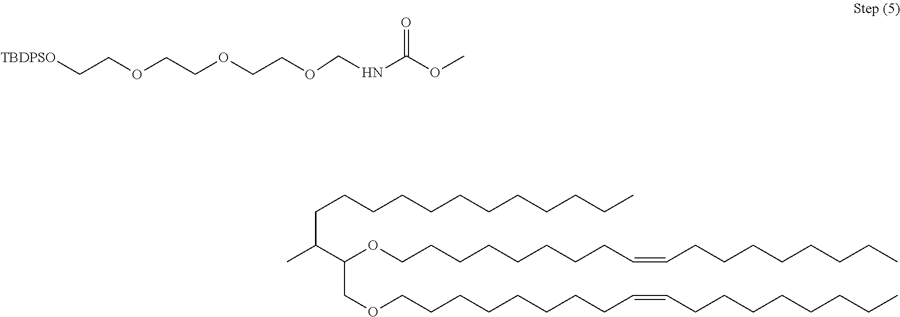

To a solution of 2-[2-[2-[2-[tert-butyl(diphenyl)silyl]oxyethoxy]ethoxy]ethoxy]ethanamine (276 mg, 0.64 mmol) in DCM (8 ml) was added 1-[1,2-bis[(Z)-octadec-9-enoxy]ethyl]tridecyl (2,5-dioxopyrrolidin-1-yl) carbonate (576 mg, 0.64 mmol), TEA (97 ml) and 4-Dimethylaminopyridine (8 mg). The mixture was concentrated and purified by flash (20% EA in PE), to give 1-[1,2-bis[(Z)-octadec-9-enoxy]ethyl]tridecyl N-[2-[2-[2-[2-[tertbutyl(diphenyl)silyl]oxyethoxy]ethoxy]ethoxy]ethyl]carbamate (561 mg, 0.45 mmol, yield 70.7%) as a colourless oil.

¹H NMR (500 MHz, CDCl₃) δ 7.71-7.65 (m, 4H), 7.44-7.34 (m, 6H), 5.40-5.31 (m, 3H), 4.85 (s, 1H), 3.81 (t, J=5.3 Hz, 2H), 3.74-3.24 (m, 22H), 2.06-1.91 (m, 7H), 1.55-1.23 (m, 70H), 1.05 (s, 9H), 0.88 (t, J=6.9 Hz, 9H).

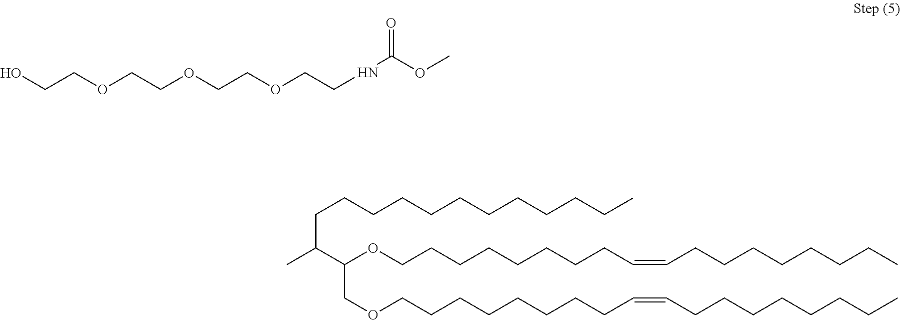 [SMILES: COC(=O)NCCOCCOCCOCCO ; CCCCCCCC/C=C\CCCCCCCCOCC(OCCCCCCCC/C=C\CCCCCCCC)C(C)CCCCCCCCCCCC]

To a solution of 1-[1,2-bis[(Z)-octadec-9-enoxy]ethyl]tridecyl N-[2-[2-[2-[2-[tertbutyl(diphenyl)silyl]oxyethoxy]ethoxy]ethoxy]ethyl]carbamate (561 mg, 0.46 mmol) in THF (6 ml) was added TBAF (0.92 ml). The mixture was stirred at 25° C. for 18 hr. The mixture was concentrated and purified by flash (50% EA in PE), to give 1-[1,2-bis[(Z)-octadec-9-enoxy]ethyl]tridecyl N-[2-[2-[2-(2-hydroxyethoxy)ethoxy]ethoxy]ethyl]carbamate (400 mg, 0.4 mmol, yield 86.9%) as a colourless oil.

¹H NMR (500 MHz, CDCl₃) δ 5.84 (s, 1H), 5.41-5.31 (m, 3H), 4.84 (s, 1H), 3.77-3.29 (m, 23H), 2.04-1.92 (m, 7H), 1.58-1.17 (m, 70H), 0.88 (t, J=6.9 Hz, 9H).

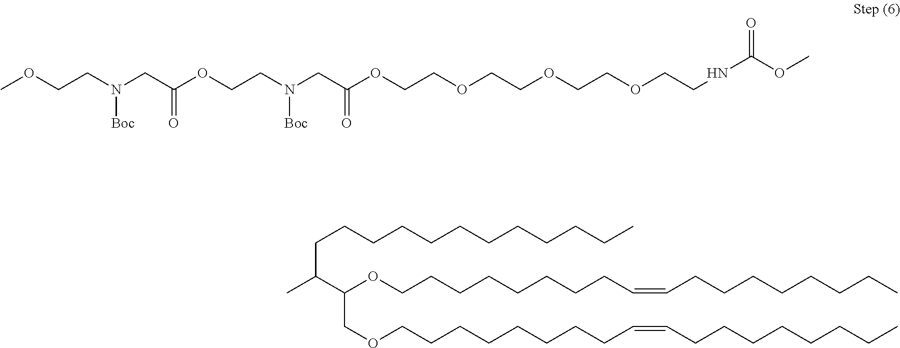

To a solution of 1-[1,2-bis[(Z)-octadec-9-enoxy]ethyl]tridecyl N-[2-[2-[2-(2-hydroxyethoxy)ethoxy]ethoxy]ethyl]carbamate (495 mg, 0.5 mmol) in DCM (10 ml) was added 2-[tertbutoxycarbonyl-[2-[2-[tert-butoxycarbonyl(2-methoxyethyl)amino]acetyl]oxyethyl]amino]acetic acid (329 mg, 0.76 mmol), DIEA (163 mg, 1.26 mmol), DMAP (12 mg) and EDCI (194 mg, 1 mmol). The mixture was stirred at 25° C. for 18 hr. The mixture was dealt with EA (50 ml), washed with water (50 ml), brine (50 ml) and dried over Na₂SO₄. The organic was concentrated and purified by flash (50% EA in PE), to give 2-[[2-[2-[2-[2-[2-[1-[1,2-bis[(Z)-octadec-9-enoxy]ethyl]tridecoxycarbonylamino]ethoxy]ethoxy]ethoxy]ethoxy]-2-oxo-ethyl]-tert-butoxycarbonylamino]ethyl 2-[tert-butoxycarbonyl(2-methoxyethyl)amino]acetate (460 mg, 0.3 mmol, yield 63.9%) as a colourless oil.

¹H NMR (500 MHz, CDCl₃) δ 5.43-5.31 (m, 4H), 5.19 (s, 1H), 4.85 (s, 1H), 4.36-3.25 (m, 37H), 2.04-1.93 (m, 7H), 1.41 (ddd, J=84.3, 38.0, 7.1 Hz, 88H), 0.88 (t, J=6.9 Hz, 9H).

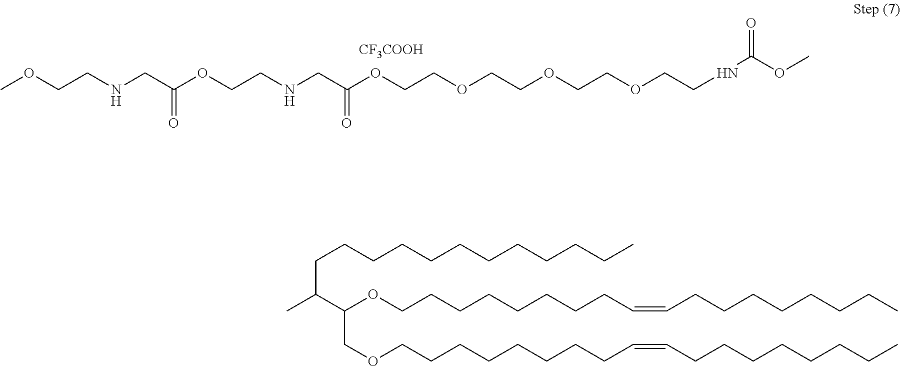

To a solution of 2-[[2-[2-[2-[2-[2-[1-[1,2-bis[(Z)-octadec-9-enoxy]ethyl]tridecoxycarbonylamino]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (460 mg, 0.33 mmol) in DCM (3 ml) was added TFA (0.7 ml). The mixture was stirred at 25° C. for 2 hr. The mixture was concentrated and purified by flash (10% MeOH in DCM), to give 2-[[2-[2-[2-[2-[2-[1-[1,2-bis[(Z)-octadec-9-enoxy]ethyl]tridecoxycarbonylamino]ethoxy]ethoxy]ethoxy]ethoxy]-2-oxo-ethyl]amino]ethyl 2-(2-methoxyethylamino)acetate;2,2,2-trifluoroacetic acid (299 mg, 0.2 mmol, yield 67.9%) as a white solid.

¹H NMR (400 MHz, CDCl₃) δ 5.34 (t, J=5.2 Hz, 4H), 4.85 (s, 2H), 4.59 (s, 2H), 4.38 (s, 2H), 3.98-3.24 (m, 39H), 2.01 (d, J=5.6 Hz, 8H), 1.67-1.10 (m, 70H), 0.88 (t, J=6.8 Hz, 9H).

### Example 22: Synthesis of 2-[[2-[2-[2-[2-[2-[2-[1-[1,2-bis[(Z)-octadec-9-enoxy]ethyl]tridecoxy]-2-oxoethoxy]ethoxy]ethoxy]ethoxy]ethoxy]-2-oxo-ethyl]amino]ethyl 2-(2-methoxyethylamino)acetate;2,2,2-trifluoroacetic Acid (Compound (XXV))

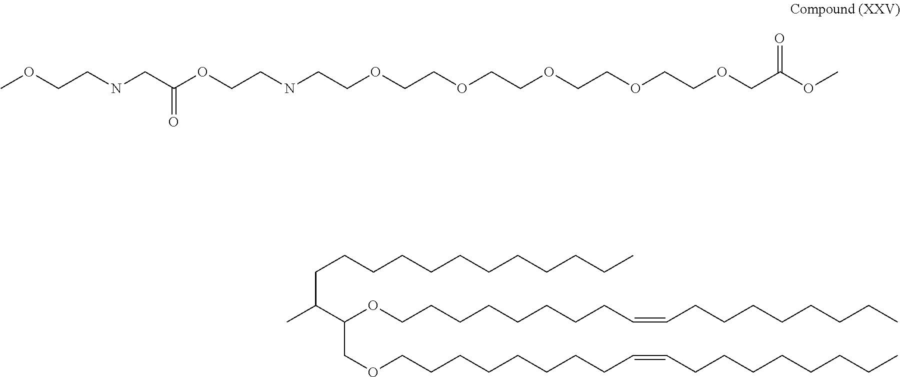

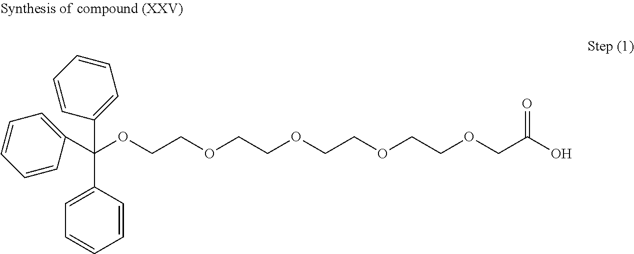 [SMILES: O=C(O)COCCOCCOCCOCCOC(c1ccccc1)(c1ccccc1)c1ccccc1]

To a solution of methyl 2-[2-[2-[2-(2-trityloxyethoxy)ethoxy]ethoxy]ethoxy]acetate (1.41 g, 2.7 mmol) in THF/MeOH/Water (4 ml/4 ml/4 ml) was added LiOH·H2O (1.13 g, 27 mmol). The mixture was stirred at 25° C. for 18 hr. The mixture was dealt with EA (50 ml), washed with water (50 ml). The aqueous phase was added 1N HCl while PH=2. The aqueous phase was washed with EA (50 ml×2), brine (50 ml) and dried over Na2SO4. The organic was concentrated, to give 2-[2-[2-[2-(2-trityloxyethoxy)ethoxy]ethoxy]ethoxy]acetic acid (1.15 g, 2.3 mmol, yield 84.5%) as a colourless oil

¹H NMR (500 MHz, CDCl₃) δ 7.49-7.43 (m, 6H), 7.29 (dd, J=10.3, 4.8 Hz, 6H), 7.25-7.19 (m, 3H), 5.30 (s, 1H), 4.11 (s, 2H), 3.73-3.64 (m, 14H), 3.25 (t, J=5.1 Hz, 2H)

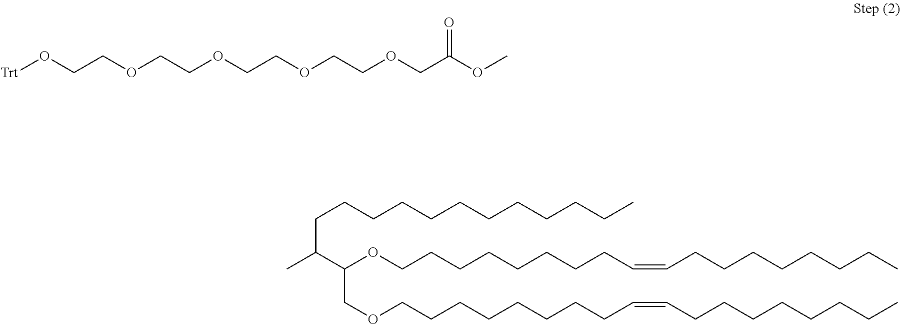

To a solution of 1,2-bis[(Z)-octadec-9-enoxy]pentadecan-3-ol (1.1 g, 1.45 mmol) in DCM (15 ml) was added 2-[2-[2-[2-(2-trityloxyethoxy)ethoxy]ethoxy]ethoxy]acetic acid (550 mg, 1.11 mmol), DIEA (359 mg, 2.78 mmol), DMAP (27.2 mg, 0.22 mmol) and EDCI (426 mg, 2.22 mmol) at 0° C. Then the mixture was stirred at 25° C. for 18 hr. The mixture was dealt with DCM (50 ml), washed with water (50 ml), brine (50 ml) and dried over Na2SO4. The organic was concentrated and purified by flash (20% EA in PE), to give 1-[1,2-bis[(Z)-octadec-9-enoxy]ethyl]tridecyl 2-[2-[2-[2-(2-trityloxyethoxy)ethoxy]ethoxy]ethoxy]acetate (461 mg, 0.37 mmol. yield 32.8%) as a colourless oil

¹H NMR (400 MHz, CDCl₃) δ 7.46 (d, J=7.4 Hz, 6H), 7.29 (t, J=6.1 Hz, 6H), 7.23 (d, J=7.2 Hz, 3H), 5.35 (dd, J=12.5, 7.2 Hz, 4H), 5.10 (s, 1H), 4.12 (d, J=7.3 Hz, 2H), 3.74-3.61 (m, 14H), 3.45 (ddt, J=25.2, 12.9, 6.6 Hz, 7H), 3.23 (t, J=5.2 Hz, 2H), 2.06-1.93 (m, 7H), 1.26 (d, J=4.9 Hz, 70H), 0.88 (t, J=6.8 Hz, 10H).

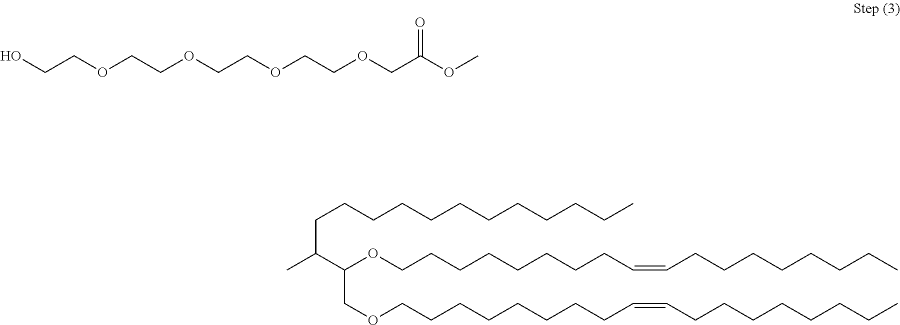 [SMILES: COC(=O)COCCOCCOCCOCCO ; CCCCCCCC/C=C\CCCCCCCCOCC(OCCCCCCCC/C=C\CCCCCCCC)C(C)CCCCCCCCCCCC]

To a solution of 1-[1,2-bis[(Z)-octadec-9-enoxy]ethyl]tridecyl 2-[2-[2-[2-(2-trityloxyethoxy)ethoxy]ethoxy]ethoxy]acetate (461 mg, 0.37 mmol) in THF/MeOH (5/5 ml) was added Toluene-4-sulfonic acid (461 mg, 0.37 mmol). The mixture was stirred at 25° C. for 18 hr. The mixture was dealt with EA (50 ml), washed with NaHCO₃ aq (50 ml×2), brine (50 ml) and dried over Na₂SO₄. The organic was concentrated and purified by flash (5% MeOH in DCM), to give 1-[1,2-bis[(Z)-octadec-9-enoxy]ethyl]tridecyl 2-[2-[2-[2-(2-hydroxyethoxy)ethoxy]ethoxy]ethoxy]acetate (77 mg, 0.07 mmol, yield 14.3%) as a colourless oil.

¹H NMR (500 MHz, CDCl₃) δ 5.39-5.30 (m, 3H), 5.09 (dd, J=11.9, 6.2 Hz, 1H), 4.14 (d, J=8.9 Hz, 2H), 3.80-3.32 (m, 23H), 2.55 (s, 1H), 2.06-1.94 (m, 7H), 1.34 (dd, J=82.3, 60.0 Hz, 70H), 0.88 (t, J=6.9 Hz, 9H).

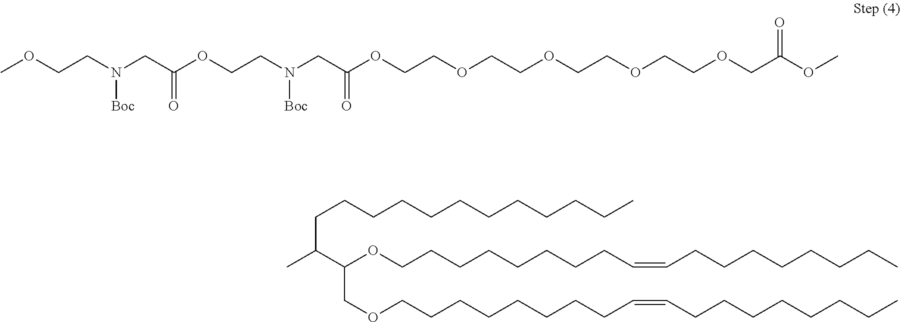

To a solution of 1-[1,2-bis[(Z)-octadec-9-enoxy]ethyl]tridecyl 2-[2-[2-[2-(2-hydroxyethoxy)ethoxy]ethoxy]ethoxy]acetate (340 mg, 0.34 mmol) in DCM (5 ml) was added 2-[tertbutoxycarbonyl-[2-[2-[tert-butoxycarbonyl(2-methoxyethyl)amino]acetyl]oxyethyl]amino]acetic acid (297 mg, 0.68 mmol), DIEA (110 mg, 0.85 mmol), DMAP (8 mg, 0.07 mmol) and EDC HCl (131 mg, 0.68 mmol). The mixture was stirred at 25° C. for 18 hr. Then the mixture was dealt with EA (50 ml), washed with water (50 ml), brine (50 ml) and dried over Na₂SO₄. The organic was purified by flash (5% MeOH in DCM) to give 2-[[2-[2-[2-[2-[2-[2-[l- [1,2-bis[(Z)-octadec-9-enoxy]ethyl]tridecoxy]-2-oxo-ethoxy]ethoxy]ethoxy]ethoxy]ethoxy]-2-oxo-ethyl]-tertbutoxycarbonyl-amino]ethyl 2-[tert-butoxycarbonyl(2-methoxyethyl)amino]acetate (410 mg, 0.29 mmol, yield 83.3%) as a colourless oil.

¹H NMR (500 MHz, CDCl₃) δ 5.40-5.31 (m, 3H), 5.09 (d, J=5.2 Hz, 1H), 4.37-3.21 (m, 40H), 2.07-1.90 (m, 7H), 1.54 (s, 4H), 1.45 (dd, J=22.0, 10.4 Hz, 18H), 1.38-1.04 (m, 66H), 0.88 (t, J=6.9 Hz, 9H).

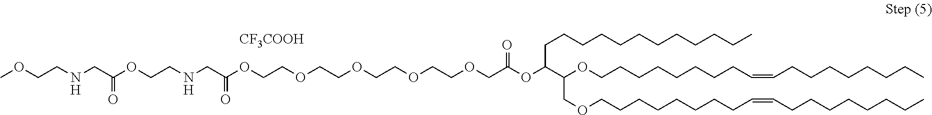 [SMILES: CCCCCCCC/C=C\CCCCCCCCOCC(OCCCCCCCC/C=C\CCCCCCCC)C(CCCCCCCCCCCC)OC(=O)COCCOCCOCCOCCOC(=O)CNCCOC(=O)CNCCOC ; "CF3COOH"]

To a solution of 2-[[2-[2-[2-[2-[2-[2-[1-[1,2-bis[(Z)-octadec-9-enoxy]ethyl]tridecoxy]-2-oxoethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (410 mg, 0.29 mmol) in DCM (5 ml) was added TFA (0.43 ml). The mixture was stirred at 25° C. for 2 hr. The mixture was concentrated and purified by flash (10% MeOH in DCM), to give 2-[[2-[2-[2-[2-[2-[2-[1-[1,2-bis[(Z)-octadec-9-enoxy]ethyl]tridecoxy]-2-oxo-ethoxy]ethoxy]ethoxy]ethoxy]ethoxy]-2-oxoethyl]amino]ethyl 2-(2-methoxyethylamino)acetate;2,2,2-trifluoroacetic acid (298 mg, 0.22 mmol) as a colourless oil.

¹H NMR (400 MHz, CDCl₃) δ 5.35 (d, J=4.8 Hz, 4H), 5.09 (s, 1H), 4.59 (s, 2H), 4.37 (s, 2H), 4.18 (d, J=14.7 Hz, 2H), 4.02-3.61 (m, 23H), 3.49 (s, 2H), 3.46-3.21 (m, 13H), 2.01 (d, J=5.9 Hz, 7H), 1.33 (dd, J=59.3, 53.8 Hz, 70H), 0.88 (t, J=6.7 Hz, 9H)

### Example 23: Synthesis of 2-[[2-[2-[2-[2-[2-[1-[1,2-bis[(Z)-octadec-9-enoxy]ethyl]heptadecoxycarbonylamino]ethoxy]ethoxy]ethoxy]ethoxy]-2-oxo-ethyl]amino]ethyl 2-(2-methoxyethylamino)acetate;2,2,2-trifluoroacetic Acid (Compound (XXVI))

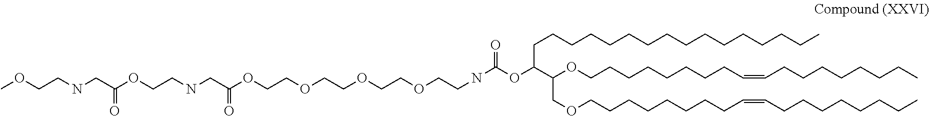 [SMILES: CCCCCCCC/C=C\CCCCCCCCOCC(OCCCCCCCC/C=C\CCCCCCCC)C(CCCCCCCCCCCCCCCC)OC(=O)NCCOCCOCCOCCOC(=O)CNCCOC(=O)CNCCOC]

Synthesis of Compound (XXVI)

To a solution of 2-[[2-[2-[2-[2-[2-[1-[1,2-bis[(Z)-octadec-9-enoxy]ethyl]heptadecoxycarbonylamino]ethoxy]ethoxy]ethoxy]ethoxy]-2-oxo-ethyl]-tert-butoxycarbonylamino]ethyl 2-[tert-butoxycarbonyl(2-methoxyethyl)amino]acetate (400 mg, 0.28 mmol) in DCM (5 ml) was added TFA (0.41 ml). The mixture was stirred at 25° C. for 3 hr. Then the mixture was concentrated and purified by flash (10% MeOH in DCM), to give 2-[[2-[2-[2-[2-[2-[1-[1,2-bis[(Z)-octadec-9-enoxy]ethyl]heptadecoxycarbonylamino]ethoxy]ethoxy]ethoxy]ethoxy]-2-oxo-ethyl]amino]ethyl 2-(2-methoxyethylamino)acetate (285.7 mg, 0.21 mmol, yield 74.4%) as a colourless oil.

¹H NMR (400 MHz, CDCl₃) δ 5.34 (t, J=5.1 Hz, 4H), 4.85 (s, 1H), 4.59 (s, 2H), 4.39 (s, 2H), 4.02-3.23 (m, 38H), 2.04-1.95 (m, 8H), 1.64-1.00 (m, 78H), 0.88 (t, J=6.8 Hz, 9H).

### Example 24: Synthesis of nonyl 8-[3-[2-[2-[2-[2-[2-[2-(2-methoxyethylamino)acetyl]oxyethylamino]acetyl]oxyethoxy]ethoxy]ethoxy]propanoyl-[8-(1-octylnonoxy)-8-oxo-octyl]amino]octanoate;2,2,2-trifluoroacetic Acid (Compound (XXVII))

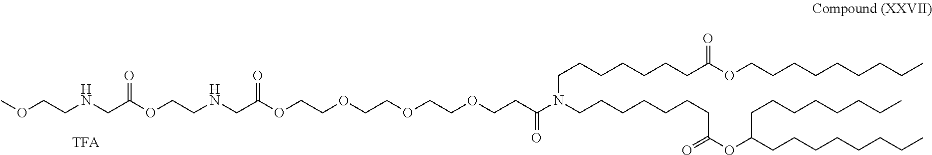 [SMILES: CCCCCCCCCOC(=O)CCCCCCCN(CCCCCCCC(=O)OC(CCCCCCCC)CCCCCCCC)C(=O)CCOCCOCCOCCOC(=O)CNCCOC(=O)CNCCOC]

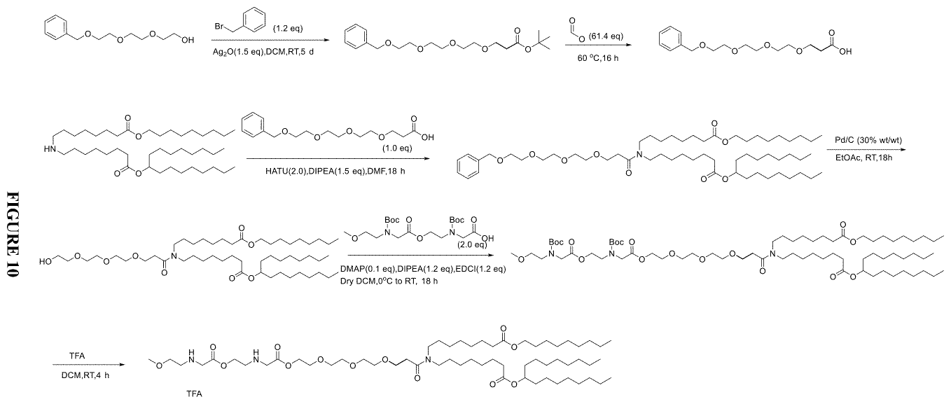

Compound (XXVII) was synthesized based on the chemistry shown in Scheme (12) presented on FIG. 10.

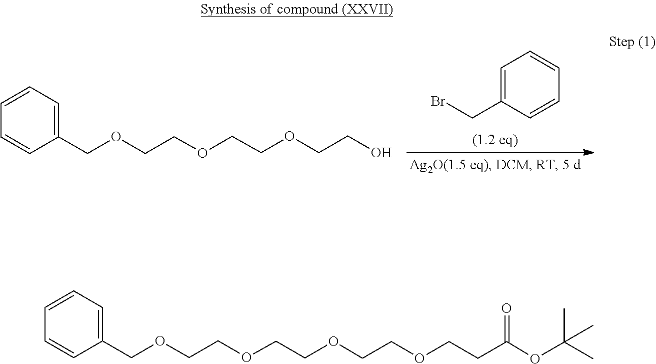 [SMILES: OCCOCCOCCOCc1ccccc1 ; BrCc1ccccc1 ; CC(C)(C)OC(=O)CCOCCOCCOCCOCc1ccccc1]

To the suspension of SILVER OXIDE (0.593 g, 2.56 mmol) in dichloromethane (25 mL) was added tert-butyl 3-[2-[2-(2-hydroxyethoxy)ethoxy]ethoxy]propanoate (0.5 g, 1.71 mmol) and the mixture was stirred at room temperature for 30 min. Then bromomethylbenzene (0.35 g, 2.05 mmol) was added into the above mixture and the mixture was stirred under dark at room temperature for 5 days. TLC indicated that around 50% conversion to the product. The mixture was filtered through celite and washed with dichloromethane. The solvent was removed and the residue was purified through flash chromatography eluted with 20 to 80% ethyl acetate in petroleum ether to give tert-butyl 3-[2-[2-(2-benzyloxyethoxy)ethoxy]ethoxy]propanoate (0.21 g, 0.51 mmol, 31.7% yield) as colorless oil.

¹H NMR (400 MHz, CDCl₃) δ 7.37-7.27 (m, 5H), 4.57 (s, 2H), 3.73-3.59 (m, 14H), 2.50 (t, J=6.6 Hz, 2H), 1.44 (s, 9H).

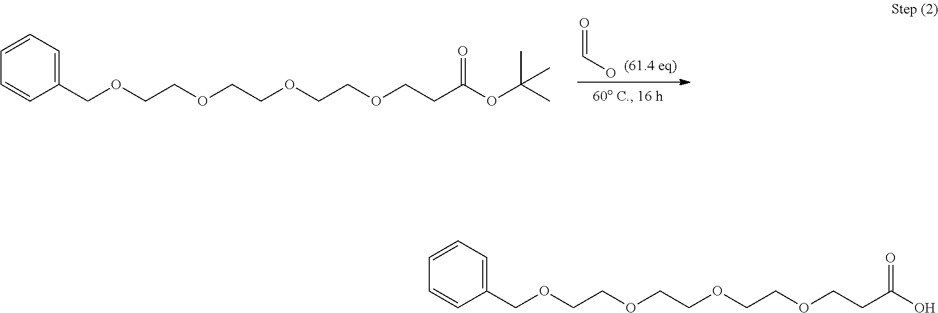

In a 250 mL round-bottomed flask was added tert-butyl 3-[2-[2-(2-benzyloxyethoxy) ethoxy]ethoxy]propanoate (3.25 g, 8.38 mmol) in FORMIC ACID (20 ml, 530 mmol) to give a colorless solution. The reaction was heated to 60° C. for 16 hr. The solvent was removed and residue azeotroped with toluene and dried under vacuum to provide 3-[2-[2-(2-benzyloxyethoxy) ethoxy]ethoxy]propanoic acid (2.7 g, 7.35 mmol, 85% yield) as yellow oil.

¹H NMR (400 MHz, CDCl₃) δ 7.37-7.27 (m, 5H), 4.57 (s, 2H), 3.73-3.59 (m, 14H), 2.50 (t, J=6.6 Hz, 2H), 1.44 (s, 9H).

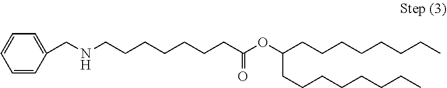 [SMILES: CCCCCCCCC(CCCCCCCC)OC(=O)CCCCCCCNCc1ccccc1]

A solution of heptadecan-9-yl 8-bromooctanoate (250 mg, 0.542 mmol) in phenylmethanamine (1.2 mL, 10.83 mmol) was allowed to stir at rt for 6 hrs. The reaction was cooled to room temperature and solvents were evaporated in vacuo. The residue was taken-up in ethyl acetate and washed with saturated aqueous sodium bicarbonate. The organic layer was separated and washed with brine, dried over Na2SC>4 and evaporated in vacuo. The residue was purified by silica gel chromatography (20-100% (mixture of 1% NH4OH, 20% MeOH in dichloromethane) in dichloromethane) to obtain heptadecan-9-yl 8-(benzylamino)octanoate (200 mg, 0.41 mmol, 76%).

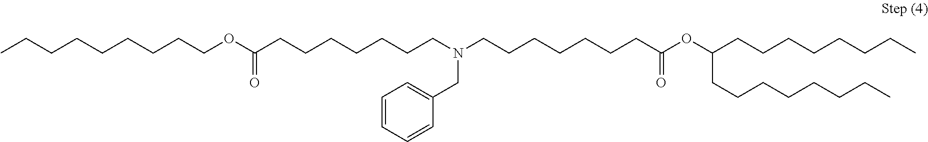 [SMILES: CCCCCCCCCOC(=O)CCCCCCCN(CCCCCCCC(=O)OC(CCCCCCCC)CCCCCCCC)Cc1ccccc1]

A solution of heptadecan-9-y18-(benylamino)octanoate (200 mg, 0.41 mmol), nonyl 8-bromooctanoate (172 mg, 0.49 mmol) and N, N-diisopropylethylamine (100 μL, 0.57 mmol) were dissolved in ACN and was allowed to stir at 62° C. for 96 h. There action was cooled to rt and solvents were evaporated in vacuo. There residue was taken-up in ethylacetate and washed with saturated aqueous sodium bicarbonate. The organic layer was separated and washed with brine, dried over Na₂SO₄ and evaporated in vacuo. The residue was purified by silicagel chromatography (0-100% (mixture of 1% NH40H, 20% MeOH in dichloromethane) in dichloromethane) to give nonyl 8-[benzyl-[8-(1-octylnonoxy)-8-oxo-octyl]amino]octanoate (3.94 g, 4.95 mmol, 69%).

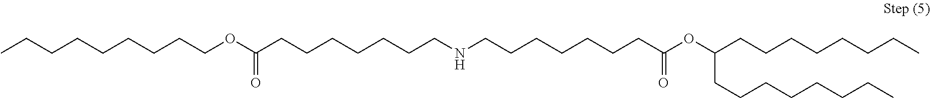 [SMILES: CCCCCCCCCOC(=O)CCCCCCCNCCCCCCCC(=O)OC(CCCCCCCC)CCCCCCCC]

To a solution of nonyl 8-[benzyl-[8-(1-octylnonoxy)-8-oxo-octyl]amino]octanoate (3.94 g, 4.95 mmol) in ethyl acetate (50 mL) was added Pd/C (0.79 g) and the mixture was stirred at room temperature under hydrogen for 18 hrs. TLC indicated that the starting material was consumed and one new spot formed. The reaction was filtered through celite and washed with ethyl acetate. The solvent was removed and the residue was dried under vacuum to give nonyl 8-[[8-(1-octylnonoxy)-8-oxo-octyl]amino]octanoate (3.94 g, 4.95 mmol, 69%) as light yellow oil.

¹H NMR (500 MHz, CDCl₃) δ 4.89-4.82 (m, 1H), 4.05 (t, J=6.8 Hz, 2H), 2.61-2.54 (m, 4H), 2.31-2.24 (m, 4H), 1.62-1.59 (m, 4H), 1.52-1.45 (m, 8H), 1.38-1.18 (m, 51H), 0.90-0.83 (m, 9H).

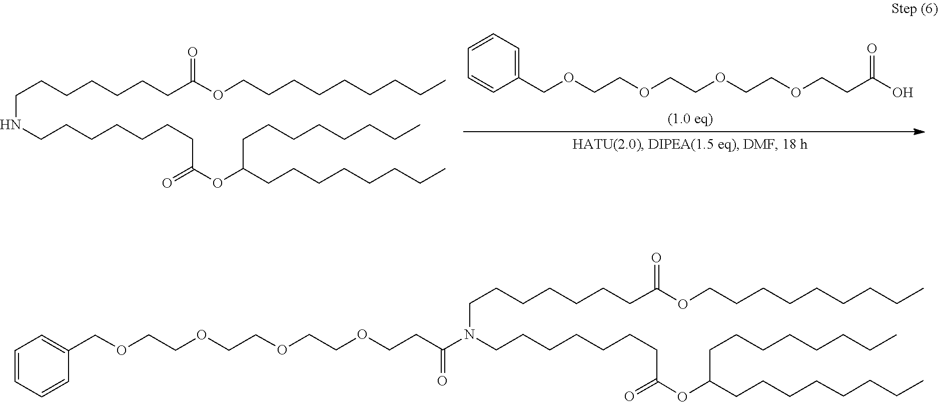

To the solution of nonyl 8-[[8-(1-octylnonoxy)-8-oxo-octyl]amino]octanoate (0.3 g, 0.45 mmol) in dry DCM (10 mL) was added to 3-[2-[2-(2-benzyloxyethoxy)ethoxy]ethoxy]propanoic acid (0.176 g, 0.45 mmol), HATU (0.257 g,0.676 mmol) and DIPEA (0.116 g, 0.901 mmol) then the mixture was stirred at room temperature for 18 hrs. TLC (4% methanol in DCM) indicated that the reaction was finished. The DCM was removed by rotary evaporation. The residue was re-dissolved in ethyl acetate (50 mL) and washed with H₂O (50 mL*3). The organic layer was washed with brine (50 mL), dried over Na₂SO₄. The residue was purified by flash chromatography eluted with 0% to 3% (2%) methanol in CH₂Cl₂ to give nonyl 8-[3-[2-[2-(2-benzyloxyethoxy)ethoxy]ethoxy]propanoyl-[8-(1-octylnonoxy)-8-oxo-octyl]amino]octanoate (0.932 g, 0.970 mmol, 71.8% yield) as pale yellow oil.

¹H NMR (500 MHz, CDCl₃) δ 7.36-7.27 (m, 5H), 4.90-4.82 (m, 1H), 4.57 (s, 2H), 4.09-4.02 (m, 2H), 3.78 (t, J=7.1 Hz, 2H), 3.70-3.59 (m, 12H), 3.30-3.15 (m, 4H), 2.60 (t, J=7.1 Hz, 2H), 2.31-2.24 (m, 4H), 1.64-1.45 (m, 14H), 1.34-1.22 (m, 48H), 0.91-0.85 (m, 9H)

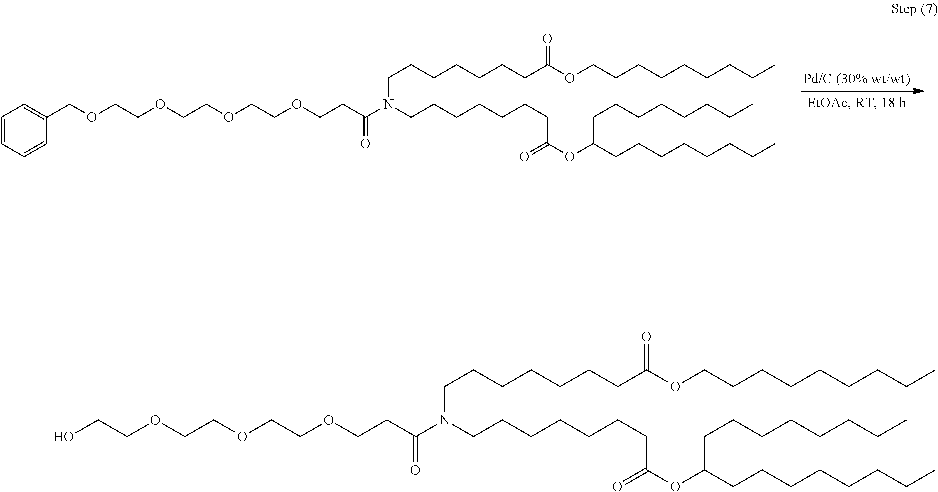 [SMILES: CCCCCCCCCOC(=O)CCCCCCCN(CCCCCCCC(=O)OC(CCCCCCCC)CCCCCCCC)C(=O)CCOCCOCCOCCOCc1ccccc1 ; CCCCCCCCCOC(=O)CCCCCCCN(CCCCCCCC(=O)OC(CCCCCCCC)CCCCCCCC)C(=O)CCOCCOCCOCCO]

To the solution of nonyl 8-[3-[2-[2-(2-benzyloxyethoxy)ethoxy]ethoxy]propanoyl-[8-(1-octylnonoxy)-8-oxo-octyl]amino]octanoate (0.932 g,0.970 mmol) in EtOAc (10 mL) was added Pd/C (280 mg) and the reaction was next stirred overnight at room temperature under an atmosphere of H2. TLC (5% CH3OH in DCM) indicated that the reaction was finished. The slurry was filtered through celite and the celite was rinsed with EtOAc several times. The combined organics were concentrated in vacuo to give nonyl 8-[3-[2-[2-(2-hydroxyethoxy)ethoxy]ethoxy]propanoyl-[8-(1-octylnonoxy)-8-oxo-octyl]amino]octanoate (0.802 g, 0.921 mmol, 95.0% yield) as colorless oil.

¹H NMR (500 MHz, CDCl₃) δ 4.90-4.82 (m, 1H), 4.09-4.03 (m, 2H), 3.80 (t, J=7.0 Hz, 2H), 3.75-3.58 (m, 12H), 3.30-3.17 (m, 4H), 2.61 (t, J=7.0 Hz, 2H), 2.33-2.24 (m, 4H), 1.65-1.45 (m, 14H), 1.36-1.22 (m, 48H), 0.88 (t, J=6.9 Hz, 9H).

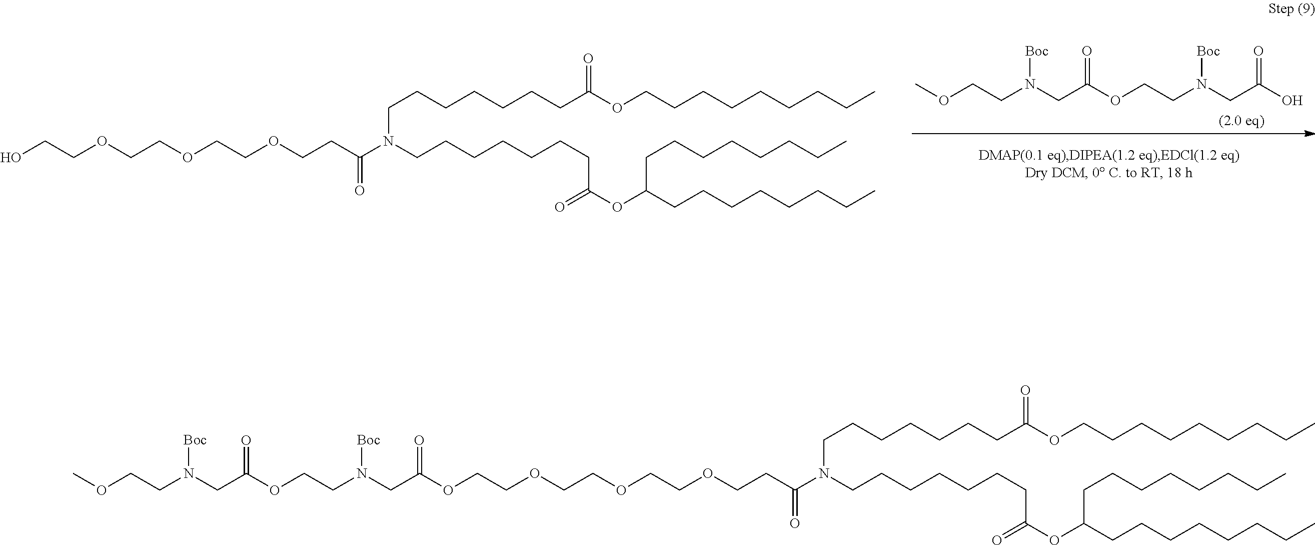

To a solution of nonyl 8-[3-[2-[2-(2-hydroxyethoxy)ethoxy]ethoxy]propanoyl-[8-(1-octylnonoxy)-8-oxo-octyl]amino]octanoate (0.802 g,0.921 mmol) and 2-[tert-butoxycarbonyl-[2-[2-[tert-butoxycarbonyl(2-methoxyethyl)amino]acetyl]oxyethyl]amino]acetic acid (0.801 g,1.84 mmol) in dry DCM (10 mL) was added DMAP (12 mg, 0.0921 mmol), DIPEA (0.143 g,1.11 mmol) and then added EDCI (0.212 g,1.11 mmol) at 0° C. The reaction was allowed to stir at rt for 18 h. Water (20 mL) was added to quench the reaction and more DCM (50 mL) was added. The organic layer was washed with saturated sodium bicarbonate and dried over Na2SO4, filtered and concentrated. The residue was purified by silica gel chromatography (eluted with 0-5% methanol in DCM (3%)) to give nonyl 8-[3-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]propanoyl-[8-(1-octylnonoxy)-8-oxo-octyl]amino]octanoate (0.970 g, 0.724 mmol, 78.5% yield) as pale yellow oil.

¹H NMR (400 MHz, CDCl₃) δ 4.91-4.82 (m, 1H), 4.32-4.19 (m, 4H), 4.10-3.94 (m, 6H), 3.79 (t, J=7.1 Hz, 2H), 3.75-3.60 (m, 10H), 3.57-3.43 (m, 6H), 3.32-3.16 (m, 7H), 2.61 (t, J=7.1 Hz, 2H), 2.35-2.22 (m, 4H), 1.65-1.23 (m, 80H), 0.94-0.82 (m, 9H).

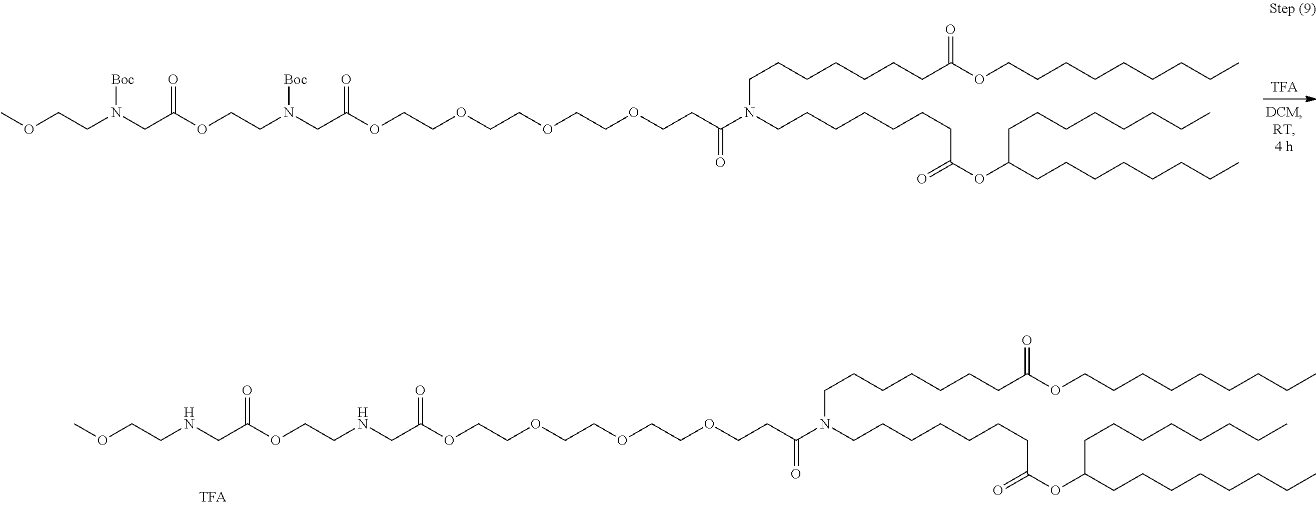

The mixture of nonyl 8-[3-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]propanoyl-[8-(1-octylnonoxy)-8-oxo-octyl]amino]octanoate (0.970 g, 0.754 mmol) in 10 mL DCM was added TFA (5 mL) and the mixture was stirred at room temperature for 2 h. The solvent was removed and azeotroped with dichloromethane three times (50 mL DCM*3). The residue was purified by silica gel chromatography (0-12% methanol in DCM (9%)) to give nonyl 8-[3-[2-[2-[2-[2-[2-[2-(2-methoxyethylamino)acetyl]oxyethylamino]acetyl]oxyethoxy]ethoxy]ethoxy]propanoyl-[8-(1-octylnonoxy)-8-oxo-octyl]amino]octanoate;2,2,2-trifluoroacetic acid (0.5411 g, 0.437 mmol, 58.0% yield) as pale yellow oil.

¹H NMR (400 MHz, CDCl₃) δ 4.91-4.81 (m, 1H), 4.62 (s, 2H), 4.35 (s, 2H), 4.10-3.90 (m, 6H), 3.82-3.61 (m, 14H), 3.47-3.16 (m, 11H), 2.61 (t, J=6.4 Hz, 2H), 2.33-2.23 (m, 4H), 1.66-1.44 (m, 14H), 1.28 (d, J=15.7 Hz, 48H), 0.93-0.83 (m, 9H). LCMS: Find peak: MS(ESI) m/z=1086.9 (M+H)+at 2.499 min

### Example 25: Synthesis of 6-[3-[2-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]ethyl-octylamino]-2-[6-(2-hexyldecanoyloxy)hexoxy]-3-oxo-propoxy]hexyl 2-hexyldecanoate; 2,2,2-trifluoroacetic Acid (Compound (XXVIII))

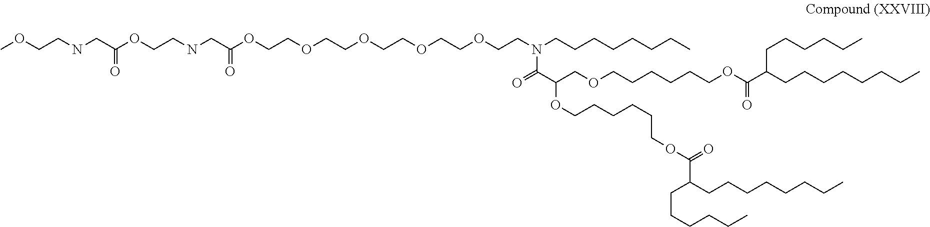 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCOC(=O)CNCCOC(=O)CNCCOC]

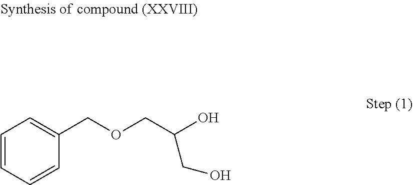 [SMILES: OCC(O)COCc1ccccc1]

The mixture of 4-(benzyloxymethyl)-2,2-dimethyl-1,3-dioxolane (38 g, 0.171 mol) in AcOH (160 mL) and H2O (160 mL) was stirred at room temperature for 18 hrs. TLC indicated that all the starting materials was consumed. The solvent was removed under vacuum and azeotroped with toluene several times. 3-benzyloxypropane-1,2-diol (31.0 g, quant.) as light yellow oil was obtained which was used without further purification.

¹H NMR (400 MHz, CDCl₃) δ 7.39-7.27 (m, 5H), 4.54 (s, 2H), 3.88 (tt, J=5.9, 4.0 Hz, 1H), 3.74-3.24 (m, 6H).

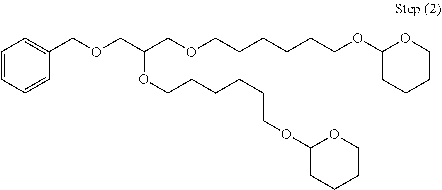 [SMILES: c1ccc(COCC(COCCCCCCOC2CCCCO2)OCCCCCCOC2CCCCO2)cc1]

To the solution of 3-benzyloxypropane-1,2-diol (10 g, 0.055 mol) in dry DMF (200 mL) was added NaH (11 g, 0.274 mol) several times at 0° C. then the mixture was heated to 80° C. for min. Then the reaction was cooled to room temperature and 2-(6-bromohexoxy)tetrahydropyran (36.4 g, 0.137 mol) in dry DMF 100 mL was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 0 to 20% EA in PE (15%) to give 2-[6-[1-(benzyloxymethyl)-2-(6-tetrahydropyran-2-yloxyhexoxy)ethoxy]hexoxy]tetrahydropyran (11.0 g, 36.4% yield) as colorless oil.

¹H NMR (400 MHz, CDCl₃) δ 7.38-7.27 (m, 5H), 4.59-4.53 (m, 4H), 3.90-3.82 (m, 2H), 3.77-3.69 (m, 2H), 3.64-3.31 (m, 14H), 1.86-1.71 (m, 3H), 1.65-1.46 (m, 17H), 1.41-1.33 (m, 8H)

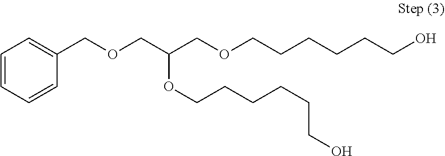 [SMILES: OCCCCCCOCC(COCc1ccccc1)OCCCCCCO]

To a solution of 2-[6-[1-(benzyloxymethyl)-2-(6-tetrahydropyran-2-yloxyhexoxy)ethoxy]hexoxy]tetrahydropyran (11.0 g, 0.02 mol) in EtOH (250 mL) was added p-Toluenesulfonic acid (5.16 g, 0.03 mol) in one portion at room temperature and the mixture was stirred at room temperature for 18 hrs. TLC (4% CH3OH in DCM) indicated that the starting material was disappeared completely. After the reaction was quenched with dilute sodium bicarbonate solution (300 mL), the solvent was extracted with EA (2*300 mL). The organic layers were washed with brine, dired over sodium sulfate, filtered and concentrated. The residue was purified by flash chromatography eluted with 0% to 5% CH3OH in DCM (%) to give 6-[3-benzyloxy-2-(6-hydroxyhexoxy)propoxy]hexan-1-ol (7.0 g, 91.6% yield) as colorless oil.

¹H NMR (400 MHz, CDCl₃) δ 7.38-7.27 (m, 5H), 4.55 (s, 2H), 3.64-3.41 (m, 14H), 1.74 (s, 2H), 1.57 (dt, J=9.6, 6.9 Hz, 8H), 1.40-1.33 (m, 8H).

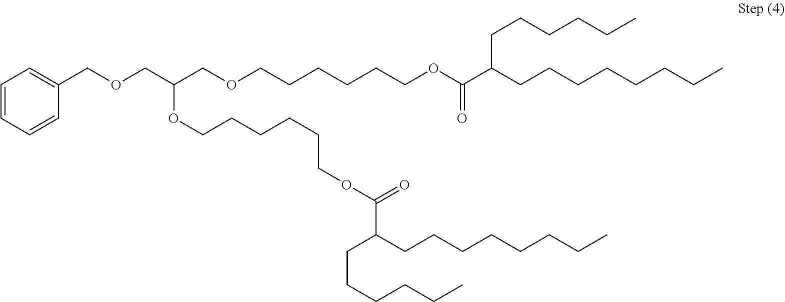 [SMILES: CCCCCCCCC(CCCCCC)C(=O)OCCCCCCOCC(COCc1ccccc1)OCCCCCCOC(=O)C(CCCCCC)CCCCCCCC]

To the solution of 6-[3-benzyloxy-2-(6-hydroxyhexoxy)propoxy]hexan-1-ol (7.0 g, 0.0183 mol) and 2-butyloctanoic acid (14.10 g, 0.0549 mol) in dry dichloromethane (100 mL) were added DIPEA (14.2 g, 0.110 mol), DMAP (0.894 g, 7.32 mmol) and under ice bath added EDCI (9.12 g, 0.0476 mol). The mixture was stirred at room temperature for 18 hrs. The reaction was quenched with NaHCO₃ (100 mL) and washed with brine. The organic layer was dried over sodium sulfate, filtered and concentrated. The residue was purified by flash chromatography eluted with 0% to 5% (3%) CH₃OH in DCM to give 6-[3-benzyloxy-2-[6-(2-hexyldecanoyloxy)hexoxy]propoxy]hexyl 2-hexyldecanoate (15.0 g, 95.4% yield) as colorless oil.

¹H NMR (400 MHz, CDCl₃) δ 7.38-7.27 (m, 5H), 4.55 (s, 2H), 4.05 (td, J=6.6, 1.6 Hz, 4H), 3.59-3.40 (m, 8H), 2.35-2.26 (m, 2H), 1.65-1.53 (m, 12H), 1.45-1.34 (m, 12H), 1.25 (s, 40H), 0.87 (dd, J=7.0, 5.9 Hz, 12H)

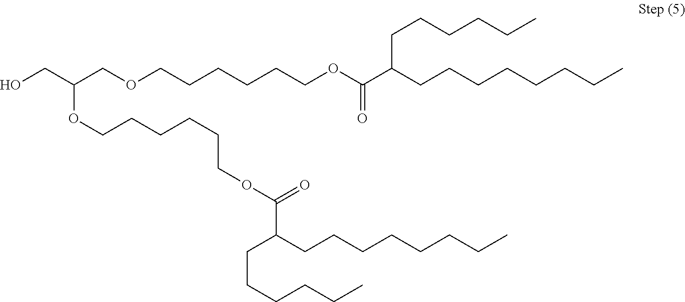 [SMILES: CCCCCCCCC(CCCCCC)C(=O)OCCCCCCOCC(CO)OCCCCCCOC(=O)C(CCCCCC)CCCCCCCC]

A solution of 6-[3-benzyloxy-2-[6-(2-hexyldecanoyloxy)hexoxy]propoxy]hexyl 2-hexyldecanoate (15.0 g, 0.0175 mol) in EtOAc (250 mL) was purged for 10 minutes with N2 followed by addition of Pd/C (20% wt/wt, 3 g) and the reaction continued purging with N2. The reaction was next evacuated under vacuum and backfilled with H₂ 3 times. The reaction was next stirred overnight at room temperature under an atmosphere of H₂. TLC (5% CH3OH in DCM) shows that the reaction was finished. The slurry filtered through celite and the celite was rinsed with EtOAc several times. The combined organics were next concentrated in vacuoto give 6-[2-[6-(2-hexyldecanoyloxy)hexoxy]-3-hydroxy-propoxy]hexyl 2-hexyldecanoate (12.7 g, 94.6% yield) as colorless oil.

¹H NMR (400 MHz, CDCl₃) δ 4.06 (t, J=6.6 Hz, 4H), 3.76-3.41 (m, 9H), 2.31 (tt, J=9.0, 5.3 Hz, 2H), 2.18 (s, 1H), 1.69-1.52 (m, 14H), 1.39 (ddd, J=14.5, 13.1, 6.5 Hz, 12H), 1.27 (d, J=14.9 Hz, 40H), 0.87 (dd, J=7.0, 6.1 Hz, 12H).

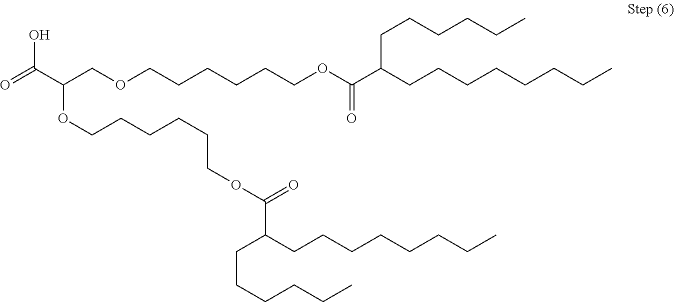 [SMILES: CCCCCCCCC(CCCCCC)C(=O)OCCCCCCOCC(OCCCCCCOC(=O)C(CCCCCC)CCCCCCCC)C(=O)O]

To a solution of 6-[2-[6-(2-hexyldecanoyloxy)hexoxy]-3-hydroxy-propoxy]hexyl 2-hexyldecanoate (3.0 g, 4.0 mmol) in Acetonitrile (AcCN, 40 mL) and PH=4-buffer solution (20 mL, AcOH: AcONa: water=92 mL:33 g:1000 mL) was added sodium chlorite (2.0 g, 21.4 mmol) and sodium hypochlorite (0.14 g, 2.0 mmol) and followed by TEMPO ((2,2,6,6-tetramethylpiperidin-1-yl)oxyl) (0.30 g, 0.20 mmol). The reaction became black and stirred at 20° C. for 4 hrs. LCMS indicated a clean reaction. The reaction was quenched with 40 drops of methanol and was poured into water (80 mL) and extracted with ethyl acetate (120 ml×3). The organic layers were combined, washed with brine (120 mL), dried over anhydrous sodium sulfate, filtered and concentrated. The residue, combined with above batch, was purified through flash chromatography eluted with 0% to 10% (7%) methanol in dichloromethane to give 2,3-bis[6-(2-hexyldecanoyloxy)hexoxy]propanoic acid (2.32 g, 75.9% yield) as light yellow oil.

¹H NMR (400 MHz, CDCl₃) δ 4.12-4.01 (m, 5H), 3.81-3.40 (m, 6H), 2.37-2.26 (m, 2H), 1.70-1.52 (m, 12H), 1.47-1.34 (m, 12H), 1.27 (d, J=14.8 Hz, 40H), 0.87 (dd, J=6.9, 6.2 Hz, 12H).

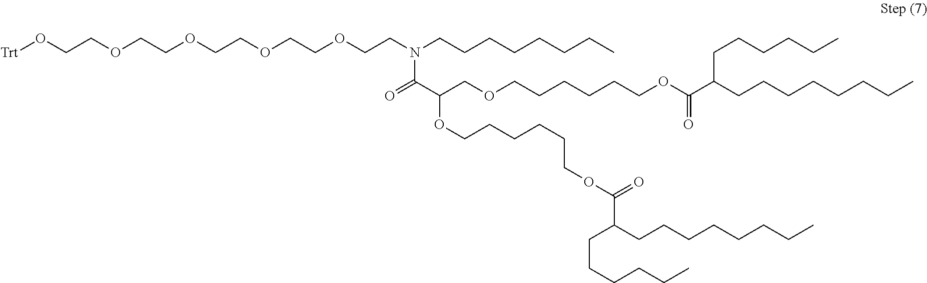

A mixture of 2,3-bis[6-(2-hexyldecanoyloxy)hexoxy]propanoic acid (2.32 g, 2.96 mmol), EDC HCl (0.85 g, 4.44 mmol), N-Hydroxysuccinimide (0.511 g, 4.44 mmol) in DCM (40 mL). The reaction mixture was stirred for 2 hrs at room temperature. Then N-[2-[2-[2-[2-(2-trityloxyethoxy)ethoxy]ethoxy]ethoxy]ethyl]octan-1-amine (2.10 g, 3.55 mmol) and DIEA (1.15 g, 8.89 mmol) were added. The mixture was stirred for 16 hrs at room temperature. The mixture was poured into DCM (300 mL) and washed with NaHCO₃ solution and brine, dried over Na2SO₄ and concentrated. The residue was purified by column chromatography on silica gel eluting with MeOH in DCM (4%) to afford 6-[2-[6-(2-hexyldecanoyloxy)hexoxy]-3-[octyl-[2-[2-[2-[2-(2-trityloxyethoxy)ethoxy]ethoxy]ethoxy]ethyl]amino]-3-oxopropoxy]hexyl 2-hexyldecanoate (2.1 g, 52.2% yield) as light yellow oil.

¹H NMR (400 MHz, CDCl₃) δ 7.46 (d, J=7.4 Hz, 6H), 7.28 (dd, J=11.9, 4.0 Hz, 6H), 7.22 (t, J=7.2 Hz, 3H), 4.45-4.28 (m, 1H), 4.05 (t, J=6.4 Hz, 4H), 3.73-3.20 (m, 29H), 2.36-2.25 (m, 2H), 1.70-1.33 (m, 28H), 1.25 (s, 50H), 0.93-0.82 (m, 15H).

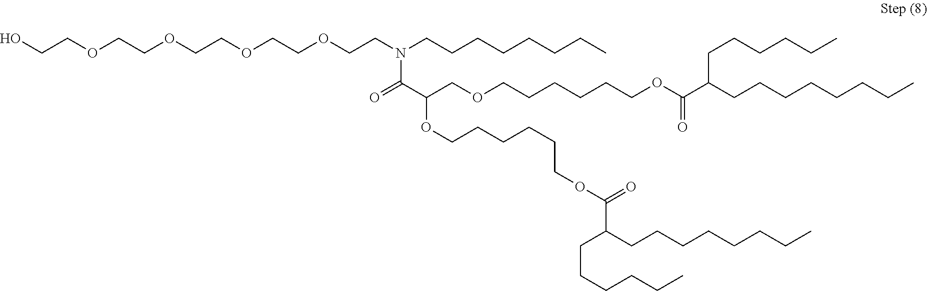 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCO]

To a solution of 6-[2-[6-(2-hexyldecanoyloxy)hexoxy]-3-[octyl-[2-[2-[2-[2-(2-trityloxyethoxy)ethoxy]ethoxy]ethoxy]ethyl]amino]-3-oxo-propoxy]hexyl 2-hexyldecanoate (21 g, 1.55 mmol) in methanol/THF (40 mL, 1/1 v/v) was added 4-methylbenzenesulfonic acid (1.47 g, 7.44 mmol) in one portion at room temperature and the mixture was stirred at room temperature for 18 hrs. TLC indicated that the starting material was disappeared completely, 40 mL triethylamine was added to quenched the reaction and the solvent was removed under vacuum. The residue was purified by flash chromatography eluted with 0% to 5% MeOH in DCM (4%) to give 6-[2-[6-(2-hexyldecanoyloxy)hexoxy]-3-[2-[2-[2-[2-(2-hydroxyethoxy)ethoxy]ethoxy]ethoxy]ethyl-octyl-amino]-3-oxo-propoxy]hexyl-hexyldecanoate.(1.41 g, 81.7% yield) as colorless oil.

¹H NMR (400 MHz, CDCl₃) δ 4.47-4.27 (m, 1H), 4.09-4.00 (m, 4H), 3.75-3.31 (m, 28H), 2.37-2.23 (m, 2H), 1.68-1.32 (m, 28H), 1.27 (d, J=16.0 Hz, 50H), 0.87 (dd, J=6.6, 5.7 Hz, 15H).

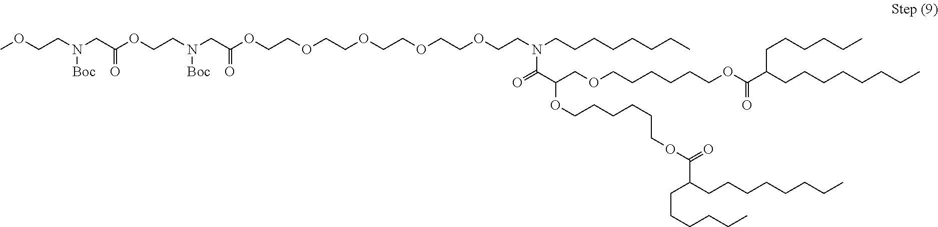 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCOC(=O)CN(CCOC(=O)CN(CCOC)C(=O)OC(C)(C)C)C(=O)OC(C)(C)C]

6-[2-[6-(2-hexyldecanoyloxy)hexoxy]-3-[2-[2-[2-[2-(2-hydroxyethoxy)ethoxy]ethoxy]ethoxy]ethyl-octyl-amino]-3-oxo-propoxy]hexyl 2-hexyldecanoate (0.4 g, 0.36 mmol) and 2-[tert-butoxycarbonyl-[2-[2-[tert-butoxycarbonyl(2-methoxyethyl)amino]acetyl]oxyethyl]amino]acetic acid (0.24 g, 0.54 mmol) in dry dichloromethane (10 mL) then added DIPEA (0.06 g, 0.43 mmol), DMAP (0.005 g, 0.04 mmol) and added EDCI (0.09 g, 0.43 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% to 10% (7%) MeOH in DCM to give 6-[2-[6-(2-hexyldecanoyloxy)hexoxy]-3-[2-[2-[2-[2-[2-[2-[2-[2-(2-methoxyethylamino)acetyl]oxyethylamino]acetyl]oxyethoxy]ethoxy]ethoxy]ethoxy]ethyl-octylamino]-3-oxo-propoxy]hexyl 2-hexyldecanoate (0.45 g, 81.9% yield) as light yellow oil.

¹H NMR (400 MHz, CDCl₃) δ 4.43-4.19 (m, 5H), 4.10-4.02 (m, 6H), 4.01-3.95 (m, 2H), 3.70-3.39 (m, 32H), 3.30 (d, J=5.6 Hz, 3H), 2.35-2.25 (m, 2H), 1.63-1.50 (m, 14H), 1.50-1.39 (m, 22H), 1.35 (s, 8H), 1.25 (s, 50H), 0.92-0.82 (m, 15H).

Step (10)

6-[3-[2-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]ethyl-octyl-amino]-2-[6-(2-hexyldecanoyloxy)hexoxy]-3-oxo-propoxy]hexyl 2-hexyldecanoate (630 mg, 0.411 mmol) in DCM (8 mL) cooled to 0° C. was added TFA (1.5 mL) and the mixture was stirred at RT for 4 h. TLC indicated that the starting material was disappeared. The solvent was removed and the product was azeotroped with DCM several times and then dried under vacuum for 2 h to give 6-[2-[6-(2-hexyldecanoyloxy)hexoxy]-3-[2-[2-[2-[2-[2-[2-[2-[2-(2-methoxyethylamino)acetyl]oxyethylamino]acetyl]oxyethoxy]ethoxy]ethoxy]ethoxy]ethyl-octyl-amino]-3-oxopropoxy]hexyl 2-hexyldecanoate;2,2,2-trifluoroacetic acid (500 mg, quant.) as light yellow oil.

¹H NMR (400 MHz, CDCl₃) δ 4.65-4.31 (m, 5H), 4.12-3.95 (m, 8H), 3.85-3.55 (m, 22H), 3.54-3.13

### Example 26: Synthesis of [(Z)-non-2-enyl] 8-[3-[2-[2-[2-[2-[2-[2-[2-[2-(2-methoxyethylamino)acetyl]oxyethylamino]acetyl]oxyethoxy]ethoxy]ethoxy]ethoxy]ethyl-octyl-amino]-2-[8-[(Z)-non-2-enoxy]-8-oxo-octoxy]-3-oxo-propoxy]octanoate;2,2,2-trifluoroacetic Acid (Compound (XXIX))

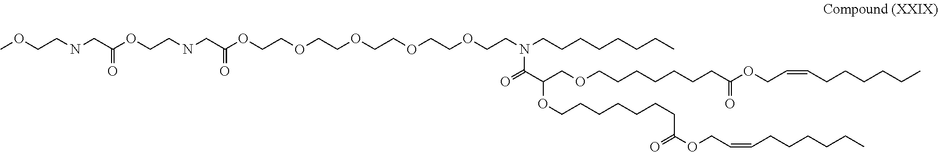 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCOC(=O)CNCCOC(=O)CNCCOC]

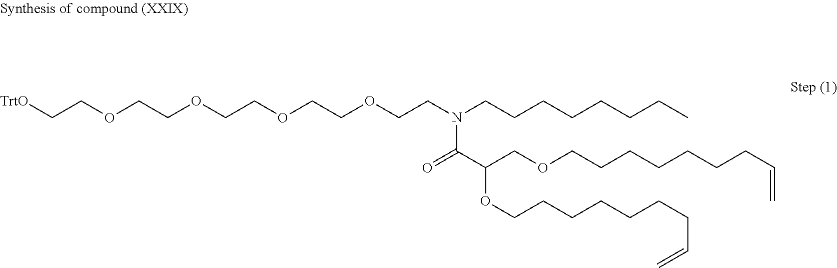

To a solution of N-[2-[2-[2-[2-(2-trityloxyethoxy)ethoxy]ethoxy]ethoxy]ethyl]octan-1-amine (9.52 g, 16.1 mmol) in DCM (150 ml) was added 2,3-bis(non-8-enoxy)propanoic acid (5 g, 14.1 mmol), [dimethylamino(triazolo[4,5-b]pyridin-3-yloxy)methylene]-dimethyl-ammonium;hexafluorophosphate (8.04 g, 21.2 mmol), N,N-diethylethan amine (2.85 g, 28.2 mmol). The mixture was stirred at 25° C. for 18 hrs. Then the mixture was dealt with EA (300 ml), washed with water (300 ml×2), NaCl sat.aq (300 ml) and dried over Na2SO4. The organic was concentrated and purified by flash (25% EA in PE), to give 2,3-bis(non-8-enoxy)-N-octyl-N-[2-[2-[2-[2-(2-trityloxyethoxy)ethoxy]ethoxy]ethoxy]ethyl]propanamide (8.22 g, 8.69 mmol, yield 61.5%) as a colourless oil.

¹H NMR (500 MHz, CDCl₃) δ 7.46 (d, J=7.5 Hz, 6H), 7.29 (t, J=7.6 Hz, 6H), 7.22 (t, J=7.3 Hz, 3H), 5.86-5.74 (m, 2H), 5.03-4.89 (m, 4H), 4.43-4.29 (m, 1H), 3.70-3.22 (m, 29H), 2.03 (dd, J=13.5, 6.5 Hz, 4H), 1.57-1.51 (m, 4H), 1.40-1.23 (m, 28H), 0.88 (q, J=6.9 Hz, 3H).

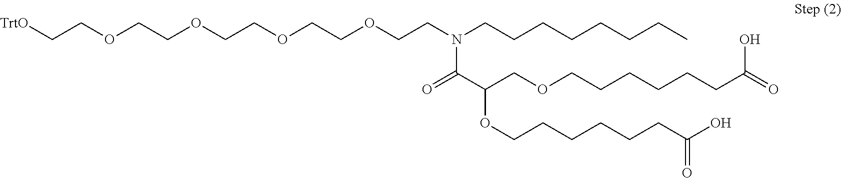

To a solution of 2,3-bis(oct-7-enoxy)-N-octyl-N-[2-[2-[2-[2-(2-trityloxyethoxy)ethoxy]ethoxy]ethoxy]ethyl]propanamide (8.22 g,9.13 mmol) in ACN/CCl4/H2O (80 ml/80 ml/80 ml) was added NaIO4 (15.6 g,73 mmol) RUTHENIUM(III) CHLORIDE HYDRATE (412 mg, 1.83 mmol). The mixture was stirred at 25° C. for 18 hr. The mixture was filtered and dealt with EA (500 ml), washed with Na₂S₂O₃ aq (300 ml), brine (300 ml) and dried over Na₂SO₄. The organic was concentrated and dealt with tert-butyl alcohol/water (120 ml/40 ml). Sodium chlorite (2.48 g, 27.4 mmol), 2-Methyl-2-butene (16 g, 228 mmol) and sodium dihydrogen phosphate (3.29 g, 27.4 mmol) was added to the mixture. The mixture was stirred at 25° C. for 2 hr. Then the mixture was dealt with EA (500 ml), washed with water (500 ml), brine (300 ml) and dried over Na₂SO₄. The organic was concentrated and purified by flash (10% MeOH in DCM), to give 7-[2-(6-carboxyhexoxy)-3-[octyl-[2-[2-[2-[2-(2-trityloxyethoxy)ethoxy]ethoxy]ethoxy]ethyl]amino]-3-oxo-propoxy]heptanoic acid (5.11 g, 5.19 mmol, yield 56.8%) as a grey oil.

¹H NMR (400 MHz, CDCl₃) δ 7.48 (dd, J=15.2, 13.8 Hz, 6H), 7.29 (dd, J=10.1, 4.8 Hz, 6H), 7.22 (dd, J=8.3, 6.1 Hz, 3H), 4.38 (ddd, J=35.0, 7.3, 4.4 Hz, 1H), 3.80-3.33 (m, 26H), 3.23 (t, J=5.2 Hz, 2H), 2.50-2.24 (m, 4H), 1.56-1.18 (m, 28H), 0.87 (q, J=6.8 Hz, 3H).

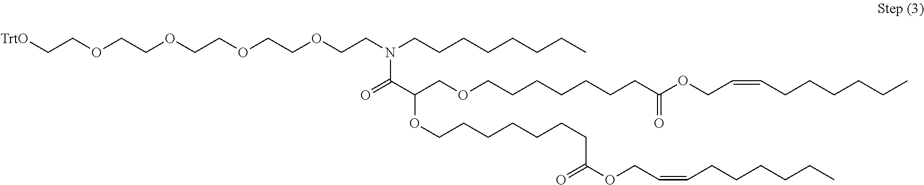

To a solution of 7-[2-(6-carboxyhexoxy)-3-[octyl-[2-[2-[2-[2-(2-trityloxyethoxy)ethoxy]ethoxy]ethoxy]ethyl]amino]-3-oxo-propoxy]heptanoic acid (5.1 g, 5.45 mmol) in DCM (80 ml) was added (Z)-non-2-en-1-ol (1.86 mg, 13.1 mmol), EDC HCl (3.13 g, 16.3 mmol), DIEA (2.46 g, 19.1 mmol) and DMAP (333 mg). The mixture was stirred at 25° C. for 18 hrs. Then the mixture was concentrated and purified by flash (25% EA in PE), to give [(Z)-non-2-enyl] 8-[2-[8-[(Z)-non-2-enoxy]-8-oxo-octoxy]-3-[octyl-[2-[2-[2-[2-(2-trityloxyethoxy)ethoxy]ethoxy]ethoxy]ethyl]amino]-3-oxopropoxy]octanoate (2.27 g, 1.83 mmol, yield 33.7%) as a colourless oil.

¹H NMR (500 MHz, CDCl₃) δ 7.46 (d, J=7.4 Hz, 6H), 7.29 (t, J=7.5 Hz, 6H), 7.22 (t, J=7.3 Hz, 3H), 5.64 (dd, J=18.3, 7.5 Hz, 2H), 5.55-5.47 (m, 2H), 4.61 (d, J=6.9 Hz, 4H), 4.42-4.28 (m, 1H), 3.69-3.38 (m, 26H), 3.23 (t, J=5.2 Hz, 2H), 2.29 (t, J=7.5 Hz, 4H), 2.09 (q, J=7.3 Hz, 4H), 1.52-1.24 (m, 48H), 0.88 (t, J=6.8 Hz, 9H).

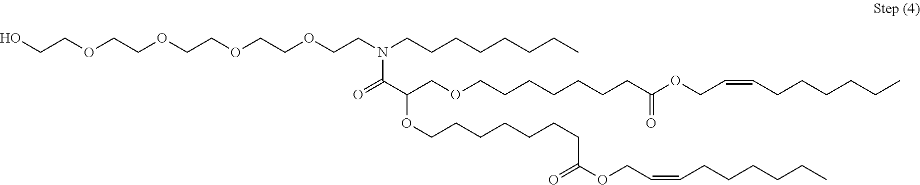 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCO]

To a solution of [(Z)-non-2-enyl] 8-[2-[8-[(Z)-non-2-enoxy]-8-oxo-octoxy]-3-[octyl-[2-[2-[2-[2-(2-trityloxyethoxy)ethoxy]ethoxy]ethoxy]ethyl]amino]-3-oxo-propoxy]octanoate (2.38 g, 1.96 mmol) in THF/MeOH (15 ml/15 ml) was added Toluene-4-sulfonic acid (560 mg, 2.94 mmol). The mixture was stirred at 25° C. for 2 hrs. Then the mixture was dealt with EA (150 ml), washed with water (150 ml), brine (150 ml) and dried over Na2SO4. The mixture was concentrated and purified by flash (5% MeOH in DCM), to give [(Z)-non-2-enyl] 8-[3-[2-[2-[2-[2-(2-hydroxyethoxy)ethoxy]ethoxy]ethoxy]ethyl-octyl-amino]-2-[8-[(Z)-non-2-enoxy]-8-oxo-octoxy]-3-oxopropoxy]octanoate (1.51 g, 1.52 mmol, yield 77.7%) as a colorless oil.

¹H NMR (400 MHz, CDCl₃) δ 5.69-5.46 (m, 4H), 4.62 (d, J=6.8 Hz, 4H), 4.43-4.29 (m, 1H), 3.74-3.40 (m, 28H), 2.29 (td, J=7.7, 1.5 Hz, 4H), 2.10 (dd, J=14.1, 7.0 Hz, 4H), 1.61-1.21 (m, 48H), 0.88 (td, J=6.7, 4.3 Hz, 9H).

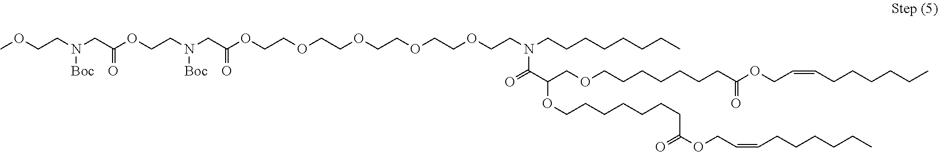 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCOC(=O)CN(CCOC(=O)CN(CCOC)C(=O)OC(C)(C)C)C(=O)OC(C)(C)C]

To a solution of [(Z)-non-2-enyl] 8-[3-[2-[2-[2-[2-(2-hydroxyethoxy)ethoxy]ethoxy]ethoxy]ethyl-octyl-amino]-2-[8-[(Z)-non-2-enoxy]-8-oxo-octoxy]-3-oxo-propoxy]octanoate (400 mg, 0.41 mmol) in DCM (10 ml) was added 2-[tertbutoxycarbonyl-[2-[2-[tert-butoxycarbonyl(2-methoxyethyl)amino]acetyl]oxyethyl]amino]acetic acid (269 mg, 0.62 mmol), 3-(ethyliminomethyleneamino)-N,N-dimethyl-propan-1-amine;hydrochloride (119 mg, 0.62 mmol), N-ethylN-isopropyl-propan-2-amine (107 mg, 0.82 mmol) and N,N-dimethylpyridin-4-amine (5 mg). The mixture was stirred at 25° C. for 3 hrs. The mixture was dealt with EA (50 ml), washed with water (50 ml×2), NaCl sat.aq (50 ml) and dried over Na2SO4. The organic was concentrated and purified by flash (0-30% EA in PE), to give [(Z)-non-2-enyl] 8-[3-[2-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]ethyl-octyl-amino]-2-[8-[(Z)-non-2-enoxy]-8-oxo-octoxy]-3-oxo-propoxy]octanoate (320 mg, 0.23 mmol, yield 54.9%) as a colorless oil.

¹H NMR (400 MHz, CDCl₃) δ 5.58 (tdd, J=17.8, 11.0, 7.2 Hz, 4H), 4.62 (d, J=6.8 Hz, 4H), 4.32-4.22 (m, 4H), 4.07-3.92 (m, 4H), 3.73-3.28 (m, 36H), 2.29 (tt, J=14.7, 7.4 Hz, 4H), 2.10 (q, J=7.0 Hz, 4H), 1.64-1.53 (m, 8H), 1.44 (d, J=19.0 Hz, 18H), 1.37-1.22 (m, 40H), 0.88 (td, J=6.8, 4.3 Hz, 9H).

Step (6)

To a solution of [(Z)-non-2-enyl] 8-[3-[2-[2-[2-[2-[2-[2-[tert-butoxycarbonyl-[2-[2-[tert-butoxycarbonyl(2-methoxyethyl)amino]acetyl]oxyethyl]amino]acetyl]oxyethoxy]ethoxy]ethoxy]ethoxy]ethyl-octyl-amino]-2-[8-[(Z)-non-2-enoxy]-8-oxo-octoxy]-3-oxo-propoxy]octanoate (320 mg, 0.23 mmol) in DCM (5 ml) was added 2,2,2-trifluoroacetic acid (0.34 ml). The mixture was stirred at 26° C. for 18 hrs. The mixture was concentrated to give [(Z)-non-2-enyl] 8-[3-[2-[2-[2-[2-[2-[2-[2-[2-(2-methoxyethylamino)acetyl]oxyethylamino]acetyl]oxyethoxy]ethoxy]ethoxy]ethoxy]ethyl-octyl-amino]-2-[8-[(Z)-non-2-enoxy]-8-oxo-octoxy]-3-oxo-propoxy]octanoate;2,2,2-trifluoroacetic acid (330 mg, quantitative) as a colorless oil.

¹H NMR (400 MHz, CDCl₃) δ 5.64 (dd, J=18.4, 7.7 Hz, 2H), 5.55-5.47 (m, 2H), 4.62 (d, J=6.9 Hz, 4H), 4.38 (t, J=29.5 Hz, 4H), 4.00 (d, J=5.2 Hz, 4H), 3.75-3.27 (m, 36H), 2.30 (t, J=7.5 Hz, 4H), 2.09 (dd, J=14.3, 7.1 Hz, 4H), 1.72-1.16 (m, 48H), 0.88 (dd, J=8.5, 5.1 Hz, 9H).

### Example 27: Synthesis of (Z)-26-(((Z)-octadec-9-en-1-yl)oxy)-5-oxo-6,9,12,15,18,21,24,28-octaoxa-3-azahexatetracont-37-en-1-yl (2-methoxyethyl)glycinate bis(2,2,2-trifluoroacetate) (Compound (XXX))

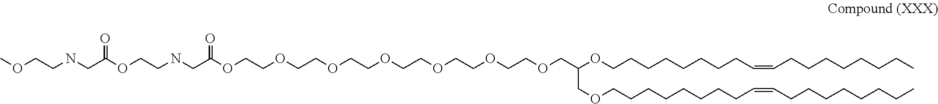

Compound (XXX) was synthesized based on the chemistry shown in the following Scheme.

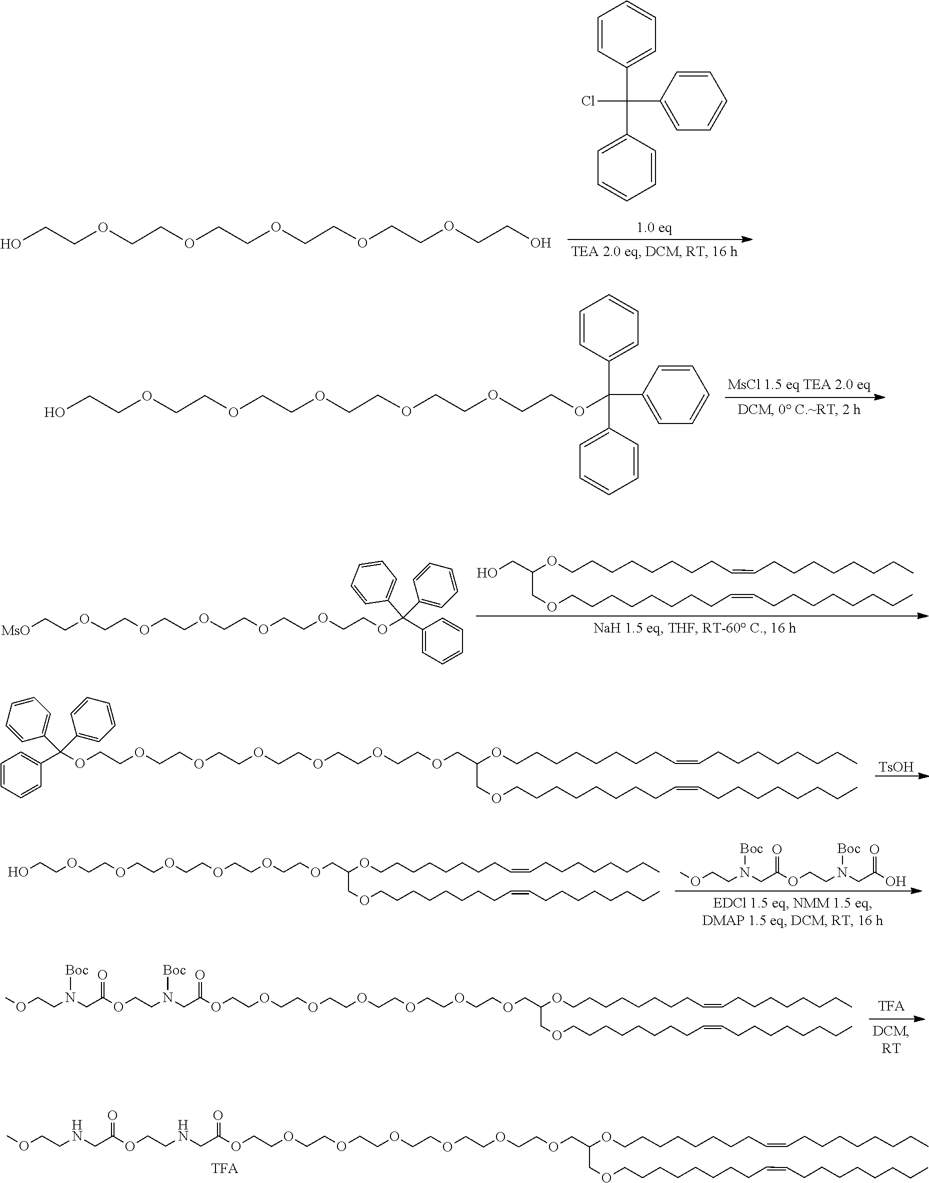

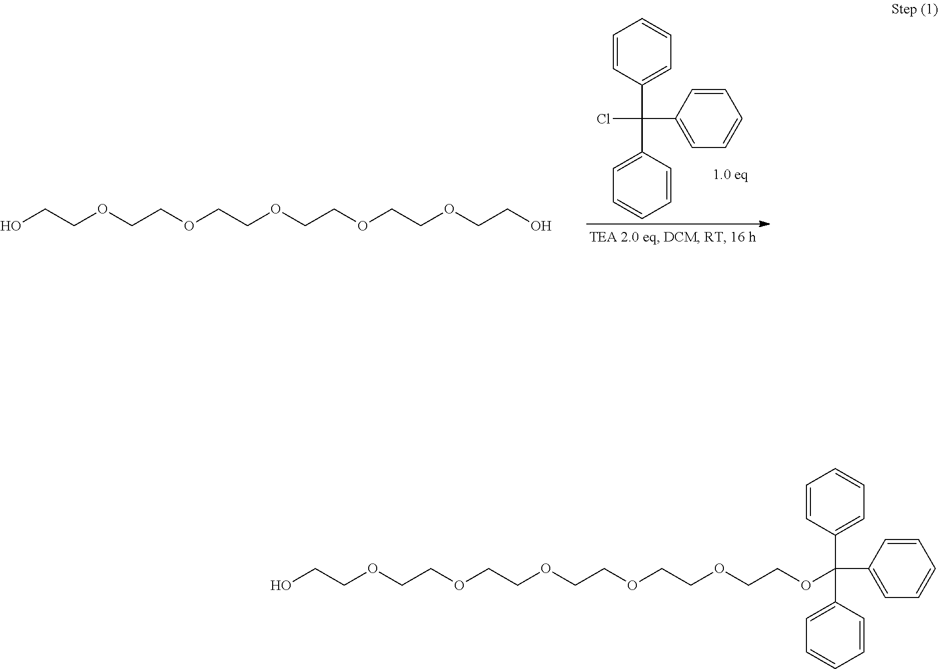 [SMILES: OCCOCCOCCOCCOCCOCCO ; ClC(c1ccccc1)(c1ccccc1)c1ccccc1 ; OCCOCCOCCOCCOCCOCCOC(c1ccccc1)(c1ccccc1)c1ccccc1]

A mixture of 2-[2-[2-[2-[2-(2-hydroxyethoxy)ethoxy]ethoxy]ethoxy]ethoxy]ethanol (5.0 g, 17.7 mmol) and N,N-diethylethanamine (3.58 g, 35.4 mmol) in DCM (100 mL) was added [chloro(diphenyl)methyl]benzene (4.94 g, 17.7 mmol). The mixture was stirred for 16 h at room temperature. The mixture was added DCM (100 mL) and washed with water, brine, dried over Na2SO4 and concentrated. The residue was purified by flash chromatography column on silica gel eluted with 0%-20% MeOH in DCM to afford 2-[2-[2-[2-[2-(2-trityloxyethoxy)ethoxy]ethoxy]ethoxy]ethoxy] ethanol (4.2 g, 45%) as colourless oil.

LCMS 542 (M+18), 99% UV:214 nm

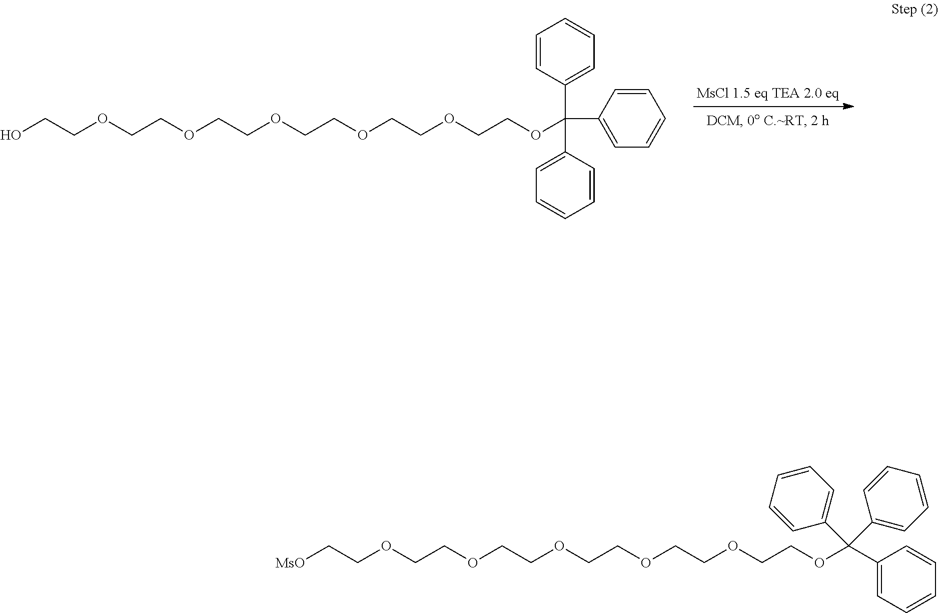 [SMILES: OCCOCCOCCOCCOCCOCCOC(c1ccccc1)(c1ccccc1)c1ccccc1 ; CS(=O)(=O)OCCOCCOCCOCCOCCOCCOC(c1ccccc1)(c1ccccc1)c1ccccc1]

To a mixture of 2-[2-[2-[2-[2-(2-trityloxyethoxy)ethoxy]ethoxy]ethoxy]ethoxy]ethanol (4.2 g, 8.01 mmol) and N,N-diethylethanamine (1.62 g, 16 mmol) in DCM (60 mL) cooled to 0° C. was added methanesulfonyl chloride (1.38 g, 12 mmol). The reaction mixture was stirred for 3 h at room temperature. The mixture was diluted with DCM (100 mL) and washed with water, brine, dired over Na2SO4 and concentrated to give 2-[2-[2-[2-[2-(2-trityloxyethoxy)ethoxy]ethoxy]ethoxy]ethoxy]ethyl methanesulfonate (4.6 g, 95.3%) as yellow oil.

LCMS 620 (M+18), 97% UV:214 nm

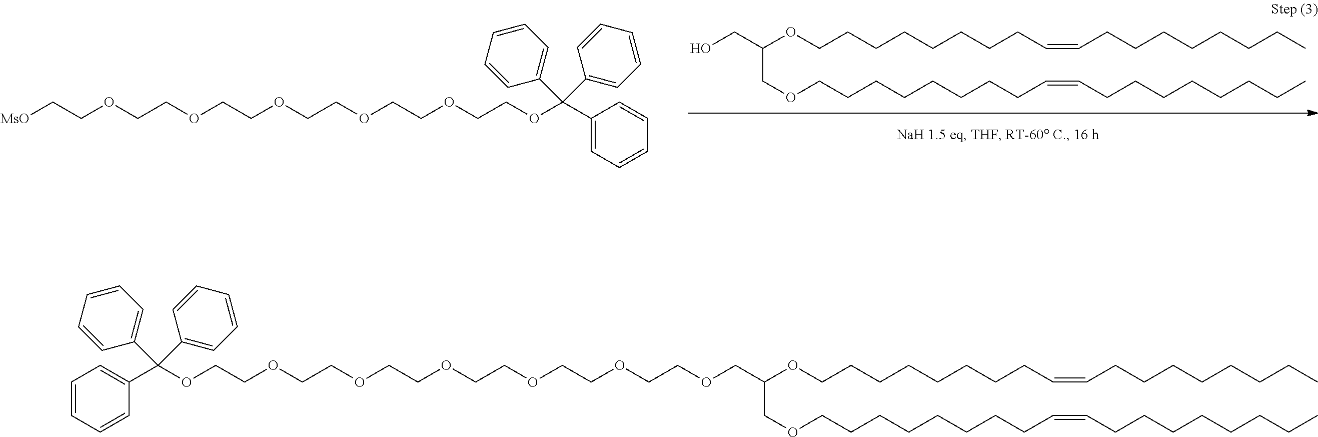 [SMILES: CS(=O)(=O)OCCOCCOCCOCCOCCOCCOC(c1ccccc1)(c1ccccc1)c1ccccc1 ; CCCCCCCC/C=C\CCCCCCCCOCC(CO)OCCCCCCCC/C=C\CCCCCCCC ; CCCCCCCC/C=C\CCCCCCCCOCC(COCCOCCOCCOCCOCCOCCOC(c1ccccc1)(c1ccccc1)c1ccccc1)OCCCCCCCC/C=C\CCCCCCCC]

To a suspension of NaH (60% mineral oil dispersion, 95 mg, 2.36 mmol) in 15 mL of THF was added 2,3-bis(((Z)-octadec-9-en-1-yl)oxy)propan-1-ol (0.7 g, 1.18 mmol) dissolved in 15 mL of THF. The resultant suspension was stirred for 2 h at room temperature. 2-[2-[2-[2-[2-(2-trityloxyethoxy)ethoxy]ethoxy]ethoxy]ethoxy]ethyl methanesulfonate (1.07 g, 1.77 mmol) was added to the suspension, and the reaction mixture was brought to reflux overnight. The reaction mixture was then cooled to room temperature, and water (40 mL) was added. The organic phase was collected and the aqueous phase was extracted with 3×40 ml EtOAc. The organic phases were combined and washed successively with 40 ml of 1N HCl, 40 ml of 5% (w/v) NaHCO₃ and 40 ml of brine and dried on MgSO4. The solvent was evaporated under reduced pressure and the resulting oil was purified on a silica gel column eluted with Petroleum ether/AcOEt (1:1) to give [2-[2-[2-[2-[2-[2-[2,3-bis[(Z)-octadec-9-enoxy]propoxy]ethoxy]ethoxy]ethoxy]ethoxy]ethoxy]ethoxy-diphenyl-methyl]benzene (700 mg, 48.5%) as a yellow oil.

1H NMR (500 MHz, CDCl3) δ 7.48-7.43 (m, 8H), 7.29 (dd, J=10.3, 4.8 Hz, 9H), 7.22 (t, J=7.3 Hz, 4H), 5.34 (t, J=5.2 Hz, 3H), 3.69-3.59 (m, 26H), 3.58-3.38 (m, 9H), 3.23 (t, J=5.2 Hz, 3H), 2.05-1.92 (m, 7H), 1.60-1.50 (m, 4H), 1.36-1.19 (m, 45H), 0.88 (t, J=6.9 Hz, 6H).

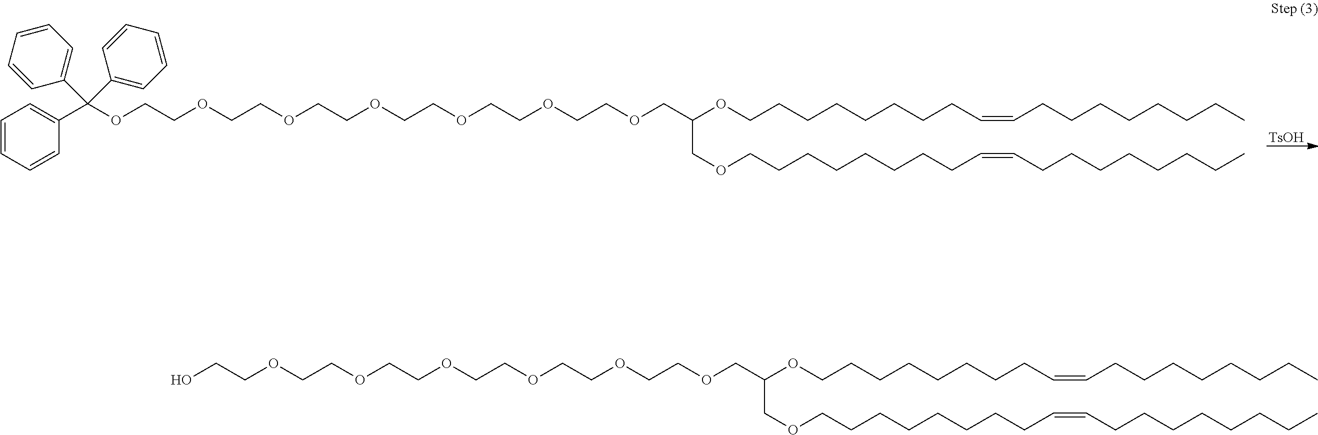

To a mixture of [2-[2-[2-[2-[2-[2-[2,3-bis[(Z)-octadec-9-enoxy]propoxy]ethoxy]ethoxy]ethoxy]ethoxy]ethoxy]ethoxy-diphenyl-methyl]benzene (0.7 g, 0.573 mmol) in THF (5 mL)/MeOH (5 mL) was added Toluene-4-sulfonic acid (0.545 g, 2.86 mmol) and stirred overnight at room temperature. Et3N (1 mL) was added to the reaction mixture and concentrated. The residue was purified by flash column chromatography on silica gel eluting with 2:1 ethyl acetate/petroleum ether to give 2-[2-[2-[2-[2-[2-[2,3-bis[(Z)-octadec-9-enoxy]propoxy]ethoxy]ethoxy]ethoxy]ethoxy]ethoxy]ethanol (0.5 g, 100%) as colourless oil.

1H NMR (400 MHz, CDCl3) δ 5.40-5.30 (m, 3H), 3.74-3.34 (m, 32H), 2.08-1.90 (m, 8H), 1.62-1.47 (m, 4H), 1.42-1.17 (m, 44H), 0.88 (t, J=6.8 Hz, 6H).

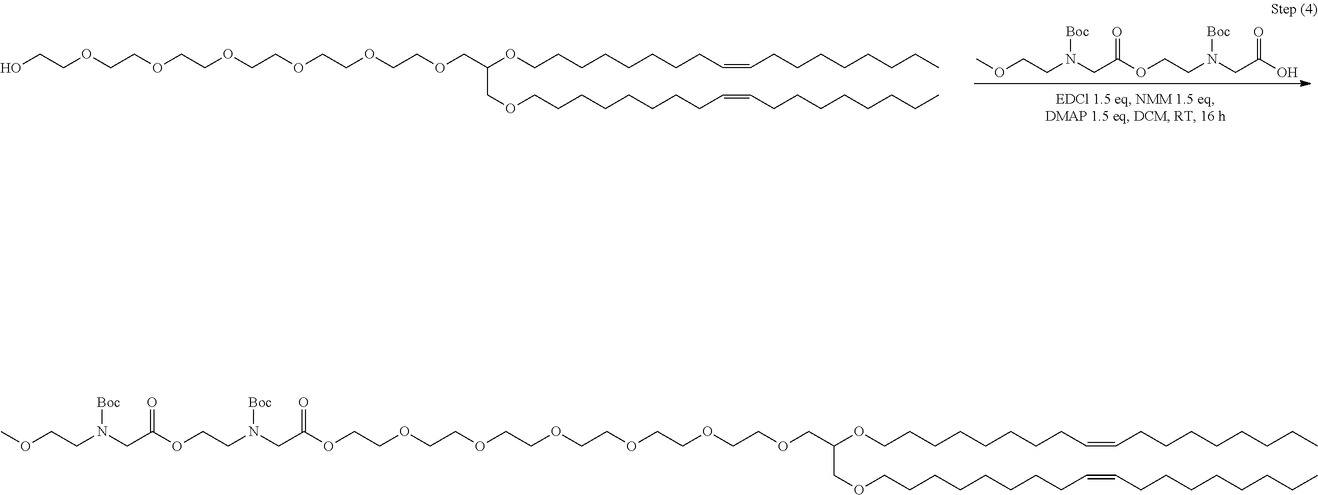

In a 100 mL round bottom flask, 2-[tert-butoxycarbonyl-[2-[2-[tert-butoxycarbonyl(2-methoxyethyl)amino]acetyl]oxyethyl]amino]acetic acid (400 mg, 0.921 mmol), 2-[2-[2-[2-[2-[2-[2,3-bis[(Z)-octadec-9-enoxy]propoxy]ethoxy]ethoxy]ethoxy]ethoxy]ethoxy]ethanol (789 mg, 0.921 mmol), N,N-dimethylpyridin-4-amine (169 mg, 1.38 mmol) were dissolved in DCM-Anhydrous (10 mL) in presence of 4-methylmorpholine (140 mg, 1.38 mmol). The reaction was cooled to 0° C. and EDC HCl (265 mg, 1.38 mmol) was added to the mixture. The reaction was warmed to room temperature and stirred overnight. Water was added and the organic layers were extracted with DCM (3×15 mL), washed with brine, dried over Na2SO4, filtered and concentrated. The residue was purified by flash column chromatography on silica gel eluted with 4:1 ethyl acetate/petroleum ether to give 2-[[2-[2-[2-[2-[2-[2-[2-[2,3-bis[(Z)-octadec-9-enoxy]propoxy]ethoxy]ethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.6 g, 51.2%) as colorless oil.

1H NMR (400 MHz, CDCl3) δ 5.40-5.31 (m, 3H), 4.36-4.17 (m, 4H), 4.06-3.94 (m, 4H), 3.72-3.61 (m, 22H), 3.58-3.39 (m, 15H), 3.30 (d, J=5.5 Hz, 3H), 2.05-1.94 (m, 7H), 1.79-1.70 (m, 3H), 1.61-1.50 (m, 4H), 1.44 (d, J=18.7 Hz, 18H), 1.38-1.19 (m, 45H), 0.88 (t, J=6.8 Hz, 6H).

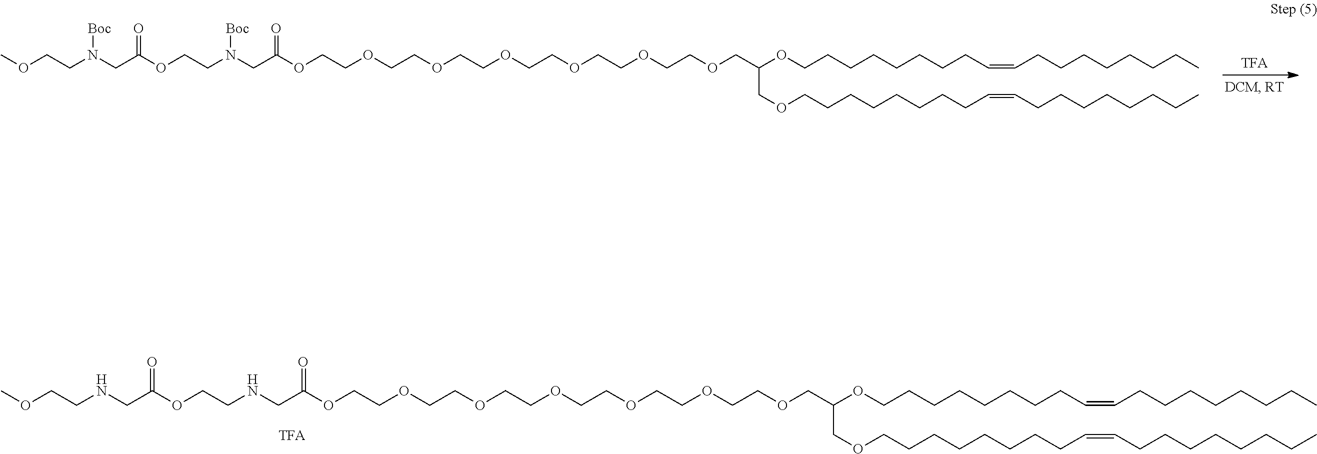

A mixture of 2-[[2-[2-[2-[2-[2-[2-[2-[2,3-bis[(Z)-octadec-9-enoxy]propoxy]ethoxy]ethoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]acetate (0.6 g, 0.471 mmol) in DCM (5 ml) was added TFA (0.269 g, 2.36 mmol). The mixture was stirred for 3 h at ambient temperature. The mixture was concentrated to give (2-[[2-[2-[2-[2-[2-[2-[2-[2,3-bis[(Z)-octadec-9-enoxy]propoxy]ethoxy]ethoxy]ethoxy]ethoxy]ethoxy]ethoxy]-2-oxo-ethyl]amino]ethyl 2-(2-methoxyethylamino)acetate;2,2,2-trifluoroacetic acid (0.433 g, 70.6%) as brown oil.

1H NMR (500 MHz, CDCl3) δ 5.38-5.30 (m, 3H), 4.58 (s, 2H), 4.37 (d, J=4.3 Hz, 2H), 4.05 (s, 2H), 4.00 (s, 2H), 3.76-3.42 (m, 36H), 3.40-3.30 (m, 5H), 2.05-1.91 (m, 6H), 1.54 (d, J=6.1 Hz, 4H), 1.38-1.19 (m, 46H), 0.88 (t, J=6.9 Hz, 6H).

### Example 28: Synthesis of 2-[[2-[2-[2-[2-[2-[2,3-bis[(Z)-octadec-9-enoxy]propoxy]ethoxy]ethoxy]ethoxy]ethoxy]-2-oxo-ethyl]amino]propyl 2-(2-methoxyethylamino)acetate;2,2,2-trifluoroacetic Acid (Compound (XXXI))

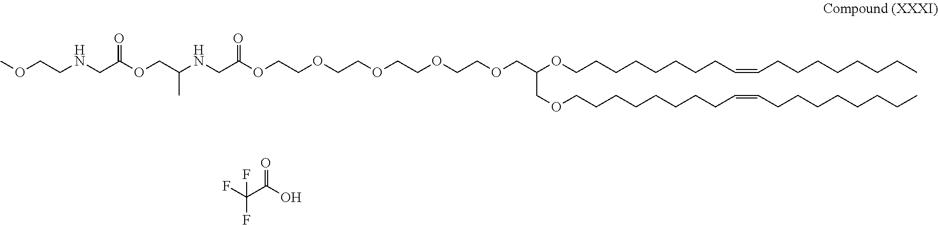 [SMILES: CCCCCCCC/C=C\CCCCCCCCOCC(COCCOCCOCCOCCOC(=O)CNC(C)COC(=O)CNCCOC)OCCCCCCCC/C=C\CCCCCCCC ; O=C(O)C(F)(F)F]

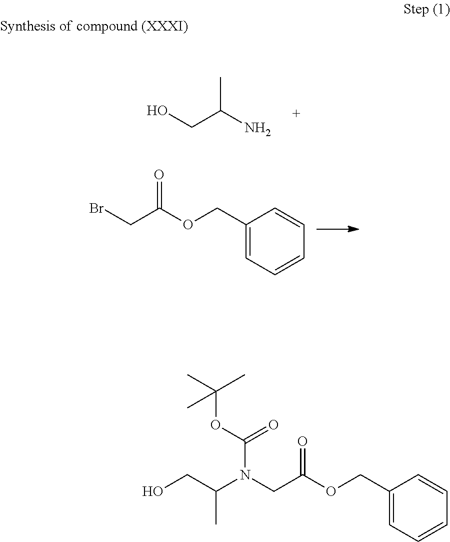

### Benzyl 2-[tert-butoxycarbonyl-(2-hydroxy-1-methyl-ethyl)amino]acetate

In a 250 ml round bottom three necked flask equipped with a thermometer and addition funnel, dry Potassium carbonate (9 g, 65.482 mmol) is suspended in acetonitrile (60 mL) under nitrogen. 2-aminopropan-1-ol (1.64 g 21.827 mmol) is then added and the mixture cooled to 0° C. in a sodium chloride/ice bath. Then a solution of benzyl 2-bromoacetate (5 g, 21.827 mmol) in acetonitrile (10 mL).

The reaction mixture is allowed to warm to 20° C. over a 6 h period and the mixture is filtered, the solids are washed with 2×20 ml acetonitrile and Di-tert-butyl pyrocarbonate (4.8 g, 22 mmol) is added to the filtrate and stirred for 16 h at 20° C.

Reaction mixture is concentrated under vacuum and the crude mixture purified by flash chromatography on a ISCO Redisep 220 g silica gel column using a Cyclohexane/ethyl acetate gradient.

5.2 g of the desired compound is obtained as an oil.

¹H NMR (CDCl3, 400 MHz) d(ppm): conformers 7.29 (m, 5H), 5.07 (m, 2H), 4.31 and 4.16 (2m, 1H), 3.99 and 3.81 (2m, 1H), 3.77-3.22 (m, 4H), 1.38 and 1.24 (2s, 9H), 0.963 (m, 3H)

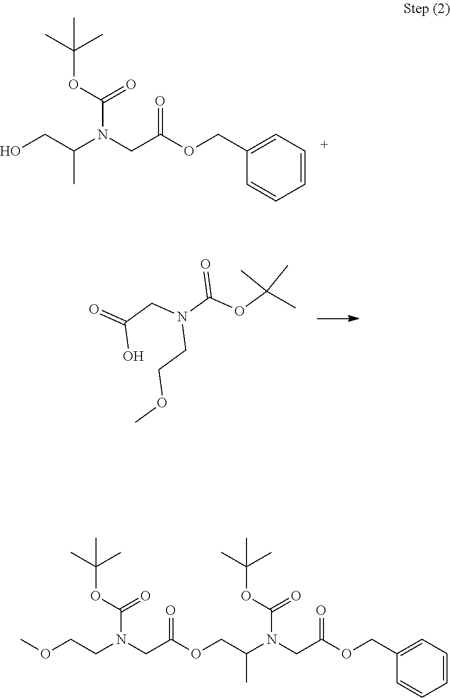 [SMILES: CC(CO)N(CC(=O)OCc1ccccc1)C(=O)OC(C)(C)C ; COCCN(CC(=O)O)C(=O)OC(C)(C)C ; COCCN(CC(=O)OCC(C)N(CC(=O)OCc1ccccc1)C(=O)OC(C)(C)C)C(=O)OC(C)(C)C]

### 2-[(2-benzyloxy-2-oxo-ethyl)-tert-butoxy-carbonyl-amino]propyl 2-[tert-butoxy-carbonyl(2-methoxyethyl)amino] Acetate

In a 100 mL round bottomed flask, benzyl 2-[tert-butoxycarbonyl-(2-hydroxy-1-methyl-ethyl)amino]acetate (1.49 g, 4.61 mmol) and 2-([(tert-butoxy)carbonyl](2-methoxy ethyl)amino)acetic acid (1.13 g, 4.61 mmol) are dissolved in dichloromethane (8 mL) under nitrogen. N-methyl morpholine (1 ml, 9.21 mmol) and 4-(dimethylamino)-pyridine (1.1 g, 9.21 mmol) are then added and the mixture cooled to 0° C. in an ice bath. Then 1-éthyl-3-(3-diméthylaminopropyl)carbodiimide (1.7 g, 9.21 mmol) is added by portions and the mixture stirred over-night. The reaction mixture is the diluted with dichloromethane (30 ml), washed with water (20 ml) and brine (20 ml). After drying over NaSO₄ and filtration the concentrated crude product is purified by flash chromatography on a ISCO Redisep 80 g silica gel column using a Dichloromethane/methanol (0% to 5% methanol gradient in 35 min. 50 ml/min).

2 g of the desired compound is obtained as an oil.

¹H NMR (CDCl3, 400 MHz) d(ppm): conformers 7.25 (m, 5H), 5.06 (s, 2H), 4.41 and 4.18 (2m, 1H), 4.14-3.70 (b m, 7H), 3.66 (b m, 6H), 3.19 (b m, 3H), 1.34-1.13 (4s, 18H), 1.05 (m, 3H)

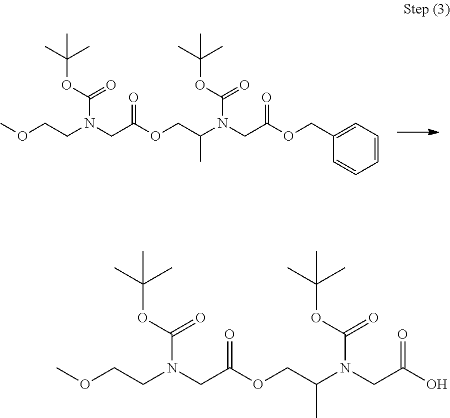 [SMILES: COCCN(CC(=O)OCC(C)N(CC(=O)OCc1ccccc1)C(=O)OC(C)(C)C)C(=O)OC(C)(C)C ; COCCN(CC(=O)OCC(C)N(CC(=O)O)C(=O)OC(C)(C)C)C(=O)OC(C)(C)C]

### 2-[tert-butoxycarbonyl-[2-[2-[tert-butoxycarbonyl(2-methoxyethyl)amino]acetyl]oxy-1-methyl-ethyl]amino]acetic Acid

In a 100 mL round bottomed flask, 2-[(2-benzyloxy-2-oxo-ethyl)-tert-butoxy carbonyl-amino]propyl 2-[tert-butoxy carbonyl(2-methoxyethyl)amino]acetate (2 g, 3.713 mmol) and ammonium formate (700 mg, 11 mmol) are dissolved in abs. ethanol (40 ml) and the resulting solution is degassed with nitrogen. Then 5% Pd/C (400 mg) are added under nitrogen and the mixture is refluxed for 3 h.

The reaction mixture is then filtered on Decalite™ and concentrated under vacuum. The residual oil is taken up in dichloromethane (40 ml) and washed with 0.1 M hydrochloric acid (10 ml), brine and dried over NaSO₄, filtered and concentrated.

1.5 g of the desired compound is obtained as an oil.

¹H NMR (DMSO,d6, 400 MHz) d(ppm): conformers 12.46 (s, 1H), 4.36 and 4.19 (2m, 1H), 4.14-3.70 (b m, 7H), 3.45-3.35 (b m, 6H), 3.20 (2s, 3H), 1.34-1.13 (4s, 18H), 1.08 and 1.03 (2d, 3H)

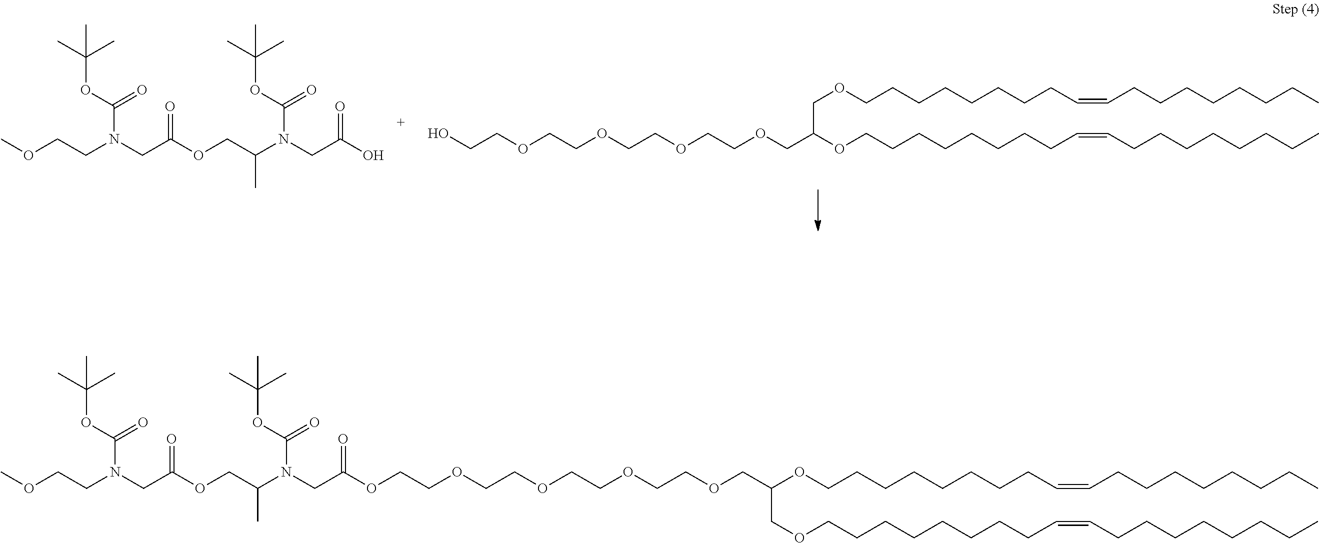

### 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]propyl 2-[tert-butoxycarbonyl(2-methoxyethyl)amino]acetate

In a 50 mL round bottomed flask, 2-[2-[2-[2-[2,3-bis[(Z)-octadec-9-enoxy] propoxy]ethoxy] ethoxy] ethoxy] ethanol (300 mg, 0.3900 mmol) and 2-[tert-butoxycarbonyl-[2-[2-[tert-butoxycarbonyl(2-methoxyethyl)amino]acetyl]oxy-1-methyl-ethyl]amino]acetic acid (20ç mg, 0.4680 mmol) are dissolved in dichloromethane (5 mL) under nitrogen. N-methyl morpholine (0.043 ml, 0.39 mmol) and 4-(dimethylamino)-pyridine (47 mg, 0.39 mmol) are then added and the mixture cooled to 0° C. in an ice bath. Then 1-éthyl-3-(3-diméthylaminopropyl)carbodiimide (112 mg, 0.585 mmol) is added by portions and the mixture stirred over-night. The reaction mixture is the diluted with dichloromethane (30 ml), washed with water (20 ml) and brine (20 ml). After drying over NaSO₄ and filtration the concentrated crude product is purified by flash chromatography on a ISCO Redisep 80 g silica gel column using a Dichloromethane/methanol (0% to 5% methanol gradient in 35 min. 50 ml/min) and then with a dichlorometane/ethyl acetate gradient (0% AcOEt to 100% AcOEt in 35 min. 50 ml/min).

240 mg of the desired compound is obtained as an oil.

)

¹H NMR (CDCL3, 400 MHz) d(ppm): conformers 5.35 (m, 4H, vinylic protons), 4.51 and 4.19 (2m, 1H), 4.26 (m, 2H) 4.2-3.60 (b m, 7H), 3.6-3.35 (b m, 22H), 3.31 (2s, 3H), 2.02 (m, 8H allylic protons), 1.6-1.18 (m, 73H), 0.87 (t, 6H)

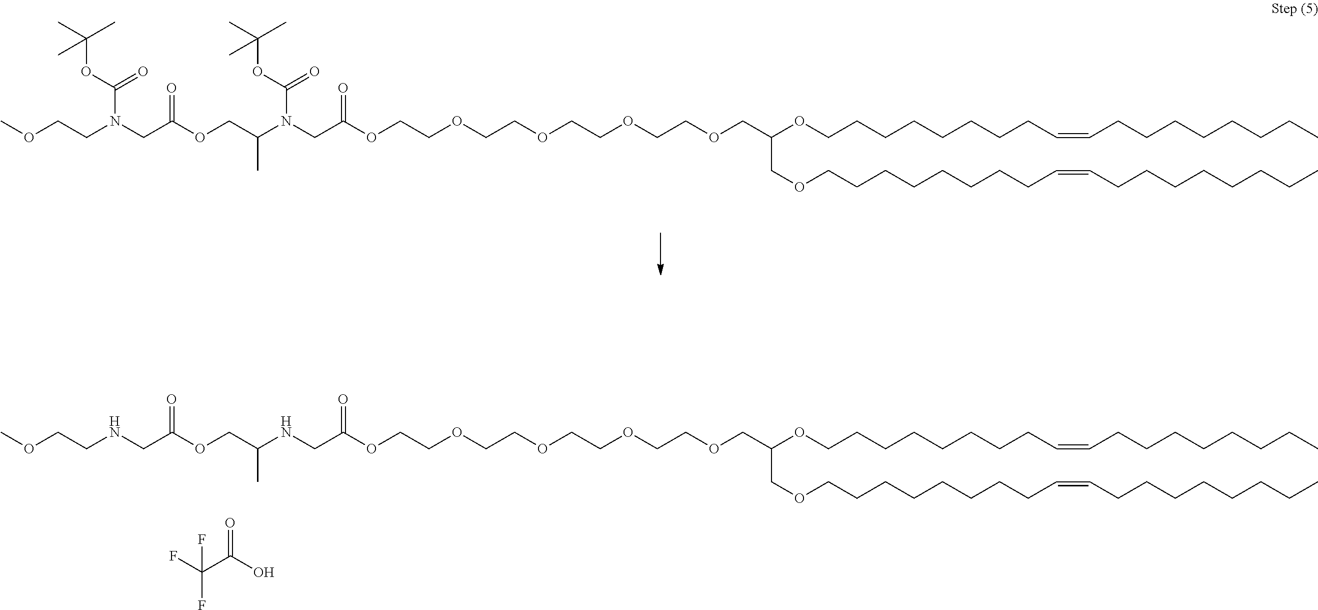

In a 50 mL round bottom flask, 2-[[2-[2-[2-[2-[2-[2,3-bis[(Z)-octadec-9-enoxy]propoxy] ethoxy] ethoxy]ethoxy]ethoxy]-2-oxo-ethyl]-tert-butoxy carbonyl-amino] propyl 2-[tert-butoxy carbonyl (2-methoxyethyl)amino] acetate (240 mg, 0.20 mmol) is dissolved in dichloromethane (5 mL). The mixture is cooled to 0° C. under nitrogen and TFA (0.5 ml) is added. The mixture is stirred between 0° C. and 20° C. overnight and concentrated under vacuum.

The crude product is purified by reverse phase chromatography on a Phenomenex Jupiter PFP column (5 μM, 250×5 mm).
Solvent A: H₂O/CH₃CN 95:5
Solvent B: Acetonitrile
Solvent C: 1% TFA in H2O
Gradient: t=0 to t=3 min, isocratic 10% A, 70% B, 20% C, then linear gradient to 100% B in 16 min (t=19 min). from t=19 min to t=35 min 100% B.
Flow: 100 ml/min/P=116 bars

The fractions containing the pure compound are concentrated under vacuum at T<30° C. and then lyophilized to give an oil of 2-[[2-[2-[2-[2-[2-[2,3-bis[(Z)-octadec-9-enoxy]propoxy]ethoxy]ethoxy]ethoxy]ethoxy]-2-oxo-ethyl]amino]propyl 2-(2-methoxyethylamino)acetate;2,2,2-trifluoroacetic acid

m=100 mg (40% yield of the bis TFA salt)

¹H NMR (DMSO d6, 400 MHz) d(ppm): 9.2 (b, 4H), 5.3 (m, 4H), 4.42 (m, 2H), 4.31 (m, 2H), 4.2 (m, 2H), 3.6-3.1 (b m, 29H), 2.02 (m, 8H), 1.4 (m, 4H)-1.30 (m, 51H), 0.82 (t, 6H)

### Example 29: Synthesis of 2-[[2-[2-[2-[2-[2-[2,3-bis[(Z)-octadec-9-enoxy]propoxy]ethoxy]ethoxy]ethoxy]ethoxy]-2-oxo-ethyl]amino]propyl 2-(2-methoxyethylamino)acetate;2,2,2-trifluoroacetic Acid (Compound (XXXII))

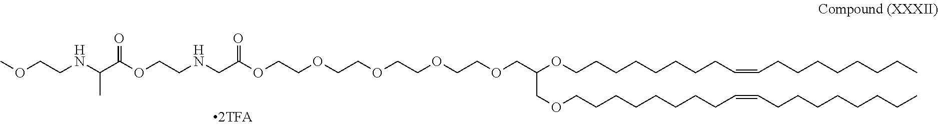 [SMILES: CCCCCCCC/C=C\CCCCCCCCOCC(COCCOCCOCCOCCOC(=O)CNCCOC(=O)C(C)NCCOC)OCCCCCCCC/C=C\CCCCCCCC]

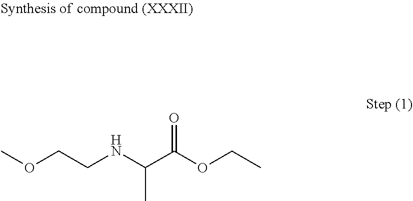

### Ethyl 2-(2-methoxyethylamino)propanoate

Ethyl 2-bromoacetate (650 μL, 4.9 mmol) in Acetonitrile (5 ml) was added dropwise to the solution of 2-aminoethanol (440 μL, 5.01 mmol) and Triethylamine (700 μL, 5.00 mmol) in Acetonitrile (20 ml) at 0° C. over 1 h period. The mixture was stirred 30 min at 0° C. and 20 h at room temperature. The solution is evaporated under reduced pressure and the resulting slurry suspended in dichloromethane. The precipitate is filtrated and the filtrate evaporated under reduced pressure. The crude product is purified by flash chromatography on 40 g silica gel column (eluted with 0%→10% methanol in dichloromethane) to give ethyl 2-(2-methoxyethylamino)propanoate as a colourless oil (452 mg 2.58 mmol Y=51%).

1H NMR (CDCl3, 400 MHz) d 4.17 (q, J=7.1 Hz, 2H), 3.51-3.43 (m, 2H), 3.34 (q, J=7 Hz, 1H), 3.34 (s, 3H), 2.84-2.78 (m, 1H), 2.68-2.62 (m, 1H), 1.82 (s, 1H), 1.30 (d, J=7.0 Hz, 3H), 1.26 (t, J=7.1 Hz, 3H).

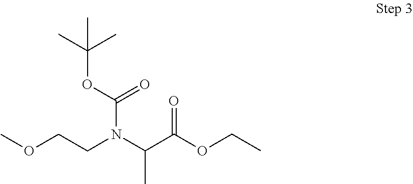 [SMILES: CCOC(=O)C(C)N(CCOC)C(=O)OC(C)(C)C]

### Ethyl 2-[tert-butoxycarbonyl(2-methoxyethyl)amino]propanoate

To the solution of ethyl 2-(2-methoxyethylamino)propanoate (783 mg, 4.47 mmol) in Dichloromethane (19 mL) cooled to 0° C. are added Triethylamine (630 μL, 4.5 mmol) and DMAP (55.9 mg-0.45 mmol). A solution of Boc2O (1.209 g-5.43 mmol) in Dichloromethane (3 mL) is then added over a 10 min period. After 7 h30 at room temperature, Boc2O (306 mg-1.37 mmol) in Dichloromethane (1 ml) is added. After overnight stirring, the mixture is evaporated under reduced pressure and the residue is purified by flash chromatography on 40 g silica gel column (eluted with 0%→5% methanol in dichloromethane) to give ethyl 2-[tert-butoxycarbonyl(2-methoxyethyl)amino]propanoate as a colourless oil (792 mg−2.88 mmol−Y=64%)

1H NMR (DMSO-d6, 400 MHz, rotamers at ambient temperature: spectra recorded at 90° C.) d 4.19 (q, J=7 Hz, 1H), 4.08 (q, 2H, J=7 Hz), 3.46-3.40 (m, 2H), 3.36-3.30 (m, 2H), 3.25 (s, 3H), 1.37 (s, 9H), 1.35 (d, 3H, J=7 Hz), 1.19 (t, 3H, J=7 Hz).

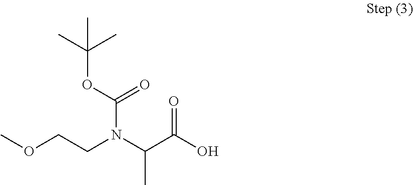 [SMILES: COCCN(C(=O)OC(C)(C)C)C(C)C(=O)O]

### 2-[tert-butoxycarbonyl(2-methoxyethyl)amino]propanoic Acid

To the solution of ethyl 2-[tert-butoxycarbonyl(2-methoxyethyl)amino]propanoate (198 mg, 0.72 mmol) in Methanol (5.4 mL) cooled to 0° C. is added a 2M solution of Lithium Hydroxide Hydrate in Water (1.8 mL-3.6 mmol). The solution is stirred 30 min at 0° C. and 7 h at room temperature.

The pH is then decreased to 3-4 by addition of DOWEX 50W X8 acidic resin. The resin is filtrated and the filtrate partially evaporated under reduced pressure. The resulting slurry is extracted by Dichloromethane (×3). The organic phases are dried over Na2SO4 and filtrated. The filtrate is evaporated under reduced pressure to yield 2-[tert-butoxycarbonyl(2-methoxyethyl)amino]propanoic acid as a colourless oil (184 mg−0.73 mmol−Y=quant)

1H NMR (DMSO-d6, 400 MHz) Rotamers at ambient temperature d 12.41 (sl, 0.8H), 4.25 (m, 0.4H), 4.04 (m, 0.6H) 3.49-3.24 (m+H2O, 4H), 3.23 (s+H2O, 3H), 1.42-1.27 (m, 12H).

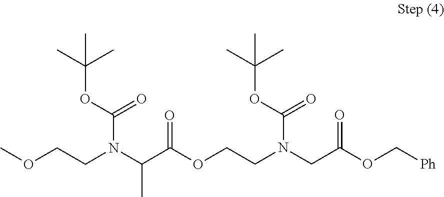 [SMILES: COCCN(C(=O)OC(C)(C)C)C(C)C(=O)OCCN(CC(=O)OCc1ccccc1)C(=O)OC(C)(C)C]

### 2-[(2-benzyloxy-2-oxo-ethyl)-tert-butoxycarbonyl-amino]ethyl 2-[tert-butoxycarbonyl(2-methoxyethyl)amino]propanoate

To a solution of 2-[tert-butoxycarbonyl(2-methoxyethyl)amino]propanoic acid (170 mg-0.674 mmol) in Dichloromethane (5 mL) are added DMAP (82 mg-0.665 mmol) and 3-(ethyliminomethylamino)-N,N-dimethyl-propan-1-amine;hydrochloride (131.5 mg-0.672 mmol). The mixture is stirred 5 min then a solution of benzyl 2-[tert-butoxycarbonyl(2-hydroxyethyl)amino]acetate (197.7 mg-0.639 mmol) in Dichloromethane (1.4 mL) is added. After 3 h30 at room temperature, the reaction mixture is evaporated under reduced pressure. The resulting slurry is purified by flash chromatography on 24 g silica gel column (eluted with 0%→5% methanol in dichloromethane) to give 2-[(2-benzyloxy-2-oxo-ethyl)-tert-butoxycarbonyl-amino]ethyl 2-[tert-butoxycarbonyl(2-methoxyethyl)amino]propanoate as a colourless oil (253 mg−0.470 mmol−Y=73%)

1H NMR (DMSO-d6, 400 MHz) d 7.40-7.32 (m, 5H), 5.15 (d, 2H, J=8.8 Hz), 4.29-3.97 (m, 5H), 3.47 (m, 2H), 3.39 (m, 2H), 3.29 (m, 2H+H2O), 3.23 (s, 3H), 1.45-1.16 (m, 21H).

)

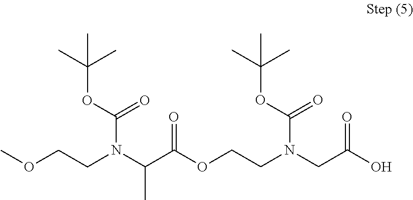 [SMILES: COCCN(C(=O)OC(C)(C)C)C(C)C(=O)OCCN(CC(=O)O)C(=O)OC(C)(C)C]

### 2-[tert-butoxycarbonyl-[2-[2-[tert-butoxycarbonyl(2-methoxyethyl)amino]propanoyl oxy]-ethyl]amino]acetic Acid

To the solution of 2-[(2-benzyloxy-2-oxo-ethyl)-tert-butoxycarbonyl-amino]ethyl 2-[tert-butoxycarbonyl(2-methoxyethyl)amino]propanoate (236 mg-0.438 mmol) in ethyl acetate (15 mL) is added 10% Pd/C (45.8 mg-0.043 mmol). The mixture is stirred under 50 psi of hydrogen for 5 h30. TLC indicated that all the starting material has been consumed. The mixture is filtrated to remove the catalyst. The filtrate is evaporated under reduced pressure to give 2-[tert-butoxycarbonyl-[2-[2-[tert-butoxycarbonyl(2-methoxyethyl)amino]propanoyl-oxy]ethyl]amino]acetic acid as a colourless oil (211 mg).

1H NMR (DMSO-d6, 400 MHz, large signals at room temperature: spectra recorded at 110° C.) d 4.29-4.20 (m, 1H), 4.20-4.05 (m, 2H), 3.87 (s, 2H), 3.51-3.43 (m, 4H), 3.39-3.33 (m, 2H), 3.28 (s, 3H), 1.44-1.39 (m, 211H).

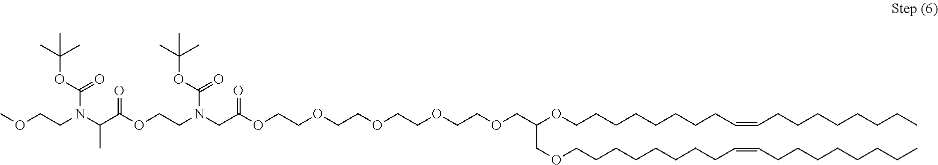 [SMILES: CCCCCCCC/C=C\CCCCCCCCOCC(COCCOCCOCCOCCOC(=O)CN(CCOC(=O)C(C)N(CCOC)C(=O)OC(C)(C)C)C(=O)OC(C)(C)C)OCCCCCCCC/C=C\CCCCCCCC]

### 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl) amino]propanoate

To a solution of 2-[tert-butoxycarbonyl-[2-[2-[tert-butoxycarbonyl(2-methoxyethyl)amino]propanoyloxy]ethyl]amino]acetic acid (145.2 mg-0.291 mmol) in Dichloromethane (1.5 mL) are added DMAP (36.2 mg-0.293 mmol) and 3-(ethyliminomethylamino)-N,N-dimethyl-propan-1-amine;hydrochloride (57.8 mg-0.295 mmol). The mixture is stirred 5 min then a solution of 2-[2-[2-[2-[2,3-bis[(Z)-octadec-9-enoxy]propoxy]ethoxy]ethoxy]ethoxy]ethanol (190.1 mg-0.246 mmol) in Dichloromethane (1 mL) is added. After 24 h at room temperature, 2-[tert-butoxycarbonyl-[2-[2-[tert-butoxycarbonyl(2-methoxyethyl)amino]propanoyloxy]ethyl]amino]acetic acid (14.7 mg 0.030 mmol) in Dichloromethane (400 μL), DMAP (3.9 mg-0.032 mmol) and 3-(ethyliminomethylamino)-N,N-dimethyl-propan-1-amine;hydrochloride (6.1 mg-0.031 mmol) are added. After 17 h at room temperature, the mixture is evaporated under reduced pressure. Two flash chromatographies on 24 g silica gel columns are necessary to purify the residue: first chromatography eluted with 0%→5% methanol in dichloromethane and second chromatography eluted with 0%→50% Ethyl acetate in heptane. 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]propanoate is isolated as a colourless oil (68.7 mg−0.057 mmol−Y=23%)

1H NMR (DMSO-d6, 500 MHz) Rotamers at ambient temperature d 5.37-5.31 (m, 0.6H), 5.31 (t, 3.4H), 4.32-4.00 (m, 5H), 4.00-3.94 (m, 2H), 3.62-3.59 (m, 2H), 3.56-3.31 (m, 26H), 3.24 (s, 3H), 2.02-1.91 (m, 8H), 1.50-1.41 (m, 4H), 1.41-1.20 (m, 65H), 0.85 (t, 7H).

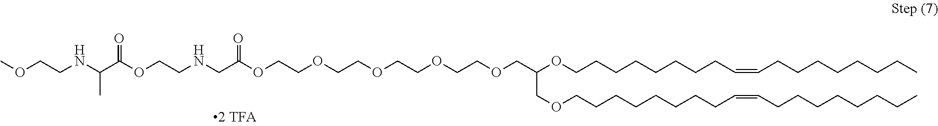 [SMILES: CCCCCCCC/C=C\CCCCCCCCOCC(COCCOCCOCCOCCOC(=O)CNCCOC(=O)C(C)NCCOC)OCCCCCCCC/C=C\CCCCCCCC]

### 2-[[2-[2-[2-[2-[2-[2,3-bis[(Z)-octadec-9-enoxy]propoxy]ethoxy]ethoxy]ethoxy]ethoxy]-2-oxo-ethyl]amino]ethyl 2-(2-methoxyethylamino)propanoate;2,2,2-trifluoroacetic acid

Trifluoroacetic acid (120 μL-1.56 mmol) is added to a solution of 2-[[2-[2-[2-[2-[2-[2,3-bis[(Z)-octadec-9-enoxy]propoxy]ethoxy]ethoxy]ethoxy]ethoxy]-2-oxo-ethyl]-tert-butoxycarbonyl-amino]ethyl 2-[tert-butoxycarbonyl(2-methoxyethyl)amino]propanoate (93 mg-77.4 μmol) in Dichloromethane (1 mL). After 5 h at room temperature, the reaction mixture is evaporated under reduced pressure. The crude product is purified by reverse phase chromatography on a Phenomenex Jupiter PFP column (5 μM, 250×5 mm) (Solvent A: H₂O/CH₃CN 95:5; Solvent B: Acetonitrile; Solvent C: 1% TFA in H2O; Gradient: t=0 to t=3 min, isocratic 10% A, 70% B, 20% C, then linear gradient to 100% B in 16 min (t=19 min). from t=19 min to t=35 min 100% B; Flow: 100 ml/min/P=116 bars)

The fractions containing the pure product are concentrated under reduced pressure at T<30° C. to yield 2-[[2-[2-[2-[2-[2-[2,3-bis[(Z)-octadec-9-enoxy]propoxy]ethoxy]ethoxy]-ethoxy]ethoxy]-2-oxo-ethyl]amino]ethyl 2-(2-methoxyethylamino)propanoate;2,2,2-trifluoroacetic acid as a colourless wax (46 mg-0.037 mmol-48%)

1H NMR (DMSO-d6, 500 MHz) d 9.23 (br s, 1.8H), 5.38-5.28 (m, 4H), 4.44-4.38 (m, 2H), 4.33-4.27 (m, 2H), 4.15 (t, J=7 Hz, 1H), 4.10-4.03 (m, 2H), 3.68-3.64 (m, 2H), 3.62-3.58 (m, 2H), 3.58-3.45 (m+H2O, 19H), 3.44-3.38 (m+H2O, 12H), 3.18 (t, 3H), 2.03-1.90 (m, 8H), 1.52-1.40 (m, 8H), 1.37-1.19 (m, 49H), 0.86 (t, 7H).

### Example 30: Lipid Nanoparticles (LNPs) Preparation

Preparation of the Organic Solvent

Lipids were dissolved in ethanol at molar ratios of 50/10/38.5/1.5 or 35/2.5/46.5/16 (ionizable lipid:neutral lipid:cholesterol:PEGylated lipid)

PEGylated lipid may be PEG2000-PE or DMG-PEG2000.

Neutral lipid may be DPSC or DOPE.

Two ionizable cationic lipidic compounds were used to manufacture the LNPs: L319 (DLin-MC3-DMA) and lipidic compound of formula (III) (or DOG-CLEAVE).

Preparation of 1.5 mL of Organic Solvent for L319-Containing LNPs (LNPs 319)

Formulation 3.9 mg of DSPC (1,2-distearoyl-sn-glycero-3-phosphocholine—Avanti Polar Lipids: 850365), 2 mg of PEG2000-PE (1,2-dimyristoyl-sn-glycero-3-phosphoethanolamine-N-[methoxy(polyethylene glycol)-2000] (ammonium salt)—Avanti Polar Lipids: 880150P) and 7.4 mg of cholesterol (Sigma Aldrich: C3045) were dissolved with 1319 μL of ethanol. Then 167 μL of L319 stock solution (100 mg/mL in ethanol—DLin-MC3-DMA—Maier et al., Molecular Therapy, 2013, 21 (8): 1570-1578) was added to obtain 20 mg/mL of lipid phase solution.

In some preparations PEG2000-PE was replaced with DMG-PEG.

Preparation of 0.7 mL of Organic Solvent for Lipidic Compound of Formula (III)-Containing LNPs (LNPs (III))

15 μL of an ethanolic solution of DSPC at 100 mg/mL, 7 μL of an ethanolic solution of PEG2000-PE at 100 mg/mL and 2.7 mg of cholesterol were mixed and added with 604 μL of ethanol. 91 μL of lipidic compound of formula (III) stock solution at 100 mg/ml were added to obtain the final 20 mg/mL lipid mix solution.

In some preparations PEG2000-PE was replaced with DMG-PEG.

In some preparations DSPC was replaced with DOPE.

Preparation of the Aqueous Solvent

Preparation of 1.8 mL of Aqueous Phase for LNPs 319

A non-replicative mRNA encoding A/Netherlands HA (SEQ ID NO: 1) was used. The influenza HA mRNA was produced by in vitro transcription (IVT) as an unmodified mRNA transcript from a linear DNA template generated by PCR, using wild type bases and T7 RNA polymerase (Avci-Adali et al (J. Vis. Exp. (93), e51943, doi:10.3791/51943 (2014) and Kwon et al., Biomaterials 156 (2018) 172e193). The mRNA was 3′ polyadenylated (A120) and 5′ capped (Cap 1).

After Dnase and phosphatase treatment, the mRNA was purified to high degree of purity by silica membrane filtration followed by HPLC. mRNA was packaged as 1 mL aliquots of 2 mg/mL solution in 1 mM Sodium Citrate, pH 6.4.

mRNA concentration to be used in aqueous phase was calculated to obtain a cationic amino group/anionic phosphate group ratio of 6 (N/P=6). This concentration was determined from the cationic lipid concentration assuming 1 μg mRNA corresponds to 0.003 pmol of phosphate. Since 1.5 mL of aqueous solution is needed to make 2 mL of LNPs when using a ratio of aqueous solution to ethanol solution of 3:1 with the NanoAssemblR® (Nanoassemblr Benchtop from Precision Nanosystem; Belliveau et al., Molecular Therapy-Nucleic Acids (2012)), the required mRNA concentration was calculated to be 305 μg/mL.

The mRNA solution was prepared in 50 mM citrate buffer pH 4.0.

Preparation of 1.8 mL of Aqueous Phase for LNPs Lip. (III) (Lipidic Compound of Formula (III)-Containing LNPs)

mRNA was prepared as above described and the calculated concentration was 0.243 mg/ml to obtain a cationic amino group/anionic phosphate group ratio of 12 (N/P=12) by considering that lipidic compound of formula (III)contains two positively charged amines at pH 4.0.

The mRNA solution was prepared in 50 mM citrate buffer pH 4.0.

In some preparations, the amount of mRNA was adjusted to have a cationic amino group/anionic phosphate group ratio of 6 (N/P=6) or 10 (N/P=10).

LNPs Preparation

LNPs were prepared using a NanoAssemblR equipment according to manufacturer recommendations.

The aqueous and organic phases were each loaded in a syringe suitable for NanoAssemblR according to manufacturer recommendations. The flow rate was set up at a ratio: 3:1 and total flow rate: 4 ml/min. The aqueous and lipid phases were then mixed to obtain the LNPs.

LNPs L319 Purification and Harvest

The obtained LNPs were dialyzed against a citrate buffer (50 mM-pH 4.0) to remove residual ethanol and then twice against a PBS buffer (pH 7.4). Each dialysis was carried out at least during 12 hours at 4° C. The LNPs were then filtered through a 0.22 μm filter and store under nitrogen at +4° C.

LNPs Lip. (III) Purification and Harvest

The obtained LNPs were dialyzed against a citrate buffer (50 mM-pH 4.0) to remove residual ethanol and then twice against a citrate buffer (10 mM-pH 6.3) containing sucrose (8%). Each dialysis was carried out at least during 12 hours at 4° C. The LNPs were then filtered through a 0.22 μm filter and store under nitrogen at +4° C.

The dialysis against an aqueous solvent at pH 6.3 allowed the hydrolysis and self-rearrangement of the ionizable lipid as disclosed herein into a neutral lipid and the removal of the cationic polar head. The zeta potential of the obtained LNPs is therefore closed to 0 mV.

### Example 31: Characterization of LNPs

RNA Titration/Encapsulation Rate

The percentage of encapsulated mRNA and concentration of mRNA in LNPs were measured using the Quant-iT Ribogreen RNA reagent kit according to manufacturer recommendations (Invitrogen Detection Technologies) and quantified with a fluorescent microplate reader or a standard spectrophotometer using fluorescein excitation and emission wavelength.

For quantification of non-encapsulated RNA, LNPs were diluted in Tris/EDTA assay buffer (10 mM Tris-HCl, 1 mM EDTA, pH 7.5).

For quantification of total amount of RNA, LNPs were diluted in Tris/EDTA assay buffer (10 mM Tris-HCl, 1 mM EDTA, pH 7.5) containing 0.5% (v/v) Triton X100.

Ribogreen dye (200× diluted) was added to the samples (50/50 mix; sample/Ribogreen reagent), mixed thoroughly and incubated 5 min at room temperature in the dark. Fluorescence was measured on the plate reader.

Lipid Quantification

It was assumed that there was no loss of lipids during the formulation process. The total lipid concentration before dialysis was then assumed to be 5 mg/mL. The final lipid concentration was defined by taking into account the dilution factor occurring during the dialysis step.

Particle Size Distribution, Polydispersity Index Zeta Potential, and Osmolarity,

Zeta potential and particle size distribution of LNPs were measured by using a zeta sizer Nano ZS light scattering instrument (Malvern Instruments) according to manufacturer recommendations. Particle sizes were reported as the Z-average size (harmonic intensity averaged particle diameter) along with the Polydispersity Index (PDI), an indicator of the “broadness” of the particle size distribution. Samples were diluted to 1/100 inphosphate buffered saline (PBS) before measurement. For accurate particle sizing with the Nano ZS, the viscosity of the buffer and the refractive index of the material had to be provided to the equipment software (PBS: v=1.02 cP, RI=1.45). For zeta potential measurements, samples were diluted in water (v=0.8872 cP). Data were analyzed using the Zetasizer Software V 7.11 from Malvern Instruments.

The osmolarity and the pH of formulations were measured by using respectively a micro-sample Osmometer (Fiske Associates model) and a pHmeter (Mettler Toledo) according to the equipment manufacturer instructions.

Results

The characterizations of LNPs L319 and LNPs Lip. (III) are described in the TABLE 1 below.

TABLE 1
Characterization of the LNPs

| Ionizable | | Size | Calculated | Zeta Potential | | Osmolarity |
|---|---|---|---|---|---|---|
| lipid | LNPS | (d · nm) | ratio N/P | (mV) | pH | mOsmol/kg |
| L319 | 50/10/38.5/1.5 | 130 | 6 | −13.9 | 7.3 | 301 |
| | DSPC/Chol/PEG-PE | | | | | |
| L319 | 50/10/38.5/1.5 | 151 | 6 | ND | 7.3 | 293 |
| | DSPC/Chol/DMG-PEG | | | | | |
| Lip. (III) | 50/10/38.5/1.5 | 127 | 12 | −0.39 | 6.2 | 250 |
| | DSPC/Chol/PEG-PE | | | | | |
| Lip. (III) | 50/10/38.5/1.5 | 152 | 6 | ND | 6.3 | 317 |
| | DSPC/Chol/PEG-PE | | | | | |
| Lip. (III) | 50/10/38.5/1.5 | 94 | 12 | ND | 6.3 | 309 |
| | DSPC/Chol/PEG-PE | | | | | |
| Lip. (III) | 35/2.5/46.5/16 | 63 | 10 | ND | 6.4 | 320 |
| | DOPE/Chol/DMG-PEG | | | | | |
| Lip. (III) | 50/10/38.5/1.5 | 80 | 12 | ND | 6.3 | 309 |
| | DSPC/Chol/PEG-PE | | | | | |

| Ionizable | | Encapsulated mRNA | Total mRNA | Encapsulation |
|---|---|---|---|---|
| lipid | LNPS | conc. (μg/mL) | conc. (μg/mL) | rate (%) |
| L319 | 50/10/38.5/1.5 | 114 | 114 | 100 |
| | DSPC/Chol/PEG-PE | | | |
| L319 | 50/10/38.5/1.5 | 112 | 118 | 94 |
| | DSPC/Chol/DMG-PEG | | | |
| Lip. (III) | 50/10/38.5/1.5 | 100 | 121 | 83 |
| | DSPC/Chol/PEG-PE | | | |
| Lip. (III) | 50/10/38.5/1.5 | 179 | 298 | 60 |
| | DSPC/Chol/PEG-PE | | | |
| Lip. (III) | 50/10/38.5/1.5 | 178 | 210 | 85 |
| | DSPC/Chol/PEG-PE | | | |
| Lip. (III) | 50/10/38.5/1.5 | 136 | 156 | 88 |
| | DSPC/Chol/PEG-PE | | | |
| Lip. (III) | 50/10/38.5/1.5 | 120 | 139 | 87 |
| | DSPC/Chol/PEG-PE | | | |

ND: not determined

### Example 32: Immunogenicity of LNPs Comprising Influenza HA mRNA in Mice

The aim of the study was to compare the immunogenicity of different doses of natural, non-replicative mRNA encoding full-length hemagglutinin (HA) of influenza virus strain A/Netherlands/602/2009 (H1N1) formulated in 2 different lipid nanoparticles, LNPs L319 and LNPs (111)/DOG-CLEAVE.

The LNPs were prepared as described in Example 30. The LNPs contain lipids in the following molar ratios of 50/10/38.5/1.5 (ionizable cationic lipid/neutral lipid/Chol/PEG-ylated lipid). The ionizable lipids were formulated with DSPC/Chol/PEG-PE.

Mouse Immunization Procedure

2 groups of 8 BALBc/ByJ mice (8 week-old at DO) received two intramuscular (IM) injections, given three weeks apart (DO and D21), of 5 g of mRNA with each the 2 LNP formulations.

The L319 was used as benchmark.

As negative control group, 4 mice were immunized with PBS buffer and as positive control group, 8 mice received 10 μg of monovalent Flu vaccine A/California/07/2009 (H1N1) strain derived from Vaxigrip™, according to the same immunization schedule.

Blood samples were collected on D21 (post-1) and D42 (post-2) for antibody response analysis by hemagglutination inhibition assay (HI).

Determination of Hemagglutination Inhibiting Antibody Titers (HI Titers)

This technique is used to titrate the functional anti-HA antibodies present in the sera of influenza immunized animals, on the basis of the ability of a serum containing specific functional antibodies directed against HA to inhibit the influenza virus hemagglutination activity.

Serial dilutions (2-fold) of virus (clarified allantoic fluid) A/H1N1/California/7/2009 strain were performed in PBS in order to calibrate the viral suspension and to obtain 4 HAU (Hemagglutination Unit) in presence of cRBCs (0.5% in PBS). Calibrated virus (50 μL) was then added to the V shaped well of a 96 well plate on 50 μL of serum serial dilutions (2-fold) in PBS starting from 1:10 and incubated one hour at room temperature.

In order to eliminate serum non-specific inhibitors directed against the HA, each serum was treated with a receptor-destroying enzyme (RDE) (neuraminidase from Vibrio cholerae—Type III—Sigma Aldrich N7885) and with chicken red blood cells (cRBCs). Briefly, 10 mU/mL of RDE was added to each serum. The mix was then incubated 18 h at 37° C., followed by 1 h inactivation at 56° C. To cool, the mixture “serum-RDE” was placed in a time range from 30 min to 4 hours at 4° C. The “serum-RDE” mixture was then absorbed on 10% cRBCs in PBS for 30 min, at room temperature, and then centrifuged at 5° C., 10 min at 700 g. The supernatant corresponding to 10-fold diluted serum was collected to perform the HI assay.

Chicken red blood cells (0.5% in PBS) (50 μL) were then added to each well and inhibition of hemagglutination or hemagglutination was visually read after one hour at room temperature.

The titer in HI antibody is the reciprocal of the last dilution giving no hemagglutination. A value of 5 corresponding to half of the initial dilution (1:10) was arbitrary given to all sera determined negative in order to perform statistical analysis.

Statistical Analysis

HI titers were log 10 transformed prior to statistical analyses.

To compare the 2 LNP formulations (LNPs L319 vs LNPs (III)), an ANOVA (Analysis Of Variance) model with drug substance as fixed factor was performed. For the comparison between the different the 3 products (LNPs L319 vs LNPs (III) and Vaxigrip), a Tukey's adjustment for multiple testing was performed. The model's residuals were studied to test the model's validity (normality, extreme individuals, etc.).

All analyses were done on SAS SEG v9.4®. The nominal level of statistical significance was set at α=5% for effects of the main factors and 10% for the interaction.

Results

The antibody responses elicited against A/California/7/2009 (H1N1) were measured by HI assay in individual sera collected from all animals at D21 and D42.

Mean HI Titers Measured in Mice after One and Two IM Injections of LNPs

Results analyzed according the total mRNA content per dose showed the following: Following two injections with the LNPs, high mean HI titers was observed. After the second injection of mRNA (post-2 immunization (D42)), animals administered with mRNA/LNPs (III) showed detectable HI responses as observed with mRNA/L319 LNPs. Although the mean response obtained with mRNA/LNPs (III) is slightly below the response obtained with the mRNA LNPs L319, it may be noticed that half of the tested mice have a HI response above 1000, and close to 3000.

Reactogenicity Observed in Mice Injected with LNPs

After one immunization, a transit moderate inflammation clinical signs (observed D2 post prime) were observed in only 1 out of the 8 mice injected with LNPs (III). After two immunizations, a slight inflammation was observed in two mice which received 5 μg of total mRNA in LNP L319 at the injection site on D22, D23 or D24, i.e. 1 to 3 days following the booster injection. This inflammation was not seen any more on the following observation time point on D35 i.e. 14 days post booster injection. By contrast, only the single same mouse which already displayed a transit moderate swelling post-prime LNPs (III) suffered also from a slight inflammation at the injection site on the day after the booster injection, which lasted again only one day (D23).

mRNA/LNPs (III) are well tolerated and better tolerated than mRNA/LNPs L319.

TABLE 2
Local reactogenicity

| | | Site observations post-1 | | | Site observations post-2 | | | |
|---|---|---|---|---|---|---|---|---|
| | Mouse | (swelling) | | | (swelling) | | | |
| Group | # | Day 1 | Day 2 | Day 3 | Day 1 | Day 2 | Day 3 | D14 |
| LNPs | 1 | | | | | | | |
| L319 | 2 | | | | | +/− | +/− | |
| | 3 | | | | | | | |
| | 4 | | | | +/− | +/− | +/− | |
| | 5 | | | | | | | |
| | 6 | | | | | | | |
| | 7 | | | | | | | |
| | 8 | | | | | | | |
| LNPs | 1 | | | | | | | |
| (III) | 2 | | | | | | | |
| | 3 | | | | | | | |
| | 4 | | | | | | | |
| | 5 | | | | | | | |
| | 6 | | + | | | +/− | | |
| | 7 | | | | | | | |
| | 8 | | | | | | | |

Blank cell: no swelling
+/−: low swelling
+: medium low swelling
++: medium high swelling
+++: high swelling

### Example 33: LNP Delivered Luciferase mRNA Imaging

The purpose of the study was to determine the liver transduction efficiency and tissue biodistribution of different LNP formulations in normal mice.

Materials and Methods

LNPs Reagents

1,3-dioleoyl-sn-glycero-3-phosphocholine (DOPC), 1,2-distearoyl-sn-glycero-3-phosphocholine (DSPC), 1,2-dimyristoyl-rac-glycero-3-methoxypolyethylene glycol-2000 (DMG-PEG) were purchased from Avanti Polar Lipids (Alabaster, Albany). 3β-Hydroxy-5-cholestene, 5-Cholesten-3β-ol (Cholesterol) and Acrodisc Syringe Filters with Supor Membrane, Sterile-0.2 μm, 13 mm, were purchased Sigma Aldrich (St. Louis, Missouri). Coatsome SS-OP (SS-OP) was purchased from NOF America Corporation (White Plains, NY headquartered in Japan). Nuclease-free water, 10×PBS Buffer pH 7.4, Sodium Citrate, Dihydrate, Citric Acid, Sodium Chloride, Sucrose, Ethyl Alcohol, BD Vacutainer General Use Syringe Needles (BD Blunt Fill Needle 18G), BD Slip Tip Sterile Syringes (3 ml & 1 mL), Amicon Ultra Centifugal Filter Units, DNA-free microcentrifuge tubes (1.5 mL), Invitrogen RNase-free Microfuge Tubes (0.5 mL), Invitrogen Conical Tubes (15 mL) (DNase-RNase-free), Fisher Brand Semi-Micro Cuvette, Quant-iT™ RiboGreen® RNA Assay Kit and RnaseZap™ were purchased from Thermo Fisher Scientific (Waltham, Massachusetts). mRNA encoding luciferase was purchased from TriLink™ Biotechnologies (mRNA-Luc—Ref.: L-7602).

All LNPs were manufactured using the NanoAssemblr Benchtop from Precision Nanosystems (British Columbia, Canada).

LNPs Manufacturing

The following buffer systems were used:
Phosphate Buffer Saline (PBS): 8 mM Na₂HPO₄, and 2 mM KH₂PO₄, 137 mM NaCl, 2.7 mM KCl, pH 7.4
Citrate Buffer-10 mM: 5 mM Sodium citrate, 5 mM citric acid, 150 mM Sodium Chloride, pH 4.5
Citrate Buffer-50 mM: 25 mM Sodium citrate, 25 mM citric acid, pH 4.0
Citrate-Sucrose Buffer: 5 mM Sodium citrate, 5 mM citric acid, 8% (w/v) sucrose, pH 6.3

The following LNPs were prepared according to the procedure described in Example 30.

Lipid Phase

LNPs SS-OP: SS-OP/DOPC/Chol=52.5/7.5/40, DMG-PEG2000 1.5 mol %

533 μL of mixed lipids solution were prepared (total lipid concentration 9 mM) in a 1.5 mL conical tube. (total lipid=SS lipid+DOPC+Chol) by mixing the following solutions:

TABLE 3
LNP mix working solution for LNP SS-OP

| | Concentration in | |
|---|---|---|
| Lipid | Ethanol (mM) | Volume (μL) |
| SS-OP | 8.52 | 222 |
| DOPC | 10 | 27 |
| Cholesterol | 20 | 72 |
| DMG-PEG 2000 | 1 | 54 |
| Ethanol | — | 158 |
| Total | 4.58 | 533 |

LNPs (III): lipidic compound (III)/DSPC/Chol/DMG-PEG=50/10/38.5/1.5

533 μL of mixed lipids solution (total lipid concentration 10 mM) was prepared in a 1.5 mL conical tube by mixing the following solutions:

TABLE 4
LNP mix working solution for LNPs (III)

| | Concentration in | |
|---|---|---|
| Lipid | Ethanol (mM) | Volume (μL) |
| Lipidic compound | 10 | 162 |
| (III) | | |
| DSPC | 10 | 8 |
| Cholesterol | 20 | 51 |
| DMG-PEG 2000 | 1 | 40 |
| Ethanol | — | 272 |
| Total | 5 | 533 |

Aqueous Nucleic Acid Containing Phase

The following mRNA containing aqueous solutions were used for manufacturing the LNPs.

TABLE 5
mRNA working solution

| | | Target | Volume | | Volume | |
|---|---|---|---|---|---|---|
| | | mRNA | from | Citrate | of citrate | Total |
| | | concentration | stock | buffer | buffer | Volume |
| | N/P | (μg/mL) | (μL) | type | (μL) | (μL) |
| LNP | 6 | 90 | 43.5 | Citrate | 1306.5 | 1350 |
| SSOP | | | | Buffer-10 | | |
| | | | | mM | | |
| LNP | 12 | 45 | 21.8 | Citrate | 1328.2 | 1350 |
| (III) | | | | Buffer-50 | | |
| | | | | mM | | |

Procedure

The LNPs were manufactured with the NanoAssemblr™ according to manufacturer's recommendations the following parameters:

TABLE 6
NanoAssemblr ™ parameters

| | |
|---|---|
| Volume | 1.37 mL |
| Flow Rate Ratio | 3:1 |
| Total Flow Rate | 4 mL/min |
| Left Syringe Size | 3 mL |
| Right Syringe Size | 1 mL |
| Start Waste Volume | 0.25 mL |
| End Waste Volume | 0.05 mL |

The LNPs were formulated to encapsulate 4.25 μg of mRNA (final content)

LNPs Harvest and Purification

The harvested LNPs SS-OP were three-time washed by dilution in PBS and filtration (100KD MWCO). The resuspended LNPs were then filtered on 0.2 μm filter.

The harvested LNPs (III) were three-time washed by dilution in 10 mM citrate/8% sucrose buffer, pH 6.3 and filtration (100KD MWCO). The resuspended LNPs were then filtered on 0.2 μm filter.

Animals Study

2 groups, each of 4 of SKH1 hairless mice, female 10-12 weeks old, were tested with LNPs SS-OP and LNPs Lip. (III) encapsulating a luciferase encoding mRNA. A last group of 2 mice was injected with PBS and used as control.

The LNPs were formulated to encapsulate 4.25 μg of mRNA (final content).

TABLE 7
Design of experiment

| | | Animal per | |
|---|---|---|---|
| Group | LNPs | group | Timepoints |
| A | LNPs (III) | 4 | Radiance measured 24 h post- |
| | | | injection |
| B | LNPs SS-OP | 4 | Radiance measured 24 h post- |
| | | | injection |
| C | PBS, IV | 2 | Radiance measured 24 h post- |
| | | | injection |

Radiance of liver, spleen, kidney, lung and hear were taken at 24 h, ex vivo according to the following procedure.

Ex vivo bioluminescence of isolated organs was performed immediately after euthanasia of the animals by CO₂, around 15 min after subcutaneous injection of luciferin (200 mg/kg). Dissected organs were placed on a black sheet and imaged with IVIS Spectrum CT (PerkinElmer, Hopkinton, MA). To quantify bioluminescence emission signal, identical regions of interest (ROI) were positioned to encircle each organ region, the imaging signal was quantitated as average radiance (photons/s/cm2/steradian).

Results

Ex vivo organ analysis was performed twenty-four hours post injection. The organ data is summarized in Table 7.

Significant transduction and luciferase expression were observed in the spleen for LNPs (III) (200-folds higher) compared to other organs (spleen, kidney, heart and lungs). The specificity of LNPs (III) targeting spleen compared to liver was 43 folds higher than the LNPs SS-OP (Table 8). The LNPs SS-OP although demonstrated higher absolute expression in the spleen compared to LNPs (III), the relative expression in the liver was significantly higher showing that the LNPs SS-OP formulation is better suited for targeting liver than spleen. The findings thus support the claim that LNPs (III) can be effectively used for delivery of the nucleic acid specifically to the spleen via intravenous administration.

TABLE 8
Radiance per organ

| | Liver | | Spleen | | Kidney | |
|---|---|---|---|---|---|---|
| GROUP | Average | SD | Average | SD | Average | SD |
| A | 4.02E+03 | 2.61E+03 | 1.72E+05 | 1.23E+05 | 1.86E+03 | 3.58E+02 |
| B | 1.92E+07 | 1.34E+06 | 1.23E+06 | 1.52E+05 | 8.01E+03 | 4.30E+02 |
| C | 1.11E+03 | 1.02E+03 | 6.05E+02 | 4.52E+01 | 5.79E+02 | 1.50E+02 |

| | Lung | | Heart | |
|---|---|---|---|---|
| GROUP | Average | SD | Average | SD |
| A | 9.53E+02 | 1.36E+02 | 3.92E+02 | 2.58E+02 |
| B | 1.36E+04 | 1.29E+04 | 1.95E+04 | 9.25E+03 |
| C | 4.62E+02 | 4.72E+02 | 4.94E+02 | 4.36E+01 |

TABLE 9
Distribution of LNPs to other organs with respect to liver

| Ex Vivo | Ratio with respect to liver expression | | | | |
|---|---|---|---|---|---|
| (24 H) | Liver/ | Spleen/ | Kidney/ | Lung/ | Heart/ |
| Formulation | Liver | Liver | Liver | Liver | Liver |
| A | 1 | 43 | 0 | 0 | 0 |
| B | 1 | 0 | 0 | 0 | 0 |
| C | 1 | 1 | 1 | 0 | 0 |

### Example 34: BioImaging Following IM Injection

The purpose of the study was to determine in vivo protein expression after injection of LNP formulations in normal mice.

Materials and Methods:

Ten weeks-old female BALB/c ByJ mice were obtained from Charles River lab (Les Oncins, Saint-Germain-Nuelles, 69210, France). At TO, animals were injected by intramuscular (IM) route with 50 μl of LNPs 319 or LNPs (III) (prepared as described in Example 30) containing 5 μg of mRNA encoding luciferase (mRNA-Luc—Ref.: L-7602 TriLink™ Biotechnologies). Luciferin potassium salt (D-luciferin, K+salt Fluoprobes, Interchim) diluted in PBS was injected through intraperitoneal (i.p) route at 3 mg per mouse which is in large excess relative to the luciferase amount.

Optical imaging was performed using the IVIS Spectrum CT device (PerkinElmer Inc., Paris, France). Bioluminescence acquisition was initiated 15 min after the injection of the substrate. The luminescence level was evaluated by an ROI applied to the injection site zone (Living Image software, PerkinElmer Inc., Paris, France). Results are expressed as total flux (ph/s) in function of time (hours) post the injection of LNPs/mRNA-Luc.

Results

The protein expression (luciferase) was measured with bioluminescence imaging at 6 h, 24 h, 48 h and 78 h post LNPs/mRNA-Luc injected in the quadriceps muscle. Mice were injected by i.p. route with 3 mg of Luciferin and bioluminescence signal acquisition was performed with IVIS CT camera. LNP (III) (n=5) was tested in comparison with L319 LNP (n=3); a positive control. TRIS Sucrose (n=2) was the negative control. Results are expressed as total flux (ph/s) in function of time (hours). Mean±SD.

The protein expression was observed in the two groups as compared to the TRIS Sucrose (white box) with an expression peak at 6 h post IM injection of LNPs/mRNA-Luc. At this time point, the bioluminescence signal in LNPs (III) group (grey box) was lower than L319 group (black box). Otherwise, for the other time points, the signal was similar.

### Example 35: Formulation of hEPO mRNA in LNPs Comprising Cleavable Lipids of the Invention

Lipid Nanoparticles:

CleanCap® EPO mRNA (5moU), a non-replicative, highly purified, mRNA encoding the human erythropoietin was obtained from TriLink Biotechnologies, San Diego, CA (catalogue number L7209; hEPO mRNA). This mRNA is capped using CleanCap, TriLink's proprietary co-transcriptional capping method, which results in the naturally occurring Cap 1 structure with high capping efficiency. It is polyadenylated, modified with 5-methoxyuridine and optimized for mammalian systems. It mimics a fully processed mature mRNA.

LNPs comprising lipidic compound of formula (III) (or DOG-Cleave) or one of the compounds (VI), (XVII), (XIX), (XXI), (XXII), (XXIV), (XXVII) and (XXVIII), DSPC, Chol and DMG-PEG2000 at a molar ratio of 50:10:3.5:1.5, and hEPO mRNA at a N/P charge ratio=12 (unless otherwise specified in the table), were prepared as described in Example 30 by using the NanoassemblR. An Aqueous/Ethanolic phase volume ratio of 3/1 and at total flow rate of 4 mL/min was used. LNPs were prepared at a concentration of 60 μg of hEPO mRNA/mL in PBS 1×.

Intramuscular injection of mice with LNPs Lip. (III), or LNPs with one of the compounds or one of the compounds (VI), (XVII), (XIX), (XXI), (XXII), (XXIV), (XXVII) and (XXVIII), containing hEPO mRNA and detection of hEPO expression in the serum was thereafter carried out.

Animals

Female Balb/c ByJ mice (7 weeks of age at receipt) were purchased from Charles River Laboratories (Saint-Germain-Nuelles, France) and housed for one-week acclimation before starting the study. Mice were identified individually by fur coloration. Experiments were approved by Sanofi Pasteur's animal ethics committee and followed European guidelines for standards of animal care.

Study Schedule

Four 8-week-old mice per group were injected on DO via intramuscular route in the quadriceps with 1 μg-dose of hEPO mRNA formulated in LNPs Lip. (IV)/DSPC under a final volume of 50 μL. As negative control, 4 mice received the same volume of PBS (for accelerated stability study and lipid screening) or Citrate Buffer (lipid screening)

Blood samples were collected 6 hours post-injection to measure the expression of hEPO in serum using a specific ELISA assay.

Blood Samples

Blood samples were collected 6 hours post-injection by carotid section under deep anesthesia with Imalgene/Rompun (1.6 mg of Ketamine/0.32 mg of Xylazine) in serum-separation tubes (BD Vacutainer #BD367957). Sera were aliquoted and stored at −20° C. until hEPO determination.

hEPO Determination in Mouse Serum

hEPO expression in mouse sera was assessed using human Erythropoietin Quantikine IVD ELISA kit (R&D Systems #DEP00). The ELISA was performed following supplier's instructions. Briefly, sera were added in pre-coated plates and incubated for one hour at room temperature under orbital shaking. After sera removal, Erythropoietin conjugate was added for one hour at room temperature under orbital shaking. Plates were washed and Substrate solution was added for 20-25 minutes at room temperature before stopping the reaction with Stop solution. Absorbance at 450 nm with 650 nm signal subtraction was determined in a microplate reader. Data were analyzed using SoftmaxPro software and expressed in log 10 of the concentration of hEPO measured in mouse sera in pg/mL.

Results

TABLE 10
hEPO expression in mice having received intramuscular
injection of LNPs containing hEPO mRNA

| LNPs WITH COMPOUND | |
|---|---|
| No. | EPO secretion IM (Log10 pg/ml) |
| III | 1.5 |
| VI | 2.1 |
| XVII | 2.4 |
| XIX | 2.4 |
| XXI | 2.3 |
| XXII | 2.5 |
| XXIV | 1.9 |
| XXVII | 1.9 |
| XXVIII | 2.3 |

### Example 36: HPLC Analysis to Confirm Cleavage of Lipidic Compound (I)/DOG-CLEAVE During LNP Formulation Process

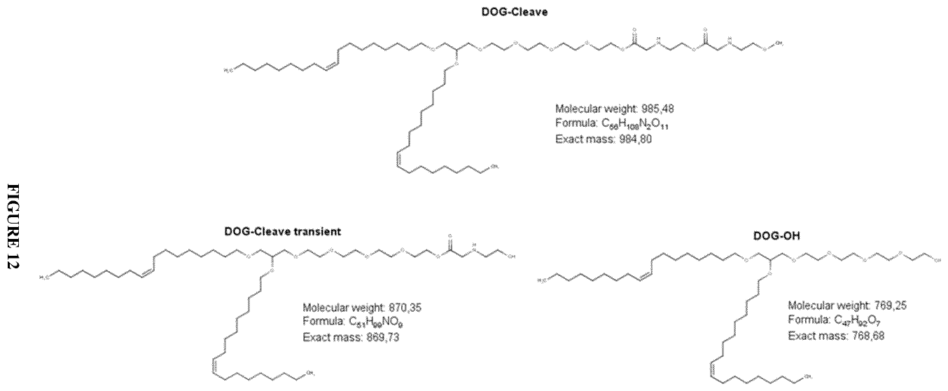

The aim of this analysis was to demonstrate that the lipidic compound of formula (III) (DOG-Cleave) was cleaved through a cyclisation process generating successively “DOG-cleave transient” and the uncharged lipid “DOG-OH” in the final LNP formulation (see Scheme (14) on FIG. 12).

To do so, an UHPLC-CAD-MS method was used to analyze the lipid constituents at different steps of the LNP preparation process described in Example 30, i.e., in the starting ethanol solution, after the first dialysis step and in the final LNP.

UHPLC-CAD-MS Method for LNP Lipid Content and Integrity Analysis

For the separation and analysis of the different lipid components in LNPs, a RP-UHPLC method with Charged Aerosol Detection (CAD) and mass spectrometry detection (MS) was used. The equipment and chromatographic conditions are described in the tables below.

TABLE 11
Equipment and chromatographic conditions

| | |
|---|---|
| Equipment | UHPLC/CAD from Thermo vanquish and |
| | MS from Thermo Q Exactive Focus |
| Buffer A | Acetonitrile/Water (60:40) with 10 mM ammonium |
| | formate and 0.1% formic acid |
| Buffer B | Isopropanol/acetonitrile (90:10) with 10 mM |
| | ammonium formate and 0.1% formic acid |
| Column | Waters Acquity Premier CSH C18 Column 130 Å, |
| | 1.7 μm, 2.1 mm × 50 mm (Part Number: 186009460) |
| Flow rate | 0.4 mL/min |
| Column | 55° C. |
| temperature | |
| Autosampler | 5° C. |
| temperature | |
| Injection Volume | 1 μL |
| Detector | 210 nm |
| Wavelength | |
| CAD parameters | Temperature: 50° C. |
| | Power Function: 1 |
| MS parameters | Source: ESI (Electro Spray ionization) |
| | Scan type: Full MS |
| | Polarity: positive/negative |
| | Sheath gas flow rate: 60 |
| | Aux gas flow rate: 20 |
| | Sweep gas flow rate: 0 |
| | Spray Voltage (\|kV\|): 3.00/2.50 |
| | Capillary temperature: 380 |
| | Aux gas heater temperature: 0 |
| Run time | 25 minutes |

The column was eluted with a gradient of solvent system B in A according to the following steps:

TABLE 12
Gradient of solvent system B in A

| No | Time (min) | % B | Curve |
|---|---|---|---|
| 1 | 0.0 | 40 | 5 |
| 2 | 15.0 | 70 | 5 |
| 3 | 23.0 | 99 | 5 |
| 4 | 23.1 | 40 | 5 |
| 5 | 25.0 | 40 | 5 |

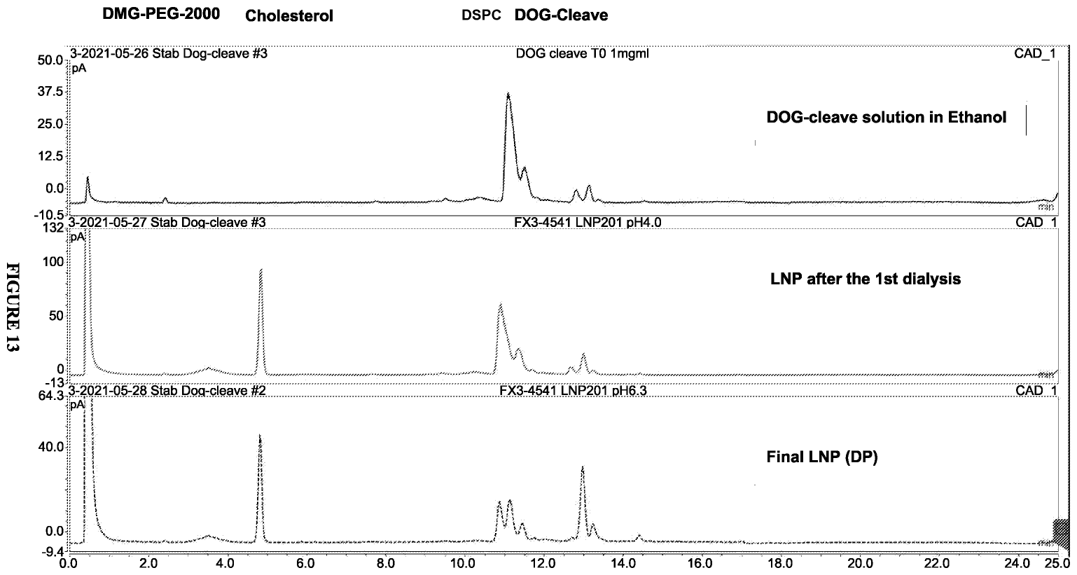

By using this method, lipidic compound of formula (III) (DOG-Cleave), DOG-Cleave transient, DOG-OH, DSPC, Chol and DMG-PEG2000 are well separated on the C18-HPLC column and could be detected at different steps of the formulation process as shown on the chromatogram on FIG. 13.

Results

The relative percentage of the different species, DOG-Cleave, DOG-Cleave transient and DOG-OH, was estimated from the extracted ion chromatograms (EICs) assuming these different species have the same response factor.

Results are shown in the table below showing the relative amounts of DOG-Cleave, DOG-Cleave transient and DOG-OH during the LNP formulation process.

TABLE 13
Relative amounts of DOG-Cleave (III), DOG-Cleave transient
and DOG-OH during the LNP formulation process

| | | DOG-Cleave | |
|---|---|---|---|
| | DOG-Cleave | Transient | DOG-OH |
| In process | (M = 984.8) | (M = 869.7) | (M = 768.7) |
| DOG-Cleave | 75% | 8% | 17% |
| EtOH solution | | | |
| LNP after 1st | 59% | 17% | 24% |
| dialysis | | | |
| Final LNP | 25% | 10% | 65% |

Conclusion

The UHPLC-CAD-MS analysis confirm the cleavage of lipid compound of formula (III) (DOG-Cleave) and its transformation into DOG-OH during the LNP formulation process. This result is consistent with the zeta potential result provided in example 31, indicating that the net surface charge of an LNP prepared from lipid compound of formula (III)/DSPC/Chol/PEG-PE 50:10:38.5:1.5 mol/mol is slightly negative (−0.39 mV) at neutral pH.

### Example 37: Immunogenicity of LNPs Comprising Influenza HA mRNA (1MpU-Modified from Amptec) in Non-Human Primates

LNPs comprising lipidic compound of formula (III) (DOG-Cleave) or L319 in the control group, DSPC, Chol and DMG-PEG2000 at a molar ratio of 50:10:3.5:1.5, and 1MpU-modified HA mRNA at a N/P charge ratio=12 (respectively N/P=6 in the L319 group), were prepared as described in Example 30 and used for the immunization of cynomolgus macaques.

Groups of 5 female cynomolgus macaques were immunized twice four weeks apart (D0, D28) with 50 μg of mRNA in LNPs injected IM into the biceps under a volume of 500 pl.

Blood samples were collected on D-33 (pre-immunization) and at different time intervals following immunization for antibody response analysis by HI assay (as described in Example 32).

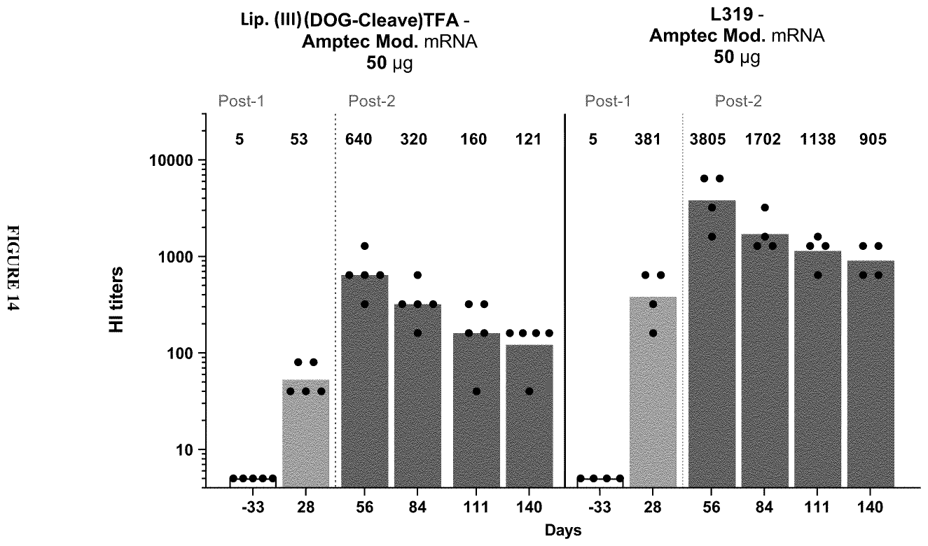

The results are shown on FIG. 14.

Conclusion

LNPs made from lipidic compound of formula (III) (DOG-Cleave) containing 1MpU-modified HA mRNA induce strong HI responses in macaques after two IM administrations.

### Example 38: Immunogenicity of LNPs Comprising Influenza HA mRNA in Mice

The aim of the study was to evaluate the immunogenicity induced with different LNPs made with different lipidic compounds as disclosed herein and containing non-replicative mRNA encoding full-length hemagglutinin (HA) of influenza virus.

BALBc/ByJ mice (8 weeks old at DO; 8 per group) were immunized as described in Example 32 with 5 μg of natural, non-replicative mRNA encoding full-length hemagglutinin (HA) of influenza virus strain A/Netherlands/602/2009 (H1N1) formulated in 4 different lipid nanoparticles, LNPs L319, LNPs (III) [DOG-Cleave], LNPs (XXI), and LNPs (XIX).

LNPs were prepared as described in Example 31 and were always composed of ionizable or cleavable lipid/DSPC/Chol/DMG-PEG2000 at a 50:10:38.5:1.5 molar ratio. Ratio N/P was 6 for LNPs L319 and N/P was 12 for the cleavable lipid-containing LNPs.

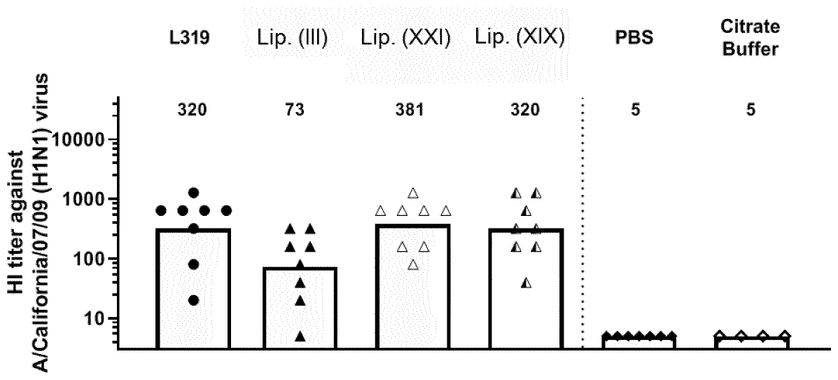

HI titers were measured 3 weeks following the second immunization as described in Example 32 and reported on FIG. 15.

1. A cleavable lipidic compound comprising at least one terminal radical of formula (I):
Y—(CHR)ₙ—Z—(CHR′)ₚ-Q * (I)
wherein:
* is the end linked, directly or not, to one C₁₀ to C₆₀ and preferably to C₁₀ to C₅₅ lipophilic or hydrophobic tail-group,
Y is a radical selected in the group consisting of a C₁-C₅ alkyl, a C₁-C₅ alkoxy, a C₁-C₅ acyl, a C₁-C₅ hydroxyalkyl, a C₁-C₅ aminoalkyl, a C₁-C₅ alkylcarboxyl ester, an acetamido, an N,N—C₁-C₅ alkylamido, a C₁-C₅ fluoroalkyl, for example a C₁-C₅ perfluoroalkyl, for example a trifluoromethyl, an imidazolyl, a triazolyl, a squaramidyl, an ureido, a cyano or is one hydrogen; and preferably is a C₁-C₅ alkoxy and more preferably a methoxy;
Z is a radical —NH—CH₂—CO—O—** or a radical —CR″(NH₂)—CO—O—** with ** that is the end closest to Q and R″ that is selected in the group consisting of hydrogen, methyl radical and trifluoromethyl radical
Q is a radical —NH—CH₂—CO—O—*** or a radical —CR″(NH₂)—CO—O—*** with R″ selected from the group consisting of hydrogen, methyl radical and trifluoromethyl radical and *** that is the end linked, directly or not, to said lipophilic or hydrophobic tail-group;
R et R′ are, independently one from the other, one hydrogen, one methyl radical or one trifluoromethyl radical; and
n et p are independently one from the other 0, 1 or 2;
or one of its pharmaceutically acceptable salts and said compound is in all the possible racemic, enantiomeric and diastereoisomeric isomer forms.

2-4. (canceled)

5. The compound of claim 1 is a compound of formula (II)
Y—(CHR)ₙ—Z—(CHR′)ₚ-Q-A-R₁ (II)
wherein:
Y, R, n, Z, R′, p and Q are as defined in claim 1;
R₁ is a C₁₀ to C₆₀ and preferably to C₁₀ to C₅₅ lipophilic or hydrophobic tail-group; and
A is a spacer arm having from 2 to 24, from 2 to 18, from 4 to 12, or from 2 to 12 carbon atoms, in a branched or unbranched linear saturated or unsaturated hydrocarbon chain, said chain is interrupted by one or several atoms of oxygen and/or moieties selected among —S—S—; (C═O)—O—; —O —(O═C)—; —(C═O)NH—; —NH—(C═O)—O—; —S—; —NH—(O═C)—; and O —(O═C)—NH—, and/or optionally having a terminal atom of oxygen or one moiety like —(C═O)—O—; —O —(O═C)—; —NH —(C═O)—; NH—(C═O)—O— or —O—(O═C)—NH—; to its end to be linked to the lipophilic or hydrophobic tail-group,
or one of its pharmaceutically acceptable salts and anyone of its racemic, enantiomeric and diastereoisomeric isomer forms.

6. The compound of claim 1 is a compound of formula (IIa)
Y—(CHR)ₙ—NH—CH₂—CO—O—(CHR′)ₚ—NH—CH₂—CO—O-A-R₁ (IIa)
wherein:
Y, R, n, R′ and p are as defined in claim 1;
R₁ is a C₁₀ to C₆₀ and preferably to C₁₀ to C₅₅ lipophilic or hydrophobic tail-group; and
A is a spacer arm having from 2 to 24, from 2 to 18, from 4 to 12, or from 2 to 12 carbon atoms, in a branched or unbranched linear saturated or unsaturated hydrocarbon chain, said chain is interrupted by one or several atoms of oxygen and/or moieties selected among —S—S—; (C═O)—O—; —O —(O═C)—; —(C═O)—NH—; —NH—(C═O)—O—; —S—; —NH—(O═C)—; and O—(O═C)—NH—, and/or optionally having a terminal atom of oxygen or one moiety like —(C═O)—O—; —O— (O═C); —NH (C═O)—; —NH—(C═O)—O— or —O—(O═C)—NH—; to its end to be linked to the lipophilic or hydrophobic tail-group;
or one of its pharmaceutically acceptable salts and anyone of its racemic, enantiomeric and diastereoisomeric isomer forms.

7-9. (canceled)

10. The compound of claim 5, wherein the hydrophobic or lipophilic tail is an optionally substituted branched or unbranched linear saturated or unsaturated C₁₀ to C₆₀ and preferably C₁₀ to C₅₅ hydrocarbon radical, and which hydrocarbon skeleton that is optionally interrupted by one or several atoms of oxygen or nitrogen and/or one or several —O—CO— or —CO—O— groups and if one nitrogen atom is present in the skeleton it is present under a form that cannot be protonated and is linked, directly or not, and preferably directly linked to the spacer, A.

11. The compound of claim 1, wherein the C₁₀ to C₆₀ and preferably C₁₀ to C₅₅ lipophilic or hydrophobic tail-group is selected from the group consisting of: 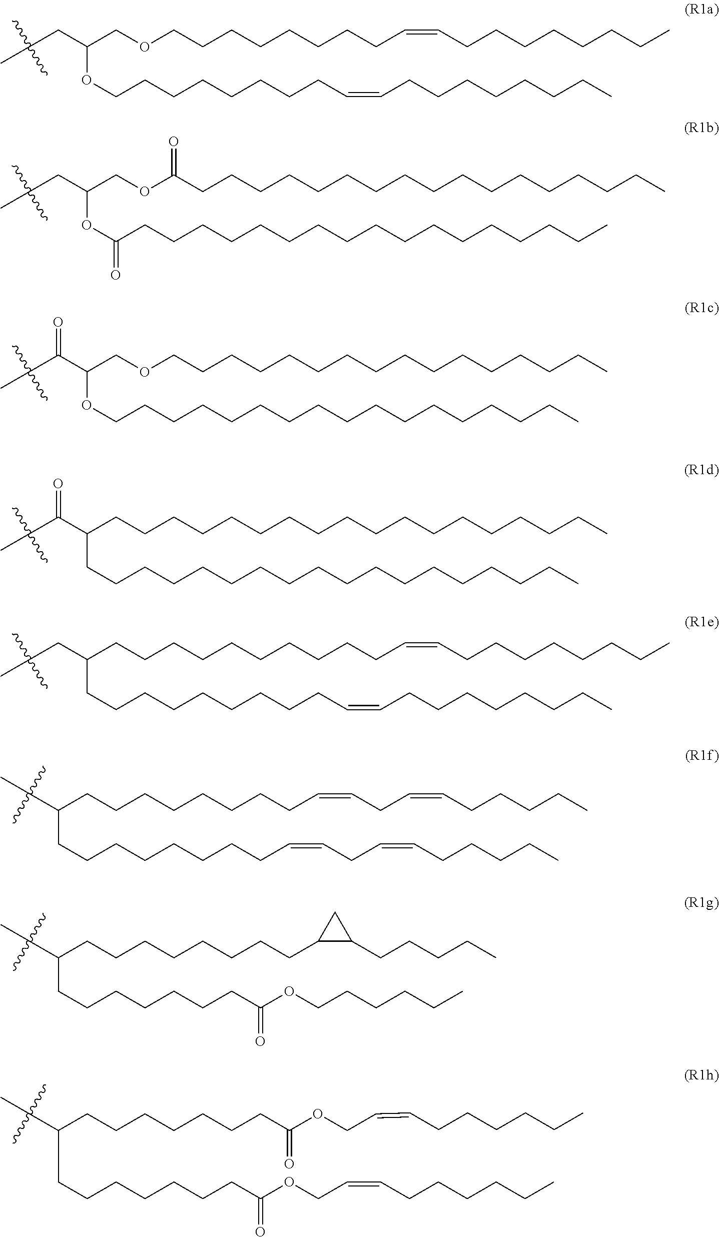 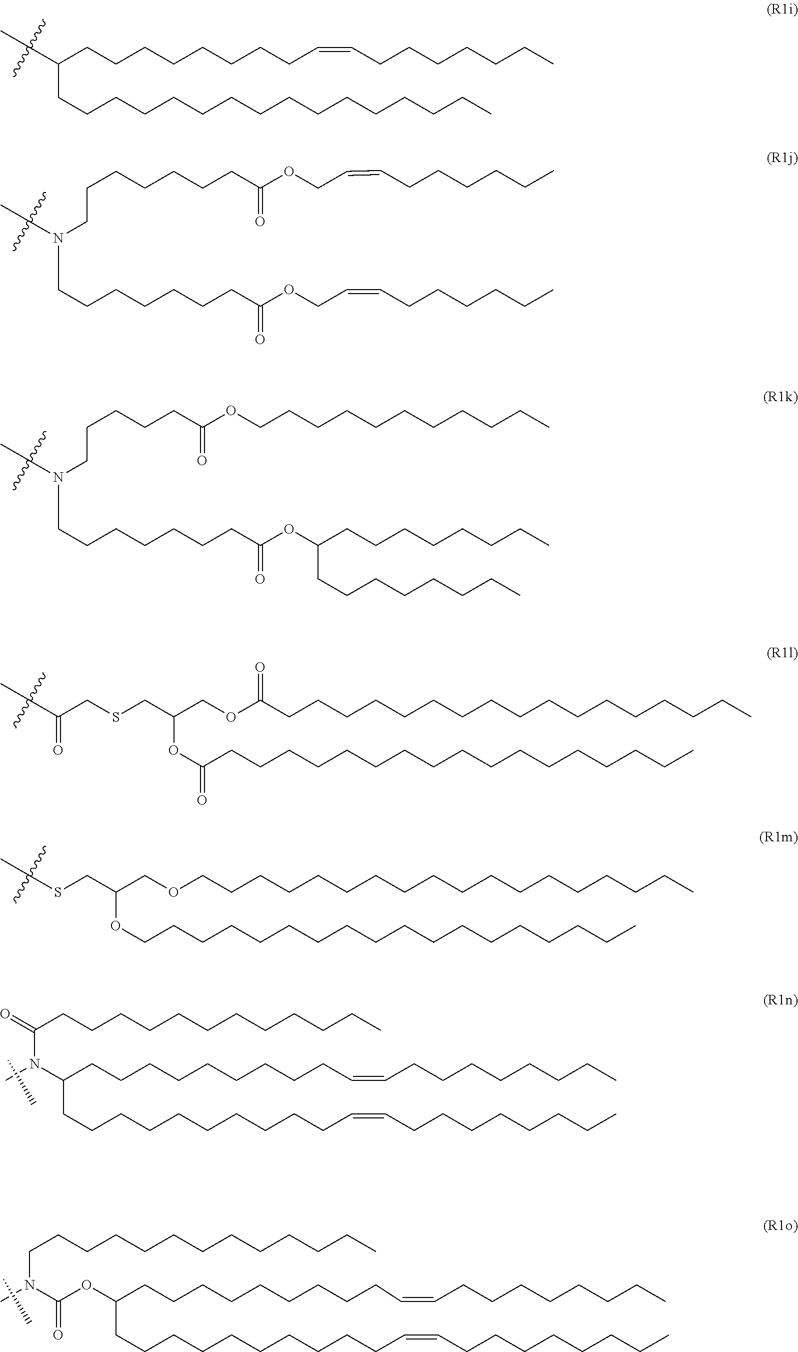 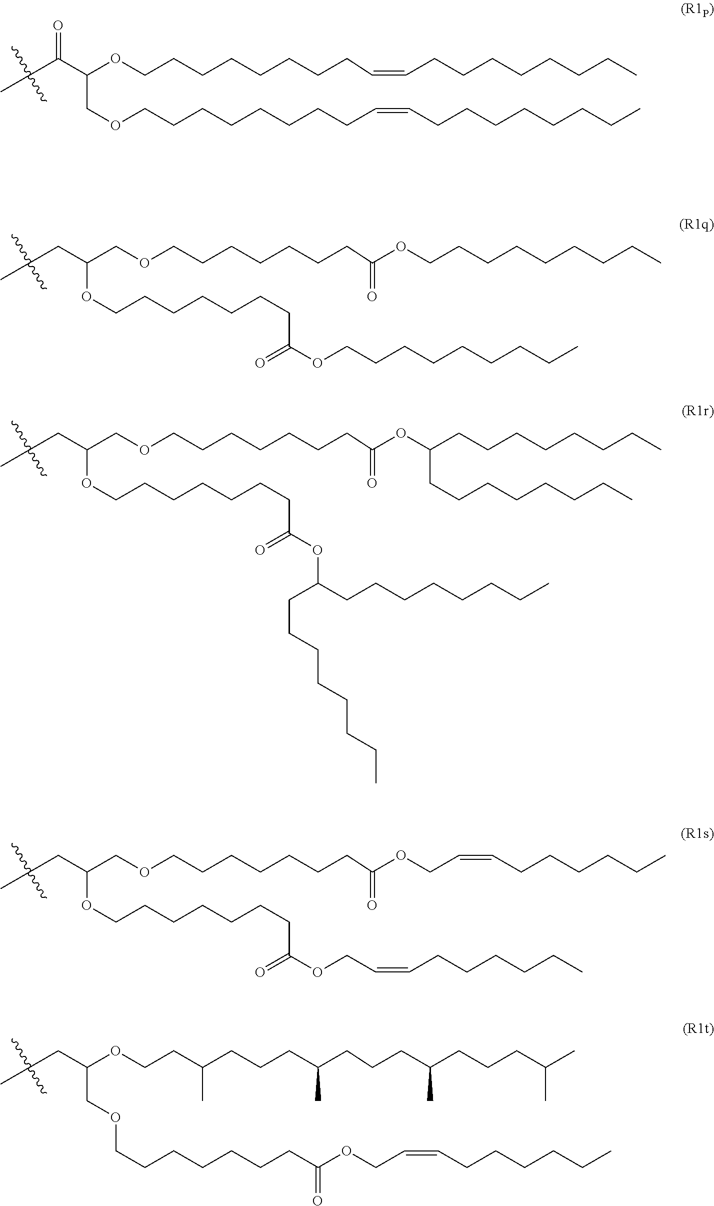 [SMILES: *C(=O)C(COCCCCCCCC/C=C\CCCCCCCC)OCCCCCCCC/C=C\CCCCCCCC ; *CC(COCCCCCCCC(=O)OCCCCCCCCC)OCCCCCCCC(=O)OCCCCCCCCC ; *CC(COCCCCCCCC(=O)OC(CCCCCCCC)CCCCCCCC)OCCCCCCCC(=O)OC(CCCCCCCC)CCCCCCCC ; *CC(COCCCCCCCC(=O)OC/C=C\CCCCCC)OCCCCCCCC(=O)OC/C=C\CCCCCC ; *CC(COCCCCCCCC(=O)OC/C=C\CCCCCC)OCCC(C)CCC[C@H](C)CCC[C@H](C)CCCC(C)C] 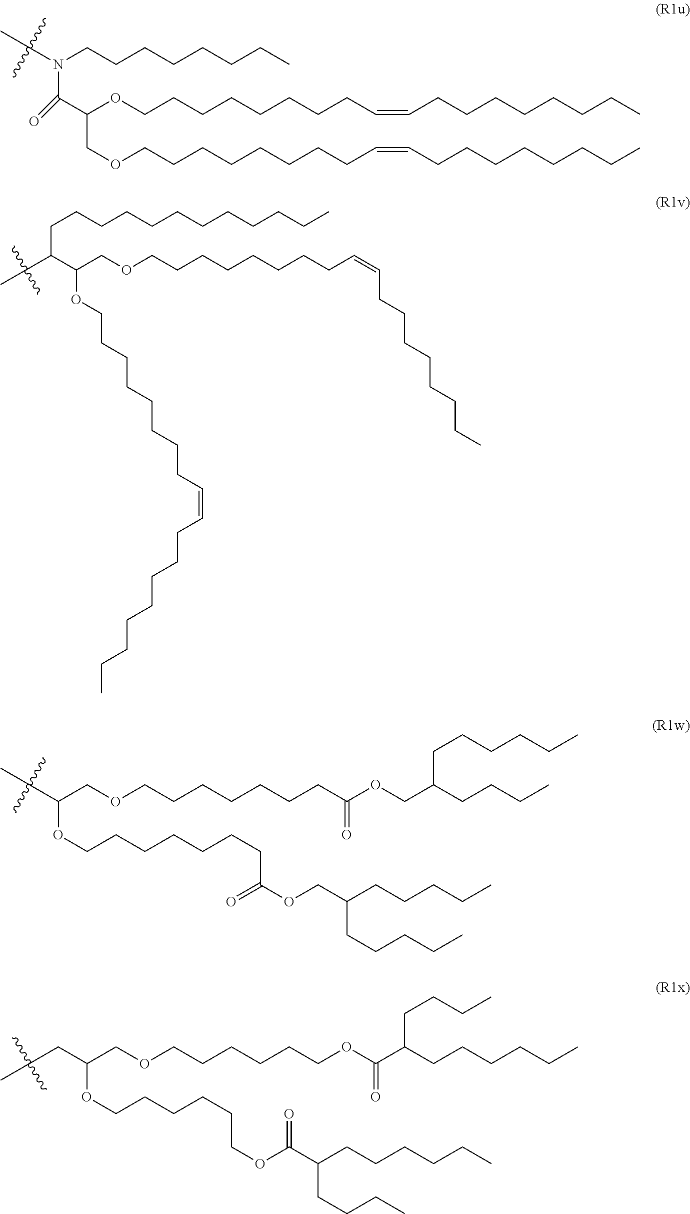 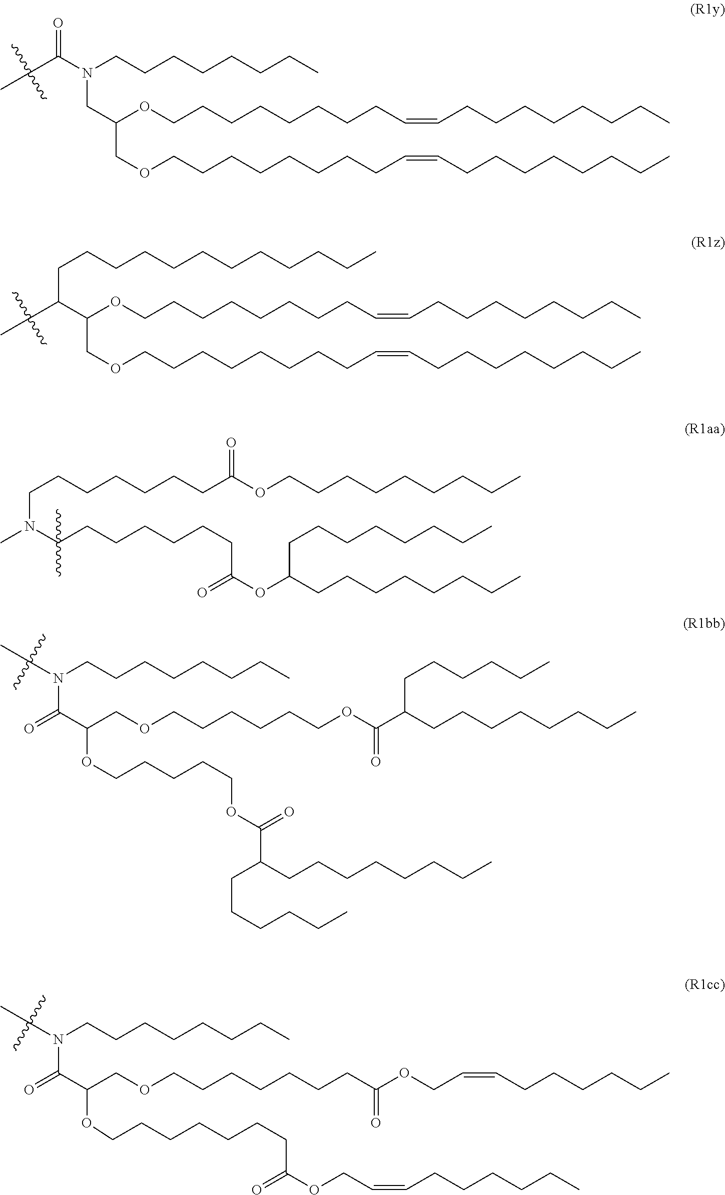
and preferably is Ria or Rib.

12-16. (canceled)

17. The compound of claim 5, wherein the spacer arm A is of formula (A1) 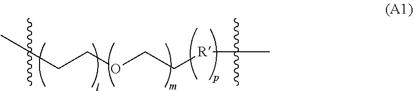
which right end being the one linked to the hydrophobic or lipophilic tail and wherein:
l is 0 or 1;
m ranges from 2 to 12, preferably from 2 to 10 or for example is 2, 3, 4, 5, 6, 7, 8, or 9
p is 0 or 1; and
R′ represents, when p is 1, a group selected from —O—(O═C)—; —(C═O)—O—; —NH—(C═O)—O— or O (O═C) NH; —NH (C═O) —O—, -; O—CH₂ C(═O)—O—; —O—C(═O)—(CH₂)₂—C(═O)— and —S—S—.

18. The compound of claim 5, wherein the spacer arm A is selected from the group consisting of and wherein which right end being the one to be linked to the lipophilic or hydrophobic tail-group:

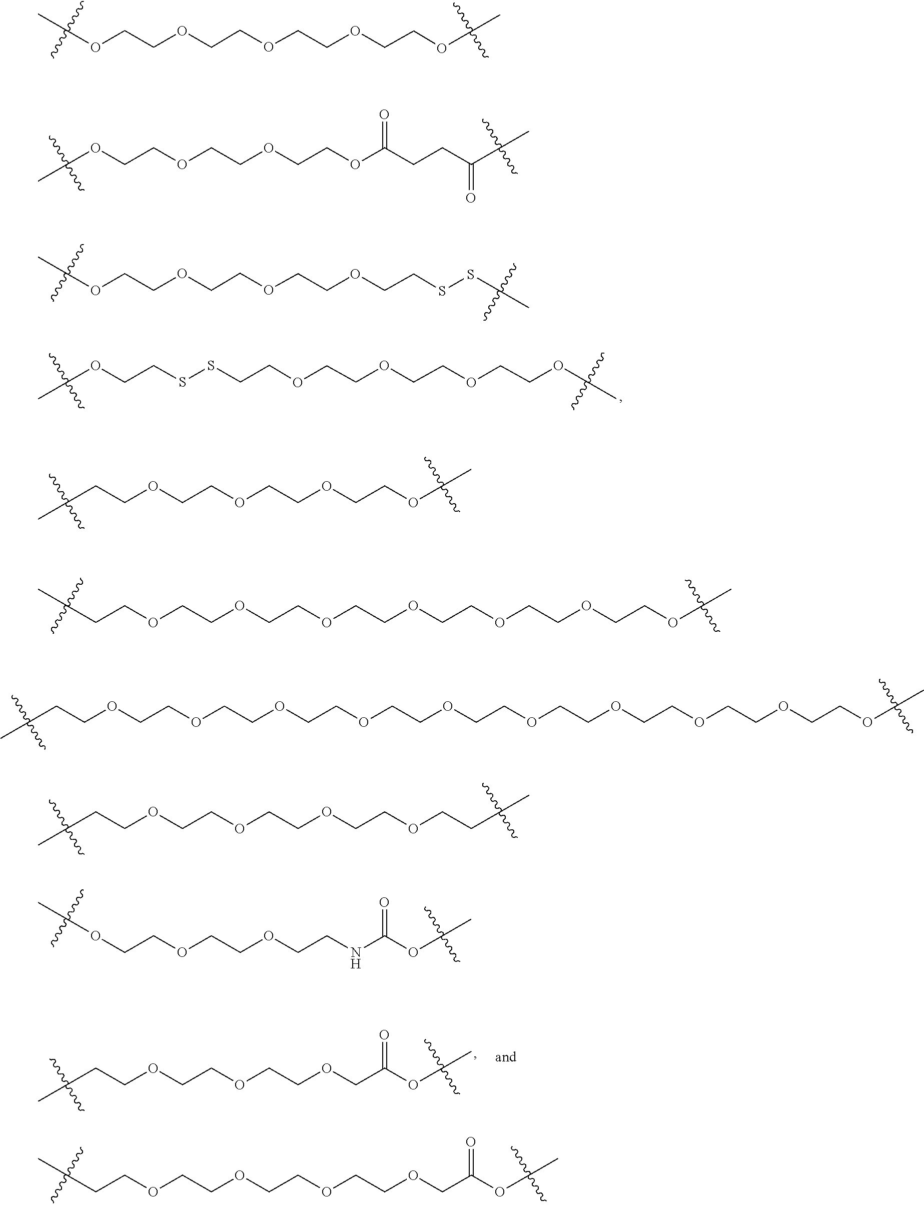

19. The compound of claim 1 is selected from compounds (III) and (VI) to (XXXI) 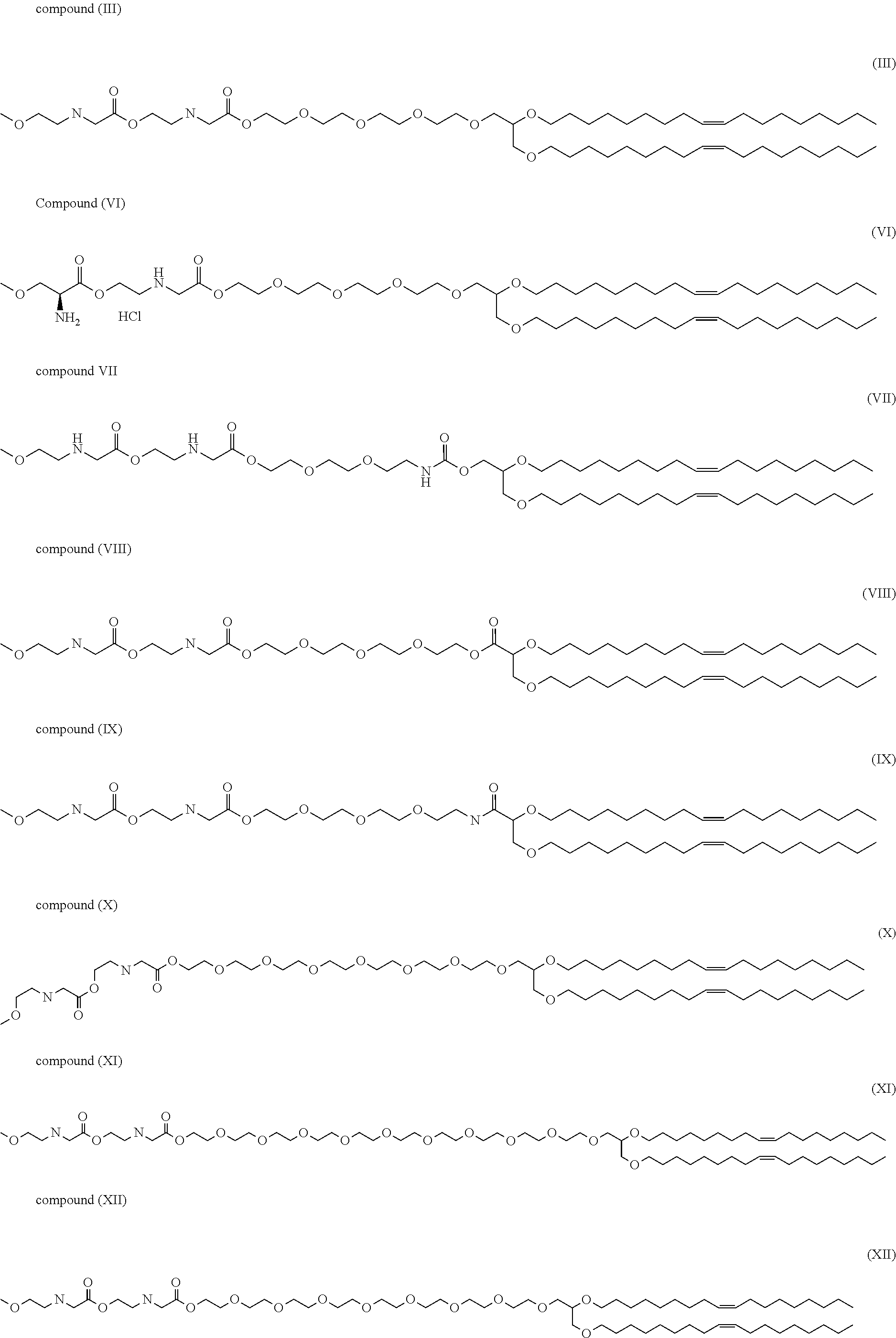 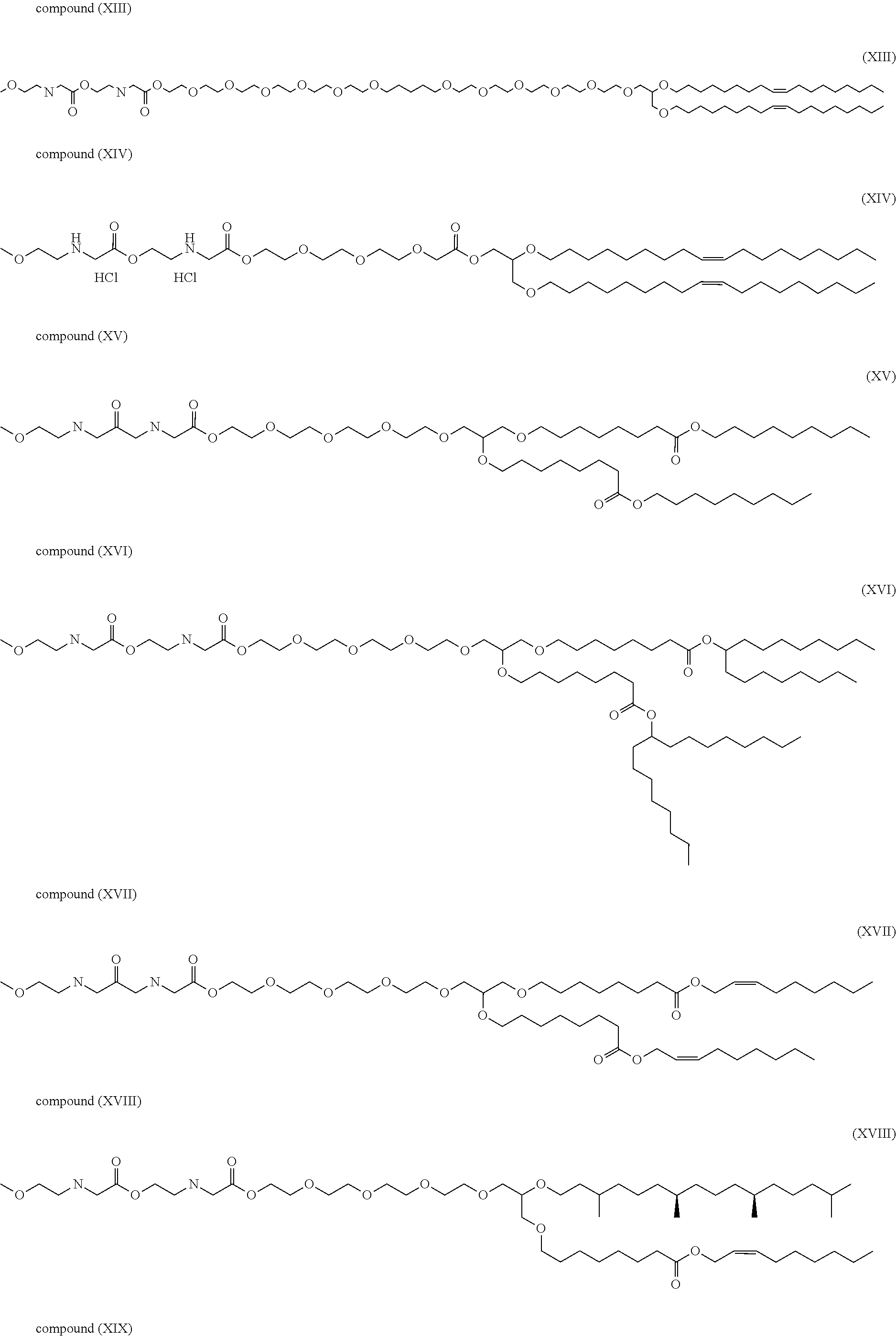 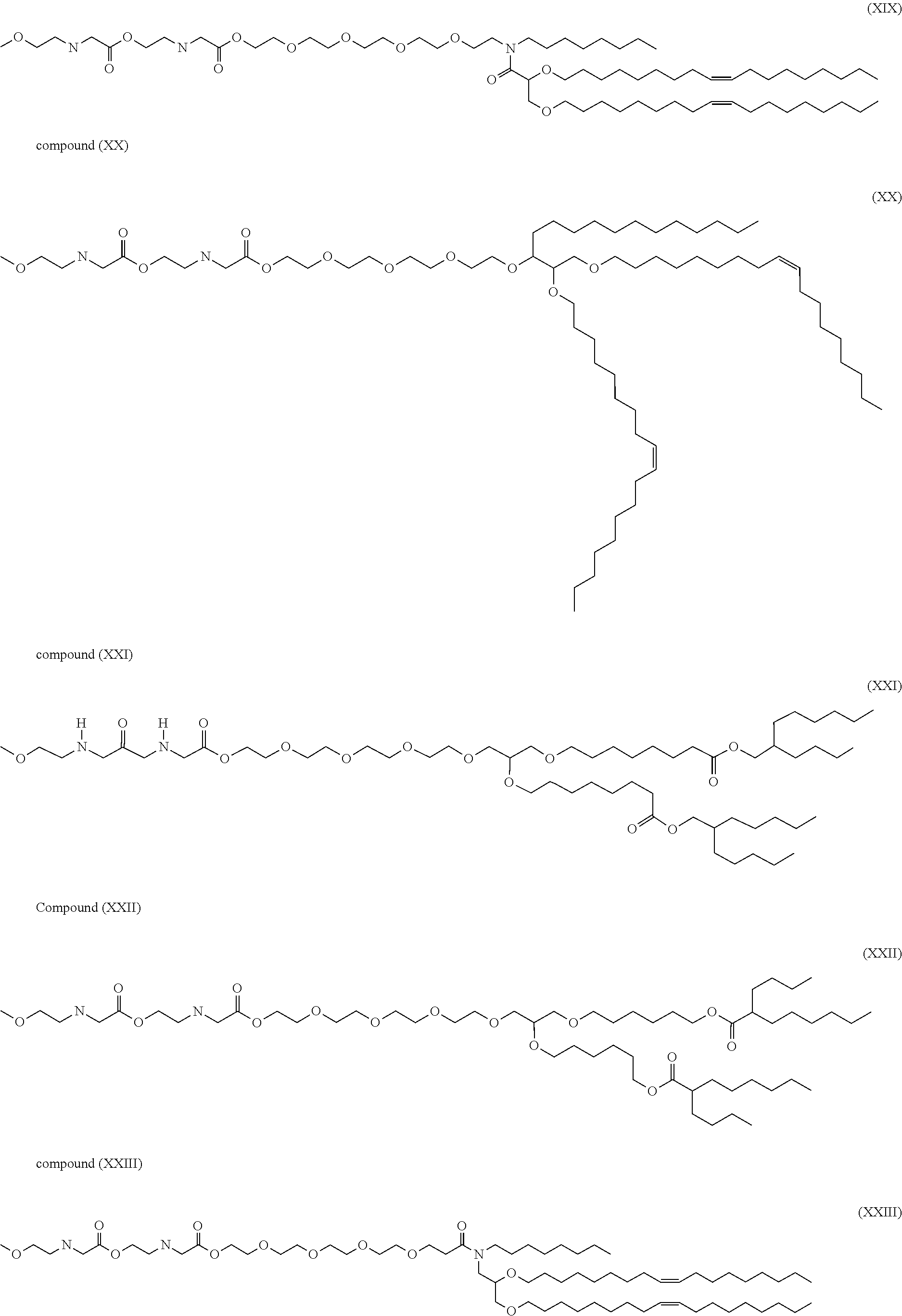 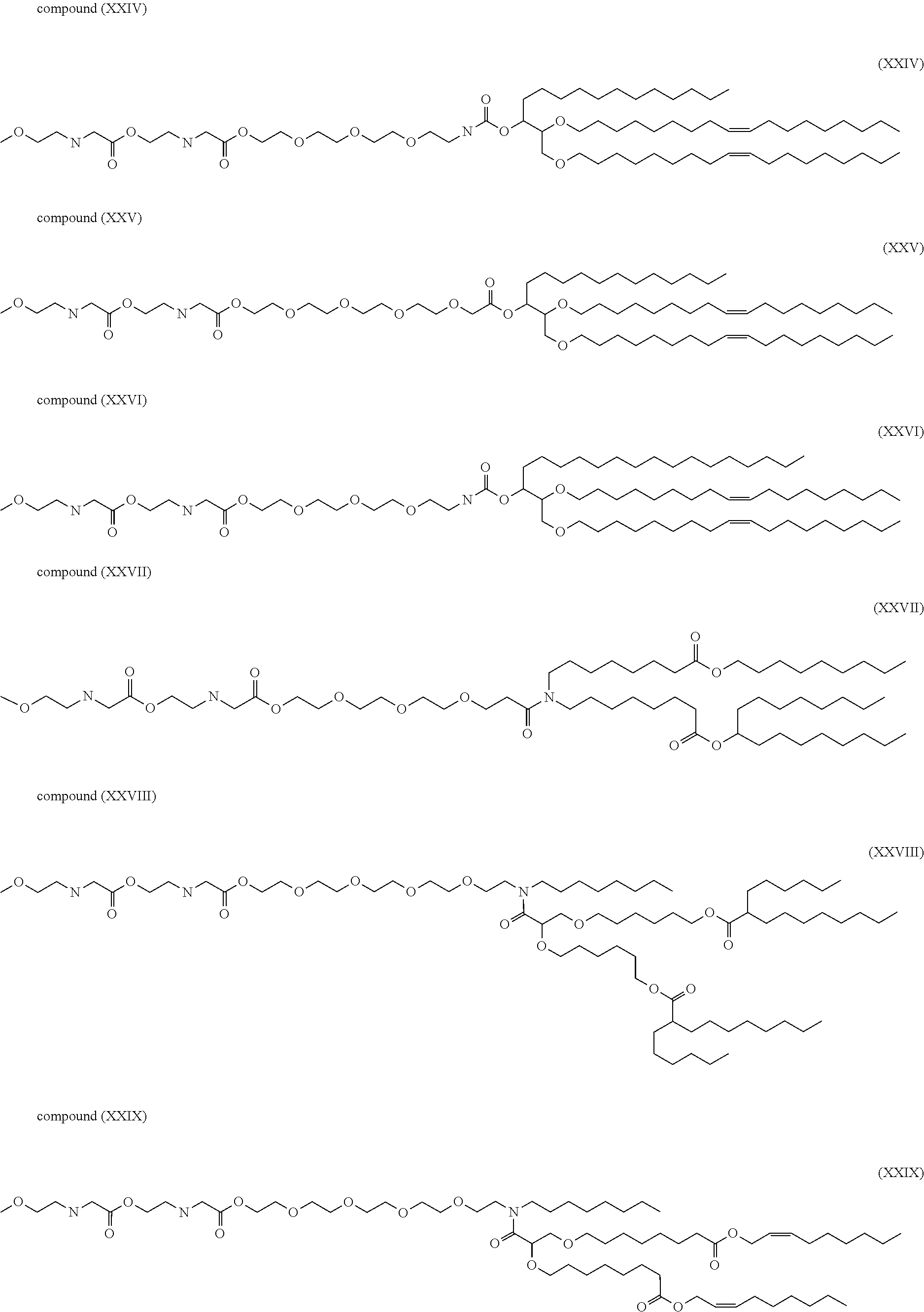 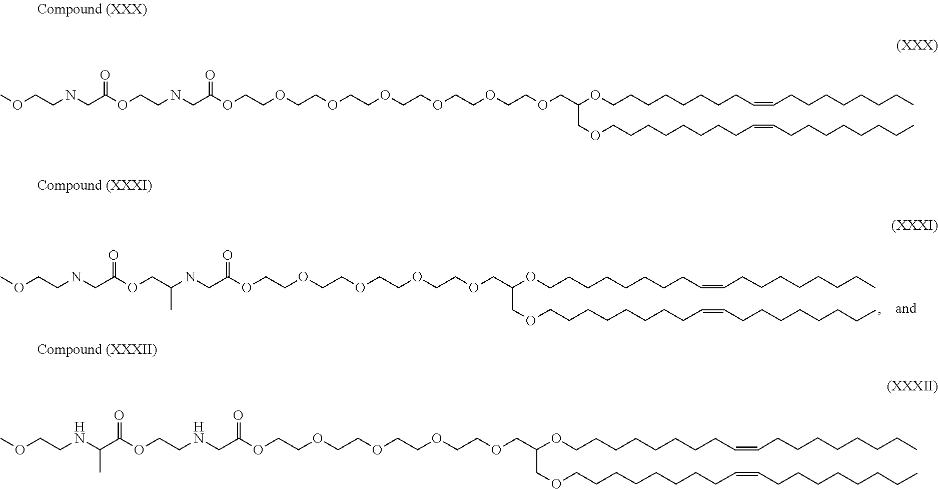
and their salts, or their trifluoroacetate salt, and/or their racemic, enantiomeric and diastereoisomeric isomer forms and preferably the compound of formula (III) or one of its salts or one of its racemic, enantiomeric and diastereoisomeric isomer forms.

20. A method for manufacturing lipid nanoparticles containing a nucleic acid, wherein the method comprises at least the steps of:
a) solubilizing, in a water miscible organic solvent, at least one lipidic compound of claim 1,
b) mixing the organic solvent obtained at step a) with an aqueous solvent buffered at a pH ranging from about 3.0 to about 4.5 and comprising at least one nucleic acid, and
c) obtaining said lipid nanoparticles containing the nucleic acid in the aqueous solvent.

21-25. (canceled)

26. Lipid nanoparticles obtainable according to claim 20.

27. A method for manufacturing a pharmaceutical composition comprising at least the steps of:
i) manufacturing at least one lipid nanoparticle according to the method of claim 20, and
ii) combining the lipid nanoparticles obtained at step i) with at least one pharmaceutically acceptable excipient or carrier.

28. A method for manufacturing an immunogenic composition comprising at least the steps of:
i) manufacturing at least one lipid nanoparticle according to the method of claim 20, said lipid nanoparticle containing at least one nucleic acid encoding for at least one antigen, and
ii) combining the lipid nanoparticles obtained at step i) with at least one pharmaceutically acceptable excipient or carrier.

29. A lipid nanoparticle comprising at least one lipidic compound of formula (IV):
HO-L (IV) wherein L is a lipophilic or hydrophobic tail-group as defined in claim 1, and at least one nucleic acid.

30. The lipid nanoparticle of claim 29, comprising at least one lipidic compound of formula (Va) or (Vb):
HO—R₁ (Va) or
OH-A-R₁ (Vb)
wherein R₁ is a C₁₀ to C₆₀ and preferably to C₁₀ to C₅₅ lipophilic or hydrophobic tail-group; and
A is a spacer arm having from 2 to 24, from 2 to 18, from 4 to 12, or from 2 to 12 carbon atoms, in a branched or unbranched linear saturated or unsaturated hydrocarbon chain, said chain is interrupted by one or several atoms of oxygen and/or moieties selected among —S—S—; (C═O)O—; O—(O═C): —(C═O)—NH; —NH—(C═O)—O—; —S—; —NH—(O═C) and O (O═C) NH, and/or optionally having a terminal atom of oxygen or one moiety like —(C═O)—O—; —O—(O═C)—; —NH—(C═O); —NH—(C═O)—O— or O—(O═C)—NH—; to its end to be linked to the lipophilic or hydrophobic tail-group.

31-32. (canceled)

33. The lipid nanoparticles of claim 29, wherein the at least one nucleic acid encodes for at least one antigen.

34. The lipid nanoparticles of claim 1, wherein it further comprises at least one nucleic acid.

35. A pharmaceutical composition comprising at least one lipid nanoparticle according to claim 26, and at least one pharmaceutically acceptable excipient or carrier.

36. An immunogenic composition comprising at least one lipid nanoparticle according to claim 26, wherein the at least one nucleic acid encodes for at least one antigen.

37. A composition comprising at least one lipid nanoparticle according to claim 26 as a medicament.

38. A composition comprising at least one lipid nanoparticle according to claim 26, for use in a therapeutic method for preventing and/or treating a disease selected in a group consisting of infectious diseases, allergies, autoimmune diseases, rare blood disorders, rare metabolic diseases, rare neurologic diseases, and tumour or cancer diseases.
